SEQUENCE LISTING (29 sequences)

SEQ ID NO: 1 - DNA, 20 bp, Homo sapiens
  source 1..20: mol_type = genomic DNA
ggcaatgacgaaggaggtta

SEQ ID NO: 2 - DNA, 20 bp, Homo sapiens
  source 1..20: mol_type = genomic DNA
atccggtggtatggatgaga

SEQ ID NO: 3 - DNA, 21 bp, Homo sapiens
  source 1..21: mol_type = genomic DNA
caacacaatggagagggaaga

SEQ ID NO: 4 - DNA, 20 bp, Homo sapiens
  source 1..20: mol_type = genomic DNA
ttaaaaacacgctgggcttc

SEQ ID NO: 5 - DNA, 20 bp, Mus musculus
  source 1..20: mol_type = genomic DNA
gatgctcacgtgtcagagga

SEQ ID NO: 6 - DNA, 20 bp, Mus musculus
  source 1..20: mol_type = genomic DNA
caacgatgacgacgactgtt

SEQ ID NO: 7 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 8 - protein, 292 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 9 - protein, 305 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 10 - protein, 311 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 11 - RNA, 5259 nt, Homo sapiens
  source 1..5259: mol_type = mRNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 12 - RNA, 5292 nt, Homo sapiens
  source 1..5292: mol_type = mRNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 13 - RNA, 5234 nt, Homo sapiens
  source 1..5234: mol_type = mRNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 14 - DNA, 48772 bp, Homo sapiens
  source 1..48772: mol_type = genomic DNA
ttggactgatttaaaacttttaattaaaactctgctctaattaaaagcttattgggtattatactcaaaaaattcataattaatattttaaatcattctttacattgttacctagaatataagcaacttaaaaatacattaataaattaaatttttcagaaatgtgcttgtggtctgctgtagcagcaatagaaatagcccaacaaaatagcttaaaatgttttattaagtatatataatattacagggtaatatttacaaagttatgtttttttttaaataaaaaagtaccttggcaaagattgcagatattcaaagctttaaacagtgataaattgatttaatacatataaaaaaaaaaacctttaacggtaacacagctgtaaaacaactttggtcttcaaatcactgcaaaaaaatggctactgacaaatagcacacctttaattgctatgcaaaaaagctccaagagaggatcttgcacatagcaacactggagttctctttatttagaatggttaatagatatttatacgatgtgggatctaattctcaacaatggctttttgtaaataatgctccatttaataaaaacaaaatgttttcttctctcaccagtcatgataacaacacttttaagatttaagatgttgtcttacagaggagcttagtacaaatgctttctttttttcaaaaagagaaatttatgagaaagctggtcttcaatgatcttaattaagaactgtcaaagctaaaggcagaaaaactgcctggtcactcactgctcatctcttcaaagttacctggattatccctattagtcactgaaaatgacctaacaaaggaccccagcaggtgatggcagttagtaattttaaaatatgacacaagtaaaactgtttataaaaaaatccctcaaccaaataaaatacaataaaaaataaacggttgcccgacaatcatttctccagtttccaacaacaggtaaattaaggagtatgtgtttccatacatacaccacagatccccatttttgaatacccattttaagacaagagaaacctagaaggttgattacagcttaatttttattactgagatggaggagtaaacttatcgtgttttgagctttgttagtgcaaataacaatttggtggtcacttactaaattgactatagcatcctgaaaaaagaaatatttccaattacgggatagccctgttattttaattctgacattcttagggatttaaacagaatggacctggagtttccaggagaaaaataatcacctttgaaggtttttagagcatgtgaaattagtcaaaaaaaaaaaaaaaaaaaaaaaaaaaaaaaacaaaaaaaaaaaacagaaagaaagaaaactctcaagctcaccctattaaaaagtgataaatctaaatacagccaactctcaatcatttaacttatgtacctagcttctgatgtatgcaaaacactgcaggaagagagaatgaaagaaagaaaagctctagctatagaaggaattttaaaatcaaggagaagagcaaggccaaaaaactaatagaactaagcagagatgcataaagtactataggaatggtaagttaagagacaaaagagcagctacacagatgaagaggaactgcaaaggagaatgttttgaaggagaagacaaatagaatttggcaaaagtagctggaaaggttgttgcagaaaaggacaaatggtgtcactgtgagaacacacaataaataaaactgaatggaagagtcaacagcagaaactgggatcagggagtaaaaagcatattatgggacaaaacagatacatgatgtgcaaaacactgtctgcaatttaaatgtgatgtgatggaatttgggagccattacaaatctgatttaaagagaaggaactggcccaatgatgcccaagcaggaggactcctgcttctgtggtcacttatccagagaagctgacaactagtgagagcaagagttggtttcatctggcatttcccccactatctctggtggcattgtctagggcagtgaatgcctcaaaaggcaggcagcattaaagagaacacaatggatggatgagatccaagagcgaaccccaaataaatgagagtttactacatattaaaaggtacaactttagtttataaaaaattttactataaagattttcataccttttctaataggagcttggatctttttcaagtccatctgcttttccctcctttcatcaaaactgatttttaaagtaacaaactaggttagcaaaatgtaaatattttcagaaggaaaaagtgctgcaggagccatcagatttgtataaaggaatacagctccctctatatatgttattattttggacagctacacaatcaaggaagaatatacatgtgtgtatatacatacgtatatatagatatacgtatatatttgtgtatacatattttttttttcaagaagagctgtcttgctagtatgctcagttttctgagaggcctcagcaggtcataaatttaggtttgccatgggcaaactacttggtcccaacatgaaatatgacaatcaatttggcataaaagaggcacacgggaacatctgatggactaagaaataactattattaatgcaactacaaatatgaatatcttattacacaaacaggaagaattacgtatttttacagggtattggtgagcagtcaaaaagcgtggcaaattacctagaagttttaaaagttttaagtgatcaaatatttgcatcaaatataattccacccaataaagaactttgtatttaaatgtttttactaaaagcacaaaattaacctttgctctcctgtaggtaccccacctttgtccatacagaagatgcatgtgcctatctcatacgtatgcacatacaaacacacatccacaaacaggtaaaaaacgaatgctaaaagtctaaaagtactccaggcgatcacaactgtcagaacattaattttcttctaaccaggataggaaatgtgggttttgtttacttttcaatcataggggaaggggagtacgaggcaggactacaggaggaaggtgatgaatgtttatcatgcaacatagatacaacagtaataaatccccattcaagggctggcctgttggcctgatggtgacacacattttgttgccatgcaggagactcatagttcaaatttcaatcttgcttttttcatgccccagatcagaaaagaagtgaagaaacactgtagatttttgccttcaaatcagaggaagggaatacatatgttacaaccatagctatttttgctgaaaagtgtgaacacagtgctctgagaacaagtctggctccttcctgcaactggtgctaccgtgtcactagaccatgttcaaagaggagaaaaggtgctacatcttgtacagcagaagcaagtaacaggaggacaagacagggtttcaaggggttatgtgaataaaaggaaaacatatccatcaataaaagtaccctaaatctgggaaggacctgttacactgtttgttgaagggggaatgtgggaatggcaacccccaagaatgaggaccactatctccatcaatatcacatcatacggagatcatacaagatgctcccaaccaaattcctgcctgtacttaatttattattccattgacccagaagtggcagacacctctcagctaatgccatactgccataactgcccaaagctgctgtgaacctgagttggcaaaggtttgatggaagccactggttttcatagatatctctgggtaatcaccagggcagtgctaagtagttatcaccctgaaccaatttaaactggttacctagaggccagaggccctaggacctgaaaggcatcattcttggaaattgtccattctactattgcccctaattgattaaaaataacaacaaaaaactggatctcaattgggcaaacaacatagatcataattattttattaactaaattacagctgctttgaataaaaacttaactaacaatcacgtaagggtctcataggtgacaaccagttacttttcttgtttcttctccccaacagtgaatcatcaaggccatggtgcttaaacacaagtgtattcctttcacgtcttactactctccaaatcggagtccatcacttcacttcagttattctggaaaattgaaaaatgaacacataatcactattcgtgtctattttctatccattaaaaagttgacacattaaaaaaagtttcagatatatagtatctaaaaattaagaaaatagacctgcaaaggtcagagacagaaaccaggcaaatcattagcctttttaatttcagtaagaaaaaggccaatgccaaatgaaagtaaaaaatcaacagagtctgataatctctgtttgctgggttggctgaggaaaagccaagagatgttgatttttcaaagcaaaagatgttgatcctagaataatattctataatattcactttggatctccatcattcatttaatgtaagtaaaataaacatctgatcagctgtagatgaaagagaaaaatggtactgactctgatccctaggaagcaccctgaacctcattcattgacaaggcagacatactgtaagcaataagcagttgtcagtgggaagtacccaccagccagacattttaaaaacaaattctgatcagctgaatattagcaaaattaagagaatcttgaagatcatcgtagatttcatcacaaatattcaaggcaaaactgttttacaatatttcctgatgcagtagtttaaaaaagtcacatcttaagagaaattaacaaaggaagaggcactcaagattcttagattgctttcctgaaaatggagaaaagtttagtttcttcatacagctacagaacaatgacaccaccaaggaaaaggaaaggacatagaaattaaaaaaaaaaaataggacatcagaatgtaaacatgaacaaaccatgccacaattcctggaacaaaccatgccatgccacagttaccagaacaaaccatggtcacaattcctggtactagtcattttatgttttgttatgtcttttaacttattcctgtgaactatagtcttttttaggagggtaatggcttatataacaagacatgcttttaaaggctgtgacacaaacatacaaaagtaggatcattctgtgactccatgctgaatacacgttgttttaaactttgttcaccaatgcatgactggagtgttttttttttttttttttttttttttttcttgagacggagtcttgctctgtcacccaggctggagtgtagtggcacgatcttggctcactgcaagctctgcctcccgggttcacgccattctcctgcctcagcctcccaagtagctgggaccacaggtgcccgccaccatgcccggctaattttttgtagttttagtagagacggggtttcaccgtgttagccaggatggtctcgatctcctgaccttgtgatccgcccgcctcggcctcccaaagtgctgggattacaggcgtgagccaccgcgcccggccgactggagtgtttttaatcttatgcaaatattaatgacacttggggggaatatagcttcctgagttaaatgctgcactttgctacagaagagggcaggtaaggtcacagactttaaaacagtaaaatataaagcaaactatttacatgaccgaggagtgattataaatatcaaaggtaagaagacattttttgataagataaaaacataaacataattactaaatgccaacaaaactgaggaatcataatatgttaaggtaaaaccaaaataaatgcttgtagcactcaatatctcaaacctatgtatctttaagaacaaatatatttctttgaaatattctataaagattagaagttattagcccagagttcttaaaaaaaaaatggaagcaaaagctgaggacgaaacatctctctctctctctctctctctctctctctctctctctctctctctctcaacctctctctttaatcatctcccaaaaggttagaaccaagctcctgattggaaaagcagagatgttaagtccccatgggttaaatgcaacaaaatcaccaacacaagcataaaagaaactctgtcaaaatgataaagatttgtgccagattatttatgtgccttataaaaggcatcttaaagttacagtcaccagacacggaatctgaaaaacattttacaaaaagtggtaatttctctttgctgggagccatatgactggagtctgattcgtgacaccctacagtttttacagtagtagttttgcaatttttagcaaagtagatttaagttgtatacatcaagaaatgtgaacctttatcatctgacagtgtcccttttactaaaaaagagccacattttcaagaagcttttttgttttccaatgtccatgttttaactgatctataattattaagtgcattttatacttaccatcattcatcattccttttgattctttgaatgctaggattagtaacaagccaagcaggcagttagtgaggttccacaattggaaatcatttctaaataaacaaatatgcaatgatatgctaactagtctacaactctacagccaaattaattgtaaagctatcatttcattatgtagcctttctgtggatcatacccacagcattcacaaaggaaacagacttagttgggaaatcagatctaggtttacaaagaaagaaaatgagatgttttggtctatctctgtggcaatagcctgtatctttctaaaatgtgtacatcacaaattggaagtaattgggatccttctctactagggggtgccatgcttcgtctcaaccagtagcaaaaggcaacgatccccttgcccaaatatcacttggttaaggtaagcagctacttagacacagaatagtgcatttttcccagtggtgagaatattataaaagtaatattcataattcttttttattttttttgagacagagtctcactctgtcaccaggctggagtgcggtggcatgatctcaactcactgcaacatctgccttccgggttcaagcaattctcctgcctcagcctcccgagtagctgggactgcagatacacaccaccatgcccaggtaatttttgtattttcagtagagacagggtttcaacatgttggccaggatggtctcaatctcttgacctcgtgatctgcccgccttggcctcccaaggtgccgggatcgcaagcgtgagccaccgcacccagcccataattcttgatgaataatcaagagtagagatgccaaggaaagaaaaaaagaggaaaaaaacctagattcttcctaaggcatagacagggcattctaactgttttgcgggccagatcaaattatgatctggtaaaggattaagagtgaaataaccacataccattaaggggttcctctacagctttctgaaaaatatttaaaatatatttacataaaaatataatttatgtcatgtcattttatagtaaaacttatatatgctaatatttaaattacgtagtaagcagtaaatcctacagtcaaaaaaaaaagaaaaaacaaaacaacgaaataagcctgtaaatgttaaagcaaagcttgccaagtcgggttaatagtttaataataactgtattattctcttaatattatttttctaaacagaatttcccacttcaccactctcagctcaagaaattctaacttctaaaaatatgaaagtgcaagcataaatagcaacaacagaataaataacacagtgaagaggtggaagcaaaacagaatcatctttaagtatcgcaaattgaactcagtaactgggaaagaaaatgtgttaggattcacaggtccagttaagttgctggcctcttgtgaccaacatagctatggcaccaatctttctagaatatacccctgtggtagacacacatacccgccccacctgccctttaggcacaatgaggcttgtcagacttagaagcatatgtgacctctacctccggccacagacagaagactctctttggcattccagatacatctatcatcactccgttataggaagctaagtacaatataatcctttccagagcttatgtccacaacatacctcttgggcctgtcactgtgataagtctatgtttataaatatgcttttctcttcatttatgccctcctaaaattactcctttcttgcaatttcctcagatggcttaataaggtatctaaggtacaatgaaaccttaagcacagttttaagggaaaaaatgtttataaaacatttgtatgggagaggatcgaatcataactccgaacagatgttttcagcaatatttacatttattttatagattataattatcagtgatgtcaacttagtatagatttctcctcatttttaagcctgaaattgtcattctgtgtatgcacatccaatgaatccaacaggtgaggtgatatattggaatcagcttccttctgattgcagagataatatagccattattcctactttccagttttgggtgtgagaagtcagtacaatcttaagaaatcatttattccatcccattgacactaaaatgaccaaacacaggttttaaaatagaactggacccattcatgcaaaggtcatgcaatgacttcaacattctcttaatttgacgtgagacaaaacagatagcaaaatatctcaattaccaagttcctcagtctaatccctcaaatgatcattcacaattcatttaataaactttaacttaccctaggaggttgtatagtcttctgattggaagctgatataaataacaaataagaatataaatttaataaatgaaataaactaaggcatttggtaatgtttaattttaagataaaatgttcagggctaaaactaaaggtgccagagatgttaatgaaagagttccagctctgacagtctattcaaacatgctgaatattcttgattatatacactgatttgaaaaatgaagcaaataacccatctacatcaaatagtaaacacattctctcatttgtttatgccatctttcccccaaatcagccacttgttacactggctggtgggaaaatgctgtcaccaacatacccccaccatgggtgctccatcctccctcctctatctacccagagtgtagacagaatgtgccagaatttgtccccactactccatctcccttagctcactgctcctgtgtcttacaagctcagtctctttctccacatcactccctgggtctttccagtttcaaagctccaaagagctaacaaactaaagaaaatactcagatagctctgagtattcctctatctatttcttcttagctttctgagttgcctttttcactgcttccaaatggattcattaatgaagttgaaggaatacagtacatgagcaaagtgacgaaagggaaaaagaataccctagttggggagggaagaagagatagcttccttataaaaaaaacaaatgagaaggctggccgtggtggctcatgcctgtaatcccagcactttgggaggccaaggcaaatagattgcttgagctcaggcattcaagaccagcctgggcaacatgatgcaaccctgtctctaccaaaaatacaaaaattagctggcgtggtggtgcatgcctgtggtgcatgcctgtagtcccagctacgtggaaggctgaggtgggaggatctccctcgtcctgggaggcagaggctgcagcgagctggggtcgtgccactgcactccagcctgggcgacagagcgagactccatctcaaaaaataaaaaaacccaaaaaaacaaataaatacagaataagaatcctacagatggaaaagcgttgaacagaaacatgccatgaaccactcagcaggagggggatcctactgaacagagaggcccattccgctgggacctgggctggatgacagcactggattttctcatcagcagatggaggcacgcctccaggactgctctgtgttccttactatttatttaccaccactgcacactcgaattctgtcaagcttccaaagctggcagggaaaagggtgggtcctgacccaaggaaagccaatctgtgagccagccaaaagctggtgagtctttctcaaacaggaaaaatggcaacagactggactgacaagtttctccctgccactggactctaagaagaaacagaaaaaaacccagaaagttggtgatgggaagggaaagtgaaaaagaaacagaaaggagacaaggatgagtccaaagctgtaggtaagaaaagttgtctgtgagaaggcatgaataggataaaaaacagtggcataaacaagagggtgagctagctggctaataggcaaagaatcaaagacagaggaactcaggaactataactgacaggggcatcaccttactcttgctgctgagagggcaacacgatgcttcagttctttgatatggaatggatccttccaattcctagactacatactgtcccacccctgtgaggcccagttatgtatctgagctcataggagtccagtttctgttagtgccatgggcggctttaaataggcctcccttactgtggaggaacctgggggagtgacttggtccttgtatccaaacaagaagccaacctctttttaaagtagaacagggtaaaatgggttgggatgcactacatttagcagcctccccaataaactaagcagaaatacagagctttatttgggaacatcaggatagttacatgtatatacaactgacttcaatgaaaccatgccttatatctatgttctacaaagaaaaagagaactaaacaactatctaaatttctgatggtaatatggaagttcagagataaggttttagagttacatttttgagctcataaatgtatttatcagaataaacactgccaggtgtagtggcacatgcctgtagtcccagctacttgggaggctgaggtgggagcattgcttgagcccaggagctcgaggccagcctggtcaacataatgagaccctgtctctaaaaacaaaacaaaacaaaatagaatattgctaaccgccaagtaactccttttcttccctaataatgtagaaagaatactttctagttctaaatctccgatccaagggatgtcacagttggctggcccattctgaaaaaaaaagatttatgaagaatggtacttcaaagggacatcagtcagttgtgatccctctaaatattataaaaccaaacagtgtacaagttaatttcacctgagttaattctctccaatatggatataacccatgtgacaaaagaattattacgatttctgctgctaccatgagcctaagacattgacagccatatatatatattttttaaccacaagccttaagcattccatatgagcatacttgcaatgcttcttgatttgttcttctctccctgaaacatctgactccatgccataaatacatttccagggtttaaatttttcagaaacattttttctattgataccaaatttacctctccttaaattgaacaagaaaatatctgaatttgtatctatacatagatataaattctacgtatatattctgcatctgcatcattctaaaatatctgggatgttgtggtaaatatggtacttactgctaccagattgatttgtatcaaaacactatatgcccttatattgaactaactggtaatggctttaaaaatgagaaagtattgatccttataattatataaacgttctctacaaacaacaagcccaactttagtgagtttggttaggttcttgtaatcttttctatgctttcacattaagttattgtgcccagtcctttttctcatcttataaaattaaaatgcttccaataaaagaaaaaaaatacaacacagtttaaaaagttaaaaaatagaaagtgaaagtcactttcacagaaagttgtagaaagtgaaaggcctactctttaaaatagcaaattaaggctacaaatttaaagttggaaataataaaaattataaaagttaaaaatctcatagcgacattaacttcttcttgaagagaaagcttggatttttaactatttgaggattttattgcatttaaatcaaaacacaatcattaggcttttctaaaacagaacacgtgcaatgagtaaaaattattttggagacacttaaaattttaacttcctaatataaacgctcaaatttgcaaccttaatgttacatagattcagatttacacagtcagattatataagcaaaccatattaatgttcctcctagtctaccatgttatctaaagagcaactttcctgtaccttgtctcttcttgcctttcaactgactgtaggtgtccctctgaaccctggcatcagacaagttgatttaaaaagttaatttaggatactatcaataaaattaggacactatcaataaaattgcactaaattttaaacatatagcataaattttagcatatagtatacttatatgctaacctatgacaaagaaaaaataatttggggtacttgtcaagatttaagtcattttaattttaatgtcagaacagtgacaggtacagtacagtggtttcacaggcttggaggaacgtaagtgatggagaaggcaggagagtgaagttaaggaggtggcagatagacaatgcttagttccacttgagacattgtcaccagacctgctggcaactgtactgaagatcattctatgctagatatggaaggcatggggtggcatgggagtaaaagggaggacaacccagatagtgaggaaaaaataagagcgtgctgcccgtcagccaaaggtatctcatactacttcaccagagttgtatggatactgcaaatcacacactattctcaggcatttgtggggagcaacaaaaccaggagtgtcatcttccagaaccagacacatggtgattggaagatgtaacaaaaatagaatatgcaccaacattcaagatgttcactgtagctaccacatgaattccattctgctttagatgtctccattgtattttatggcattatgtggtataaaattagaaaatttcattgagcaacaactaagacttatgaactcatatgctaaggatacgcagattttagttaagtattttaattatttatctaataagcattaattaaagaaagacacagaatatatgacagaattgaattcaattaacaatcatcaagaaccaaagttcagaaataaaggtaaaagttttttgaaactattatgtgagtcatatgagaaattatttgactataacaattcaagtttcgtattaagttttagaaacacatacagcaaagtttacctaaagcaaagcagaaacactatttttttactccattaatttataacttttgattattttgataggtggttaagagtatgttttatttcataaaaatataatttttttaaaaaacggaaactaatataaattacattattgggaacatcttgagcttaggtaagataacattagtttcaggcacaattgaaaatatctctctgtgtacatatatctcaataaaaaataagaatttatgcagaaatgttacttgaaaattggcagattaaatatacaagttagaaacaatgaagtaaattccatacttactaataattgattttaaaaggagtaaaagcctaaattttgaaactcaatgttttcattatattggttttaactagaactgttttaaagtcatgcattagaggtcactgctctttaaatgaacaaaattgatagattgtgaattttaattcctgtctggggtgtctatgtctgcaagaaaaagagcaagtgacaaacaaacaggtctgtgcaggacagctctatcacagaaacactggaacaattaaaaatcctattctattacttaaaatggatcctcaaagatctctccccttttacttagtataaaaaggtacacattctaatagataaagccattttcctttaaactactttggaaaccaaattttcattaatcctatatgtcttctaccctctttaattaccctcaaatcattccaattgtattttctgacttctggtcttaaactggaagaaccaactaaaaatcctatatgtcttctaccctctttaattatcctcgaatcactccaattgtattttctcatttttggtcctaaactggaagaacaaactaaaaagcattttctaagtgatatatctgaaattattggcataggttaatgaaaataagattgacttacccacaaatttcatataaactagtccaagtaattgtgctagagctaagatactcaaacctgaaatcagaagagggccatgcattggtatacacgctgtaaaaacaaataaaattctgtattagaaaagcatatgaaataacttactaaaaaacagtaataatcccaagttttaagagagttattctaacgatcaaaaaacaaaaattatccatatatgtttattaacttaaccagtaaaatgactcaaaattattagaagaacaaagccctcctttgcataacacataccttatcatatcaagaaatgggaactggtgtttcaagtctaaggctccaaccaatttgagcccagttattttaaaagctcttccacaaatacaggcatatcagtttgggtgctagaacaatcagccaccaagccatatatacagctaatatctttgtagcgaggaggaaagcactattaccactggggtaaattggaagtttaaaaagaaaagctcagaatgctaaatgctgtcaagtatatgaatgaacaaaaactcttatttattggtggtgataatacaaaatggtacagctattttgatacagagagtttggcagtttatttcaacatactctttacagtcccgtaattgcactccttggtatttgcccaaatggaattgaaaacttatgtccacataaaaatatccatgtgaatgtactttccactcaattttgccatgaaccaaaaactgcactaaaaaaaaaaatcaagtctatatatttttaaaaagagagaaagaaaaagaaaaaggaagaagagctctgtccaaaagtaacccaaattaggtctacagaaagatgactcttacccgcaatacagagactcagtccaaccacagcgaggatataggctatcacctgaataaccaatatggcaatgacgaaggaggttaatccaatcgctggaggaaggaaaaggatgtaacagaagcatgtcaagagtatttaaaaagcaataacttccacttgcatttggtataatcagggatctcctaagaccaaagactgactctggagttcattgtttgacggtagatgttaactcagtgatctaacccttccacacaagtagtcagctccaaattcacacattcggcagcctttaattttcacagtcagctaagctggactggaagtttttaccctaaatcttagaacaatgctgcttttaccacacttgtgcattttacaatatgaatggctattatgaatgtcttctccacatgcacatggaattgctcttgctgagggaaccattatgcaaatacctgactgtaaacatcatgctctctatctctagatgtcattttataacaaactaatacctttccaatgtacaacgagccagagataggctctaagaccaagtgttaaatgtgactctttaatagtaataactaaatatggtagtgatctactttcaaaatgcagtgaaaatgatactgattatagtttggatttccataccatctcttaaaactacagtattaaggtttgctaataattaaaacaaatatgtttctactatttgtgattcataattccattttatagaaccagtcttcttaatgcagaagcagaagcttagcaaaagaagagaaagactgcgtttaacatccacttataacttcaaaaaactgaaattagaatatgaaaataatcattaagaagttatatggcataatttaaaattatgctgattggcatcaaatacatatgaaagtcactttttacaaaacagctaacctagaataaccttaaaattaatatgaatgggaaaatgtcactaagaatagcatgaaaaatcttttaataaacctactcttattgagttgtaaaaaatttatatgagcattttcaccaaaactgctgttttgtaaatgaaaaaggtagacaaaatcatactgtaacaatgaaaactcacctgtactaaacacatagtagtgaagtaatattaatatccctgtagaagtcacaattaaaccaaggccagtagcattctttaatgaatattcacctgtcaataaataaaaacatttaaaatattttcctacatactttttaaatgtactaatctaaattctgtgcttgacaactgtgaaaattttaaaaatatacgtttccctgaaaacctcacacaaaattagtgactattttttgattaagaccaaacttaagacatctaaatctcaacatttaagtttcaacatgctgaaaccttgaattggctctgcctccttcttaactagtttacagaattccagtgagaaactgtgtattcatcttttactgttcccttcatctgctgtattcagtatgccccaaagtcaaatcaattgttccttcaagttgtcttctgaattcacctccttcttcctgctgtcttcactaacttcagataccaatagagctgccaagcccatgttcctaaatattacacctgcactgcattcttctccacaaacgatggtgtgctatagttattaggttgagtcccagccttcctttgcttttctttacatgtgctacatatcctcatttcccactcacattcccatcctccaaatgcaactgccaatttccagacttggttcccacattcattctactttcatgtcttggccaatgaagtttgcctctgtagtgagttgaatgatggccctaaaattatttctacctcctaatccctaaaacctgtgaatattacctaatacagcaaaagagtgaatactgcattatatggcaaaagatatgatgaagttaaggatctgaaaggaggagcttaccctggattatatgagtgggctctaattccaatgacaaatgtgctcataagagagaggctgagggagatttaacaaaaacacagaggcaaagacatagaggaaggaggaggcaatgtgaccagagaggcagagggtggagttatgctgccgcaaactaaggaatgataaacactgtcattcttcagcaaattccagaagctagaagagccaagaaatggattcttccatacagccttgagagggagtgtggccctgatgacgtcctgatttcagacttatggcctccagcatggaaagggaattgctattgtttaagccacccagtttgtggtcatctgttccaacagccctaggaaactaatgcagccctcctcaccttggaatgcactcatctacctcctgcgttcctgcctaggaactgcacatcttacctgggacgaaaagaatggctccaacaatgacaatgacagtgatcactagtccagcaacaagtaaagcaattgttttctcatccataccaccggatctatatttaagtgcttaaaaaagaaaaacaaagaattatatgaactcaatcacaagtgaagccattttaataacttgtaaggtacagagacaaactgacagaagtactataatgtaataacttatgaggaacaactccttaatatcatcaagtcacctcacatctctttcataagcaaatggataacgtcacggcagtataaatatggatggtgaatttataggtgacaccttattttcctacactaacaaatgaggttctatacatcagtctttctcggttgatgtattactaataaattgagctagataagtttttggggggaggggggacactgctctgcataatatagaatgttgaggagcatccctggcctctacatactagatgctagtaactagatgccagttatgttaatcaaactgttttcagacattgtcaatgcccccgatgagcaaaaccactccaagtgacaacacaacactgatgtacataatactaatgggaaataatgggttaattaacaatattgagaaaaagagaattttgtcctaccttataaaaaattagtaaacaatctggaaatctagttattttgttttgaaaaaaatgacagaataaaactgggcagattacacccttgtgaaatcctgatgaagaaaaaaagcttaaattgttcaattaaaatattttaataaaaataaactgcaaagactcaatgggattcatcatacatgttattgtttctgctgctagctccactgtcaagtatctcataccttctctgaaagaatggaatgggtcaatctgcgaatattagttcttttactatatatgaatcttttaaattttggttaatattgaagttgattttttcaccaaggaagttgtttaaggcaacctaaaagcaaactgagtagcggaaagaaacagcgtgtgattgaaatgctgctgtgtgtcatccacatagagatagtctccatgacagtaatcaattggtggctgtcatgaaggggaaaatattttaaatgttatatttagcatttagttttttttcttctacagtgacaaacattaaataaagtaaaagcaataaataagcccctaaatgattgtgataaaaatctatgtttaaggtatatgacaggtacctaaatatttctggaatttctatctacccataatttataacctcaattttagtctctttatggggagataagtcctaagcaataaataaccaatttattaatgtggatttcagttacacagttgagtaactgaaatccacattaatcagagaagagaaacccatattaatcagaaaacatcctatggcaagttccccaagtttttatttgattcattacttccgcccagccctggacttatgtgtctaatactcagtcatagccttgccttcttataaggcacagagttttcataacttccctattttctaggctcaaatgctggctagaatgaaactgagtgtataagtcacaaaaggcacatacacagaaaaaggaactttctctagacatatgtcacattttccattttaatcccatatatattgttatctaaatagagcttgaaagttctgcaaagggttataaagtagaagctgtttcaaatgcacctcacagtcaatcaaacattcataagaaactcttccaggtttaagtcaatgcataaaaaagctactaagatcaagcaacaagaatttgccttagagtacagtataatttcatggaaaaaggttgacttatgttcttcttctatatttttctttttcttttctttttttttttttttggagacagagtctctcactccgttacccaggctggagtgcagtggcacaatctcggctcactgcaaactctgcctcccaggttcaatgattctcctgcctcagcctcccgagtagctgggactacaggcatgcgccaccaggcctggctaatttttgaattcttttttttttttttttagtagagacggggtttcacattgttggccaggctgatctcgagctccttacctcatgatccacctgccacggcctcccaaagtgctgggattacaggtgtgagccaccatgcctggtgacttacatccttcttaacaaaaataagagtttgaatctcatataaaatttatcctcttaaggcttagaagagctgttataggagaaaacagtgcaaacaatagcaacaaaaataaaacatcacaaaatactaccatagacagggttccaccataaaatggtccgtctctctttgagtataagtacactatatattgaaagtagttttggttcctgttgattagttcaactactacttattttccaggccaccttaaacttttccttaaattaagtagccatttccaacaaatacacatgttgaggatcccaagtgtgaaaaaatctgaaatccaaaacacttctggtcccaggcattctggataacggacactccacctgtctctgtggggtctactaatgaaagagtatctccaggttttcttcagtgaatcttcagaaagagtttcttttggctcattttctttgctcacatggtcacatttcagcaggtaccacagctaatgtggggtaggtatcttgttgctagcccagtcgtcaaatttctcagtatagtgaaagggcaaggaatttgatataatagatgccatagaacccaataacctcaaggatctcttattgatcttttcttgtccttcagtaagtggatttatccttatttattgaatgtatcaaaacattaaatgcagcaaaaaaaataaattcaagtaattctccacttagcattaagataaaaataactcaatgtgttgttaacagcaggctcttatcaaattttctagtatgactttcagacggcttggcaaggcaatctatgtaatttttaaaatctgcatgctcttaaggattaccctcagctacaactactgaaatcaatacacaaaataaagtacatgagaaataacagtaattacatgcttaacaaaaaatgtataaccttagtgctcagcctgacacaggaataccaagtatcaggggagacaatataacatttttgtgaacaaattaggtattctaaaattttcttttatatgttctataaaataattagtaaccaaaagcaggctacttctaagaaatctgttgggttggttcattcatctgttctacaaatatttactgtgtacctacaatgtaccagccccttttgctagtctaaatcaaggaacaagtagattactttatttgacaataattgattgagcactatccatctatatgactatcactattctaaatgctgacagtgaacacgaaaagcaaacacaaggcctgcctcaaggcccttacattctaatgggtgagtgtgtgtgtttgaggggaggggggtgtgagtgatgagtatgttactcccaaaacttctttcccagttaaagagatggtgaaggcctactctactctaagaaaattcctttatgcatttaatctttcaaattcatgcagatgaagcaatgacatgtccctctagccacctgaaaaaacagctaatgttatgaatggacaagaaggttttctgattttgtgaccaatagacagcacatacaagataaaatgcaggttgtgactgatgaaatctaatgaaattaccaagtcaacttggggataaaatcaagtctatcatgctaagtttccatgacaccccaaagactggatttgacacacccatataggctcacacagactgcagtccatccaacaaaacccaaaacccaagaatttaatgcctgcccaaagacatgacatttggagtgattggtaagtaagcagagagacaggaaggcttaccaggctgatgcgtatcagactaagaataaatcataaagaaagatgggtttgatttcattattacagtgctgttttctgataactctaggaaagtaattttagaatatatttggtgtctgttgagctgtaaatgaatattgttttcagtgcagaataaagcacatcagacacctttacacataaaattacaatttcattttttaaattgtttaaatttaaattactatttttcaagtatctaatacacttatttaacacacacatgcatacacaaatgaaagcttctgaaactggtactggaaaaaaatgaacattaataccaattataaaattacttgacagaagacaagttaaatcattttataataatctccatacactatccaacttgctctaatgaaacaattttgtcacaatgaatactgactagacatcagtctataggacagagataaaagagtccaggctttgtgcatcaagtactttgaacataaaagatgaaagaagaggatgtccaacagaccaaacttcctttttttcattatcaactttccctgccttgtggatttcacaatttttgagtatatggagaaggttgtgatgtctggatttggaaagggtcaagggatttctacaaaagagtaaaatgctgagctatagatttgttcaattcctcctacaataaaatcagcacataagcacattatttaataacagaatgtgatcattatatggacaggaccaagggaaagcactgggagttcaagattattgaacagaattcactgtaaggggccatgcatggtggctaatgcctgtaatctcagcactttgggaggccgagacagacggatcacgaggtcaggagttcgagaccagcctggccaatatggtgaaaccccatcactactaaaaatacaaaaattacccaggcatggtggtgcacgccagtagcctgtagctactcgggaggctgagacagaagaatcgcttgaacctgggaggcggaggttgcagtgagccgagatcacgccactgcactccagcctgggcaacagagcgagactccgtctcaaaaaaaaaaaaaaaaaaaaaaaaaaaaaaaaagaattcacagtaaacttaataacaaagtcttattaccaggggccatcaaccaggtcacagaatgattctttgtgataatatttattgtatattttactttcttggatggaaatttcctagtccaagataggctatgtcttcactttttcagaataatttttctaatctatgaagaaatttcctgttccattacttattttaaatatgctttcatggctgaattactttcattattaagctgtcctcaagtcaaaactcaaggacttggtaggatctcaaggtagtgtaatgagaacttttttccttgaattttctaacaatgatttctacaggaacatctactcaaagacatctgagccgtaaccaaaatctctgtggagtaattttgagcagggaggggaaagttgaaaaaaaaaaattaaagatgcctagaatacagttttaaatttggaatcaagtacagcagtcctcactttgcactattctgatatgaattccagttaccatggtttaatcaaataaatccctcataaacagtccaaatttcaattaccacaatatattaactgagtaactgcataaagtacaacttcactgctatttcttcagttcaaaaataactatgtaaatagcagatgtcccatcatgatcagtgatcaaccatgtcacttcttttgaagactgttggtgactagtcactgtacatctgatatttaggtcatgcacagacagcaaagtatggagttttgttacctccttgtttcccagtgataaacccatgtgacattttataatggataaatcaaaagaggaaactaggcaagaaagatgaaagagtaaagaaataaaaactgacaaatgctaggagtgaaatttgaatagaccataaatggagtaagagaagaaatagctgattgtgcgaatgtcgacattactgcaatgcaagaaactctagagatgaagccagaagaactcagcgaaggtggacttactgatataaatgaggaaagtgaggaggatgtaacaaaaagaataaagatgtcccagaggaagtgactggcaataaaataaactttaaacattcaaaaaaacattaaaggaattctttcatgacattgagagtgcaaaggataaaacggtagaaaccgatccaaacttagaaaggggtatgacaattcaccaagccatagagaagatgcttgctctgtatccaaagttatgcaagaagaacacacaagtgttgttcaaactactcttgataacacactttcattctcgatgtttctaatgatttaatttaccacatactaaatactagctttactatttttcatttcccaatgccgttgttattgataataacagagtttttaaggttttaacaacaaaaaaaattttaaaggtcatggaacaatataataatagtttttcccattcatcatgaagattgttttgcagaatttcagcttacatggtcatgtttatggtcttgcggttttgtgcgaagccagtattgcctgtataaaacaaagttacaaaggcaaagaaagaacagataggcctatctcaaagaatttcaaaatgttagccagttctttacatacagttggaccttaatcctgcaggaactggaggactaggtggacagcacttaaaatgatatcagagtgagaaagaagaatcctgaaaatcagccaggaacttaattgtcagtagagtggcaaactgtgagttggtgtagttttcagaacacaaacaaagcgtctgaaattcaatactatcagttaagccttggtgtttgattctagcaatctctgagctgtttgctaagcatagagattctgcttaaaagttcctaagttaaagctaagctaaaggtgaaggtatttaggaacatttccctgtaagaggcctttatctttgaaacgtggtccagggctcaccaagtgctggctgggctctgggagtttaagagtcttctcaggcttcttcctcagtaggaccaatggcatcctgagacatctttcaagatacagctctggtataaagaaactctttctcctcagagtgagcaaatgagggcataacctgctgtctttactgaaagtttagcctggtaaaggtccaggtttgtcttggaccacaaagactcacatgcagagctggaaaaaccactgagagaaatcccctgtagccaagtacagaccataaatggtaatcaggaaaagttaagcagaactagaaatctataaaataaaagtcatgggtctgagttattatgaatgctttagaagatttaccatactggattctaaaaaatatacatctaagcaggtggagaatagaggtgctttctatttctatttggtggaatgctcaggccatcaacagaggcactaacagaaaagttagatttggatccatgactatctcaaagatgtgacaaggcatgggatgcacatacattcgtgtctccctggcttaaatgtgggtgctacaaaaccagggcaatatgcacatcacacccacatttcaggcatgtagcttggtgtccagcaatcagtaggcaccctgtcactaggtatttgcagaataagtaaacaaatcaggtcaacattctgctctcctaataagttttagctactcagtattatgtgcttagatataggttcacccttcatggaaataaaatcctcaaaataaatgcaaaacggggaggcagtacagtgcagtggtaaacagtactagttcaaatcccagtactatcaggaaacggctgggtgattgcgggcaaattgcctaatctatctgtgcctgtttcttatctacaaaacgggaataacaacagtgtctatgcccatagagtttctgggagaaataaatgtcatttcagatatagagtactttgcacagaacctgtcaagcagagcaagagctccacaaatggttagctactattatcatcaacacctttaaatgtgagcgcaggctgggtgaatcatgtatagacatacctcttccatgcacatacacacacatctacctgtttcaagatgagccaaaacaaaaaattcccaccaaaatatttctttaggtttataagcacgttccttaaaatatgaccagtacaaaaaatataaaaaagaacattccaccttactaacacaaaaattacaattacaataagatgctttttcacccatcaaatttgcagagattttagcgttataatatcagaattataactgaggtgaaagacaggtattctcatctgcaatagtagttgatacaaacgtttctggaaaacattttgccagtatgtatctagagtcttaaaatatctctgccaattccaggcttcatatctttagcagagttattttcatagtatcaaaaaaattggaaacatccttaaaatccatcaagatgagagttaagtaaattatagtgcatccacaaaagtagaacacaggcagctataacaagatttctgaagacatgggggaacatcagaacgtacaagcatataagctgtacgagtccatttatggttacctttttttaaaaaaaagataacaaaaatgaatagaaaaagacagaaagggaaacaggaaattcaccagaatataccaatagtttatttctaggtttggggactacagatgagttttttttttttttttttaaataactccctactttgtacgaggtgttctaaagtgatatgtatgacttttttaagtcagaaaaaatattgtaaagctcattttgtaaaaatacagccaacagtgaacaaactcactcctggttcatttcactaaaaataatgtcaccaatgataacgtctggatcagcaaacatccatcaagccaaactcaaaaaatcgcactatgtgaaataatttcatgatctttgaaaaaggctgttccctgcccaagtcatatgacttagcccttaaagtctggctctgccaatatggttaaaagctttccctaatatcattgatgccagaataggtcttaatgctacaccagtccttttcttgacataaaaaactaagagattataagaaaatttccaaaagaagtctagacactatcttgaaagtaaaagttataaaatgtcttgacagttgccactgccacccctttcttcccccttttttcctttcagacaaaagttgagaacatttctgaaatcactaagcatttccagaatgccatacactgagaaaagcgcttaaaacatataatcaatcagtcatctcccttccatgtttatacaccaccatagatatagacagcttaaactgctgagtaaaagtaaaattcataaaatatacaacagggcaatccactgcaatagtttactagcttttgtgtgtttaaaggttgcagaataaattaaataagcagtatttattccttgcttatataaatgtttactggtcttcaggatgctacaaggaaagaaggaataaatcttgtatttaaaaagcttaaaagtttagactgtcacctcagaacttcattaattttgtaaattttctttaccaaaatatgaaaatatggttgattaaacattaaccgtaatgcatattacctatttgcctagtaatgacaattctgtgttgttgaagctaaatctaatgcagcatcaagatctgccttcagaagtctggttacgttactattataggaaagcaagatcaaagaacctaactctctggtggctgcttctttagaggctgcagaactgcacgcagaggatgacaaaacatagttccgactggacttttccaagggtgaacacgtgtacacagagaaaatcaaactgggaaaataaaaagaggggaatctaaatgctccaaagtataacgtcctaccttaaaactaatgcaggctgtcaccacctgtttacctaataacataatgtgtgtgtagatgttctgtgaatattaaaatgtgacatgagtttaaggtgttggtactaatattcctactattcattcttaagagattaaaaaaacaccaagtgaaaacaaccttcttaggaattaaaagtgaactgcagcaaaatgtgggcgacaacatatacttcaaggagactagtagtattcaaattacatactcatatttatcataatagcacttttcctagtacaactttaaatatttcgctgacaatgtctcgtagaaataactatatcatcactgaaacataaatgaaaagtagaaataatacttacttttaataccaaactgtccccagaacaggagtatagcaaaaattgggaaaataacaataagaatattttcatttggagaaaaccatgaaactggggaagaagaaaacaaaagtcacaattaatatttactataacactttaaattctgctaatggtctataatacatgctttattatagagatggaaaaaaattaaaactgtggccttatttttacatgtatttatttatacatacacacatatacataaaacaaattacacataatgtacttatgtaaattttatttatgaataaaaactacacttacttaagcatttctcacatttttcttatgtttcacgggaggaggtagtgcagtgcagtggctgagaacaaggctctgtggccacactgccagggtttgaacccaactctaacttaacactctatggaaccgtgaccacgtgtcaacacctctttgttctttggtgtcctcaactgttacactagtataacacagtacatgtatcttgaaggactgttgtaactattaacaaagttaataaactacatgcaaagggcttaaaacaaagcttagcactcactaagtactcagttaactaaaataagtatgtattcatgtgttgagtggctgggtgtgtaaagttccaagaatgtaccttcaagtaacaatgaagtcttcctgtaaagacagactcagggaacctaagcgaggaagatgcaccactaggtactctcttctcattgtggttaaaatggagagagatgtgcccggttggtcttttcccaaactcactctgcaattatccatgcataattaggcttcagaaggactgtttatatgtaaagtaagatgagtggctttaggatattaacgaagctgttattaagttggtcacatgttacttttatcataaaaatatatccacatatcctatataaaggaaattaagatattaagacttcaaataagatttgtgagaacttttaaaaattaagtttaattgcatcttataaacccctaagaccataattttagctgtgaagtttctgctctaacaataaagccaagtaaatgtactgaaagtattccggagtttgcctgtacaaggggctgtgtgattggaagagttcttctgaatactgccctaagcaagcactagcattcctctgaaatcaacacttctttcaaattctaccacgcactatctctcaggtcacaggtgaggaattttccagcctttcactccataatgatagccacatatttcttgtgttaagcttataatgctataataagtcctggtaaaatgcttacatcaatccccagaatataacaacatcataaactctgttttatagatattagcttttcagaatctagcattttcctctatatgtagtagagactccatatgtttgctgttgagggttgcaatggtcataagacagttttgcgagtgaatggaagacttttttttatgcttctttggttgtggatctctactgagagatgtttttggatactccaaatcaatacctttatttccaactgtataactgaatatcttcttatgccaaagaaaagtcacttcagtatagcctaaaaccctaccaggaaaaaattttggagatcatgcttggatttttgtatctaatccccaatggatttggtaaggctgtatggtctaatagccatgcttaattgatgtatttttccttacttcccctgtgcttttgtttttaattgtgtacctcaactcctcaaatcctcacaattatgatgtgagaaattattaacccctccttatagttgtagaaactgaggaacagtgaattcaggctcttcagttaatcttaactgttcatgttccacaatgaatcctgatattttcatcaccccatcccccactaaactccccctctcattttcctcatttcagtcgtggtgaccccatttcttcagttgcttgaggctgaaaaccttggagttatacctgactccctgatttctcccacatcccactctagttaaacagcaaatcctgctgactctaccttcaaaatatagccaatatctgatctcatagtcaagatgagctgacaatgaaatgttctttcattggtccctgcccctatacacaacaaaattttaggatatattaatagaatattaatatattaatattaatttgaatatattaacaaaagtaagaggtgcattcgatcctggtgtgaccactcatttttcacttattccacaaacacttattgaaagcccaccatggggaggaactcctctaggctcttgaaggccagcagtgaacaatgcagaggaccctgagtgtgcagagtgggagaagctgaggcagtaaggaaaggcctctagggagaggaacgaaactaagcctaggatgggaaggagcctacattgtgaagacagggaggaagagatgccaaaggaacggcaactttgagaaaccaaaggatggccattgtgactggaattcagtgatgagatcaaatgggaagagtttgctggggcaagaccatgtagagtcttaaaagcttgttgggagagtttggattttattctaagagcaatgacaagcggctggaaggctttaaatagggaagaaaaataacctgacttatttcaaatcttctttggcagccacatggagaatggattacagggaagcaggacctgtcagaaagttacagcagcagtcatttggaagggagaagggtgtgaccaacaaacgcagtcctgagctcagttttactcatcacatttgaagatctaacagggaattgctccaaaggaagatgactagaatggggatcccgaaaccatgagcaatggctaaaagaacagagattttcatcacagagaggtataaacttgagagaaatactacagccctcccagtgagtgagaatagtagcaagttttagcttgatttaaggaagaactgaaattgcctgataacagaccaagatcctctgggaagtaataatgtctcatcatgtcaagctttcagtaaaaaatctgaatcgccagctacttggggtgtgatagaaataattcgtaaattagatgatgagatagactagatgactacttgatgactcccagattctatgtgaaacaagaaatatgagacatttcaatgacagagaggaagattacagaacaaaaaatactgtctaagtgattcttttttttttatttttgagatggagtctcactctgttgcccaggctggagtgcagtggtgtggtctcagctcactgcaacctccacctcccgggttcaagcaattctcctgtctcagcctcccaagtagctgggattataggcaggcgccaccatgcccagctaaattttgtaattttggtagagacagggttttatcatgttggccaggctggtctcaaattcctgacctcaggtgatccacctgcctcagcctcccaaagtgctggtattacaggcgtgggctgccacacctggcctgtctaagtgattcttaactggaacttatcagtttgtcactctcccttctcattattgtgccaggattttaaattgttccctgatatgtggggcgggtggggggggggggcacacaatggagaagggcatccaggattctctcagtttgaaatcactgctgcacatttctgctgtatttgaactacagtcactgatgaacatgttcctcctctctctaatttgttacagtgggaaaagtttgaggatcatggaattagatatttaagaaccaactgagatgctgggggaaaaaaaccaacattttatataaagccacttatatgcctattaagctattgatttgttatggtagtactgtcagatgacgttaactcttgttcctcaaggggagagtgagacatgatacttctttcagctctgaatttctatgccactaagaacataaattaagcttaaaatggactaagaacaatgtcagatatttttccatcacaagattaataaagataagccaaggacattcacatccttattctacccaagctatggctgacaactgtgtctatcactctcctccaattcctggacaggcagaatggcttccacgaaaatgaacttcttgctagtggaaaggcaaagaagtgatattacaagggtgtagtcaatgaataagtgtttaatttttgctttcaagctgaagtgttattaaaacccaggacctcatttgtcttatcactgtacaaaaatgagacaacctaaaaaagtgaagacaccattcctatcaacaaagtgtctccttcaaactcccacaaacatagggaacaccaggttacacacacacacacacgcatacaccacagtaacatttacaatcctaaaatgcaaaccaaatttttacttgatgtttcaggattgtgatggcttttctgatgtgtcgacttgctaggctacagtcccattatttatttaatcaaacccagaattctaaggtggtcccaaaacccctgccctagccatgcatcttttataattccctccccttgtgtgtgggagactaggactgtgaatgtattggatttcattttcatgattatatcacccagcaaaagggagtttgctgatgtaattaagatcccaaatcagctgactttaagaaagagagattatttttgagggagagagagctgacctaaccaagtggtctcttaaaagggtctgggttcttcctggcaaaataaactcaatatgtgagagggattcaacaagaggaagattctcctttgctggctttaaatatgcaaggggccatggggcaagaaatgagggcaatctctaggagctgctacaattccttggcagatacttagcaaagaaacagggaccccagtcttacaatcacaaggaattgaattctgtcacaattatgtgagcctagaacaagaccccaaactccaaataaagtatgccctgggcaacaccttgattttagtcttttaagactttgatcagagaacccagtcatgccatgaccaggcttgccacctacaaaactttgagctaaaaaatgggtagtgttttaagctgctatatttgtagttgttatgcagcactagaaaactaatacaaagatcttgcaatgccttcatgtcttcattataaacagaccatatgtcctgttgataataatggaattggcaacactgcggaacttgaactgacctttctaaacaaaactctaatagcttagagaaaaataaactttaagttttcaagtttttgtattgctaatagaacagaaatattaatgataaattttatgattactagtatttgcattcagtatttagttatttacaaggtgttttcacatgaaccacatattacggtggtaaaaatactgggttttgatgccaaagagatctaggtttaattcttggctattactctgaccttgaacaataccttgaatctcttttgcttccctatttgttaaatggtaagaactccatctatctcctagagttcttgagaggataacaggacacgacttacataaagtatgtggcacatgtgcaaataaatgttaggtcacttcactcttcatatttgtactgcaaccacttccttgaaatcagcaataatgtcttattcatcacagaatctgtgatgcctagtacggtgactggcacatggtaggcacttagtaaaattttgttaagtaagagaggtattatttatatctcaaacatttccatgatggtgatgccatctttactgtactaagaagaatttgagggacagataggttgagttttttatccaacttttcttaagctattaaacagtgaagtctagaaaacagctttctaatttcaagtccagtgctccttctaatggactatatcatacataggcacagaggtatataaaactatatcttatagtgtttccatgtattcagtcctagtcctccaaaaaatttaaaaaggcacaaagaatggtcactattatctgcattctattaagtagaattcacttctctattgagcaaattctgtttctcatttatgcctgtatttgcaaaaatcacgacatagtaaaaatggtttctaatgcttaaatcctcaaagtccgtgacaagtaagtattcttattagaaaagaacttatactccatagtaagagtcctgccccatttaacctttttgtgcagataactttttaactggtttttgtttgtttgttgtttttttcagttaaaaatggttacaacatagtggcaccagatactggtgaggatgtggagaaaccagatcactcttacattgttgatggaaatgtaaaatgaaatagccactctggaaaagtctggcagtttctttaaaaactaagcatatacttaccatacagcccagtaaccatacttctgagcatttatcccagagaaatgaaaactgtgtctacacaagaacctgtacacaattgttcagagcagtttcatttgtaacagcccaaacttggaaacaaccaaaatgtcctactataggtaagtggttaaacaaactgtgatatattcatactatggactactactcagcaaaaaaaaaagtaaggaaaaaagaactattgatagatgcaaaaacttggatagatctcaagggcattgtgcttagtgaaaaagcccttctcaaaaggtcacatcttgtatcattccatttatatagcattgttgaaatgacaaaattatagatatggaggacaaattggtgcttgccaggttagggacagtgggaggtggcggtatgactagtaagaataagcatgagggagatgtttgttatgatggataggtctatgtcctgattgtgtaggtagttacgcaaataaacacgtgataaaatgagagcgaaccattaacacacactgcaccattgtgaacttcctgattttcatattgtgtcacataagaggaaaccattggggggactgggtgaagggcacatggaagctctctatctttgaatcgtcctgtaaatcaataattatttcaaaaaattaaaatggatgcaaagccaaatgtctcaaacctctggtagaattttctacattctctataataaaacaatactttctccttttaatggctcccaaaataagtctagtttttatttttatagggtattttgcaaacaaagataaaacagtctttcaatcacacgcaaaataataaactgtttttatctcagatcctggcattcctttctcttccatcttcatttcatcttctggcctttcaaatctcatattttgagttttccagtttacctctcagtgacctgcattaccccccaaaagttactgttcccccatatcttattctttcatttccacaaaattgtacattacctcagaaaaatttcaactaggtattagatgatctaataccagttagttgcagtggatatgactgactgattacacccttcattaaaggtatacttaacaacaacaaaaaaagctcctttgcccaaattaatatcaatagtaaaagctacaatgtaagagagatttctgagcagaaagacctttcaaattctccttgcctactcggagccatcccttgcccattcctcactgcacacaaatccctctcacaatgctctaggctaacttcaacaacttgataccttacttcataaaataaacatcccaggaaaacaactaacatttacaacacactttggtttatgctcattagactgtagaatacataaaagcaagtatctattgtcatctccatttcggacatgaggtaacttggactcggttaagcaccttctcaagatcacacaggtgaatagtaagttgctgaacaggggctgtaactcagacctcctgattctcaatcctaagtaatgtgcaaacatttcatcaaaccaaacaccccattcctaatctggtagtagattattttataaaagctaggcaaatagttgatccaactgaacagaactgaacctatctagagttaaggttctcaaaatgtggttaagccatgatcggtaatatgacaggtaattctaaatagagatttcaaaacagaaagaacatactcatatggggttgaagaactcagctagcacagcaggaaacagcaaggagaggagactatttacttcagctaattttactaccttctacacctcagcaacccaagtttgtgataaaaagggcagaatcatggtgctatcatcaagtctgaatttgaataaatgaatttgaatctctagttctctcatattttggccaacaaaaagaagaccaaagtaaggctaagcacagaaaatctgaagaaatcatccatgataacgtgatttaatctcctgactctcctctattctctccagagggagcatggctgggacttgtcatcccttggtcaaagtgtaaggttaaaaaaaaaaaaaaaaaaaaaaaaaaaacacaaaaaacagaaggctgaataattggaaagagacttgaaaattagtcacaggaaagaaagcacttggatggcagcaaacagaaatgaagagtacattaccccaggaccaatcagccaaataaaatggtccttagaccaaaagtttgagaaccacacaatatacaaattaacaagtaatacaaattaaatagtaaatgtgttttaccatgaagagtagattaacaaataggatagaaatcacaatcttaaacttgctaaacattacatcaacatttattaataacagcctgctttttgattcaaaggagtacctatccccatttggcctcctctcgaaagaggatcaggttgcaccaggacaaataaaaaaagaagctttcatagaagtcttaccaacacgatattttagctcgatgatcgtttcaccttctctggttaattctgttacttcacaagtgtagtttcctgtgtgtgagacagcatcactcttatccatcttcaaagaggcatctccttttagtaattgtgagacttcaatttttgcactactaaagtcagtggggacagtggacttgtttagagctccatcaaaggtgtaaatatctcttcctttaaatttccactttacgtatacttcagtagtgttttgtgcctccatattagtaacaaagcatggaatgacgacagtgtcattacaaaacgtgaattctacagattttgttttattaaatagtagctgagctgatcctggaaaggaaaaagaaaaatgtttcattaattatagaagtctgtactgtaagatcttaggcatgttacaaatccttaagataaacagcaacatttccattgttcacttaagtaaacagtatagcaatgaagaagacctattaggtgaaatatgcaaacaaattatattttaatattaacttgcacagctggaaggcatcattatgacttttctttattcattctcaataatacaaagtatacattacatgcatcttgggaataaattcagcaactatgttggaacatgcaggtgtagtatctggctttggaagctttgaagtaatcattactgcctactgatatatgtagaaaaatattgtccttatcaaggtaaaattttgaacagaaaaccaaaccaaatattttattcagtctcaacttcaacacactgctaaatagaacttgctgaactgatatgaaatttatctttatccttatgctgcatttccaaaaaaagtggcaggttctactgtactttaatatacaaagtaaataggatgaatcaaatcatatctttccactaggatggaaaatcttaaaatactaatgaggaaaaaaaaaacattatctgtggctactttcccatagcaaaatataggcagcaatagacattttgtatgggagtcttgaatgacattttcccacccccacatttcattgacactacagaacaacagtctccaaatgtatattatgggtattcttcctccaagaaaccacaaaacggtgaaaaagctaagtaagatgcaagttgaaaccaagagcagttactgctaataagcatcctatctacgcttctcaataggataaactaccagttgttgccttatagaagaggcactgggtaaataaatgtaatcactcattgttcaactaaaatgtacaagaatgtgtaggaattgtagttgtcttccatattcttcagccactcagcaactcttttacctaagtagctacttggccttcccctcaaaacataaacaaaaacaaaacacaaattactaacaaatataaataaaatatcaactttgttaatagttcacacttaagatttacttctacataaactggaataaaaatcagaggcatagaatgtttctattgtaagaacactatgtttactcaataaataccccatgaattgaatgcaaggatgcatactggaatataggtactggacaatgttcaaaacacctgagtaaaaatactatttctgcccctttttaatatgctgccttaagcaactcacttttctatgccttggtttactctctaaaataggaggatctcttaagtaggaggatcaaaaacaaacaccgggctacttcacagaactgtgttgagcattaacatggaaaacatagcggcaagtgctctctaaacaatactatgctgtaaaaatgtagggtgatactataaaaactagataatctggcattttaatgtaatgatatgggccctctttgactagtccaaattgttatatttctgggaaattgcagtctgaacctatattgtgggcatccacagctcacctcttcccagttttctacatttctagccacccaaatactaggaagcccaaggcaaaacctggacttccgttgcattctttaaaactttggaattgtgaattatttcccctgagagagaaacactgagggagagaggacatagtataccagagaagaataaaggggtgaagatgttcaaattataaaggaaggaaagtatcagaaaatatggtgaagattttgaaagggatacaaagactactgggtatcctttcagtcaagtgctgggcaatgtctaacagacgctttggaattatctgactctatagaagcctgtgcctttcactgactttgactgggctgttttacagtttcataagttgtagaaaactgagagtaattcaaatggattgctgtccatacagataaaaataaatcttgttctgcaattgatttccacctggtggaagagaggattccaaaagactacattttactgccctgaggatattgaccaattttgcatttattagcaaattcatttaggcattcttgattgcctataaatgtgtctgtttttccaattttcttttaaaagggttttgccttcttgctagtttgcttgttaattaattagtgaaataaaatctttgacacatgttcattttatatttgtatgaatcatggtttgactttcaagagattattcttaaaaaaaagaggtacgaatgaagaactcagtcatcctatgaaactttttgcaaaggctttaattaagtaagtaagaagtagttggtgaacaatccaccatttcataacatatgtttagagaatgaattttcaaaggaatacagaaagcaataattatactgcattatttgttacaataaaaacattttatagctgaagcaatttaagtagaataaatgctattagtaaaaataacagtaaaattgacctggataaaccacaatttgaataacctttgcactagtaattaataaataagggcaggccaaaaatatcactctagctgttaactacaacttcaatctctataaagatgcattaaaaaactattaagtagtaaaactgtctgatttggggttttttccttttgcctactcccttctcataaaacagcaaagtgaagaaactatttaaaatgtagagagcaaataaagaaacacagataattaacatgaataagaaaactctgaataaacaagatatgcttatagcagtctaaatcagatagcagcagacaaatgtaaccaagagtaatgccagagttatagataaattagcaaataaaatgggaaaatgattttatatcaaatgccaaaagtattagacttgaaaccttcattggaactgtacaagtattggtgaatatagactatcagcccaaaccatctcatatcatagtctagaaaagagctttgtagtggcagagaagactcaatccatggtggtcacagcacacaagttcaggttagaaattttaaaaatgcaatcaacatttgtcaagaattacctgtgatgaaatattgtgataggatgtggggatatagtgatgaaaaaatatagttattatcttttaggagcttacaattctcaaatgggcgatgataagtatataatacaaggttacaagtgttatcttttctgcatggacaacgtccttatggatgggcagaagagtaattggtcttacagatttctaagaaaattcatgttgctaataggcaaaaacagaagaatgtcatagtccctgctcctccgggccttcaatctttaccattttacatatttcatgtaaatgttcgcttctttttaacaactcttaaattactcccttctgccctttatcccatactctccaacagtcctctctccctatttcttaatacaaactgaaaccttcagaggagaatcctgaagcttccaatactccttccctataaacctacctgtaaccccaactatccacaccttttttttttttttgcttccttttctgaaatgaatcccatcacctgaaagctagtttccatttctgcctccatacccatcagtctcctcaacgactttccttagttgattatcagcctcccacatcccccattccatttacaaggtgttgtttttgtgtgtgtctttaaaaattgcttgaatatttgccaacttaaaaaaaatccctcatactttctctctccctagcatcccaaattaacaatctataagcatttcctcacttccaattcactccttaaatctgttacgtggcctccacccattcacccctacttcttcattactacagctcttgccaaagtcaccaccaatcccttgtgaaaaatcaaatatgcatggttcactttctgtcttgacttttccacagttcttgagccagtgggcagtctctttcttgctcttctctcttggcctccatggtatctcctgattacgaatttcctcctgctttgtccaccccatcctagtttgctttctttgttgaaatctattccttgactcacactaaatgttagtgtccctaagggcttcagattcagtaccctctgccccctagtattttccctgactcccatggttttcttcacctgcaaacttttaaataaaaaactgaatcatgcactgcctttcttctaaactgcagatgttggacagccaactgtctgctgtgcacttccacttggatgtaatacagttacttcaaagtcagtatgtccaaaaacttacgtacccccacttcttaccctcagcctaaatcactttgctaaagtattttctatcatctaccagtgaaccagacctggacctaagaatcacattagattactccctctccttagccatggagaattcttagtaggttcaacctaattaatagttcctaaatccaatgtctgttgaatgaatgaattagacactcatcctcccccctcgaactactgccatacccttctaaccagctccctgcctccatcctcaaggtttctccatcctggccctgatgtattttctacagttcccaaaataactgttctaaatgcaaatgtgcactcctttgcctaagatactttaacaacaccctcaacccttttcaggctaaattctaagtacattagcatggaatacaaacgaggtccttcaagatcctttccagccccacctcctgtactccctcatttccacactaagctccaatcataatgacgttattgcaattccatgactatgcaaatgcactctcatccccatgccttagcacatactgttcctcaaacctaaatagcctcttaactgttatacactattaccccactgagtacatagggtgtataatagttaagtgcacacattaaggggtcgaccctagtttaagattcacctcatgtacttcttagctgtataaccttaggcaagttacttacctctgctcctcagtttcctcatctataaaatgtgcatagtgatggtatatacctcatagagatattgttaagatttaataagataacatgtttttgtatcacctttcacatatacagcaagtactcaataaatggcagctacagtttgctaacttttgctcttcctactaaagcacagttgagttgttcaatcttgttccccttccctaccattactcaattgtattcttcccctccttattgctcccattgccttgtgttcaaatgttgtcactgtacctactactgtgtaattactattttacttaactgatctctccaggctcagaaagtaagacactgtcttcatttggtatcctaagaatctaacacagggtccagaaagtaggattcaataccttccttgtgaatgtaaaaagtttaaagtataaattccatagaagaaaaatctaaaaatcatgtgggagaagatagatttttcttctgtatttcaagagaacctgccactgcttctcacttgtatagacttgattatgaaatcatgaagctgcatattttcatggaaaatctcaagtcacttgtatggttggatagaaaagttccttaaaaatcacaagaatgtgtctatgaatgatcttcctagagggtgatgtttaaactgggtcttaaaggatgagtaagaatgttatgggaaagaaggggctttctaggcagagtaacgtgtacagccagataaagaaagtgaagaactgaaagcagagaattatttaggtgagctctaggaaatgaggttggaaaagatgaataagaaaaattctaaagggcttccttgaatgtcatgctgagtttagacttgataacataaacaactgtaaaccactcagatttaatttcattaaaaaaaaaacattgacagcagtgtaaaggaaggactgagggcagcaagggaaaagagaaagggagatccgttaacagactactgcaatagtccaggtaaagaacaactggagagaagacctaaaacttacagtaatcaacagaagttagttgtgggaagcaggggagataaagaggggaaaattaaaattgtgaaccaaaatagtatgtgtagctagttagatggtaggctaattatagagaaaagaggcttaaatatttggggagaatatctttccatctgtcaatggactagtgagatctgatgcacactatgtttgagaaatgagagagaaacaagatggctttatcaagcacttaaaatatattaagtggcagtgattcttttacaataaatttggcagtgtaagaaagcagagaaggcaacaactgacaatgtgcaggcaggaacaagggcaaggacattttggttcttgaggttttttgttttattttttaatatggggtaaaatttgatcatgtgtgcaagctacggggaaggagtcaatggagagggaaagatctaagatgcaagactgaagaattatgctgactgagcaggtctccaagatggtagaagtgaaaaggtggtagacaggttgtcctttcgttggaaaagtgagtaggttttcttctgaagctggatgacggaagagaatgagacgaaggttatctaagatgttatagatcagtttagtaatggtgggagggaagcagagggagtccaaaccacacaccactattttttgcagtagagtaggaagttaattatctgctgggaatggatgttcaggaatggagtaagaagtctcagcagcctcttaggtaaagaagcaaagatcatcagtaaagacacatgccagaaagcaatcctacctacttttgccccttagaactagtgtctggcttgaagaactggagtctctagctacactgggttacgtacaaaaaggatgaagtcagatctctcctcaatggacactaaaataaaggtctggggtagagtggggtgtccacaagttgtacttagttctgacccaacgcaatccccctaatgaatttaaaagtggccttataagaatggaagaggccaacttagaattaaaaagggcagggcagtttaaagtcaataacatccagaattatggccttaacaagcacctaagtaccatgagggaataaaaacaagatatccctactgtctatatactacatcttaagtcattaagagacatactgacatactgtctgaaatttgtctatatgccaaagagggctttgcaaactgcttggtgtgtagtataggctcatacttgcttgaaatggaaaaaagtggttttgacaggccttgaactagccaaaccccttgctttaccttttaacctacaagtgtgatatatattcaaatatgcacattaattctcctaatctgaatctcaaaccatgagattgaccagattctgagtcttatgcaactgctccatggatttggaatgaaatattcagaggtgggtgtagcttgatttctgtttcaacactgtataatcgtaaagaggaaaaacccagattctattgtcttagtctaggtatatcagaatcaaatttcctgggtgctctgtattttctactttagtactacaatgagtttaacaaaactgatcctacactattattcttgttctttttatcacagaagaaatatctacactagtatacaaagtgaaagtaactttatgataacccaagacgatattattaaaatcagactgagagataagatatgaaaatagaaatgtttatcttgtataaaaaggtaggcagaaacctaagacagggaaagtgattgaaaattatgccacaaaaagaacattactatattaaaagcaatgcctatatactagctatagctgaattcatactatgaaatgcaaatctagttttgcgaatatgcgtgattattttggatttagaatatgtatcaatttggggggaaagtcatctatctctcatctttagaacttggcctttcaaatctccatgaacaactcagtgacaaagataaaacatctgccaaaacacagatgacagcatgagcaagtgtcctaatggtaggcatttagtgtaatgctttaaaaaacactgcctctagactgctggggcggccccataaatacgtacttttaaataggcatttacttccttaaaatggcaacttgtaattaaaaataatatattctttaagagaaaacatttttatttaaacaagccaaattactactagacttagacgatggaaaaaaattttatttaatcatatgtaccctgatttaaaaaaaaaaacaactcattttgaacaaagaggaatcattcaaccactgaaaagacagttcacaacaccatcattatttatttaaagttttatttgctagctaataaataccactgaaactatgcacttttaaacaaagtataattgtacatgagtgggggcagagtggaatgcaacatagtaattcatttcacatcttgcataccattaaggtgccttccagtttgcctgtaattgcatgaaatgagtatctgagataacttcttttaccacaaaggccactaacactgtagagggtgccaggaaaggtccctgattcccggggcttaattaacgaaactgaaagcaaaatcaaacttcaatacaaatgaatgtacagatccatcactttcttactcattctagattctaaagtcagtctctcaaacccttggtttataactacctatgtgctgagacatctttcttcatacatgtccttttgaagctatatattaaacacatacaaaaacatattttataacaaatactatcctgttttacagtccacacttaacacaactcaactgcataatgcctgtattttgctttttttttaggttaatattaactaagtaaaataaaatactataaattctgaattcgatgactctagggattcgaacacattaaaattgtgtgtaaacgagtgctaattacatgatattaactattactaaagaaggcctaacataaatcctatcactcaaagtaccattccaacagttctaaactacgtgatttaaatctaacctacctgttttaaaaatagtatatggtttatactttttactttttaggcagtttgaataaatgatactgtatcacttcatatctggttcaatatgaaggcgttctgaaattattatgcgattatacaacacatctctgaaagcagtattattttagctataatgatagtaaaggcaacaagcatactaaacgtgatgacaggaaacactaacatcaaccatttagataacggagacctggattcacagtcccaacatccaggatgctcggggagggaggaaagtggtaagcagagacttaagggcatcttggaaatacctaaagtcacaagcctttaggaacggcacaccgaggctaataatcagtttaaaatcgaattaatttagcattctagaataaatgtaacaattgctgtcactggtgctcaagctgaattctgaatcttataaaagttcacaaaaggctctatggatgtgaagtgtgacaggcactcactgtagagctacgaatgtttctattccttgctcagtttttacagacgaagcacacgttttacaaaagcgaaatggattccaaaagctcggggtgggggtgggggggtggaggttctttggcgtcgaaaaacaccaaatcccattctgctgcacatgttttgactggaggttactgtcttcctatttcaggcgaaaaccagattacttttaaaacggttcccccaaataaagtgggaaaaacttcaacgtacaatttagaatgtatgcaagtggctgatggggcgtgagggtcacgagaaaggaagagtttccagcaagcttatttttaggattaaaaacgacagcaaaaattacacggccggaagattcttctgattgaatgctgagttgaaaatgaaacttaattgtgtcctttacgtcctcttcgctcctatgttcatgtgggaggcaaacggcagaaaccccttctcaccgacttcgccaaaatgcgcacttccatcgcaagaggcccgggccctcccactctccctccctcaaccccaaatgcgtctaagcactgagaagggagaggggtgcgaaagaaggggatccctagcagctgcagccccttgcctctgatcgcgccctttcttgctagttaatttttgcgcgaggtgcgcggtttggagtcttcctgtgtgtgtgcatttggagatggagaaccgggtggaggagatgagccccgctctcacccagggggaagaagacgaccagggaaaggaagggaggaagaaggggggcgcccgattccccgtggggccctcccacgtctgcacttcgggcgctcagggcccgagtgttctgcgccccggccaccctcttcagggcgccgccgggctggctggggcgcagcccacacgcccgcgggggcgaagcgacagcagccgcagggctgggagcgaggagccactcaccgcagcacgccgagcccagcaacagcgccgctaccaggggccacatctccgcgcccgccgcggggtcgccgccgccgccgcaggtgtccggagcagcagccgccgccgccgttacaggcaggaccgaccgccgccgcgcgtcacaggcaggacccactgcccaggctgcacggccgcgcgcacgcgcgctcccactgcttcccgcccgctcccccgcctgctccccgcccgcgagagccgcgcacgcgcactcggccccgccccgcaaccgctgctgttgacgcggcgccctccccagtcgcaggctccagacccggggcccgcctgcccgctccacggaggccggatgcctgctccagggcggtggcacgcgccgcgcttccaggtcacgtcctgtcgcacccgcccgcccctggggtccgcgtgctcgcccgccgcaggaggctgaaccctgtgttcccacgctcgccggggacctcattgcacccgttcctgtccgcgggtcggttcctgcctttcgcgcggcgctgggccccactgtcctctctctctacttttctcctctttgctttcactcccaccctcgcgcttcaggggccgggtcccgtcccgccccagcccgcgtctctccctggacccgacgtccggctgcgcgcgtggaccaggacacctaggctttcaccagcgcccgtttcctccggacgcggccgtctagcgccgaagcggtccctgagcccgaggccgccacgcccagggcagggcggtggtcgggtacctgcccgcagggagcttccactcgcccctcgcgggctctgcgttcggctcgtctactcgctctgctcttccctatgcggttctcttccctctttctcacttggttctttagtgttttcatcctctgatcagggttaatcaaacaagttccattcacggtggtaaacattgttacctgtgaacaccgccaccaccgggactagcgagcattgtcgcaaacaggcat

SEQ ID NO: 15 - protein, 1215 aa, Homo sapiens
MTSTGQDSTTTRQRRSRQNPQSPPQDSSVTSKRNIKKGAVPRSIPNLAEVKKKGKMKKLGQAMEEDLIVGLQGMDLNLEAEALAGTGLVLDEQLNEFHCLWDDSFPEGPERLHAIKEQLIQEGLLDRCVSFQARFAEKEELMLVHSLEYIDLMETTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNLRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGLAGRCLTLTPRPATEAELLTCHSAEYVGHLRATEKMKTRELHRESSNFDSIYICPSTFACAQLATGAACRLVEAVLSGEVLNGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSLLGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALTQDQPSEAATGGATLAQTISEAAIGGAMLGQTTSEEAVGGATPDQTTSEETVGGAILDQTTSEDAVGGATLGQTTSEEAVGGATLAQTTSEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHPH

SEQ ID NO: 16 - protein, 1063 aa, Homo sapiens
METTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNLRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGLAGRCLTLTPRPATEAELLTCHSAEYVGHLRATEKMKTRELHRESSNFDSIYICPSTFACAQLATGAACRLVEAVLSGEVLNGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSLLGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALTQDQPSEAATGGATLAQTISEAAIGGAMLGQTTSEEAVGGATPDQTTSEETVGGAILDQTTSEDAVGGATLGQTTSEEAVGGATLAQTTSEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHPH

SEQ ID NO: 17 - protein, 1229 aa, Homo sapiens
MSGANRGRGQASSTMTSTGQDSTTTRQRRSRQNPQSPPQDSSVTSKRNIKKGAVPRSIPNLAEVKKKGKMKKLGQAMEEDLIVGLQGMDLNLEAEALAGTGLVLDEQLNEFHCLWDDSFPEGPERLHAIKEQLIQEGLLDRCVSFQARFAEKEELMLVHSLEYIDLMETTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNLRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGLAGRCLTLTPRPATEAELLTCHSAEYVGHLRATEKMKTRELHRESSNFDSIYICPSTFACAQLATGAACRLVEAVLSGEVLNGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSLLGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALTQDQPSEAATGGATLAQTISEAAIGGAMLGQTTSEEAVGGATPDQTTSEETVGGAILDQTTSEDAVGGATLGQTTSEEAVGGATLAQTTSEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHPH

SEQ ID NO: 18 - RNA, 4147 nt, Homo sapiens
  source 1..4147: mol_type = mRNA
cacggcgcgtcgcgcgggaagtcgcggggaaaaggtcgccagaaacttggtggagcgagccaagggcggagtttgagaaaggggctgcgtccaatgagtggagcgaaccgcggcaggggccaagcctcctcaactatgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaaccttgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgatctgatggaaacaacccagtacatgaatgagggagaactccgtgtcctagcagacacctacgactcagtttatctgcatccgaactcatactcctgtgcctgcctggcctcaggctctgtcctcaggctggtggatgcggtcctgggggctgagatccggaatggcatggccatcattaggcctcctggacatcacgcccagcacagtcttatggatggctattgcatgttcaaccacgtggctgtggcagcccgctatgctcaacagaaacaccgcatccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagttccagcctcagctggtcctggtggctgctggatttgatgccctgcaaggggaccccaagggtgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgctggagtcacctggtgccccctgccggagtgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggttctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcagggggcctcagaatctcaggccccaggagaggagaacctactaggagaggcagctggaggtcaggacatggctgattcgatgctgatgcagggatctaggggcctcactgatcaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtctactgtggtcgttacatcaatggccacatgctccaacaccatggaaattctggacacccgctggtcctcagctacatcgacctgtcagcctggtgttactactgtcaggcctatgtccaccaccaggctctcctagatgtgaagaacatcgcccaccagaacaagtttggggaggatatgccccacccacactaagccccagaatacggtccctcttcaccttctgaggcccacgatagaccagctgtagctcattccagcctgtaccttggatgaggggtagcctcccactgcatcccatcctgaatatcctttgcaactccccaagagtgcttatttaagtgttaatacttttaagagaactgcgacgattaattgtggatctccccctgcccattgcctgcttgaggggcaccactactccagcccagaaggaaaggggggcagctcagtggccccaagagggagctgatatcatgaggataacattggcgggaggggagttaactggcaggcatggcaaggttgcatatgtaataaagtacaagctgttaaaaaa

SEQ ID NO: 19 - RNA, 4527 nt, Homo sapiens
  source 1..4527: mol_type = mRNA
ggttgaaggaacggggcagtcccctgaggagcggggctggttgaaacgctaggggcgggatctggcggagtggaaggtaccggtccggcccgataaggggtggagttaagtgaatcgttaagggtggagtcgaaaccggggtcggggccggggccggctgagtgaaagggtgggtgattatcccgggagataggccgaaagggcaggttcgtgaaggaatggggtctgggctgggcttagagggtctgggaatggggcttaggagagtagaaggggcggtgattggttggaaagggcaggccctaagacggactgtgtgagcccgggggctcattgctccgtgaaagggcaagaccagggaaagagaatcgtgtaaaaggggtggagtctagcgggccgggcggggccgggtagaatggccacctgagtggagggagaaggcctgagagaggcggagtttggaaggctgtggagaatcagatcgcaaccgcggcaggggccaagcctcctcaactatgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaaccttgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgatctgatggaaacaacccagtacatgaatgagggagaactccgtgtcctagcagacacctacgactcagtttatctgcatccgaactcatactcctgtgcctgcctggcctcaggctctgtcctcaggctggtggatgcggtcctgggggctgagatccggaatggcatggccatcattaggcctcctggacatcacgcccagcacagtcttatggatggctattgcatgttcaaccacgtggctgtggcagcccgctatgctcaacagaaacaccgcatccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagttccagcctcagctggtcctggtggctgctggatttgatgccctgcaaggggaccccaagggtgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgctggagtcacctggtgccccctgccggagtgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggttctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcagggggcctcagaatctcaggccccaggagaggagaacctactaggagaggcagctggaggtcaggacatggctgattcgatgctgatgcagggatctaggggcctcactgatcaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtctactgtggtcgttacatcaatggccacatgctccaacaccatggaaattctggacacccgctggtcctcagctacatcgacctgtcagcctggtgttactactgtcaggcctatgtccaccaccaggctctcctagatgtgaagaacatcgcccaccagaacaagtttggggaggatatgccccacccacactaagccccagaatacggtccctcttcaccttctgaggcccacgatagaccagctgtagctcattccagcctgtaccttggatgaggggtagcctcccactgcatcccatcctgaatatcctttgcaactccccaagagtgcttatttaagtgttaatacttttaagagaactgcgacgattaattgtggatctccccctgcccattgcctgcttgaggggcaccactactccagcccagaaggaaaggggggcagctcagtggccccaagagggagctgatatcatgaggataacattggcgggaggggagttaactggcaggcatggcaaggttgcatatgtaataaagtacaagctgttaaaaaa

SEQ ID NO: 20 - DNA, 23585 bp, Homo sapiens
  source 1..23585: mol_type = genomic DNA
cacggcgcgtcgcgcgggaagtcgcggggaaaaggtaagatgccccttcctaattccctcgcttccttgcttgctctccagacaccgccgacccgaagggctcctaagtgactggacagggacggaggggcggcggctatggcatccgggacgacagcgacgatagccgaggcaagaaaaagcttgcaccgcagccctgtcgggaaccgccggcgcctgccttttacgtaggcgcagcacagctggcgtagcacgccgacgcaccgccccgtcgcctgtgagctcgcggtgtggctcagcccagctggcgtagcacgccgacgcaccgccccgccgcttgtgagctcgcgagaggtgtggctcgcgcgaggggcgcggccttgaggcacggtcccctctggaggcgggatctgggcgggcagtcgagagacgaggcccaatggaaaagaaaagcctgagcgctcgcgtccccacaggtcgccagaaacttggtggagcgagccaagggcggagtttgagaaaggggctgcgtccaatgagtggagcggtgagtacagcgcgtcgacggcggaggcgggaagggggcggaggccgaccgaaggcggctagggactggctgaagagggttagcgaaacgttagcggtcgggtctgggcagggggtggagctggttgaaggaacggggcagtcccctgaggagcggggctggttgaaacgctaggggcgggatctggcggagtggaaggtaccggtccggcccgataaggggtggagttaagtgaatcgttaagggtggagtcgaaaccggggtcggggccggggccggctgagtgaaagggtgggtgattatcccgggagataggccgaaagggcaggttcgtgaaggaatggggtctgggctgggcttagagggtctgggaatggggcttaggagagtagaaggggcggtgattggttggaaagggcaggccctaagacggactgtgtgagcccgggggctcattgctccgtgaaagggcaagaccagggaaagagaatcgtgtaaaaggggtggagtctagcgggccgggcggggccgggtagaatggccacctgagtggagggagaaggcctgagagaggcggagtttggaaggctgtggagaatcagatcgcgtaaggaatggaatctaatagagggggcggagcctggaaaagggcagagaggtggggtcctcagggcacactagccccctcacatacccacgctcctccccccacccccagaaccgcggcaggggccaagcctcctcaactatgacctcaaccggccaggattccaccacaaccaggcagcgaagaagtaggcagaacccccagtcgccccctcaggactccagtgtcacttcggtgaggccctagacccgccctgatgagggggagagggagcaagttggtcatgccctggactctgacccttctctctgattcacagaagcgaaatattaaaaagggagccgttccccgctctatccccaatctagcggaggtaaagaagaaaggcaaaatgaagaagctcggccaagcaatggaagaagacctaatcgtgggactgcaagggatggtgagcagggcctggctgcagtttagcctcgtatgacaatcaaaacccaaaggttccccaccctggacagttccctctgactcagggcctaaaatggatctgtgtctccttttttttttcctctgcaggatctgaaccttgaggctgaagcactggctggcactggcttggtgttggatgagcagttaaatgaattccattgcctctgggatgacaggtgaggctgggtcctccaggctgtctccaaaccacaaaaatacacacacttagagcccattagcaagaataaatcttttcttaccccagcctggatactggaagcagatgatttggggaggtagggctcacacttgaaagggttcttttatgtagctaacaaacatttattgagtgcctattgtgtgccaagagttggtgatacaaatgtaccaaacaacagacaggggtccttctctaggagcggattgatgagtaggagggcagatcattaaacagccaacagcagtatgaggtgtttaatgaagacagacttaagaccaggggattcaagggtgtataatagaggatcctaaactagtatccgggcttcccagaagcctttatagaggaagagacgtttgtactgaaacagaaggatgaaacctcccgtttgctttacttttcaaaaagaatcagcaaatccttttggctatatgttcaaaatatattcaccatctgacctctcctcaccacctccactgccaccatcatcatcactcacctagacttgcagaggcctcctccctggtcccctacttccaacctcacctctattatctgttcatgcagcaacctaaaaaaatatgtatgagttgatgtctttgttaaaatactttaaggaggccaggcatggtggctcacgcctataatcccagcactttgggggggccaaggcaggtggatcacttgaggtcaggagttcgagaccggtctgaccaacgcgatgaaacactgtctctactaaaaatacaaaaattagttgggcgtggtggcacacacctgtaatcctagctacttgggaggctgaggcaggagaatcgcttgatctgggaggcggaggttgcagtgagccgagattgtgccattgcaccccagcctgggcaacaagagcaaaactctgtctcaaaaagtaaaaataaaaaaaaaaaaagtactttaagggcaaactgttgccctcaaggtaaagtccaaatttttcatcctggcctcaaaggttccacatgatctggctccacatcactttgtgacctcatcagccaatccttctcgcccacactactccccttgttcttccaacatgccaggcaccctcttgcctctggtctgtaccctgaccaacccctttcccagagattcttttcggtcttctactcttccagctgcctttcacatcccaatgagactttctcagagaaggttttgcctggccaaaccctaccccaatacctgatcttattttcctagcccatgacattgtttgctaattatcatacttgtgtgtttaccaattaatatctatatcccttatcccaacctaagactaacttactatgggaaaaagacttcttttatggaatacctatttaccatctattgaggagctagggatacagcagtaaacaaaacaaacaaaagcctgtgtcttcagggtaaagggggaagacagacaataaataaacaaaaagtaaatgatgcactgtggtagaaagagcagtgttatgaagaagcagtaaagggaggatagtaggagaaagtacagtggaggagacttcaattttaaataggctggtcagggaaggcctcactatttgagcagggatctgaagaggtgaaggagtaagtcatggagacagttggaggaagagtattctaggcgggggtgggggggccagctagagtaaagttctagggctggctgaagagtatttggttcaactgagaaccagggaagagaccagtgtaggtgaagcgaagcaaaggaaagagggaggggtaggagatggggtcagagaggtgatggaggacagattgtttagggccttctgggcatgggtaatactttggcttttactcagaatgggatgggagcctcgaagggacatgaactgattagggttttaaccagtgtctagctgctatgcacagaaaggaccgttgaggtggcgagggtcgaagcagggaggccgaggagatgccgccacagcccaggcgggagggtatggtaatagcagtggcagtggtgaggagagggtcacacaccagagctgatgggaggcaggactgacaccacagttttggggtaagcagctgtggaagggtggatggggagatgtggagagaacagatgtggagctccccagtgtcctcatgcatctgtgactgtgactccatctccccagcttcccggaaggccctgagcggctccatgccatcaaggagcaactgatccaggagggcctcctagatcgctgcgtgtcctttcaggtaaggcccccaagccaacactctgggtgaagggcagggagctgacctgacctgggtgcagcccatgccttaaccctttaccccactttattcctcatctctccccaggcccggtttgctgaaaaggaagagctgatgttggttcacaggtgaaggcttgggagccttgtagggatggggaggagcctgccctggacaaaccagggcaagctaaacgctttgggaagcaatgtcatggtctctgcagccagccttgggcaagtcgctgagcctttctgaatctgtgtcatcgcctgtaacaaggggaccagtaaacataagttcctcaggagagtttgatgtgtgtcaggtgcttagaacagggcctgatatataatcagcccatgatgagctattactgttatcatgatctgattgtgtgaatagatggtggatgtgtggacagagtgatcggggttcccccaggggcttccagatttagaggcctagtgctatgtggcttgaggaaagctagcttccagagcaggtatctgagctgaactttaagaggagaaaaagagtgagtcagatgaagagggtgactgagggaagggaagaacagacagctgaaccagtgtggccaaaggcctgaaagtaggatttggtgtggctactgcaggggagcagagaacattacctgaactgaggcgggctagggaagaaacagaatgtgtcttatatcctggctattccttcaggttgacagttgggggagccaagccctaatttgaccagggggttggtgtatggacagctcagttggggaagagacagggccctgaatctcatctcccatctgtgtctagcctagaatatattgatctgatggaaacaacccagtacatgaatgagggagaactccgtgtcctagcagacacctacgactcagtttatctgcatccggtatggatgagaactctgcgggcagggaatggtggcaatcttggggtccccatccctatgcccaccccaccctgggggaagcagctgagtggatagggctgttctctcccttactccctttctggctccccactgtctctccagaactcatactcctgtgcctgcctggcctcaggctctgtcctcaggctggtggatgcggtcctgggggctgagatccggaatggcatggccatcattaggtaggactctacagcatttgactttttacattgttttaaagtccttccttcaagtgtaaaatgtattgatatgggtaatatattcatgtgattcaagaatccaaaggtaaaaaagagaatataattaaaaatctgtctcccatccttgtcacccagacacctgcctcctctcatgggaggtagtcattggaaccactgactcatgtgtcctcccacaaagtcttattcaagaaataaataccatatttcattgattctaaggtgtacattttttcatatttgtaacatcttgaaatccagatgcatcttacaaacagtgacatctcagaattccattggcagtattttctgcttcgtttttggcatgtaaaatcatggtacatcttatcatcattggcatcccagaactgatgaaatgcaaaagctttctcctccctcccctgtttttaaccacaaatggtagtaacatgacatactcccttcgtaccatcttttcacttacacatcttggaaacctttctctaacagtatgtatcttggagcccggtagaacaccattgtgtaaatgcactctatgttaatcagtccccgactaatgggcattttggttctccccagtcatttgctgttttaaaaaaatgctacagcaaatatgctagtacctttataaaaataaaattttattttacactaacataaaataaaaatgttacttcaccctttttaaaacacaggttgtgggctaagttcctgcagcagaatgactgggtatgagaatgaatgcgttgatcattttatttttatttttatttatttatttattttgagacggagtctcgctctgtcaccaggctggagtgcagtgatgcaatcttggctcactgcaacctctgcctcccaggttcaagtgattctcctgcctcagcctcccaagtagctgggactacagtcgcccgccaccacacccagctaattttttgtatttttagtagagacggggtttcaccgtgttggccaggatggtcttgatctcttgacctcgtggtccacccacctcggcctcccaaagtgctgggattacaggcctgagccaccgcacccggcctttatcattttagtgaacattttcaaatctcctttggagacagaggggtagattggcagggggtgagagtttgagggcctccattactcagttcccggcagatttttaggcatcagcattaaaggaattggtggtccaggattagagagcaggggacagtctgtctctgcagaggaggacccctcacatactccaagctgttatttccttgtcttgcccaggcctcctggacatcacgcccagcacagtcttatggatggctattgcatgttcaaccacgtggctgtggcagcccgctatgctcaacagaaacaccgcatccggaggtcagcaacagagggcagatgtgatgggggtggcatggggtggggtgtcctggtctgggctcttcccccagattcaccttgatctccttgcacagagtcccctctctctgctgctcctagggtccttatcgtagattgggatgtgcaccacggtcaaggaacacagttcaccttcgaccaggaccccaggtataccccgactcccacctaggtgctagactcccagccacctcccacccttacaggcccagccagaaggggtccagaaggaggaaaaggcattcacattcatggggcctatcctgggatcaggatctgtgctacttttgtgtgtctgccatctctcctaatttcactctgaggcagggactgttgggttccctattttacagatagggggactgaggcttatacgttaagaggtcacacagagaagaactgggagagacgggatttgacccacaaatatttgaagatcttattcactgattaaccgttgggttagagtgaacctcaaattactgctttattttttctgattaggataatgcttcaaagtgtttgtttatcctttcagtagatagtaaaaaacaaatataaataaataaataacaaactatttgtttaaagaaattaaataatacagaagtgtctgaagcagtgagtctcctctaatttccattacctcaaaaataagtgttgacagtttaggttatatccttacagattttctctatagatgtctgaatttttattattattgttataattattaataacacactgcctaatggaataattctccacatgttcttcgacaacttgctttattcatttaacagcatatctgggacatttctccatgttagtacctcttaaagtatctcgttctttgtagtattcaattttatgactgtacctctggttatttaaccccaggacttttaagatgcctgccagtttttgctattacaggcacttctactgtgaatagtctagtaaatatgtaattctgcacttctctgagtctatctgtaggatgaatttctaacagtgggttgcttgggccagagagtcagcatactttaaagtctggtatacactgccagacttccctccaggaggggaccagcttacccttcctgcagaaagtgattgagcaagtccttgtcagtaggagacattactaagtacatgtcgagtatcccttatatgaagtgcttggaaccagaagtatttcacatttcagattttttcagattttgaaatatttatattatacttagtggttgagcatccttaatctgaaaacccaaaatccgaaataccccaatgagcatttcctttgagcatcatgttggccttcaaaaagtttaggattttagggccgggtgaagtagctcactcctgtaatcccagtactttgggaggccgaggcggatcacctgaggtcaggagttcaagatcagcttggccaacatggtgaaaccccgtctctacaaaaatacagaaaaattagccaggcgtggtggcacacacctgtatttccagctactcaggaggctgaggcaggagaatcacttgaacccaggaggcagaggttgcagtgagctgagatcgcaccactgcactccagcctgggtgatagactgagaccccatctaaaaaaaaaaaaaaagtttaggattttacagcatttcagatttcagatttctagattagggatgctcaacctgtagtagttttatctagccttcttcctagctgactacagagaatccacttatccaagatctgaagaatccaatttaggttggttgttctctagacattgctttattattttgcagcattcagtgttcctgacaggtgtctgcttttcattcctttccaagtaatctactttcctctctgcaaggttatcttggtgttcttttttttttttttttaaattattagacagggtatcactctgtcacccaggctggagtatagtagcatgatcatagcccactgtaacctcaaactcctgggctcaagcaatcctcctgcctcagcctcccaagtagctaggactgcaggcatgtgccaccacacccagctaattttttaaattatttgtagagacagggtctcactatattgcccagactggtctcgaactcctgacctcaagtgtcctcccactgcagcctctcaaagcactgagattacaggcatgagccactgtacctagccttgatgttctaaacttttaccctggccttttcactctgtttaatagtaggtagacattttacatctgaagattcatgcttttcttaaactcttggaaattattttctattagatctttgataatttcttctttgttttctctgttctctttctaggacttctgttagtcagatgttagacttcctgggttgatcctctatgtctctctcttttttattatttatttatttatttatttatttattttttttgagacaaggtcttgctctgttgcccagactggagtacagtggcacaaatacgactcactgcagcctcgacctcccgggctcaagcaatcctcatgcctcagcctcctgagtagctgggaccacaggtgcacaccagcatacccagctaattttttaatcttttgtagagatggggtctcaccatgttgcccaggcttcttattttttatcttatgttttctgtctcttttttcttttgctgtgacctgggagattttgtcaactttatcttcaagcccttctgttgaattatgtatttagatatcctgttaaactccaaaagctctttcttcctctctgtttctatgtcctctcatacctgatcttgtttttatagagtatggattagaagtgccggctgggtgcggtggctcatgcctgtaatcccagcactttgggaggccgaggtgagtggatcacttgaggccaggagttcgagaccagtctggccaacatggtgaaaccccatctctactaaaaatacaaaaattacccagacgtgatggtgcgcacctgtaatcccagctactcaggaggctgaggcaggagaatcgcttgaacccaggaggcggaggttgcagtgagacaagatcatgccactgcactccagcctgggcgatagagggagaccctgtctcaaaaaaggaagtgccttaagtttcttctgttgtctgagttatgtctctttattctgaggctggttgaattatttttttcttggtccttctcttttatgctgatggttttcctcaaatgtctagcaatccttatttggctatttatacccaagaatgaaggcccattgtagggggctgtcagaggactactctgctgcagtgtttgtaggtctgttttcctgaaaggcatttccccagaatggggacagggaatgtccactgacaggttgcaactttagcgagtgtgggcaaggacccgggcattagatgggatcctgccagatgtcagaacaaaggaggcctcttctttggggtaacatctcttttttccctcagcctttccaaagtgtatctgttgtacttatcttttccagtgtagctttgggctttgttggtcatctctactgttttattatggtgggtttttaatacttcattaatttttctggttttatctttattaatgttttcctccgacgttctttattttgttcatttctggcttctggaatttaatgcttcaacatttatcaactcattggccgatctcatttcatctatacctgtacccactgccctcttcctgttttccttcaaagctaatcccagataccatataatttctcctgtaaatatttcagaatgcatctctaaaacataagtacacttttttagaaaagcattaccttagtatcaatacctgaaaattaacaataattatttttttaatctgccaagggtctgtcattcaaagaattctttaatatcattaatcattctttttgtttgaataaatgcataggaggctatagctgtgtgttctttcatgggtacactattgattacacacacagatgtgaaacactgtgaatatattcagtgcattcttccttattaacaaacgtgtgtaaacaaacaaacaaaaagaacatgtccatgcccttggatctcccagctccccatctctcagtctctgaactccagcagcagggagacaaactggataatccttaaaatactgttgtgaagacaaggaaagcagatgatctaaaactaagtagttaaaagaaacaagtaactttgttgtattggtgattcagtgagttggctcttcagaagggattttgtgtgcattgacctggatctgtactggatttctccagaaaggaaacttcagcttatcttgggtgcccccttatcctgctcagcatgagacagagcaccctggggcccctggaatctcaaagtctactttctagaaattcattgaagtctcttattctctgacatcctttccactctctctttctgggttgctgtttgtttacaatttctttactgtattttgaatggggtagtaggaggttgaggaagtaaacctgtgctatcaatccaccatttaacttgaaggtggttgaacttattttgaatttcccttctgttttctttttaaacttagcttgagtgctagttctgctgtttattgactttgatggctctcttttagagtcagctttcttcagatcttttctttcttcctgtcttccccaatgatatttttttcttttcctttctttttttttgagacagagtcatgctctgtcgcccaggctggagtgcagtggcccgatctcggctcactgcaacctccgcctcctgggttcaagcaattctcctacctccacctcccaagtagctaggattacaggcacgcgccaccacgcccagcaaattttttgtatttttagtagagacagggtttcgccatgttggccatgctggtctcgaacttctggcctctagtgatccgcccaccttggcctcccaaagtgctgggattacaggtgtgaaccactgcgcccagccctctggtgataatttttatgtcctactgtgtttctcttcctctaatgattaacttactaaaagtttagaaccaagatttatctatcaatgtcaaaaataaaacaatcatcctccacctactcaacaccagagcttttgcttgtttttattccccaattccaagctcttttccctcaattttaagaatctttctatatcacttgcattaaatttcacaactacattatcaacccattaaaatgtgactcttaagtttgtctagattcaatgcataccactatttcttgcatttccctaaaaaagctgccttagatcctctctggaacaaagcagggggtaaatgaccagatagaaggatggatggatgatagattattcattttgctttccccagtcagaattaagccctcccatggccccttgctgggctttctgtcaaaacacgtgcatcgcatcttttcagtatctgttttgcgtgtcggtcatactcacccctctgagtgctcattctctcgcctataaaatagaagaagtagtagaggtggcctgtaaggcattggggaggaggaactgccctttcactggcctcatgacagccccagccagcacccccgctctagttccacccacagaagcaagtctcacccctaccccccaggtctctccacttcctgctagagaatcacccagctccccagcctggcttgaggctcttttcagccttagctggctccccgcagctaagagccccatgctccatttgggcctccagtgtcacagggtttgacctgctgttactactacctttgtcataggggcaggtagtgggcggacatgatctagagtaccccacagagtcaggagcccccatgatctgagatagaccctagagctgagcctgaaattgggtgggcactgagggggcagcgatgtttgctgagggagaggttctggtgagagactaaatgaatgagcgagtgaggtaatggataaataaagataagaatgaaaaaatggtaagatgattaaattggtgactgaatgtaagggtaaattaatggaagaatggattcatggattaaataattaatggggaaattaatgaatgagagaatgaacgagtggttgaatggggaaatgagtggtagaggaaactggcaactgttgggttgggggtgtctatggggattgacttctccaactctcttacctctttttcttctgcccccagtgtcctctatttctccatccaccgctacgagcagggtaggttctggccccacctgaaggcctctaactggtccaccacaggtttcggccaaggccaaggatataccatcaatgtgccttggaaccaggtcagcatctacccacctctgcccccaaagtgaggcccagccccgcccttctgtcagcggctcgggacaggtggctgaacatcccccatcacactcctgtgtctgcaggtggggatgcgggatgctgactacattgctgctttcctgcacgtcctgctgccagtcgccctcgaggtcctggggatctggggtgtgttgggaggaggagtggccttgaaggttaggctgtgagggtaggtagcctcatggagtctgttcccccttcagttccagcctcagctggtcctggtggctgctggatttgatgccctgcaaggggaccccaaggtaaggcaggctccctggggcggcaggtgggttgcatggagccaggctgaccctccatgtccccccagggtgagatggccgccactccggcagggttcgcccagctaacccacctgctcatgggtctggcaggaggcaagctgatcctgtctctggaggtgagtgactcaccttcgtccctcagcctgtggatcctggagggcttaagaacgaagctttcaagtccaacaggagttcctggcccaggaattcacttactagctggacaaccttgcataagtcacttcacttctttgtgcctccgctttccttctataaaacgagcataatgtaatcacctcatatcattgtccagtgaattcagtgaggcacccttctaagttccttgttgcttgacatgtggtatgcactctcttattatcatcattattattattccctggtcatgtctggtcctttcctgatcccccttcacaactccttgccccagggtggctacaacctccgcgccctggctgaaggcgtcagtgcttcgctccacacccttctgggagacccttgccccatgctggagtcacctggtgccccctgccggaggtgagccccggtggaggaggggaaagcgggagcctcactgcccgcccacattccttgacatcatattttctccctgccctgcagtgcccaggcttcagtttcctgtgctctggaagcccttgagcccttctgggaggttcttgtgagatcaagtaggaagtggggtgtgggcctggtgtggggtggacgtgggatgagccctgggggagactgtcttcagacttcctctgactttactgggctgccttggtgggagagggtcaggccctctctctctccctgggccctggtttcaggcctcccacccctttttctgtttgggtcacctgagttccagagtatgacggggattcagattgagagggtaggacaaacgtggcctcccccttttagctgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacaggtaggcatttggagggctggatgagtaggtgttgggggtccctccccctcaggcactaagcctctacctctcgtttccccactgctagccaccaccctgaggtaccccagcgcatcttgcggatcatgtgccgtctggaggagctgggccttgccgggcgctgcctcaccctgacaccgcgccctgccacagaggctgagctgctcacctgtcacaggtcagaccccggtgcctggggtgggtggattcccaggactgtggatgaggtctcaggggctggaacttgggtttcccctcctggggagctgctgcaggacttgagaggggctgaagtccctgtcttaggggtggggaccagggaggggacagaccctcctcactgtcctgcgggtgctcctctctgtgcttccagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggtgtgtcctctgtgggctggggagaggaggacctggggggaatggaaaaagagagccatctgctgtttctggaggctctgagagagtcaagcagggcctgaggaaaggggccatggggaggggcacgggagggggtgggcctagaggctgggggaataatggaaggaactggagaaagggaagggtggccaagtgcagtggctcacgtctgtaatcctagcgctttgagaagctgaggtgagctcaggaggtcaaggctgcagtaagccatgattgcaccactgcactctagcctgggcaacagagtgagaccctgtgtcaaaaaaaaaaaaaggtccgggcgtgatggctcacacctgtaatcccagcactttgggaggccgaggtgggcagatcacgaggtcaggagatcgagaccatcctggataacatggtgaaaccccgtctctactaagaaacatacaaaaaattagccgggcgtggcagcaagcgcctgtagtcctagctactcaggaggctgaggcaggagaatggcgggaacctggcaggcggcgcttgcagtgagccgagatggcgccactgcattccagcctgggcgacagagcgagactccgtctcaaaaaaaaaaaaaaaaaaggaagaaaggaaggtgagggcaggctgtccgatcttagcaccctgctgcttcccaggttctgaatggtgctgctgtggtgcgtcccccaggacaccacgcagagcaggatgcagcttgcggtttttgctttttcaactctgtggctgtggctgctcgccatgcccagactatcagtgggcatgccctacggtaaggactccctggggaccccccaaatcctgatccttgtggggacctggatgctccccccagcccattgtttactcatgccccatggtccagctcccaggacttccttccccagaaagaagtaagggtgcagtccttacccagagaaggaccgaggatggagcctctcagtgcttatttagataggacccccagatcatgttacctgggcacttacaaaacaggatccagggtcccaccccagcatttctggcctatagcagctgcctcgacagaacccaggtctaggctcctgatgcatactccagaagacccacgaaccagcccccagcattagctgcccaaggcccagggtccagttccctagcaatgacccagaagagcccagggcccagccccgagcacttgctcaggcctaccgggcagcccttgggaaccaggatcagacttttctccttaactgataggacccagggtcctgccactcagcactaaatgcccctgcagaccccaggggtctagcccagccctcttccagggcgtgagtatcgtcggttactgaggtgctgtctcctccccaggatcctgattgtggattgggatgtccaccacggtaatggaactcagcacatgtttgaggatgaccccaggtaagggctgcagtcaggggccgcagtgtgatcagggaggcgggtgagcacctcaggggtactggagcccctaaccagccatggtccgcttcccaccccctccctggcagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgaggtacagctcaggatagatcgcaggacgtatgtgacacgcctgtgcaggtggcagcctggacctgcctccttggcatgtgtgtgtgtgactaccatgtggctgatgatatgagacagcgcatgggtgtttaatgaggctaccagggcagttggtggctgtgatcaccttgtgtatttgtgcacagatggctgtacatgactgccttctctgtgtgacactgtgtgagtgactgtcttggctgtgtgtgtgagggccttctgactatgcaccaatggcccgtgtcctggctggatctgtgttgtatgtgcgtgccatcacaagtgcttgagtatgtgcgtgtgcgactctgtgcaggcaggtatttctcaccttttctctgtgtgatgggtgtgatactgtatgtcactaaatgcaaccatctctgtgacagcctgtgtgggaccttctggatgtggaaggctgtgtgtgacagcagtcactgtctggctgtctgccctggctgtttgtgtgtatgtctctgactgggaggtagctgccttccctctgtgtgagctaggaggctggtggcttgtgtatggctgactctgggagggagggaaagggtgtgagcaccgctctgtcaccctgggcgccggtgtgcatctttggatctgcatgtgcatctgactgtgcatgggtagccaggacagccttctgtgtgtgtacaactgtatgtgcaaataaccagctcttgtggacataccagagtgcatgtcgccattgtggctgtgtggtgatggcatgtgtatgtgactgtgcaggatggctgtgggttattgtcctctttgagaaactgtgtgtgtgtacatgactagtgcagatatatagtactgatctatatggtactgtctgtgtggcagtacgtgtggctctgtgtgaccatcttctctgtatgtggttcttgcgttgccatcttctgttttggaagtgtgcaagcacctctctttggcactatgtatataaaaccctgtatcttgtgtatgtgtgtcccacgtttgaccctggggtatgtgtgtgtgaggacatgtctctgtctctctgtttttcatgtcactctcttgacgtgtcagtctgtctcatgtccccatctgtccctttttttttttttttgaaatggagtcttgctctgtcacccaggctagagtgcagtggtgcaatctcggctcactgccagctccgcctcctgggttcatgccattctcctgcctcagcctcctgcgtagctgggactacaggctcccgccaccacgtctggctaattttttgtatttttagtagagacagggtttcactgtgttagccaggatggtctcaatctgacctcatgatctgcccgcctcggcctcccaaagtgctgggattacaggcctgagccaccatgcccggcccccatctgtccctttctgccatctccatgtctctaagtctgcatctatttcttctacctctgagtctgtatttcttttcttcccttcttcattttcagtctctgcttttgtgtctcctagtctccaggttctgtgtctccatctctctgacttgcatctccatgtcttcatttgtccttttctccatgtctctgggtcgccatcacttactgcctaccaaaagcccctaccctcatgcttatacatctcttctctccctgcctcagtttaacccagaactggtgctggtctcagctggctttgatgctgcacggggggatccgctggggggctgccaggtgtcacctgagggttatgcccacctcacccacctgctgatgggccttgccagtggccgcattatccttatcctagaggtaactttctcttggtccctcttcccacagtgggagggtagtttggaaatgttggcaccatcactcaggacagtttcctgaaatctcctttgagtgtccctgcctgggattgttggcacatactgggtagtaataagaataacagcaacattaacagccttaattgagtgctcactatgtgcagagcactcttctgagcacctcatgtgtcttattcattcagttacttggcaaagcagccttatgaggtagggtaagcttacctcattttcctctctgaagatcagggaaactgaggcacaaagaggtgtgatgactagcctgagatggcacatttactgatggcagaaccaggacttgaacccatgcagcctggctccagggtttgtgctttttactctacctgaactgcctctaacaagttcctgggacaaaggaaattcactgacctttgtaaatgactcagtatccccacgccctgtgcctcagtttcctcatctttatgaggcaggctaatcatggaggaatgcctggcatatggtaaatgctggatcttttggatctggttttttgttgttgttgttgttgttgttgtttgtgagacaagagtctcgctctgtcacccaggctagtctgcagtggcatgatctcagctcactgcagcctccacctcccggcttcaagtgatcctcccacctcagcctcctgagtcgctgggattacaggcacccgccaccacgcccagctaatttttgtatttttagtagagatggggtttcaccatgttggtcaggctggtcttgagctcctggcctcaagaaatccacccaccttagcctcccagagtgctggaattacaggcatgagccaccgcacctggcccctggttactgttatgcgttgttcagatattaacccactcagcaaccttctgtgctcttattttctttttctttttctttttttttttgagacgttgtcttgctctgtcgcccaggctagagtgcagtggtgcgattgtggctcactgggttcaagcagttctctgcctcagcctcctgagtagctaggattacaggtgcctgccaccacacccagctaatttttttgtatttttagtagagacggggtttcaccatgttggccaggctggtgttgaactcctgacctcgtgatccacctgcctcagcctcccaaagtgttgggattacaggcatgagctaccatacctgcctctttttttttttttttttttttttccttttgagacagagtctcgccgtgtcacccaggctggagtgcaatggcgctatctcggctcactgcagcctctatctcccaggctcgagcaagcaattctcatgcctcagcctcccgagtagctgggactacaggcgcatgccaccacgcctggctaatttttgtatttttagtagagacgggggtttcaccatgttggccaggctggcctcaaactcctgacctcaggtaaccttcccgcctcggcctcccaaagtgctgggattataggggtgagccaccgcgcccagcctgtgctgttattttcaccactttacagatgaggaaaataagtcactgagaaattaattggcttgcaaaaagtcgcacttactatgtgcccagtactgcttgtgacaggacacggtgggagttaagaacacagatgactccatcagagagcccaagttcaaaccccagtgctgcctcttcagctgttatgacctcggacaagtcatctaaccactctgagcctcagtttctttatctgtaaaaagaatagcaactgggattcctgtagagtgtaaaaaaagtcaatacaggagtccatgtggagaaagagtccagttgttcctgtactaggtcagggcaggggcttttgtatgggttcacagcagtcgtcttgtgcaggtcaggtcagtggttactgtatgatgtgaggccaggggttcctggacagtgtgaagacggatttctgtatggcatgaaggcaagttcctgtatagattgagcgcctgtggttgtgaggattgagttaatatgcatgaagtacttaaaatcgggtctggcacacagcaaacaataaatgtcagctgttactaatgcttttatcagtattgttatcttgcactttacatcacaatttactccccacaacaaccaccttacacagtgggtgctgcctatcttaaaggtgtgggttctgttcacttgttctcagtactccccctccctctccagacttgcagggctcagtagggaggattggggaggcaggacatgttgtcctccaaattccccaatgaacccccaccatacctttccctccccttttccttaacaaagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgagtgagtggatttgggggtgatggggggaacccagggaaggagggggctgaggggcagagatcagaggcaggaagggtgacagtacccacactcaaggatctccctccctggttccagaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcaggtaaggctcaccacacccaggtaggggcaagaaggggcaagaatcgggcttctctgatacatctcttacttctgcgtgtccagggggcctcagaatctcaggccccaggagaggagaacctactaggagaggcagctggaggtcaggacatggctgattcgatgctgatgcagggatctaggggcctcactgatcaggtgagctcagggagaggctgggactgtggcttccttctcttgccaatttgtgtctccaggtaggagcatgtgatgaataaacatataatggggagatggggtcttcagcttacctagtttcacccacccactccaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtatggagcagaggaaggggatggggcggaggttggcccgggagcaaactgcagggggatgctgacccccacctgacactcacacccccaacctcaggtctactgtggtcgttacatcaatggccacatgctccaacaccatggaaattctggacacccgctggtcctcagctacatcgacctgtcagcctggtgttactactgtcaggcctatgtccaccaccaggtgggccctgggtagacccttcgacacgtgcacactcaccccccacacacacaccccttctgtgattgggtaaagggagaccacaggcccctctgcatggctgcctcatgtgattattttgcatggggcgggggctgtgggatagtccagaagacagagtggttgagggctggagtgggggcagccctgcagccgaggtagaggggtcctcactctctctcacctgccctattcttgcctcctcctcaggctctcctagatgtgaagaacatcgcccaccagaacaagtttggggaggatatgccccacccacactaagccccagaatacggtccctcttcaccttctgaggcccacgatagaccagctgtagctcattccagcctgtaccttggatgaggggtagcctcccactgcatcccatcctgaatatcctttgcaactccccaagagtgcttatttaagtgttaatacttttaagagaactgcgacgattaattgtggatctccccctgcccattgcctgcttgaggggcaccactactccagcccagaaggaaaggggggcagctcagtggccccaagagggagctgatatcatgaggataacattggcgggaggggagttaactggcaggcatggcaaggttgcatatgtaataaagtacaagctgttaaaaaacctgga

SEQ ID NO: 21 - DNA, 24806 bp, Homo sapiens
  source 1..24806: mol_type = genomic DNA
gattaggaggacaaaccggcctcctgcagagctgacatttagacatggagtgggagttgcgggggagagggggtgcctagggacagctccccagacgcacacacactttctattactgcttgggagcagtggttacccgttggagggggcagatagggagagctgctgcccatgagaactcccatcctcaaggccattctggccctgggcactattattatcacaactttccagatgaggaaacggagcacagaactgttcagcaacttgcccaaagtcacatggttaagtcccgtaacctcagtttccttatctgcaaagcagccttaataatgggaactacctcatagaattgtggtttacaacgatgcctgtcacataatacttgctatcattaccattccctgcccccactaagactccttccactcagaatattgcggcagaatattaatagctaatattcggccagtgtttcctgtgtaccaccatagtgaatattaactcggaaaatcctcacatttagcatcgtcaccacgcctattttggccgccaccaccaacgtccagtggcaacctcgctctcggcccagccacaaccagcccgcccgcgctacgtcatcacggcgcgtcgcgcgggaagtcgcggggaaaaggtaagatgccccttcctaattccctcgcttccttgcttgctctccagacaccgccgacccgaagggctcctaagtgactggacagggacggaggggcggcggctatggcatccgggacgacagcgacgatagccgaggcaagaaaaagcttgcaccgcagccctgtcgggaaccgccggcgcctgccttttacgtaggcgcagcacagctggcgtagcacgccgacgcaccgccccgtcgcctgtgagctcgcggtgtggctcagcccagctggcgtagcacgccgacgcaccgccccgccgcttgtgagctcgcgagaggtgtggctcgcgcgaggggcgcggccttgaggcacggtcccctctggaggcgggatctgggcgggcagtcgagagacgaggcccaatggaaaagaaaagcctgagcgctcgcgtccccacaggtcgccagaaacttggtggagcgagccaagggcggagtttgagaaaggggctgcgtccaatgagtggagcggtgagtacagcgcgtcgacggcggaggcgggaagggggcggaggccgaccgaaggcggctagggactggctgaagagggttagcgaaacgttagcggtcgggtctgggcagggggtggagctggttgaaggaacggggcagtcccctgaggagcggggctggttgaaacgctaggggcgggatctggcggagtggaaggtaccggtccggcccgataaggggtggagttaagtgaatcgttaagggtggagtcgaaaccggggtcggggccggggccggctgagtgaaagggtgggtgattatcccgggagataggccgaaagggcaggttcgtgaaggaatggggtctgggctgggcttagagggtctgggaatggggcttaggagagtagaaggggcggtgattggttggaaagggcaggccctaagacggactgtgtgagcccgggggctcattgctccgtgaaagggcaagaccagggaaagagaatcgtgtaaaaggggtggagtctagcgggccgggcggggccgggtagaatggccacctgagtggagggagaaggcctgagagaggcggagtttggaaggctgtggagaatcagatcgcgtaaggaatggaatctaatagagggggcggagcctggaaaagggcagagaggtggggtcctcagggcacactagccccctcacatacccacgctcctccccccacccccagaaccgcggcaggggccaagcctcctcaactatgacctcaaccggccaggattccaccacaaccaggcagcgaagaagtaggcagaacccccagtcgccccctcaggactccagtgtcacttcggtgaggccctagacccgccctgatgagggggagagggagcaagttggtcatgccctggactctgacccttctctctgattcacagaagcgaaatattaaaaagggagccgttccccgctctatccccaatctagcggaggtaaagaagaaaggcaaaatgaagaagctcggccaagcaatggaagaagacctaatcgtgggactgcaagggatggtgagcagggcctggctgcagtttagcctcgtatgacaatcaaaacccaaaggttccccaccctggacagttccctctgactcagggcctaaaatggatctgtgtctccttttttttttcctctgcaggatctgaaccttgaggctgaagcactggctggcactggcttggtgttggatgagcagttaaatgaattccattgcctctgggatgacaggtgaggctgggtcctccaggctgtctccaaaccacaaaaatacacacacttagagcccattagcaagaataaatcttttcttaccccagcctggatactggaagcagatgatttggggaggtagggctcacacttgaaagggttcttttatgtagctaacaaacatttattgagtgcctattgtgtgccaagagttggtgatacaaatgtaccaaacaacagacaggggtccttctctaggagcggattgatgagtaggagggcagatcattaaacagccaacagcagtatgaggtgtttaatgaagacagacttaagaccaggggattcaagggtgtataatagaggatcctaaactagtatccgggcttcccagaagcctttatagaggaagagacgtttgtactgaaacagaaggatgaaacctcccgtttgctttacttttcaaaaagaatcagcaaatccttttggctatatgttcaaaatatattcaccatctgacctctcctcaccacctccactgccaccatcatcatcactcacctagacttgcagaggcctcctccctggtcccctacttccaacctcacctctattatctgttcatgcagcaacctaaaaaaatatgtatgagttgatgtctttgttaaaatactttaaggaggccaggcatggtggctcacgcctataatcccagcactttgggggggccaaggcaggtggatcacttgaggtcaggagttcgagaccggtctgaccaacgcgatgaaacactgtctctactaaaaatacaaaaattagttgggcgtggtggcacacacctgtaatcctagctacttgggaggctgaggcaggagaatcgcttgatctgggaggcggaggttgcagtgagccgagattgtgccattgcaccccagcctgggcaacaagagcaaaactctgtctcaaaaagtaaaaataaaaaaaaaaaaagtactttaagggcaaactgttgccctcaaggtaaagtccaaatttttcatcctggcctcaaaggttccacatgatctggctccacatcactttgtgacctcatcagccaatccttctcgcccacactactccccttgttcttccaacatgccaggcaccctcttgcctctggtctgtaccctgaccaacccctttcccagagattcttttcggtcttctactcttccagctgcctttcacatcccaatgagactttctcagagaaggttttgcctggccaaaccctaccccaatacctgatcttattttcctagcccatgacattgtttgctaattatcatacttgtgtgtttaccaattaatatctatatcccttatcccaacctaagactaacttactatgggaaaaagacttcttttatggaatacctatttaccatctattgaggagctagggatacagcagtaaacaaaacaaacaaaagcctgtgtcttcagggtaaagggggaagacagacaataaataaacaaaaagtaaatgatgcactgtggtagaaagagcagtgttatgaagaagcagtaaagggaggatagtaggagaaagtacagtggaggagacttcaattttaaataggctggtcagggaaggcctcactatttgagcagggatctgaagaggtgaaggagtaagtcatggagacagttggaggaagagtattctaggcgggggtgggggggccagctagagtaaagttctagggctggctgaagagtatttggttcaactgagaaccagggaagagaccagtgtaggtgaagcgaagcaaaggaaagagggaggggtaggagatggggtcagagaggtgatggaggacagattgtttagggccttctgggcatgggtaatactttggcttttactcagaatgggatgggagcctcgaagggacatgaactgattagggttttaaccagtgtctagctgctatgcacagaaaggaccgttgaggtggcgagggtcgaagcagggaggccgaggagatgccgccacagcccaggcgggagggtatggtaatagcagtggcagtggtgaggagagggtcacacaccagagctgatgggaggcaggactgacaccacagttttggggtaagcagctgtggaagggtggatggggagatgtggagagaacagatgtggagctccccagtgtcctcatgcatctgtgactgtgactccatctccccagcttcccggaaggccctgagcggctccatgccatcaaggagcaactgatccaggagggcctcctagatcgctgcgtgtcctttcaggtaaggcccccaagccaacactctgggtgaagggcagggagctgacctgacctgggtgcagcccatgccttaaccctttaccccactttattcctcatctctccccaggcccggtttgctgaaaaggaagagctgatgttggttcacaggtgaaggcttgggagccttgtagggatggggaggagcctgccctggacaaaccagggcaagctaaacgctttgggaagcaatgtcatggtctctgcagccagccttgggcaagtcgctgagcctttctgaatctgtgtcatcgcctgtaacaaggggaccagtaaacataagttcctcaggagagtttgatgtgtgtcaggtgcttagaacagggcctgatatataatcagcccatgatgagctattactgttatcatgatctgattgtgtgaatagatggtggatgtgtggacagagtgatcggggttcccccaggggcttccagatttagaggcctagtgctatgtggcttgaggaaagctagcttccagagcaggtatctgagctgaactttaagaggagaaaaagagtgagtcagatgaagagggtgactgagggaagggaagaacagacagctgaaccagtgtggccaaaggcctgaaagtaggatttggtgtggctactgcaggggagcagagaacattacctgaactgaggcgggctagggaagaaacagaatgtgtcttatatcctggctattccttcaggttgacagttgggggagccaagccctaatttgaccagggggttggtgtatggacagctcagttggggaagagacagggccctgaatctcatctcccatctgtgtctagcctagaatatattgatctgatggaaacaacccagtacatgaatgagggagaactccgtgtcctagcagacacctacgactcagtttatctgcatccggtatggatgagaactctgcgggcagggaatggtggcaatcttggggtccccatccctatgcccaccccaccctgggggaagcagctgagtggatagggctgttctctcccttactccctttctggctccccactgtctctccagaactcatactcctgtgcctgcctggcctcaggctctgtcctcaggctggtggatgcggtcctgggggctgagatccggaatggcatggccatcattaggtaggactctacagcatttgactttttacattgttttaaagtccttccttcaagtgtaaaatgtattgatatgggtaatatattcatgtgattcaagaatccaaaggtaaaaaagagaatataattaaaaatctgtctcccatccttgtcacccagacacctgcctcctctcatgggaggtagtcattggaaccactgactcatgtgtcctcccacaaagtcttattcaagaaataaataccatatttcattgattctaaggtgtacattttttcatatttgtaacatcttgaaatccagatgcatcttacaaacagtgacatctcagaattccattggcagtattttctgcttcgtttttggcatgtaaaatcatggtacatcttatcatcattggcatcccagaactgatgaaatgcaaaagctttctcctccctcccctgtttttaaccacaaatggtagtaacatgacatactcccttcgtaccatcttttcacttacacatcttggaaacctttctctaacagtatgtatcttggagcccggtagaacaccattgtgtaaatgcactctatgttaatcagtccccgactaatgggcattttggttctccccagtcatttgctgttttaaaaaaatgctacagcaaatatgctagtacctttataaaaataaaattttattttacactaacataaaataaaaatgttacttcaccctttttaaaacacaggttgtgggctaagttcctgcagcagaatgactgggtatgagaatgaatgcgttgatcattttatttttatttttatttatttatttattttgagacggagtctcgctctgtcaccaggctggagtgcagtgatgcaatcttggctcactgcaacctctgcctcccaggttcaagtgattctcctgcctcagcctcccaagtagctgggactacagtcgcccgccaccacacccagctaattttttgtatttttagtagagacggggtttcaccgtgttggccaggatggtcttgatctcttgacctcgtggtccacccacctcggcctcccaaagtgctgggattacaggcctgagccaccgcacccggcctttatcattttagtgaacattttcaaatctcctttggagacagaggggtagattggcagggggtgagagtttgagggcctccattactcagttcccggcagatttttaggcatcagcattaaaggaattggtggtccaggattagagagcaggggacagtctgtctctgcagaggaggacccctcacatactccaagctgttatttccttgtcttgcccaggcctcctggacatcacgcccagcacagtcttatggatggctattgcatgttcaaccacgtggctgtggcagcccgctatgctcaacagaaacaccgcatccggaggtcagcaacagagggcagatgtgatgggggtggcatggggtggggtgtcctggtctgggctcttcccccagattcaccttgatctccttgcacagagtcccctctctctgctgctcctagggtccttatcgtagattgggatgtgcaccacggtcaaggaacacagttcaccttcgaccaggaccccaggtataccccgactcccacctaggtgctagactcccagccacctcccacccttacaggcccagccagaaggggtccagaaggaggaaaaggcattcacattcatggggcctatcctgggatcaggatctgtgctacttttgtgtgtctgccatctctcctaatttcactctgaggcagggactgttgggttccctattttacagatagggggactgaggcttatacgttaagaggtcacacagagaagaactgggagagacgggatttgacccacaaatatttgaagatcttattcactgattaaccgttgggttagagtgaacctcaaattactgctttattttttctgattaggataatgcttcaaagtgtttgtttatcctttcagtagatagtaaaaaacaaatataaataaataaataacaaactatttgtttaaagaaattaaataatacagaagtgtctgaagcagtgagtctcctctaatttccattacctcaaaaataagtgttgacagtttaggttatatccttacagattttctctatagatgtctgaatttttattattattgttataattattaataacacactgcctaatggaataattctccacatgttcttcgacaacttgctttattcatttaacagcatatctgggacatttctccatgttagtacctcttaaagtatctcgttctttgtagtattcaattttatgactgtacctctggttatttaaccccaggacttttaagatgcctgccagtttttgctattacaggcacttctactgtgaatagtctagtaaatatgtaattctgcacttctctgagtctatctgtaggatgaatttctaacagtgggttgcttgggccagagagtcagcatactttaaagtctggtatacactgccagacttccctccaggaggggaccagcttacccttcctgcagaaagtgattgagcaagtccttgtcagtaggagacattactaagtacatgtcgagtatcccttatatgaagtgcttggaaccagaagtatttcacatttcagattttttcagattttgaaatatttatattatacttagtggttgagcatccttaatctgaaaacccaaaatccgaaataccccaatgagcatttcctttgagcatcatgttggccttcaaaaagtttaggattttagggccgggtgaagtagctcactcctgtaatcccagtactttgggaggccgaggcggatcacctgaggtcaggagttcaagatcagcttggccaacatggtgaaaccccgtctctacaaaaatacagaaaaattagccaggcgtggtggcacacacctgtatttccagctactcaggaggctgaggcaggagaatcacttgaacccaggaggcagaggttgcagtgagctgagatcgcaccactgcactccagcctgggtgatagactgagaccccatctaaaaaaaaaaaaaaagtttaggattttacagcatttcagatttcagatttctagattagggatgctcaacctgtagtagttttatctagccttcttcctagctgactacagagaatccacttatccaagatctgaagaatccaatttaggttggttgttctctagacattgctttattattttgcagcattcagtgttcctgacaggtgtctgcttttcattcctttccaagtaatctactttcctctctgcaaggttatcttggtgttcttttttttttttttttaaattattagacagggtatcactctgtcacccaggctggagtatagtagcatgatcatagcccactgtaacctcaaactcctgggctcaagcaatcctcctgcctcagcctcccaagtagctaggactgcaggcatgtgccaccacacccagctaattttttaaattatttgtagagacagggtctcactatattgcccagactggtctcgaactcctgacctcaagtgtcctcccactgcagcctctcaaagcactgagattacaggcatgagccactgtacctagccttgatgttctaaacttttaccctggccttttcactctgtttaatagtaggtagacattttacatctgaagattcatgcttttcttaaactcttggaaattattttctattagatctttgataatttcttctttgttttctctgttctctttctaggacttctgttagtcagatgttagacttcctgggttgatcctctatgtctctctcttttttattatttatttatttatttatttatttattttttttgagacaaggtcttgctctgttgcccagactggagtacagtggcacaaatacgactcactgcagcctcgacctcccgggctcaagcaatcctcatgcctcagcctcctgagtagctgggaccacaggtgcacaccagcatacccagctaattttttaatcttttgtagagatggggtctcaccatgttgcccaggcttcttattttttatcttatgttttctgtctcttttttcttttgctgtgacctgggagattttgtcaactttatcttcaagcccttctgttgaattatgtatttagatatcctgttaaactccaaaagctctttcttcctctctgtttctatgtcctctcatacctgatcttgtttttatagagtatggattagaagtgccggctgggtgcggtggctcatgcctgtaatcccagcactttgggaggccgaggtgagtggatcacttgaggccaggagttcgagaccagtctggccaacatggtgaaaccccatctctactaaaaatacaaaaattacccagacgtgatggtgcgcacctgtaatcccagctactcaggaggctgaggcaggagaatcgcttgaacccaggaggcggaggttgcagtgagacaagatcatgccactgcactccagcctgggcgatagagggagaccctgtctcaaaaaaggaagtgccttaagtttcttctgttgtctgagttatgtctctttattctgaggctggttgaattatttttttcttggtccttctcttttatgctgatggttttcctcaaatgtctagcaatccttatttggctatttatacccaagaatgaaggcccattgtagggggctgtcagaggactactctgctgcagtgtttgtaggtctgttttcctgaaaggcatttccccagaatggggacagggaatgtccactgacaggttgcaactttagcgagtgtgggcaaggacccgggcattagatgggatcctgccagatgtcagaacaaaggaggcctcttctttggggtaacatctcttttttccctcagcctttccaaagtgtatctgttgtacttatcttttccagtgtagctttgggctttgttggtcatctctactgttttattatggtgggtttttaatacttcattaatttttctggttttatctttattaatgttttcctccgacgttctttattttgttcatttctggcttctggaatttaatgcttcaacatttatcaactcattggccgatctcatttcatctatacctgtacccactgccctcttcctgttttccttcaaagctaatcccagataccatataatttctcctgtaaatatttcagaatgcatctctaaaacataagtacacttttttagaaaagcattaccttagtatcaatacctgaaaattaacaataattatttttttaatctgccaagggtctgtcattcaaagaattctttaatatcattaatcattctttttgtttgaataaatgcataggaggctatagctgtgtgttctttcatgggtacactattgattacacacacagatgtgaaacactgtgaatatattcagtgcattcttccttattaacaaacgtgtgtaaacaaacaaacaaaaagaacatgtccatgcccttggatctcccagctccccatctctcagtctctgaactccagcagcagggagacaaactggataatccttaaaatactgttgtgaagacaaggaaagcagatgatctaaaactaagtagttaaaagaaacaagtaactttgttgtattggtgattcagtgagttggctcttcagaagggattttgtgtgcattgacctggatctgtactggatttctccagaaaggaaacttcagcttatcttgggtgcccccttatcctgctcagcatgagacagagcaccctggggcccctggaatctcaaagtctactttctagaaattcattgaagtctcttattctctgacatcctttccactctctctttctgggttgctgtttgtttacaatttctttactgtattttgaatggggtagtaggaggttgaggaagtaaacctgtgctatcaatccaccatttaacttgaaggtggttgaacttattttgaatttcccttctgttttctttttaaacttagcttgagtgctagttctgctgtttattgactttgatggctctcttttagagtcagctttcttcagatcttttctttcttcctgtcttccccaatgatatttttttcttttcctttctttttttttgagacagagtcatgctctgtcgcccaggctggagtgcagtggcccgatctcggctcactgcaacctccgcctcctgggttcaagcaattctcctacctccacctcccaagtagctaggattacaggcacgcgccaccacgcccagcaaattttttgtatttttagtagagacagggtttcgccatgttggccatgctggtctcgaacttctggcctctagtgatccgcccaccttggcctcccaaagtgctgggattacaggtgtgaaccactgcgcccagccctctggtgataatttttatgtcctactgtgtttctcttcctctaatgattaacttactaaaagtttagaaccaagatttatctatcaatgtcaaaaataaaacaatcatcctccacctactcaacaccagagcttttgcttgtttttattccccaattccaagctcttttccctcaattttaagaatctttctatatcacttgcattaaatttcacaactacattatcaacccattaaaatgtgactcttaagtttgtctagattcaatgcataccactatttcttgcatttccctaaaaaagctgccttagatcctctctggaacaaagcagggggtaaatgaccagatagaaggatggatggatgatagattattcattttgctttccccagtcagaattaagccctcccatggccccttgctgggctttctgtcaaaacacgtgcatcgcatcttttcagtatctgttttgcgtgtcggtcatactcacccctctgagtgctcattctctcgcctataaaatagaagaagtagtagaggtggcctgtaaggcattggggaggaggaactgccctttcactggcctcatgacagccccagccagcacccccgctctagttccacccacagaagcaagtctcacccctaccccccaggtctctccacttcctgctagagaatcacccagctccccagcctggcttgaggctcttttcagccttagctggctccccgcagctaagagccccatgctccatttgggcctccagtgtcacagggtttgacctgctgttactactacctttgtcataggggcaggtagtgggcggacatgatctagagtaccccacagagtcaggagcccccatgatctgagatagaccctagagctgagcctgaaattgggtgggcactgagggggcagcgatgtttgctgagggagaggttctggtgagagactaaatgaatgagcgagtgaggtaatggataaataaagataagaatgaaaaaatggtaagatgattaaattggtgactgaatgtaagggtaaattaatggaagaatggattcatggattaaataattaatggggaaattaatgaatgagagaatgaacgagtggttgaatggggaaatgagtggtagaggaaactggcaactgttgggttgggggtgtctatggggattgacttctccaactctcttacctctttttcttctgcccccagtgtcctctatttctccatccaccgctacgagcagggtaggttctggccccacctgaaggcctctaactggtccaccacaggtttcggccaaggccaaggatataccatcaatgtgccttggaaccaggtcagcatctacccacctctgcccccaaagtgaggcccagccccgcccttctgtcagcggctcgggacaggtggctgaacatcccccatcacactcctgtgtctgcaggtggggatgcgggatgctgactacattgctgctttcctgcacgtcctgctgccagtcgccctcgaggtcctggggatctggggtgtgttgggaggaggagtggccttgaaggttaggctgtgagggtaggtagcctcatggagtctgttcccccttcagttccagcctcagctggtcctggtggctgctggatttgatgccctgcaaggggaccccaaggtaaggcaggctccctggggcggcaggtgggttgcatggagccaggctgaccctccatgtccccccagggtgagatggccgccactccggcagggttcgcccagctaacccacctgctcatgggtctggcaggaggcaagctgatcctgtctctggaggtgagtgactcaccttcgtccctcagcctgtggatcctggagggcttaagaacgaagctttcaagtccaacaggagttcctggcccaggaattcacttactagctggacaaccttgcataagtcacttcacttctttgtgcctccgctttccttctataaaacgagcataatgtaatcacctcatatcattgtccagtgaattcagtgaggcacccttctaagttccttgttgcttgacatgtggtatgcactctcttattatcatcattattattattccctggtcatgtctggtcctttcctgatcccccttcacaactccttgccccagggtggctacaacctccgcgccctggctgaaggcgtcagtgcttcgctccacacccttctgggagacccttgccccatgctggagtcacctggtgccccctgccggaggtgagccccggtggaggaggggaaagcgggagcctcactgcccgcccacattccttgacatcatattttctccctgccctgcagtgcccaggcttcagtttcctgtgctctggaagcccttgagcccttctgggaggttcttgtgagatcaagtaggaagtggggtgtgggcctggtgtggggtggacgtgggatgagccctgggggagactgtcttcagacttcctctgactttactgggctgccttggtgggagagggtcaggccctctctctctccctgggccctggtttcaggcctcccacccctttttctgtttgggtcacctgagttccagagtatgacggggattcagattgagagggtaggacaaacgtggcctcccccttttagctgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacaggtaggcatttggagggctggatgagtaggtgttgggggtccctccccctcaggcactaagcctctacctctcgtttccccactgctagccaccaccctgaggtaccccagcgcatcttgcggatcatgtgccgtctggaggagctgggccttgccgggcgctgcctcaccctgacaccgcgccctgccacagaggctgagctgctcacctgtcacaggtcagaccccggtgcctggggtgggtggattcccaggactgtggatgaggtctcaggggctggaacttgggtttcccctcctggggagctgctgcaggacttgagaggggctgaagtccctgtcttaggggtggggaccagggaggggacagaccctcctcactgtcctgcgggtgctcctctctgtgcttccagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggtgtgtcctctgtgggctggggagaggaggacctggggggaatggaaaaagagagccatctgctgtttctggaggctctgagagagtcaagcagggcctgaggaaaggggccatggggaggggcacgggagggggtgggcctagaggctgggggaataatggaaggaactggagaaagggaagggtggccaagtgcagtggctcacgtctgtaatcctagcgctttgagaagctgaggtgagctcaggaggtcaaggctgcagtaagccatgattgcaccactgcactctagcctgggcaacagagtgagaccctgtgtcaaaaaaaaaaaaaggtccgggcgtgatggctcacacctgtaatcccagcactttgggaggccgaggtgggcagatcacgaggtcaggagatcgagaccatcctggataacatggtgaaaccccgtctctactaagaaacatacaaaaaattagccgggcgtggcagcaagcgcctgtagtcctagctactcaggaggctgaggcaggagaatggcgggaacctggcaggcggcgcttgcagtgagccgagatggcgccactgcattccagcctgggcgacagagcgagactccgtctcaaaaaaaaaaaaaaaaaaggaagaaaggaaggtgagggcaggctgtccgatcttagcaccctgctgcttcccaggttctgaatggtgctgctgtggtgcgtcccccaggacaccacgcagagcaggatgcagcttgcggtttttgctttttcaactctgtggctgtggctgctcgccatgcccagactatcagtgggcatgccctacggtaaggactccctggggaccccccaaatcctgatccttgtggggacctggatgctccccccagcccattgtttactcatgccccatggtccagctcccaggacttccttccccagaaagaagtaagggtgcagtccttacccagagaaggaccgaggatggagcctctcagtgcttatttagataggacccccagatcatgttacctgggcacttacaaaacaggatccagggtcccaccccagcatttctggcctatagcagctgcctcgacagaacccaggtctaggctcctgatgcatactccagaagacccacgaaccagcccccagcattagctgcccaaggcccagggtccagttccctagcaatgacccagaagagcccagggcccagccccgagcacttgctcaggcctaccgggcagcccttgggaaccaggatcagacttttctccttaactgataggacccagggtcctgccactcagcactaaatgcccctgcagaccccaggggtctagcccagccctcttccagggcgtgagtatcgtcggttactgaggtgctgtctcctccccaggatcctgattgtggattgggatgtccaccacggtaatggaactcagcacatgtttgaggatgaccccaggtaagggctgcagtcaggggccgcagtgtgatcagggaggcgggtgagcacctcaggggtactggagcccctaaccagccatggtccgcttcccaccccctccctggcagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgaggtacagctcaggatagatcgcaggacgtatgtgacacgcctgtgcaggtggcagcctggacctgcctccttggcatgtgtgtgtgtgactaccatgtggctgatgatatgagacagcgcatgggtgtttaatgaggctaccagggcagttggtggctgtgatcaccttgtgtatttgtgcacagatggctgtacatgactgccttctctgtgtgacactgtgtgagtgactgtcttggctgtgtgtgtgagggccttctgactatgcaccaatggcccgtgtcctggctggatctgtgttgtatgtgcgtgccatcacaagtgcttgagtatgtgcgtgtgcgactctgtgcaggcaggtatttctcaccttttctctgtgtgatgggtgtgatactgtatgtcactaaatgcaaccatctctgtgacagcctgtgtgggaccttctggatgtggaaggctgtgtgtgacagcagtcactgtctggctgtctgccctggctgtttgtgtgtatgtctctgactgggaggtagctgccttccctctgtgtgagctaggaggctggtggcttgtgtatggctgactctgggagggagggaaagggtgtgagcaccgctctgtcaccctgggcgccggtgtgcatctttggatctgcatgtgcatctgactgtgcatgggtagccaggacagccttctgtgtgtgtacaactgtatgtgcaaataaccagctcttgtggacataccagagtgcatgtcgccattgtggctgtgtggtgatggcatgtgtatgtgactgtgcaggatggctgtgggttattgtcctctttgagaaactgtgtgtgtgtacatgactagtgcagatatatagtactgatctatatggtactgtctgtgtggcagtacgtgtggctctgtgtgaccatcttctctgtatgtggttcttgcgttgccatcttctgttttggaagtgtgcaagcacctctctttggcactatgtatataaaaccctgtatcttgtgtatgtgtgtcccacgtttgaccctggggtatgtgtgtgtgaggacatgtctctgtctctctgtttttcatgtcactctcttgacgtgtcagtctgtctcatgtccccatctgtccctttttttttttttttgaaatggagtcttgctctgtcacccaggctagagtgcagtggtgcaatctcggctcactgccagctccgcctcctgggttcatgccattctcctgcctcagcctcctgcgtagctgggactacaggctcccgccaccacgtctggctaattttttgtatttttagtagagacagggtttcactgtgttagccaggatggtctcaatctgacctcatgatctgcccgcctcggcctcccaaagtgctgggattacaggcctgagccaccatgcccggcccccatctgtccctttctgccatctccatgtctctaagtctgcatctatttcttctacctctgagtctgtatttcttttcttcccttcttcattttcagtctctgcttttgtgtctcctagtctccaggttctgtgtctccatctctctgacttgcatctccatgtcttcatttgtccttttctccatgtctctgggtcgccatcacttactgcctaccaaaagcccctaccctcatgcttatacatctcttctctccctgcctcagtttaacccagaactggtgctggtctcagctggctttgatgctgcacggggggatccgctggggggctgccaggtgtcacctgagggttatgcccacctcacccacctgctgatgggccttgccagtggccgcattatccttatcctagaggtaactttctcttggtccctcttcccacagtgggagggtagtttggaaatgttggcaccatcactcaggacagtttcctgaaatctcctttgagtgtccctgcctgggattgttggcacatactgggtagtaataagaataacagcaacattaacagccttaattgagtgctcactatgtgcagagcactcttctgagcacctcatgtgtcttattcattcagttacttggcaaagcagccttatgaggtagggtaagcttacctcattttcctctctgaagatcagggaaactgaggcacaaagaggtgtgatgactagcctgagatggcacatttactgatggcagaaccaggacttgaacccatgcagcctggctccagggtttgtgctttttactctacctgaactgcctctaacaagttcctgggacaaaggaaattcactgacctttgtaaatgactcagtatccccacgccctgtgcctcagtttcctcatctttatgaggcaggctaatcatggaggaatgcctggcatatggtaaatgctggatcttttggatctggttttttgttgttgttgttgttgttgttgtttgtgagacaagagtctcgctctgtcacccaggctagtctgcagtggcatgatctcagctcactgcagcctccacctcccggcttcaagtgatcctcccacctcagcctcctgagtcgctgggattacaggcacccgccaccacgcccagctaatttttgtatttttagtagagatggggtttcaccatgttggtcaggctggtcttgagctcctggcctcaagaaatccacccaccttagcctcccagagtgctggaattacaggcatgagccaccgcacctggcccctggttactgttatgcgttgttcagatattaacccactcagcaaccttctgtgctcttattttctttttctttttctttttttttttgagacgttgtcttgctctgtcgcccaggctagagtgcagtggtgcgattgtggctcactgggttcaagcagttctctgcctcagcctcctgagtagctaggattacaggtgcctgccaccacacccagctaatttttttgtatttttagtagagacggggtttcaccatgttggccaggctggtgttgaactcctgacctcgtgatccacctgcctcagcctcccaaagtgttgggattacaggcatgagctaccatacctgcctctttttttttttttttttttttttccttttgagacagagtctcgccgtgtcacccaggctggagtgcaatggcgctatctcggctcactgcagcctctatctcccaggctcgagcaagcaattctcatgcctcagcctcccgagtagctgggactacaggcgcatgccaccacgcctggctaatttttgtatttttagtagagacgggggtttcaccatgttggccaggctggcctcaaactcctgacctcaggtaaccttcccgcctcggcctcccaaagtgctgggattataggggtgagccaccgcgcccagcctgtgctgttattttcaccactttacagatgaggaaaataagtcactgagaaattaattggcttgcaaaaagtcgcacttactatgtgcccagtactgcttgtgacaggacacggtgggagttaagaacacagatgactccatcagagagcccaagttcaaaccccagtgctgcctcttcagctgttatgacctcggacaagtcatctaaccactctgagcctcagtttctttatctgtaaaaagaatagcaactgggattcctgtagagtgtaaaaaaagtcaatacaggagtccatgtggagaaagagtccagttgttcctgtactaggtcagggcaggggcttttgtatgggttcacagcagtcgtcttgtgcaggtcaggtcagtggttactgtatgatgtgaggccaggggttcctggacagtgtgaagacggatttctgtatggcatgaaggcaagttcctgtatagattgagcgcctgtggttgtgaggattgagttaatatgcatgaagtacttaaaatcgggtctggcacacagcaaacaataaatgtcagctgttactaatgcttttatcagtattgttatcttgcactttacatcacaatttactccccacaacaaccaccttacacagtgggtgctgcctatcttaaaggtgtgggttctgttcacttgttctcagtactccccctccctctccagacttgcagggctcagtagggaggattggggaggcaggacatgttgtcctccaaattccccaatgaacccccaccatacctttccctccccttttccttaacaaagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgagtgagtggatttgggggtgatggggggaacccagggaaggagggggctgaggggcagagatcagaggcaggaagggtgacagtacccacactcaaggatctccctccctggttccagaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccacctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcaggtaaggctcaccacacccaggtaggggcaagaaggggcaagaatcgggcttctctgatacatctcttacttctgcgtgtccagggggcctcagaatctcaggccccaggagaggagaacctactaggagaggcagctggaggtcaggacatggctgattcgatgctgatgcagggatctaggggcctcactgatcaggtgagctcagggagaggctgggactgtggcttccttctcttgccaatttgtgtctccaggtaggagcatgtgatgaataaacatataatggggagatggggtcttcagcttacctagtttcacccacccactccaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtatggagcagaggaaggggatggggcggaggttggcccgggagcaaactgcagggggatgctgacccccacctgacactcacacccccaacctcaggtctactgtggtcgttacatcaatggccacatgctccaacaccatggaaattctggacacccgctggtcctcagctacatcgacctgtcagcctggtgttactactgtcaggcctatgtccaccaccaggtgggccctgggtagacccttcgacacgtgcacactcaccccccacacacacaccccttctgtgattgggtaaagggagaccacaggcccctctgcatggctgcctcatgtgattattttgcatggggcgggggctgtgggatagtccagaagacagagtggttgagggctggagtgggggcagccctgcagccgaggtagaggggtcctcactctctctcacctgccctattcttgcctcctcctcaggctctcctagatgtgaagaacatcgcccaccagaacaagtttggggaggatatgccccacccacactaagccccagaatacggtccctcttcaccttctgaggcccacgatagaccagctgtagctcattccagcctgtaccttggatgaggggtagcctcccactgcatcccatcctgaatatcctttgcaactccccaagagtgcttatttaagtgttaatacttttaagagaactgcgacgattaattgtggatctccccctgcccattgcctgcttgaggggcaccactactccagcccagaaggaaaggggggcagctcagtggccccaagagggagctgatatcatgaggataacattggcgggaggggagttaactggcaggcatggcaaggttgcatatgtaataaagtacaagctgttaaaaaacctggagaaagctttttgactttgagcaggtggagaaccccacatccctggccagcagagccgatgagtaatgaaggaatgagggagtgggtctctgtcaggtcaatcccctgcccatccattgcccccaaaaaactaactgaaaatagagcagccatcttgactggctccacgggatataaaatggtagaatccatgagaaaatgggtgcttgccttataaataggagcctcacagccagtcccagccacacccacaccctaggctgagaaggcaagaaaactagcaggaagtatccttaccaggcctgtttccccactgtctcattggcctgactggggatggtgggaggtccaggtttaacttgaatgtggtggtcatggtagcttcttggcagaaggggtgcctgccattgaccacaggtgggaaattggaggacagtctgagagctgaatttctcccacgtggtgagaattgctttggcagctcaagggagttggatctgaggtttccaggacttttcttcatggccaccttatgatcccaagctgaccaccacagccttactgagtcaagaagaggagggatggggaagggccggggctg

SEQ ID NO: 22 - protein, 504 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 23 - protein, 508 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQVQSLDTNDITYADLNLPKGKKPAPQAAEPNNHTEYASIQTSPQPASEDTLTYADLDMVHLNRTPKQPAPKPEPSFSEYASVQVPRK

SEQ ID NO: 24 - protein, 503 aa, Homo sapiens
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 25 - RNA, 4200 nt, Homo sapiens
  source 1..4200: mol_type = mRNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatg

SEQ ID NO: 26 - RNA, 4580 nt, Homo sapiens
  source 1..4580: mol_type = mRNA
actcacggccgctctccctcctcgctccgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgagtccttccggtgttcagtattcttgtctttgtgagtgcgtcccggggccgcctcggggcctgcctgccctcctgccaaagcctggaagaggattgaatggaccccagggtttggaaacaacctacagcatttgagcccctcacgtaggttttagagacgtacaatttttgtttgccctggctcagaaggagccggtgtaaggttgagataaaattccatatagacaactgagtttggatctcggctctgctgctttgtagctgtgggagttcaaacagcacctctttgagacttgggccccgcgtcggcacaatggggcaggaatagtccctgccagggtgactgtgtggattcaaggaggttggaagcactgagcccacgcttggcactgggtaggccatcactgaggcttgtgtctttccttcctgccctgaccctgcatagctgagtctcgtggcctctcaccactgatgggatctcggacacccggctcaccctcctcctggggcctcactttccttccccaccaaatgcagacatctaatgcttgtcctgtaatatccttgccagatgactgatgtcaagtgctggtttatagtgggtgctcaataaatgttagtttcctttctatccctccattagatcattaatgatattagtgctaattatgtaataaagttaaggcataaatatgttaa

SEQ ID NO: 27 - RNA, 4200 nt, Homo sapiens
  source 1..4200: mol_type = mRNA
tccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggtacagtccttggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttc

SEQ ID NO: 28 - RNA, 4603 nt, Homo sapiens
  source 1..4603: mol_type = mRNA
gcagccctcgctccctcccccggggcgcgcaggcgggcgctcgctcgcagagaagccgcggcccatggagcccgccggcccggcccccggccgcctcgggccgctgctctgcctgctgctcgccgcgtcctgcgcctggtcaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcgccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctgagaacactggatctaatgaacggaacatctatattgtggtgggtgtggtgtgcaccttgctggtggccctactgatggcggccctctacctcgtccgaatcagacagaagaaagcccagggctccacttcttctacaaggttgcatgagcccgagaagaatgccagagaaataacacaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgagtccttccggtgttcagtattcttgtctttgtgagtgcgtcccggggccgcctcggggcctgcctgccctcctgccaaagcctggaagaggattgaatggaccccagggtttggaaacaacctacagcatttgagcccctcacgtaggttttagagacgtacaatttttgtttgccctggctcagaaggagccggtgtaaggttgagataaaattccatatagacaactgagtttggatctcggctctgctgctttgtagctgtgggagttcaaacagcacctctttgagacttgggccccgcgtcggcacaatggggcaggaatagtccctgccagggtgactgtgtggattcaaggaggttggaagcactgagcccacgcttggcactgggtaggccatcactgaggcttgtgtctttccttcctgccctgaccctgcatagctgagtctcgtggcctctcaccactgatgggatctcggacacccggctcaccctcctcctggggcctcactttccttccccaccaaatgcagacatctaatgcttgtcctgtaatatccttgccagatgactgatgtcaagtgctggtttatagtgggtgctcaataaatgttagtttcctttctatccctccattagatcattaatgatattagtgctaattatgtaataaagttaaggcataaatatgttaa

SEQ ID NO: 29 - DNA, 47626 bp, Homo sapiens
  source 1..47626: mol_type = genomic DNA
gccttctctcaccttgtgtcattcctgtcctcttaaccgccactgtgtgaaccaaaattctctcccattagcagcggggtcttgaacttgtgttcctcccacagtgcggagccttctgccaggtgaccacgacactggcactccccatcactgacctggctactacaggaacggcgttccctccgggaaaaccggggttctgtcctctccaactccaggcctctggccccggctccccttcccctcatcttcctcagggcttgtccttctcccggaacccaggccttcagggccccgctctcccacacacgccccggcctgggcctcccacccgcccagtccgccctccaccaccgagacacctggccagggggcctccacctccctgggaaggccccgacccttcccctctggttgcgcgccacagcccccttctcgctccctctcctccccgcgcctcttttccaggcggcaccgaagtccttcctacacacacgctatttttgtaccatcatctcttcttcctctccccttccccctgcgttcacttacttccttcttttccttcacctcggcccggctctgaaaggatcgggcccctccggcccgcacccacccccaagaggggccttcagctttggggctcagaggcacgacctcctggggagggttaaaaggcagacgcccccccgccccccgcgcccccgcgccccgactccttcgccgcctccagcctctcgccagtgggaagcggggagcagccgcgcggccggagtccggaggcgaggggaggtcggccgcaacttccccggtccaccttaagaggacgatgtagccagctcgcagcgctgaccttagaaaaacaagtttgcgcaaagtggagcggggacccggcctctgggcagccccggcggcgcttccagtgccttccagccctcgcgggcggcgcaggtccgggcccggcagggacggggaggggcagagcgggaccggtcggggctggggaccagccgcgcgggcttctgaacgggcacgctgtcggagccggaggctcgtctagtcccgagccccagcgggccgcggcggctcgcggggccgccttcccgggacagcagccggggcccgggcgccggcgcgggggaggacgctgccctccccggccgggcgcgcagctcgcagccctcgctccctcccccggggcgcgcaggcgggcgctcgctcgcagagaaggtgccggcggggccggagccccgaagcctccccgggcggcgggaagacgggaggaggggaaagggaaggagggagggggtcgggctgccgggagggcgggggcagcggccgggactgcgggccgggagcgagcccagccgcccgcgccgtgtggagcccgggcggggcaggtgggcagcggcgggggcggctccgcgcggccgggctcggccgtgctgcccctcgcccctgctcgcgcggtcctctctcccgcgcggtctgtcggtctgcgcgccgcctcgctcctcgctcctcgctctccccctctttctccccctctcgcctctccgtgtctctggctctctgcctggctcccttgggtctgtttctctctcctgccgcctctctctggccgcccctggctttatttctcgcgcgcttggggtctctcccagtctccgtctctccatttctcctggggggcggggagggggggtctccaaaaaccgcggcggcggcggcggccgctccaggcgcccgttccggagtcggggggaggcccagccgggaggggggaagggggggagccttagtcatttccccgctccagcctgctcccgcccgagcgcgcactcacggccgctctccctcctcgctccgcagccgcggcccatggagcccgccggcccggcccccggccgcctcgggccgctgctctgcctgctgctcgccgcgtcctgcgcctggtcaggtaagcacccccccgctccccaccgctgcactccccaaactgcgggctcaggcctctcagactggcactgggaccccttcggctaatgccgagcagtaatagggggagagacgcctgtaaccagggttatagatgcagaaactgaggcccagagagggccagcgacatgcccaaggtcacacagccaagggtagaacccaagtctcttctatgagagtttgtctctgtggactctgactctgtgcactgtgctgggcactgagacaaaagagccgagttgcttcactcagcccctgcccggtccaatggggaaggctgactgacagtggctttcagagcagggccgtccggcttccccagaatctgtccccatggacatacgactttccacatgtttgacttcagggtgtgaggggaggaggtgagggcagaggtcctcccgatgctgcctggaatggaacagagacaagctgttgcaattagggaagagtgagccctccctctggtgcgcagagtaaaccggtgttagtgtgtgcctgggagagagggagccgggaggccgcggtaaccccagagcgagctgctgcgagcaacagatccttatgtaaaaacacggtggattgtccctgtggttgccaaagcctctccctggtggtcttgctccttgaggcgctcccagctcccccttcctcctgccttccccgagcttccaggcagctccctgcagttcaggtgctccgcggggatgttacctgtggctctgaccatgtctttctgttggcctacatcaaacacctggctgtcgccggcaggcgggctctgaaggctactgtgcatgttcaccaaagggtgaggtcaccattcaggagtcttgcttcttaaatgggttttgcaaaaccatgggtctcctgggagggcaccgggtggggggcggggggagcagagaaatggcattaagggagttgggagggagagaaaaatggggatcttgttttttgtcgtcttgaaactgtgttgtaggcagtggggtctctctgtacctcccacccttgaggagggaggtaaagatgagctcagtgtccaacacccccttttgcgtttgactagagatggtaaccatctgatagcagggagaaagggagaactagcaaagagtgggtggaaggagaagtatggggggtagacagctgcttctctctgccctgcagtttcccctgaaacccaggaattcacataggcatccccccaccccaccccaccctccacccccagttcttttcagttatagttgcgtccttgctgggctgaagggagcactgatacagttcacgtctctccagtgagtggcatctcctatctgctccttcctgcctgagctggccatttgtaccctgcaaaagtccgagttcctgccctgccatccttcctggcctggaaatgcacaagtgtcactcacaagtgcttaattttcctgaagcagggaattgggcccgagttaatttctaaatgtctattccagacccttctccgaccaggaggctgtggttcagtccctggcaaacgagcagtgacgctgattagcaagtgctccgatgggcagcctgcctttcctggggcaccggctgggaacaccaacacctttcttgtgagcacatctcccccttttgcctataaaaacagtctataagatggtaaagattgggccataaaacttccgttccaatccattcagcatcaatagcctgaaaaagtgggagcagcagaggggccaagattagctttgaaataaataaatcttaggaggtaagaatgaatttcttggtcgagggtggtggggaagggatgaggtgcgaccatcatagctcaagtctcccattatccttcagtatctgttcttcggggtcctggtcacaccatgggcctgggcagggaagtcacctggtttggctgaaggatggggcctcaggcccctccccctattgctccagacatggaatttacaacaaagaagtagagagacaggatatgagggtaggaaggtacaaggaggcagttgtccaattttctaagaaaatggatttgttcgagtgagaaactaaaatgtcaggtttaggaaggcggctttggattgaatgactactagtgtctcaggcagaactgtcagcctgggaggtgtcacatgattcctgtgtcttcacatgatcactgttttgggggggtggggccaatcatgtgaattatcctgggtgggggaggcaagagaagaagtttctaaagattgcttttagcacaaatgctgccaaagggaaattctaaaaagatcccagtgaagtcaccaacctatgtttttggacccaatgtgaggcctgtttcctaaaacaactgcactgagggagattctgaggctgggagaagggcaagggactgaggcctggggacacctggcacctgattttgtgtcagcagggcctgacgctgaacccgcaggatgcctgcttggccccaggtaggccttgagacctctgagtcaccccccaggccgacctcagataaactcccattcatttcttctctctggcctggaggaggccaagccaagttgcagttcaggctctggggagcagcggggccccccagtgaacctctcaccgtttggtagagggtggcactctgggggctcctctgtactttcctaaatggtaaagttaatctcacttcctctttcgtaagaagagaaaacggggcgggagtgctttggctcaacacatcaggctgttggaagttcctttaaagccatagttttctctgagtactttctggcctttaccaacccctcggaaactcacggtaattcacgtctgtacttgcagtcatgcagcacctatgcagtgctggtgtgaaccatgtgttgagagaagggaggaaaaccaacattcattagatagttaatggcgttccaggccggatgctgctcctgcgtcgtctcacccgcaagacatggattggctcttctccccattctgcagatgcagaaaatcgagtcccaaactggggaactaacttataagtgatagagaggagccaggattttggatgctggccttcctgctgggtggcttctagggaagaagctcaatgacttaatcaggatgatttagtccaagctggagagggagtgagagaggcccctttctggccttgggtggaggactgtagaagagttctttctctgggcagaaggttttgatgaagtcacaggcatttctctgaaagcctgctgagggtggaaatactgacagccccttggtcctcaccagtaacaacacatgagaggtgatgtggctcctgtgagagccccccatctgggctgctgagctctggagcccccagagctggaatattcctccctgcatcttcctggtgccctttgcgacagatggagagggggtgtgcgagaggaagggagtctaaaagtgttgtacggatcccacacgcgctcgcttgctgcttccaatggctccgtgcgacaggttattttcttatttgtagtcttggactgacggaaggtttcccccctctctctctctttttaaatgtgctcagatttctggcctgtcaacacaactcagatcatgtcactccccagctcaaaactgttgagtgggtccaagttttctttaaaaaaaaaaaacaaaaaaacacaaagctgccgtgaggcctgggcttcctccctgccaggcctgatttccagccacatgttttccaacaagctgacctgttgagatgctgcaggataaccaggcttcctccctgcagaccagagctttgctggctactcagccacacgagcaccccaaagaccctgagaagccctgcaatgtagaatcccgagctgggtcacatttactccagtgttccctgaacttatgaacgagaggcatttgatttgtagtatgcccgttagcatgtggcctctctccgtttggtaaacggttttctacagatccatgccctcagctgaactctcagctctgtgccttggcccgtgctgttcctgccatcagaaatgccctccctgtacctccttgtcttcctgtgtctgaatcctactcatcctccgaggttcaatttaatgtgcctcctggggagaggtcagaggagcctctccacgcagacacacaccctggcctagcgctaagatggtgctaagacccctgggtgttgatctgagccctgctactgacttggacaatttgcaggaatctgtgtctcattttgctcatctgtaaaagaggaatggtattaaccactctattgcaaggattaagttaattaatgcgtgtataatgctggaagccatgccttgtatgtagtaagcactcaatacatattaaccagtgttgtaattagcatggctttattctggtttgattcctcttttttaacaattgggacggattttcataaaacatctaacacagttgcttacacatagggggtacttaaaaaacagcaaaggctgttcagtgaacaaatgtccttcagcttgatattttccttgtagctttttttttttcttttttttttttttgagatggagtttcactcttgttgcccaggctggagtgcaatggcgtgatctcggctcaacgcaacctccgcctcctgggttcaagcgattctcctgcctcagcctcccgagtagctgggattacagggatgcgccaccatgcctggctgattttgtatttttagtagagacaaggtttctccgtgttggtcaggctggtctcgaactcctgaccccaggtgatccacctcccaaagtgctgggattacaggctgagccaccgcaccacccagctgtccttgtagcttttatgctgttgggggatggacaagttgagtagaaacccaggctagagtgatttaattgcctgttactagggactgagtgctgtgggcacagcctgcctctaggtgtctctatctggatccacaggtctgcagttgtggattcccggacctgcaaagcagtagggggcctgagagagccctctgtgctttcatccagtgccttatggtgcggccagaagaacagagagaaaaaaactggccgggcccacgcgggagatccatggcaaggcccggctggaacgccaagcctcctgtggtccaggccaaggcttttaaaaactgggctgtgctggaactttccactgtttcagccagaaatctgacaggatcccgaattccccgggaaaacactaaagtaatactaataggaaccatctgggttctctgcttcctgggctcagcacttggtgtgtgtgaccctggtggtactcacagccacctggcgatgcacgtcgttatcttcggacccgttggatctgtgaggaaactgaggctctgaatggtgaagcagcttgcccaaggccacaccgtaagcgccagatgtggataaccactgtcggcctaatcacagagcctatgtggttaactgttgtgccattccttagccgtcactgaggcttgcttgacaggtagggaaactgaggcccagttgtgctggcaccctgctcccttagtgctcctctcatttttgtttttgttttttcttcctttctttctttctttttttttttttttttttttttttgagacggagtcttgctctgtcgcccaggctggagtgcagtggtgcgacctcggctcactgcagcctctgcctcctgggtccaagcaattctcctatctcagcctccccagtagctaggactacaggcgcacaccgtcacgcccagctaatttttgtgtttttagtagagactgggtttcaccatattgatcaggctggtctcaaactcctgacctcaggtgatcctcccgcctcggcctcccaaagtgctgggattacaggcgtgagccactgcacccggcctcctctcgtatttctatcagtgcaccttcctatgaggtgggggcagtgtgtgtccccaaggtgtgtccctgtccccatactagagaaggaagcatctattttcacagctgaaactgctttcctgttaactgctggtgttcctgggatgggtattctgatgaagccctccacgagatggattttccctgccaggtgtcttgccaggccctcagtccagagagatgcaaaacagctgaggtggaagtcggaaatgctgcagaattggttctttgtgtctgtaagatttcctggcacattcgttcgggggcgcagggtgttagatggctgcagattagcgccaagagcccaaggagatcaggtgctcggatcaggagggaaattggaagccgggagtgagcgatgggcctcccagccaggagggacggctggagcaggtgggcggtgataccagcgccgtttctacagctccattcttcaggccgaccgtcggtgttctccagaaatggaagcccgtggtggaaggaaatactaggtacacgatgtgctcggcacaggctgagccttgaacatgtgttagctcttccttatctttttattttggaaaatttcaaacatatgcaaaatgagaaggaatgatgtaatgagcctcagctttgacactgatcaactctataccatgcttgtcccctgcccctatttttgctagggagaggagaatcggggagtatataaagcaagtgctaaacatggggtatcatttcacccaccgatactctctccacatcccttcatgtgagctgttacctgaccctcaccacgagccaaacagtatggcccttcatccccactttacagataaggcaactgaggcttcccaaggccaagtggggctcatccttaggccacacagccagtgagaggtaaagccaggctccactcctcactttacccttaaccactttgcagtcattattagggtcattttaattgtaaatattcattcaacaaatatttttgagcacctactatgtgccagggatggctctaggtgcttggaggatacaaatgtgaacaagacagacaaaaacacctgctctcgtgcagcagacattctagtgaggaagagaaaaacacacccaagtaaacaaatgatgccaggtagctggggagaaaagcaggaggagggaagggagaaggttagtcaggacagagggagggccgctctaaggagcagaggcctcaagaaaatgtacaattaaggccaggtgcggtggctcacgcctataatcccagcactttgggaggctgaggtgggcagatcacctgaggtcaggagtttgagaccagcctggccaacatggcgaaatcccatctccactaaaaatacaaaaattagccgggcatggtggcgtgcgcctggaatcccagctactctggaggctgcggcaggagaatcgcttgaccccaggaggcagaggttgcagtgagccgagattgcaccactgcactcccgcctgggtgacagagcaagactctgtctcaaaaaaaaaaaaaaaaaaaaagaaaagaaagaaaagaaaatggaggagtagccacgggggatagcaagagccctccatgtgggaggaatggcagatgcaaaggccctgaggcaggaatgagcttgtatgttccaggaattgcaaagaggccagtggggctggagtgaaagtagaagaggtgggggagtgagcagaggtcagggccagaccagcaggatctggctcattgagcacctaacatggagcatgtctggcttgtgagaggatcattatgcattctctcatttaattctcaccacgggttctgtaagatgggaaataccgttaatccattggacagatgaagaaactgagactcagaaaggtaaagtgacttgcctaaggtcacacagcttttatgtgcagaactaggatttaggtccctgaatgctgcagcatccccattgctgctttccgcagccgtccttgcccccttcgggctcttcacccatagcagctggagtgaatcaagtcatctttggttctgctggaaacctttttcggtttccagcatggcatttctggaccacgtctgtctcttcatcctcacctcttaccactctcctgcctccttgggcttcagccttggcagcgtcctttctggcccttcaccagcttctttcccacccgagggcctttgcactggctgtctcttctacgggagcatcttccctggtcttcacttcctttgggtctcagctgccacctcccttccttggatgctgtcccaaccaccccattccaaaagaggttatcgcttgtgattctctatcactgggccaaatctgcctgttgcctagtgctcctagccctttctatattaattcatttcattatttcagccctgtctcctccactaggatataagctgcacgagagatgggctatgtctcttactcactgttgggcctcccaggacctcagcacagggcctgacacacagtaggtgggttaatagatactgattgaatgaaagaacaaggtgggtactaaaatcccccttttaaagaggaggcttggagttatgaggttgtacgagcaaagcttattccctaactaccacagtcccccccctgcagccagggccgggatcataccacaattccctgcccatcaagctgtcatgaatagccctaagatatcaggaagcacaaagatatcaatgccaggagggataggaggaggcggccaagaaaccccggaatttgtacttaaaattctggctacgcaaactgcttgcccaactttagacaccatgatgaatgatcaggtgctgcaaccatgaaacatgacgtaattagtgctgcactcttggctgggggttggcttggaggccccacctcgaggttagcctcccccagagggtcactgcacgaggcagtctggtgcggggtgggggttacaacagagggaggtggaatcagaccactcggcagccctgtgaccatggccaaatggttgtacctcccaaagcttcacatctggtcctccgtgaaatgggggtggcgacagccctgtcttactgggctcctgcagaggttccatgaggcctgtcttgctctgggcagcgtcgatgcttattagctattgttactgctgttatgtgattgtcatcgttttatggtttgggaattttaaaagctccatcttttgccagcggggagacctggaacccactcctctgccccctcagcctcagtttccccttctgtgaccgggaggcttggctcggctcctctcttgcaggtcccttccagccctgatgctccagcctgcatgtcctcctggccctttgtcctgatgactccacagactcctctgggaggagaggccatggaggggcttctgctctggggcacattcctgaattggagtccagcatgtgccgcttactggctgtgtgctctggggcaggtgtcttcccagctctgagcctccgtttccccatctgcaaaatggaaccgctaacactgtttcctcccaaatgggtgactgttgagggttaaggaattaagagccgtggaaagtgcttttgcaagctgcaaaatgctctataaaaggaatcagtgaatagtgtcagcttggggaaaaatcccgtcagtccggagactgtctgctgccggcagtgtgctgaaaccctggcaaatgtcgtttaataaatgcgtgttgcttttaagagggcctttatctcagctgggaaaatgtgtgcgcatccttggcaacctgagaatccagaagcctgaactggaggggacctgtcacagggagaggcgatcgcatggggtggctccagcctccagtgttggaatcccacctctgccacttacctacctctccagactctgcttcctcatctgtaggaggggacagtgacaatagcacctgccactctgggccaggatgtcctgagggctaaatgaaggcttctggttatctggcctaacccatgcatttcccccatgaaagtgtggctgtgcagcaccagggcctgtctcagggcaatggttggggcctggagattgggaggacctcccaacctcagcctctccaggccttactccaggcctcagtttcttcctctgcaaaatgaggaggaggaaaggtccttaggttgtcaaggcaccaagcctagtagatgtcaatgtcctctacctgctctctcagcctctgccggtcccccgaagcctgttaattctaaaggatatttacacagatttgcagttcttttcctgccactttgcaaacagggtcacactgcaagcatcgcttcatcacctccaggtttcatctgaacccagcaaatgctccatagggctcccagtgccgcctgtccccgctgcctccttgccagcagtgtgctcagcctgcttacaggcccatgccagaggggcctcatgcatgcatcaaggcagagctggttctgcctcggagaggatcccgagcctctcaggcttaaggacgtggtcatctgtcgcccccttcatctttagacacccatctgcatggtgacagctgctctgcattgagtacttactctgtgccaggcacgctagcccgtttcatcctcagaacaaccctagtattgttatcatcatcatcatcatcagacccattctacagataaagagccagaggtccagagtgggcgagacattccctggagtctcatgggtagcagagttggaatccaaatcaaaacatactttcagccagtgccaggcactgctccaaccctggggaagaaaactcagttcctgctctcaaaggacttataagtgagtctatgacagggagatgagcgcttttaaaaaattaacaaggaaatgcgtagctttttgagtgaggatgacatatcgaacaatgaagcagagccagggacattgaggtgggtagtattttagacagaatggccaaggaaggcctctcagataggcctgaaggagaggagcgagccagacatgtagatatttagggatgagagtgccaggcagaggcaatggcgagtgcaaaggtcatgtggtgagtggagaatctggcacgattgggggaaagcaaggctgtaggcccctggggccactgtaaaaattctgggtttcactctgagtgcagtgggggctgtgggagggttttgcacaaaggaggaacgtcacctgacttaggttttagaggcttctctggctgctgggggaatgtggctggtagagaggtgaggcagaggcagggaggtcagcgtggaggttcagtcagggtggtaagaagaggtcagggtctggccatgtgttggaagttgagccagcaggatctactaacagatgggttgtggaacacggatgaaagggagtcaaggaaatgtctcattctgagtccagcacttctaaccacagagcctctgacctggggagggagggggctgcatttcctgtttacaggtgaggaaacaggcttagagaaaagcaactttgccaagcaccgtgctacagacaagacataaaggtcatctggccccagaacctgagctctggacagtcttagcaccttagaagtttacctactgcatggatggggccaccagagtctgccccttccctgcagggatgtggcacagcactgagttctcagagctacccagggcaagggtgaaggagtggggaccctgagcccctgggccagctgtcagcttatttaccaggccacacgcatccccatttttggtggcttcaatgcaggtagcagtttctatcacagcacctggtttgtccctgcccaatgggtgtatccagtggtgccggcagaatgttcccagtgctggcctgctcttcagatgggcaaccgaaaccatccttcagggcgctaggttagcccagtcccaggtcctccccatctcagcttccttacagccagtgagcatgttccaaggactctaccctgagtcctgtcacctctgcgtctttgctcatgtggttccctcctcttggaatgccttccttcatcccagcctctgtcagcctttaagacctagctcaggggccaccagctccaggaagtcttcctgggtttcactctcttccagaaccccatccctaaacctcctcttgcaacaccgatcgccatgtgcgtggtcattgctcatgtccaggacggccccacatgctaagatagtctcaagggcaagggcctgtcttacttggctctgttttctgtggtatctaggacagggcctggcagcagagtgaggtttgagagggcatgaatgaatcattctgactccagaggaaatcactgtgtagtcctggctgttggagccccaagctctcttcttgggaactgtggcatccatgcagccatccactggtgcagccccggccatgaatgcagccccagctacctttcgccttcctgcatggggtcatgggctccagcagatggttctgggctagcctgttgacagctgtgccctgtggccagcacatagtaagtaggtgctctggaaaggttagcacctcctatcaagggcacgaccctagacagataccacacagggtgttgaccgccaggagcccatggcagacttgagggaagtgtgtctcctcacatcctccatcacccttgtgcttgtggaaacttacacagcaacctcatctattccaggcacatgcccagctggacacactcccgaacacaggcacacggacacctggggtgcacacaaatagtggcatgtgcatacctccctgcttgtcaatgcacacaccctcatagtcacatttacgcatacagcactctgtcacacacgtgccaccccccacacacaccctctacacacacatacacaccacactcacgtgcatgccgctcctgcatcactctcccacacactcacaccctcccacacatgcccaggccaccatgattcagacactctcctgcacacaattcacatgcccacgcccatgatccctagccccccattcagacacagtcctgtgttgcactctgacatattccggcacacaggcaccgtctctttcacactcctccttgtgcccacccatgtgtacatgtgtgtgcagatgcttatgctctgtacacgcatacatgtatgtacacacgtgtgttctcgtgtgcatactcttatgcaccctcagcacactcatgttcacatgttccattgtacagccctgtactacatcctgtgtgcatccgctttaatgtacactcatgttcacgtgttctattgcacagccctgtactatatcctgtgtacatcctctttaatgcactcttaatgttcacatgttctctgcatagccctttattacatcctgtttgcatccgccttaatgcacatttatgttcacatgtgctattgcaaggtcctgtactacatcctgtgagcaccctctttaatgcacactcttgcactgcatactctggcacacagcattaaatcacacccagaagatagtagaggcagcccccgatgaggagctgctataagtgcggggtggaaaatcgcagccttctgaggatggtgaataacaataatgatgagggaagaagggagccgggtatcactgcagccactttagaatgttgagccctgtgtactgccagagctccagccatagcctcccagaccctgggggtccttgagtacaaacaaagaatctacaaagaatccaaacaggtggcctcctgcctgtccctgtggcttgtgctgtggtgacccactgcttccgggggatgggcaagctcccatgcagtggcagtgagcacccgcctgggcaaggctggttcttgggaaatgtgcctgttggcttccttctccccaccccttgagacaggcttctggaagactcacaccccagggtaaggcagccctccccatccccgcctcaagcttcagttctaagttgatttttttttctattagaaaaaacatccatgtgggcagggcgcagtggctcatgcctgtaatcccagcactttgggaggctgaggcgggcagatcgcctgaggtcaggagtttgagaccagcctggccaacatggtgaaactccatctctactaaaaatacaaaaattagccaggtgtggtggcaggcgcctgcaaccccagctacttgggaggctgaggcaggagaattgcttgaacccgggaggtggaggttgcagtgagccgagattgtgccattgcactccagcctgggtgatgagagtgagacttcgtctcaaaagaaaaaaaaagtatccatgctaatttttttcaccctttatgaaatattttcatcttttaatctcaaaagccacacacatgaacattgttttttaagaaatcaaatatattttctaaatcccagtggtcagttctcagcccccatcttattgcatctctcagtggcattcaatgcaggtggtcacaccctccttggccaattcaaagaagagaaaggagatgtggcgggagaagcaggtcacaggaaggggccgggagatgggatctaagatgccagcaggagcctcttgcccaggggggttaggtacattccagatgtaatcatttgagtgatttctttgaaatccatctttgcctcgagggtggaagctccatgaggccagggacacagccatttcacagcccttagctcagggcctggcacagggaaggatccagaatgcagcactgaccgcacttgctgtggacactgagttcctcttacagcaaacatatggtgaccacccactgtgctgactgctccccttaggaaacgtgttcgtgtgttttgtttcattactttttaacttttttccaaaaccaataaatgttcactgcaaaaaaatacaatatatgatgtattataaagtaataagaaaacatgccgaccccatcctacctcagtcccatttcccagatgcaacacaccacagcatgccatgtttaacttatcatccttgcagacctttttctctagacatgcgattgaccaatcaacaaagatttatcgagcgtctacactgtgccaaccacagtagcaggagctggggaaacagtgaacaaaacagactaaatccctggaggagaggttcatttaaacagtacacatacggtaacatacatcattacatgtacaggttttttaaaaaacaaaatgagatactataaatattcttttgtgttcatccaatttagagaagctgaaacaatgcccaaagtaggaggggaaaaaaagagaaaaacctcatgaggtttagattcatgatccaaagagagttacattttatagctttgtttaataccctccagttgtttatatatagttttattttacaaagtcatgatcatacagagggtatagatttttctccatctttttttttcttacaaaagaaatgcaaacagaaaacataaacaagagcaaaatcccatgtaaatctaccactcaatcatggctaatctttaatatcattgtttttttctaaaattgggagtttgggatcacactggatctactacttggtaacagactctgatcttgtaacacatttccattgtaattctataccatcattttcatggctatagacttggcctatagctgtgaaagtagattcaccataatgggatcagccaatctcctttccttggatattaggatattttcaattttttattttcatacagacacttaagtgaatgaagcaaaatgtctgcacatattcttagttacctaaaggtatgatgttagattgtttttcttcactcatatttttactacatcagaatgttgattacagttatctttgatggtatgatgtgagattattactttgtttccatacttattttctctaaatgccttgcaatgaacaggttttatttgcataaacacaatattatttaaaatgatgccatgaaaatttctatatgaccaccgttttcatggtaatggcttgttacggctgcctgtaattccacataggggataggccgtaattcacttaaccatcccctatcattgaccggctgggctttttctaatgtctttaagatcgttatacaggaaacctactccatatctggggttacgtcctcagggagcagcccagcaaaggctggtgaacagatttcacacctttggttcatcctgccaagctgctttcccgaaaagatgttccaatttcccctcccaccggcagtgcgtgggaatgcctgcccagccgccctcctctccctgagacggttcaggcccctggacagtcgcatcctctgctaggagggcaagagaaagcaggaaataatttggccagcaagtgaccccagacccagagccaggagtgatttttaacaagcctgattctcagaacagaacctgcagagaaaaagaaagcaagcaaggaaaaaaaaaaaaaaacccctctctactggaaaaaacatacacatacacagattaaaataatatttggcacttacgtcgcacttttaaaattcttcaaacgctttataaacactagctaattaattaagccgcctaaatctggctgcagtcggtggagagagctcgagaggagctaaaaaaaaaatgggggagccagcgctacacaactccagattttattgtcgcctgcctggggtgtgattacaggcctccggctgcaccacaagaatagccctgctccaacatgctgcacgcctgactccgcagcctctcctgcgcctcctgctccctcaccaactgctgactcacagccccttgtggtgttattgataaggataataataactgcaagccaggctgagtcccgggctccgggcctgcaccctcccctggatatgccagtatcaactccacctaacacatgaagagagaggagatttgcccagggtcatggagccaacgacatccttccttccttcattcattaccgcattcattctgcatatttttatcgatgacactgtgttttaggcaggaggttggaaacagtcccttgtggatcctacatcctactattgggaatgtgacaatccagagaaaatcaaatatgtgaataatacagctatgtgataataatgggaataagaattaaaacttacccagcacctatgcagcaggcataccaccgtgtcagggtcctcatgactgtgctggggtggatgccggaaccccacttcatggatgatggaatgggggtccagggaaggtgggtacagaggtggctaagaggggagccatgctgtcacccaggcctgctggccctagcaactgtgcttccccgaggcagcctttccagttggtccagtctcgaggctgaaatacagacctatccggaaagttcctagctcagtgctgctggtgctgcctctgatgtccccgcctggatgggcctcccgggcctcccacacatttggccaccagcctcacccctctcaggccgtctgatgctggctgggcctctgtccactgtgggttctgtctgtccactgtgggttctgtttttttctcctgtgccatgaagatcttgccatgttcaacttcaaatggaaatgaaatgagaagctggacttcctgataataaaagcagttgaggtcttaagtggttgatcaggaaaaggaagtcgaaatggtaatatttacacccaaagcaggtggagtggtcttaggcactctgagactccactcacctgccatgagaaccgggccccaagggtgatcccttgccagagtcaagactgggtgaggctgggaatgggagaaagaggcaggacaatgggcagatagccctgggcttcctgcagtgagggcagataggggtctgtgagcctctttccctggctgccctgcccctgccagtggggtggtgccaggccatgggttggtgggcaaattcacagggtctctctcctccccagccagagccagaggggcacagcagctctggacatctgccgcagccttgtctgatgtacaaggcaaaggtggcccaggtcagggcaggacaaggtcacatgaagggtcagtggagagcccaactggggccccaggtcacctgtcacctgcataccaggtagctgccactcctctgtctacctggttctctccttctcccctattctagctcgctctctcactctgcgtctgtctctcaggcatgtgtttctctgaggtctttatgatcatgatctttcttttttttgtttcttgtacctcaccttgttcagaatcgaggccctggctctgccttgctctgaactgttctcacttggtccctatccatcttcaacgcatctcatgacaagctcgtcctgccctcttcctgtctgggctccccccgtgcaccccaatctctccacccagggtccctgtcttgtgtgccccagtgtctctgattggggctcaggctcctgcagccaacctctacaattccttccagctgcccatttgggagcccttgaggcctggactgggtccagctccagtgtgtccccatcacagtgaggcctggatcacagggcatcagcaggtgttggtgagagtgaagtttgagccacagccacctccttgtttattcaatgtatagtaacaacaacttcctcagggcccatctcagggctaagtgacttctatgtgtgatctcgttttcttttcagaacaaagcagaggcctgggggctgttctacccatcctgtagataagaaaactgagactcaaaactgatggataaattaaggtcccacaagaacaaggggcgagccaggtttcaaaccaggtccgatgactctaaagaccagatgctgcatttggacgctgtccccagccttggcttattttatcctcccagagatccatcgaggtggggtcatagtaactattttacagatgaagaaaccaaggctgagagcagtaagggcaaactaagggcagctcttgcatttgtgtgacactttgctagccaggctcagtcctgggttccaggtggggactcaaacacactggcacgagtctacattggatattcttggttacctgagtggtggcccaaggacacagctataggccactgatccagaactcaaactacattctcctactgcccaaaccgctgagtacagcctcccttctcggggcctgcatcctgggccaggccagaaacagaatcttaacaccttgtacagccccagatatgccaggcccctacctctgagtctttgcccaggctgttccctctgtctggaataccaggctcccttctggctcattcagattagaattagcccagctcagatgtgactcttccagagagggtccaggcattcaaacccactctctgccatgtcatttagctttctgtgcctcagtgtccccacttagaatacaggctcatgttgcaggtttgttgtgagggtcaaatgagatgatacatgcactatactgatctcacagcctgcttctggtgtgcatccagtcaatgaacgtcattgataaacacttgaggaaacacagaggatcacgtaaggatgaaaaaatgactgctttgtgctcctttccaggagtggcgggtgaggaggagctgcaggtgattcagcctgacaagtccgtgttggttgcagctggagagacagccactctgcgctgcactgcgacctctctgatccctgtggggcccatccagtggttcagaggagctggaccaggccgggaattaatctacaatcaaaaagaaggccacttcccccgggtaacaactgtttcagacctcacaaagagaaacaacatggacttttccatccgcatcggtaacatcaccccagcagatgccggcacctactactgtgtgaagttccggaaagggagccccgatgacgtggagtttaagtctggagcaggcactgagctgtctgtgcgcggtgagtacagcgtgggcctcctttgcctctggtttgtgacagtaactcaataataacaccctccattctttgagcaatgatcaggtgtggtggtaggtgctcctttccatgaattgatgtctcccccttttacaaataagggaagttgaggcctgaagacgtcatgctatttgctgaaggctccacagctggtaaagggtgggaacatctgcatccttcatctgcctggctgtgtagccctggcctcttcaaagctgacccagccctttcattccacagatgaggaaactgacagatagtgtgacttgcccaggcacaggaccagacctcacctgctgcctccagagagcacttccctctcagggcagccgagtctcctctatgtgacaggcactcactgagaacctactgtgtgccaggctccgccaagcttgtgttctcatcccttccaataagagatccacccagggcagggagcagagagggtgtgtttcctttatttcatcaccaaacaccaactgagaagattcctaaaaggcacagatttactgtttaatctgatggccctggtgagaggagaagccagagggttgggcattttctgtgggtcttttagaaaaacacttgtgtgatttagggactgattcatgcgtcactaaaggctctgaacaaagctgggtcatcatagaatgtctctcaccccagggaggaggtctcccttctaggtgactttgggttcctccgttaaggcagcaaagatgctcattttgtatttactgccagagccccaggatccttgttctaactccgaatcctgggacattagtacaggaaggaccctcagaccatccagcccaacccctagttacaggtggaaacttgaagtcaggagatgacaggggccttgtccacagtcataggcccagttagtgacaggatctggaccagagcccacatctgattccagggaggctccttcattagccattgtttacatagaagagcctggtgcatcataggtgccgactgaaagctccttgaacagaactgctaagcaggaagccagcacttctcctttccatcagtttcctaaaggttcagaatattcacattcagaccgttaggcccactggtgattggaaaaccgctcccaaatgtaagccatatcatttgatactgagatttcatatcaggatagagacctgctccattgtgtaatagctacagaatactccagcttatggaaataccagatttattttaccatcttccattgatggatacgtaacttgtccccaaacttgaactattaaaacagtcctgcagtggatgaccttatacatgtgtgaccttactcaggtgggagacgcccactgcaagtgccattgctagatcagagggggtgtgtgttttaatgtgtgtggatatggcctaatttccctcctagacattataacaatcaagtctccccacaaccgctggacataggcagggagggtggctttatcctgattgtgcagatgttacaggaaaagacgttgaagttcccagatgatgcatctcgtgcctagacaatcacagcaaatacgtggctcaggtgtgacccagatctgctgactcctagcagagtgctcactcctctttgccatgctgtcagcctaaatgatgccctggatgagaaaggagggaggtgggagggggccagggagcagcagctggattggatcctcatcctggcactttctccttttataacaagtacttatgaaggaagagccatttcttgccccctttacagataaggactctgaggctcagagaggagacctgtgtggtgagccagtgggggagccaaggtttgatccccaccagcctgactcacagcccctgtctctgtaacactcagctatgcttgtcaaagtgtggagaaatgctccaccctactcattccaaactcttgcagttctatagcaataagcataggctgaggcaggagaatgcttgaacccaggagacggagattacagtgagccgagatcgcaccactgtactccagcctgggtgatagagcaagattctgtcttgaaagaaaaaacaaaacaagtcctatgggagtaggagggactggcccccatcagaaaggtcctgcagatgtctcatgatatgggaacttgttcctaaaggacaggtctcagcacatcactgagtagcatccatggagcagagttgggcctcctgcagcccagcagaccacgtgcccacctactcacggtgctgaggactaaggatgaggagatgaggcatttcctggtgggtgcttggggctcattggagcaagagtttatattatcccctcaggctgtcaggagcccactgacttcagtttatggctatagggtaaccagatctgggtaccctggagggaggcttctccctagaactgcatgaagtcagggatctcaaagggaaaaagggaaggtggtggtcagtgtcagggttcatgaagttcaggcacttgctcaattgattgacatgagagcttggggaacactggaaatgcccattgggtacatgagggatgaggagacagtgccttcgcttttctcagcctcagtttcctcatctgtagaatggtgataatcagggactcctttggcagagtggaaaggatttgagttcctgtgtataaaacacccaatgcagagtagacacccccaaatcaaggttttttgtttgtttgtttgttttttctttttttttgagatggagtttcactcttgttgcccaggctggagtgcaatggcaccatctcggctcactgcaacctccacctcctgggttcaagcgattctgcttcaacctcccaagtagctgggattacaggggcacccaccaccacaccagctttttttttttttttttttttttgtatttttagtagagacatggtttcatcatgttggcccggctggtcccaaactcctgacctcaggtgatccccccacctcggcctcccaaagtgctgggattacaggtgtgagccactgcgccctgccatcaaggtgttattttaagatgtgttaccttgggcaacccagttccccttgctgagcctcagtttccccatgcgtagaacgtcgataattgtaccaacctttccaggatgttatgaggatgcaatgagcctggggcgacgatagaccttagtaagcaatggggttttcccaaggtcatgtgatcttgggcaaatcctggcttcccttgagtcctcagcttccctgtctatgaaatgatgatgatgattgccaaccctcacaggttctgtgaggattggagaggatgtacagagcctcggaatggtggcggtgttcgggttatcactgagctcctgcagaagtgtggttaggagagtgtggccagagtccaactgcctgggccacatcccagctctgccacctcctggctctgtgacctgggacaaggcactccaccaccatgggcctcagtgtcctcacttgtcagtgataacaccaacctcacagaccaggagttgggaccgaatgagttaaatcctgtagcgtgcttagggcagcaggcgcctggcctgcagtcaatgttcaacagatgtgagctgtgtgatgctgaagccatgcaacctcagatgggttagtgctcctccaggcctcaaggataaatggtccttaggatgtactcaggtagttttcaatcttggctgctctttaggatcatctgggaaacattgaaataaatcccaggctccaccacagaaatgcctcttcaattggcctggcatgggcctaggcatctaaagtttttaattaatccagaagattctaatacagagtcaggactgaaaacccctgggatagacacccagaggtcacggaggacaagttggaaaagatggtcctcttggggtgccagatgggagaacagcttttcagggtgtggatgcttctgaacaacgtgcacccgggacagctctttggaggccccagtggcctggccagaccccctcttatgatacaggcctcgggggccaaggtaggggccccagacaaagccctgctcagatccagcctcagtgtggggaaaccttccatcctgaaaatgtttttgtctaaatgaccgccggaatcccagcaaatccttccaattcggccccagaaccctccctgcctcgacacaccaagatgtcacggaggagtctgttccctcctcctttaaaatgcttccagcttctgtaaatggctcttctggagggaggcgcctgtgatcttgtgttttgaaagccaattcgccaaacttgtttcctgaggggattgtttagaaaaggggagagaatcctcacttggtggtttctgctccttgtatcatttgggtttgtcatgagccacagggagcagggtttgagccttttcttggtgtttctgctctgctttgttctgctgcctgaaggataaatgtgtagattcggaagcagtaatgaccagacctcctgataacagtcacaggggtcatttattaagcacctgctgcataccaggctctgtgcttgcattcatcctccctcttcttaacagggtggggtttgtatccacttttggccctgggcctctctgagctttgtcctaaaaggggctcacacgaggatcccctcacagagatggagtgaggattaaagagctgacgggcatggagggagcagccagtgtccagctccagagaagccctggataaatgggaactgattgcagcccctcagccctgtggggacatccaggccgctggcctcaatgacttgacttcttcaagtgacaccctccttactgtgccctcaaaatgtttgatgggtgtgagggagaagttaatatttacaaatgcaagtatgtgtgatatgtgcttcctggtgactgcgataggaggtatggggtaatgattagcaatttggagacacgtttacgacccatctgcagggagaaacagcccgcgttctgattcagccatgtgtgtgctttcaggacccaggaagtgcttctgcctgcttgggcgccctgtgtctgtgtaattaattgataattaaccactttggctttctccacacttggtgatctttagtgtctatctaaaactgacaccaaagaagaccgcagattcagaaaagtatcccttcctttcaaatttagcacttggcacagattagaccctgctggaatatttgtggagaaaatgaaaaatatgaagtaaattcataaatacctgaagagagagctttggatatgaataaagtctgggttaaagttagatctgtttttgcttatttttgtgaagcatcttctgtgtttatccaaagcaaaactgcttctgttgcctcatttaatcctcgctgcatcccagaaaggagaaatgctgctgttccattttacagacaaggagctgggtgtgccaggccgcccctctcatgggctgagcggcctggttccagctctcatggtgatgccacgttgctgcttactccctgcactgacctctataaacgctctctctgaggttggcactgttgccaggggaggcagggtagaaacagttaataccaaggatgagatggatcgttaattttgatcctgtgcccaaattgctggaagtcaccctgtgtggagtggagaagtttgaactcaggcctggacttccctctgggccctgcccctacactgtctgacctcacatgggtcctcacctctcccaacctttgcatcctgctctgtaaaatgacaataaggacaccgccctcattaatccttccacattattggaaggataaaaaataatgtctcagaaggttcttaacgtgtcacacacttatcgttagtgattgtcatctgagtcatgtctcctgattatcgatggtccttttgtagccaaaccctctgcccccgtggtatcgggccctgcggcgagggccacacctcagcacacagtgagcttcacctgcgagtcccacggcttctcacccagagacatcaccctgaaatggttcaaaaatgggaatgagctctcagacttccagaccaacgtggaccccgtaggagagagcgtgtcctacagcatccacagcacagccaaggtggtgctgacccgcgaggacgttcactctcaagtcatctgcgaggtggcccacgtcaccttgcagggggaccctcttcgtgggactgccaacttgtctgagaccatccgaggtagaagaccctcacccagcccaagcccacacctgaccgccaagcccaccccccaccaccctccactcatccagctctactcttgctccagggcttgaaatgcctggaacctaaattcctaactgcccacctcccatgcccctagatgtgccagccacttactaacattttaatggcagtggctgggtgaccacaccacccaccaggtgccaaggactgtgctagccgtttcatttatttcactgatagcaaacacagttaatgagtacctactatgaactaagcctctatgtgcttggtgattttctttattttgctacagttacaacattccaactgcatgccaggggtggtgcttatagatttccctcattcttagcctagtaggagctaccagtccttgagcacctactgtgtgctgggcgttgtgcttagtaatttcattcatatgcaaagagcaccctcaatgtctgagcacctgatgcctgcctagctctgtcctggtgatgacctcaccgtggtgggggagctacgttatggagccctccctgtgatctgtctgtgccacaaggtcagagcttctgccctgtgctgtttcagttccacccaccttggaggttactcaacagcccgtgagggcagagaaccaggtgaatgtcacctgccaggtgaggaagttctacccccagagactacagctgacctggttggagaatggaaacgtgtcccggacagaaacggcctcaaccgttacagagaacaaggatggtacctacaactggatgagctggctcctggtgaatgtatctgcccacagggatgatgtgaagctcacctgccaggtggagcatgacgggcagccagcggtcagcaaaagccatgacctgaaggtctcagcccacccgaaggagcagggctcaaataccgccgctggtgaggcctctatttcagctgacccagcttttttaaacttttagttttgtgggttttttaaatgttaaaagtagtaattcatgcttattatagaacaatctagaagtctataaatataaagtagaattttaatgtcagctccatttccccaaatcccacactccaagagtcaccactggtaagagtttgatgcacatcttgatagatcttttgacatagatttatatcacaggaggaaagagggagagagggggcccatctgttcatggtgtcctgcctcttgccttcttcacctgacagttgtcatgctccccattcctcactggcccagcccctcttccctactctttctagcccctgcctcatctccctggctgccattgggagcctgccccactggaagccagggtggagtcattcagggtgtttgcagcttgtctcacatacagtcagtgcttcagtgaacatccttgtgcatgttccatcccacatgtgactatttctgtgtgattgcccaaggaacactttaggccaaaatgcatactcatgtaaggaggagataatgctgtcgctcccctgagagagtgtgagggtgtcaaggtatcattttctcagcccttgcaacgtggtgttggtgtacatctcttttgttttcaaatggtggcacctgattggcatttcagtgcagccatggaatatttcatcacctgttatcaatgtggctgatgccctgtacatagtagaacccttgaatggattagaatggaagccatttctgtccttcatgcacccttcagggaagctctcaaagacagccttggggctgtgtcaaagtggccctttggtttcaccaatgccaggttttcttaggatgtgtagggcatcggccaatgctgggttttcatagtacgtgtggagcattggccaatgccgggttttcctaggacgtgtagggcatcggccaatgccaggttttcatgggatgtgtagggcattgggcaatgctaggttttcatgggatgtgtagggcattgggcaatgccaggttttcatgggatgtgtagggcattgggagacaggatgtgtggctttccacgccagctatgttgcatacttggctgtgtgaccttggacaagtcagtgcctctctctgggcctccacttgcttctggctgtgttgaaggggatgagcttcatgacccctcagctctgagtctcagctggtaggggccatgattatcttgctcagcattgtagcttcagggcctgaccacagtaagcactcagtaaatgctttttgagtgaaagaggataaaagagtcagttggttggctggttggttggttggttggttggttggttggttggttggttggttacataatccttagttagccaatgaggtagagatgactgttggccccagtaacagaagaagcagccagggtttggagggaggcttaactgacttgtccagggtcacacagctacttagtagcatagatttgaaagcacttttctgtggcttagagccctgcttctgatgaaccactgtgttctagtctatgacccccttacctgtgctgtgggcagtcaagcctccattgtattttcaagagtcctggttcagagcccctatgctaactcctgagcccagtgtgcatggcccaggtgctgggcacagaccggtcgtcagcccctgagctgtgcagagcctgctggccgcactgcccttcttccagctgctgattcagtttccagtggatctggggtgccctcatgaccgctctgtggttcactccagatgagaaggaacgtttctgttcctcaggagtgagatttctgcctgtatcctgcagcaggggttcctccgtagctgtcagctacaaatatattcccccctggcacctcagcatggtttttgtgctgttcatggatgagtctcgtgggcaagtgtgtacagacccatgagtgtgcccatgtggctcgagacatctaagaaggtccagccagatgttctcagttaatgatgcctgcttagtggtgaaaagcagtggtgggtttggttttctgtttttaatctgcatacgtgaagcctctattccatgtggtccctagagaacactggatctaatgaacggaacatctatattgtggtgggtgtggtgtgcaccttgctggtggccctactgatggcggccctctacctcgtccgaatcagacagaagaaaggtgggtgcattcccctcttcctccctaagggtttgtccctggactgtcctcggagggagacgccattaggtgctttgggttaaggacatcagcttctgccagtagcaagaagtccagaggtagtggtgctagggctggggcagtggcctcaccatgttagagtctgggtgggtcactctgtggcttccccctcatggttacaagatgttcatcactgccccaggccccgtgtcctcaaggggccactactgagcaagggagctgccagcctgggaagagaggaggcagggactcttcttacctgtctcagggaggacatcgtctttcccaggagcctcccggcagacttctgctttcctggtacaagtctaggttacatgaccagtccctggacaggaggccagagggaatggagataatagcgccaccatttgattgggaggacttaacaagtgattgcatgggaaatgttagggaagtaacctggcacacaagtgctcaatgtatgtcagccctgattattacccgtattgtatgtgagcccagaaatatgagccacctccctagaaagccccctcacagggcaaggtgcatagcctttgactgcacagcaggagggcacagggtcaaacccaggacaccgcttccccttgtttgtgtgcacgtgttacgtgtgtgtctgtgtgtgtgtgctggttgttgggagggttggttgttgctgtggcagggcctctgactttgaagtcagacaaaccagggttccagaccctggttatatactgtaaacaatcaatttggcctctacaagcctcagtttccccatctgtaaaatgggtttagcaacgcctgtcttggagaagttacaagatccaaataagcccatgtgaaataacgggtctgacacattctaggatcgccataaatacatgttctctttcccactcttggagaagacaattttaagctggtatttctgcaacctttttttattacaagagatagaaatacatttcacatctcaacccaacacatacacacaccctttgtatataaaattaaaactgatattttcttttttgtcatattcttttttctgtttaaaatgctggttgtgacccaccgcctccaaagggacaagtgctgtcccgagctgcctttctgtggcctggcacagctgagcttggttgggcacattgtacactaatcacgcatcccttcagtagctccttcacctctgcctcccaagtttccctccctcctgggctggctctgagatactggccaagcctgatgcttctgtctgggagggttggagtcaggccctgcttctccagaatctgcaagacgtcccaccaagacgtctacccaatgggttccctaaagttcctggagagccccacacagggaggactcctccttggatggggaagcaggtcccttccccaccaagggtgacagcccatcccatgggatgaagcattcatagcagccgatagtgaaatcctggccatcctttctgagtctcaggcccaggcagcacgccagctcctggcacacgtgagctcattactgtgggcttaggtgttgcagaggaatggagtttgcagggctgcagctctggtaaaggccacccagcccgagtggcaggtctgtgtgcagagcctagctctcaaacacaggctccagtgacagggacggctagtatcccccagcagttccaatgtgccaggcactgctcactgtgctttccctgtagtaacagtcagtgctcaccacagacctcatgggtgacattactaaccccactttacagatgaggaaactgaggcatgggaaagttataaaacttgttcagggcctcacagccaggaggtggtggggccagaatttaaacccaaggaccatgtctaggaccaccttgcacactacctccctccgccataactagaccccccttctccctcattaggccctggaggggtctgcccagcctctcaatgctcacagtgaaatgtcctgatttcaactcaaccactacccagctgtgtgaccctgaggagtcacctagtctctctggtgtaaaagggggtgactggtgtcctatcactggattcccagagatgacttgtgtccagcactttgcaacctgtcagcctccactcagactttcggtgctgagcacagcttccaactggtacacctcatctaaagatccaggacctgcccaggcagcaagcgttaacccagcagcaagaattcttgcactgtggcagcctccagcctattaaagtgcggtgcagagtgcatttgggtgtgcatgttgctttttaattacaattttacagcaaaatgggagtcaaacggctgctgaaccctggaggcttctctgtgggttaccccatatcacaggtttaaaacctctgaaccagatggacactttctttccttccaagatcctttttttggaaagtggtggcatgtaaataaatggatgctcagcccaaccctgcaagttatagaagctgagagagaggaagtgacccacccggggtcacatggtgagtgagtgggtagcagacccggggttcacacatgatccccgatttgcagccctgctagaatctgttttcaagccacgaagggcactgattgggacctaggcttgttggagggtctccgctgaggtttgtggttgaatggaggtgagacccagacagaggtagggcggttgtcgcctcccaggctgaaggctgcttttggacccctggcaacttccagaagtggaaaagcaggaccatctagcttactccttcccaggcttccttggtggaagaggatgcccactgggtgggcatgggggtctcctgtggttccaaggatgtgattacagcatttcctctccatgtccctggaggcaaaccttttgccaaaaaatagttacataagaaaagtgtgcttctagttaaacaactggcttttgtttcttttgtctttcagcccagggctccacttcttctacaaggtaagtgcatcattggtccagaccctcttgcagttattatttggttatttgacagccccccagactacaaagcataatccatgtccactgacctcaccaatgtgttggtcaacatgtctctttctctcctttgtaacctcctcacactgggtcacgctgtttttaattattgtcccaaatgaggggtttttttggtgcacagaggtgctaatttcttgcctgctcttcccagacaacgagacccttgtctaatcccatgtggactggggcagagacctcccgggctttctgtcctcacagacatcctgcacacgccaccggatgcatcctcccagatccaagttgtatactttactctttcctttgtttagtaaatatttattgagtaccttggcgtgctgtgaccctggaatgccgggatgagtaggatagaatctgtgccctctgcaagctcacagcctggtcagaggctcagacggtgacattacatcactgatgtacgtcaccgatggcactttagtttgttgattcacatttttagtgacttgagtccctactatgtgccagccctgctcaggaccagctggtgatatagcagtaaacaagaatcagcccaggctttcatggggctcagacgccggtgagagaaacaggcataaacgcagaaataaagtgattcatggtgtcatatcaggtggtgacaactcctaccctgcaaggagatggaggtgaatggtggggggctgtcttaggcagaggaagggactgcctctgaaaaggggatgcaaacgggtgcctgaaactagtgagctatgttggacaggagcatgctgggccagagaggaggcctgtgtgctgggccagagggaaggagaggaagaagataaggtcaggaagacccaggtcctcggcatcccggtgtcctttgcagatgcccctgcagtcagaggtgttacgaactattctgtgaaaagcctttatcggctttttggaaggcctctgatcttcatccccagcagagcagaggcagcacaccatcatctgccatgtgaacccatctgcatagaaccatgtggacatccgtgctaagagtgataaataaaactataaatagcttcatgttggttcacaccaggttttgccagcaactgcatagtgccaatcttttgatggtttagagctttctgggtttcagaattacagaataggacatgatgatctggagtggggcctgtgggtgttagaaggactcgggattttatccgcagtgggatgaggaaccaaaagatggacacgatctgacttttgttttaaaagtgtcgcctggctacatgtggagcccagactacaggaggtgaggatgtgtaggtgaccatagcggtactggtccaagcagaaaaagatggtgacctggaccaaagtggtgggatgggctgagaagtgctcagattctggatatattttgaaggtagacacaagaggacttgctgatagattggctgtagggttaaggaccacctcaaggttgggggcagtggagtgctgtatcctgagttggggggtgcaggtgatgggtggagtgatggggcaaatatcaggcaaagcagttgcatatagcaggagcagctcttctgccagagaaggctctgggaagcatcaggtgccccgctcctgcccagcagtgtgagtccaaggtactcttgggataaagccttgcttcttgctctgatctccacagggggcctcctggtctgagccctgcccagcctcctggccttttcctcccctgaacaagcagagccgcctctccagcaaaactaggctcttcactgccttctgggcacatgtgttttccttttgtcatgcttgtgcctgtcacactcccacacctaggtcactcttccaagggcaagactcgtttatatcctttgccaaaaaaccttcagtggctctacctgtccctccagataaagtccagatgcccccctgaggctcctgggccccttgtggtccagttgccttgaacagtttcacttctagccaccccctgtcctcacatttcacggctcaaccacactgtgctccccccttccctcctcccacagtctgccctcctcccccttgcacactgccctcactgctggggtgtgctccctgcccctgctcatgcactacccagggtcccccagctccaggggtggcaggcacagtgggctctgtgaatggcaccccctggagttatgcaatgtggtagctagagtgggacccctcctgtcacctgctcacctcctgcttctcctgagatcagcaagggcacgacatcctccagggtcggagggcttcctgtgaaccctcctggtgccgcagcgtggacaccctggtgtgttcaaggtgtctctgcacctgtcccatttcataacccacccctcaaggccagggccaatgtccctgaggttcactgcaaatagctgctaaacttggcaccatgcctggaatgggataggtgcttagtaaaggtttactgactgaacaagtgaacaaacgaatgaatgaatggatcagtgcataccatcctaaaccactgtagcacgtcccactctgccttctctgaatccctaaaatgctgcttcccagtggctacacagccccatctaggaggatctctcttttttgttttttttgttgttatttttttattttttatttttgagacggagttttgctcttgttgcccatgctggagtgcagtggtgcgatcttggctcaccacaacctccgcttcctaggttcagatgattctcctgcctcagcctcctgagtagctgggattacaggcatgcgccaccacccccggctaattttgtatttttagtagagatggggtttctccttgttggtcaggctggtcttgaactcccgacctcaggtaatccacctgcctcggcctcccaaagtgctggaattacaggcgtaagccactgcgcccggccctgttgtcattattttttacttgtcttacctccccgattagacaatgagtttctggaagaataggacattgtcgataatagtaacagcaatagcagctaccatttattgagcacttactgtgctcctggcactatctatgcctttatgtgtattaacccacttgtgattttaattcttgcaggaaccctatgatttatcactgtcccaagttcatagaacagtgtttttcaaacttgagtgtgctacagaatccccaaaggacttgttaaaacacagattactgggccccacccccaaagtccctgattgagaatctctatttccacaagttctcaggtggtgctgatgctgttgttccccagccacactttgggaacccctgttctagaaggttcttattccctatcctatggttgaggaaattgaggctcagagaagtcgtgacattcccgaggtcctgcggctaggaagcaccggaatgatgacttcagcctcgacatctcttgtactaaagctagtgccttcatcactgttcccatgcttcttgctgtctatttcttcttccccagagaaaccaaccgttttctgcctgttttttatttttccagcgtgagcctaccaagggagccatgagatcacttgctccctttcatctctctaaaccaaagcaatagctagtgagtgctaatggtggtttcccatgtagcagccagcttcggctttggcttgctgggatcagacattttccagaggacgttggtgtgagtaacagctgagcacctcagggaggctgcaagcggggaggctgccactttgagctcaaggggttcttcaggttgcagggctggtggagggattggctctctctaactccctgggttaggaatgtacagacagaatattgtctgtaatcttccccagctcagatgagagaaaaatgtgtctctgctcccttaatggggcactgatcattcgatcagccatttattcacttgtcagacattcactgcatttattcttggaatatttagggcttccctgagtctttcatttattcgtcgggactgagcctaactcagtgccaagccttgagcagagggttgcaaaggggagcagaaaaggcccacccctggtctcatggcacttaagagccagtgagaaagattcattcattcattcattcattcattcattcagcaaatacatatggactgctgacgatgtgccagcccagttcttggtgctggggcttcagcagtgaatgagactggcccagttcctccctgtgtgaagctcagtgtccgctgaggagacaggttaccagacaagctggaaagttaataagggatgtggtgcagctgctgactagataaaatgaagccaggtatgggatagagcagatcaaggaggtggggtctggggcgagcagggagggcttgtctgcaaggtgacatttagaagatcactgaaggatgaggaggttccagccaagggaatatctgggacaagactgcaccaatcaaagggaacagccagtgccaagaccctgagacgggagcagtcagatgcatgcacagaacgcagaggaggccagaggagctggagcagagcagaggcaggagagggatggagcctagagcagttggccgagctgagccttggagctggtgtcacttgaccaccttggagtggttatagaggggttgagcattctgagcagaaaagtaacatgacatcacttatgcatttcagatagggagaatggagggcagggaagcaggaatagaggtggggagatcaaggaggaggttcttcaggaatcaggggagttcggatggtggagaggatggggtggctggctttgatggatattttggaggtggagccgacgagccctaggttggattagatgagagctgtaaaggaagagagagaagaatcaggactgactcccacttctccagcctgagcacaggatgaccgatggtagagctattcactgaaataggaaaatccaggggagacacagatatggggaaaatgataattggttattgtctgaaattgctttgaaagagaagaacccaatgttttgagaatggagtttgggggttcagtgaatatttccccaagaaaaggctgtttaaaaatagcatctgaaggaagaaaaggattaaagcagattaagaggcagcagcatggagcatgcaaaggccctggggcagcctgactggcattcattccaagtcactgtttttgagcacatgtggtgaggaagcatggcaggcaggcaggcagacagcaatagaaaccagatatgcagatagacaggagatctgagattgtcatggaggggcagcccagccagcgcagcactgtcagcagagccatcacaaggtgacaccatagttgagtcttgaaggaaacaggaaagttagccaagaggaaaaatgatcaaaggggtccttcgaggctgctattgaagcatgaagtacataacatcaaatgccaccccccccccgaaatatctggtgggcagatgagtgggtacatgagggcagggcagtgaattagcctggaggcagcacgtcagaccacgtgggactctgagcagggcgatgagttggacttgaaccagccagccagtgggaagctccaggagggctggaggccagtagtgtggggacagatctgcgtgatcttccccagcagacgtgcccctgctcctggagggttctttctgtggccacgtggggcttgtcatgtctcagaactgctttcagagccaagcactcatgccaaatccattaataaccctgctgccaagccccactgctcatttctgacattatccatgctaacatccccggggttgccggcttgagaatttacacaacttctgccaagccagtggctctggtttgctcatcaccttgaccgtgaccctccagtgagtcaagtcatgacctccaattacagaagcaagtgcacgaacaacttctttgattaacttggacatcttggtctttttcttttctttctttaatgatgatacatagatgtttataatacagaacatttggagaatatgggaaactaaaaaggagaaaaaaaataccatgcaaagaccactgcagtttgcattttgctatgtttccttccagtgttatccacacatttttcataattgagataatcctacagagaattttcctttttcacttatcattttaatgtaagcaattttaacttaaaaaaaaaacctcctcataaaccttttaaatctttatctgtggatttatcctaatttccctaaccagtcccaattggcagagccctaagacataaacctcctcccccatccagcccttgacaaatgcctttgtgttgaaagctttttctatattttgaattcccccaaaaaaaagtgcaattatgagtcaaaggatgaggacatcttccaagctcttgattttgccagctgcttcccaaaaggattgtactggtttgcctctcccaccagtgacgtatgaaagctccagttcatttcaccatagcttccccagcaccaagtattataatttttaaagatccttgccaatagagtaggttaaaaaaatgatagtgcattgcttagaattcctttttagtgagattgacccctttgtccaaacatttgatatgcagttgtatttctgttatgagtccttcgttcatgtcctcaacccatatagaaaatggagcctaaatgttattcttatcagtttgcatgagcacttgagatagtgagggtatttttatctgtgtgtctctttcctttttaggttgcatgagcccgagaagaatgccagagaaataacacaggtacagtccttggtgagatgcccttcctgggaaactccgtggcgtggttgcttcacatcaaccggaattgcagatctggttctaaattaagactccttgtggggtggagggtggaggatcttacactcctagctttccccatgtcctgtgcatattccttctctctactgcagccaagagtggctgttgcagcctcatgactcagtgcgacccattgcacaactccccaggggcaccattcacaatccgtcctatgaaaatggtgccctggagtcaggctgcagctcccttagtgtggatcgtgccttcccatctctctacatttgcccaggcccatccctctaccaggagcgccactcccagctcactgcccgctcctcacagccaggcttttctatgctggaactgcaaagcccagcctgccagggcctctcccacctttgctgatggaaagaccccatgtcattaattacctcacacaagggcacactcactcaagctggaggtttctggtcagcattgctgagatgattttgcagcaacacggtttctgagaaacctagccctgcttatcaccggctgatgctgtcagaccttgaaggcagagggctgctcagcatcttgtaacaagacaggccctgctctgaacccggaacgagagcgcgccagctgtccttgacatttccaaggcagttgtcatctttttagatgtaatctgttgaaggagtcgggctggtgaaaggcgcagcatttgtagaccaaaatgccttggccctgcccttgtgacccatggggtccccccgcagccccggacatgagctcgagttgtaattaagaaaaaacaatttccagtcatgagcgagggagatcttctcccccatcattttacttctggacttcctcctggaatcttctgggggaaaaaaatgatggtgagaagaatgggtcatggaaacctctacgaaattcacagcagagacttactgggcacccactgtgtgccacgcactggacagtgagccagacagtcacagcccagctggcagagtgcctgcgattccagtgccaggtgccagccctggccagggagggagttgcacgctgtcaacacggatttcccagacagaagcaaatgagagcacgcagggctcaggacagaggagctgtgtgctaagagcacagtcgtagagtccaacacactttggttctttgccagccggacaactttgtccactttattgaacctcccttggtctcagtgtcttcagccgtaaagtggaatagataatccttcccaagcagggttgtggtaagagtggatggggtgatctatgtgattgtgtgctgcttgtaagccctgagaactgaaatcagccattaccgtgcaccaactaggaggtgtgccggttggtggtagctttattctgttctatcacgcaggggccagtcacagagggagcaaagtttatcattcatgatgggcgcttttgaggcggcagtggcaggttgataggattagcacatgggaggtatccaagtttgctaagtggttaatgattgcgaacatttattgaagacagatgctcaataggtaccaggctctgttctaaacgctttacctggataatctcattccatcctcacagcaaccctggcagtagacactattatcatcccctctttacagatggagaaactgaggcccagagaggttaagtgaattgctcaaaatccctgagctagaaccaggattcaaacccaggctttctgactccagaacgtttcccgcttcactaactggcgatctcgttcttatactgcccctgttctctgtctccctgtccccagctttgccctgagtgaaggtgaaattattgccatccactcatatattcattcaatatggatcagacttcttatgtgccagccttggtttctgaatcataacaaatgcccagtgaatatttgtggaataaacgcatacattaatgaatgtccccacacgtaccaccatcagcagcacctcctcaggcccccaaacagacatggtcctgggtagcgggccctgtgatggaaaacaaacatgggctgaggaagcacaaagaaggaacatggaccctgccaggagaggggagagccagggagggtcttagtatgagccatgcttcatctgaaatttacatggtggattgggatttgttacaaggagaagcaggacagacaccctcgagggatgaacatgagaaatgggtgaagactgcaggtggttcaggctaggaggaggcaggaacgggaggaggtgacactgaggcggggctgtgaggagactgcactgtgcgggtcagaaagacccttcaggcagggtgaggaggagggcagatgggaaggggcaaggctggaggcaagtaacgttggcaagagatgaggggaacatgacttatggctgagccagtgtgggccgagaggacacagaagcatccagacttggtattcagtttgagtgagtgggccaggggctatagaatgaccttctcatgactttctctttgggtatcttaaatccaggacacaaatgatatcacatatgcagacctgaacctgcccaaggggaagaagcctgctccccaggctgcggagcccaacaaccacacggagtatgccagcattcagaccagcccgcagcccgcgtcggaggacaccctcacctatgctgacctggacatggtccacctcaaccggacccccaagcagccggcccccaagcctgagccgtccttctcagagtacgccagcgtccaggtcccgaggaagtgaatgggaccgtggtttgctctagcacccatctctacgcgctttcttgtcccacagggagccgccgtgatgagcacagccaacccagttcccggagggctggggcggtgcaggctctgggacccaggggccagggtggctcttctctccccacccctccttggctctccagcacttcctgggcagccacggccccctccccccacattgccacatacctggaggctgacgttgccaaaccagccagggaaccaacctgggaagtggccagaactgcctggggtccaagaactcttgtgcctccgtccatcaccatgtgggttttgaagaccctcgactgcctccccgatgctccgaagcctgatcttccagggtggggaggagaaaatcccacctcccctgacctccaccacctccaccaccaccaccaccaccaccaccaccactaccaccaccacccaactggggctagagtggggaagatttcccctttagatcaaactgccccttccatggaaaagctggaaaaaaactctggaacccatatccaggcttggtgaggttgctgccaacagtcctggcctcccccatccctaggctaaagagccatgagtcctggaggaggagaggacccctcccaaaggactggagacaaaaccctctgcttccttgggtccctccaagactccctggggcccaactgtgttgctccacccggacccatctctcccttctagacctgagcttgcccctccagctagcactaagcaacatctcgctgtggacgcctgtaaattactgagaaatgtgaaacgtgcaatcttgaaactgaggtgttagaaaacttgatctgtggtgttttgttttgttttttttcttaaaacaacagcaacgtgatcttggctgtctgtcatgtgttgaagtccatggttgggtcttgtgaagtctgaggtttaacagtttgttgtcctggagggattttcttacagcgaagacttgagttcctccaagtcccagaaccccaagaatgggcaagaaggatcaggtcagccactccctggagacacagccttctggctgggactgacttggccatgttctcagctgagccacgcggctggtagtgcagccttctgtgaccccgctgtggtaagtccagcctgcccagggctgctgagggctgcctcttgacagtgcagtcttatcgagacccaatgcctcagtctgctcatccgtaaagtggggatagtgaagatgacacccctccccaccacctctcataagcactttaggaacacacagagggtagggatagtggccctggccgtctatcctacccctttagtgaccgcccccatcccggctttctgagctgatccttgaagaagaaatcttccatttctgctctcaaaccctactgggatcaaactggaataaattgaagacagccagggggatggtgcagctgtgaagctcgggctgattccccctctgtcccagaaggttggccagagggtgtgacccagttaccctttaacccccacccttccagtcgggtgtgagggcctgaccgggcccagggcaagcagatgtcgcaagccctatttattcagtcttcactataactcttagagttgagacgctaatgttcatgactcctggccttgggatgcccaagggatttctggctcaggctgtaaaagtagctgagccatcctgcccattcctggaggtcctacaggtgaaactgcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtatatagttgcaacttaaactttttggcttgcaaaatatttttgtaataaagatttctgggtaataatgagtccttccggtgttcagtattcttgtctttgtgagtgcgtcccggggccgcctcggggcctgcctgccctcctgccaaagcctggaagaggattgaatggaccccagggtttggaaacaacctacagcatttgagcccctcacgtaggttttagagacgtacaatttttgtttgccctggctcagaaggagccggtgtaaggttgagataaaattccatatagacaactgagtttggatctcggctctgctgctttgtagctgtgggagttcaaacagcacctctttgagacttgggccccgcgtcggcacaatggggcaggaatagtccctgccagggtgactgtgtggattcaaggaggttggaagcactgagcccacgcttggcactgggtaggccatcactgaggcttgtgtctttccttcctgccctgaccctgcatagctgagtctcgtggcctctcaccactgatgggatctcggacacccggctcaccctcctcctggggcctcactttccttccccaccaaatgcagacatctaatgcttgtcctgtaatatccttgccagatgactgatgtcaagtgctggtttatagtgggtgctcaataaatgttagtttcctttctatccctccattagatcattaatgatattagtgctaattatgtaataaagttaaggcataaatatgttaatatttagtggttactatgtgtcaacgctttaccttgatgaattcttaaaccttgtgactcaagggcaattatgttactgactgtagatgacacagagagagggtgcaacttacacaggttcacacagctaatgagtgatgttactgagcctccagttccaactggataactgtagtgcctgatgcttagccactgggttaataatcaatacgtcatgtatggaccactgactgcaaaattaaccaccatgatagctaataacagctatgccaagtgtgctgccgcgtgcttcacagttagaactcatttccttctcccagctacatgagggaggaactcttgttctctgtgctttgcagataaggaaactgagacacggagaagtcatttctccaatgtcccatggcagagcagtttgaagccagggaatctgactcttctcccccacactgtacttactgcctcatttaattctcatgactatcagatcgctgtggttgtgcccattttgcaaagaaggaaagatgaggttctggaagtgcatgtgacatgaccaatctcacgcagagtctttagtcaagttgagacttgaactcagat

(19) United States
(12) Patent Application Publication
(10) Pub. No.: US 2023/0391868 A1
(43) Pub. Date: Dec. 7, 2023

(54) COMPOSITIONS FOR AND METHODS OF TREATING CANCER

(71) Applicant: The George Washington University, Washington, DC (US)

(72) Inventors: Alejandro Villagra, Washington, DC (US); Maria del Mar Gracia Hernandez, Washington, DC (US)

(21) Appl. No.: 18/297,565

(22) Filed: Apr. 7, 2023

Related U.S. Application Data
(60) Provisional application No. 63/348,389, filed on Jun. 2, 2022.

(51) Int. Cl. C07K 16/28; A61K 31/17; A61P 35/00; A61K 45/06

(52) U.S. Cl. CPC C07K 16/2803; A61K 31/17; A61P 35/00; A61K 45/06

(57) ABSTRACT

Disclosed herein are compositions comprising HDAC6 inhibitors and CD47 inhibitors and methods of treating cancer using such compositions.

### CROSS-REFERENCE TO RELATED APPLICATION

This application claims priority to U.S. Provisional Application No. 63/348,389 filed 2 Jun. 2022, which is incorporated herein in its entirety.

### REFERENCE TO THE SEQUENCE LISTING

The Sequence Listing submitted 7 Apr. 2023 as an XML file named “GW121_091019-758421_SL”, created on 7 Apr. 2023 and having a size of 218 kilobytes is hereby incorporated by reference pursuant to 37 C.F.R. § 1.52(e)(5).

### BACKGROUND

Cancer has a major impact on society in the United States and across the world as it is among the leading cause of death worldwide. The emotional, physical, and financial toll of cancer is devastating. While compositions for and methods of treating cancer continue to evolve and improve, there remains an urgent need to bolster the ability of patient's immune system to treat and/or prevent cancer and cancerous growth.

### BRIEF DESCRIPTION OF THE FIGURES

FIG. 1A-FIG. 1I show that HDAC6 inhibition modulated macrophage phenotype. Gene expression analysis (FIG. 1A) and cell surface marker expression (FIG. 1B) of A31A7 macrophages or BMDMs that were left unpolarized (M0) or polarized to M1-like phenotypes in the presence or absence of the HDAC6 inhibitor Nexturastat A (NextA, 5 μM). Gene expression analysis (FIG. 1C) and cell surface marker expression (FIG. 1D) of M2-polarized macrophages. FIG. 1E-FIG. 1F show immunofluorescence microscopy and Western Blotting (FIG. 1G) of M1 and M2 associated markers. Gene expression analysis of THP1-derived M1 (FIG. 1H) and M2 (FIG. 1I) macrophages. Scale bars represent 50 μm. *, P<0.05; **, P<0.01; ***, P<0.001; ****, P<0.0001; ns, non-significant.

FIG. 2A-FIG. 2E show that Nexturastat A downregulated SIRPα expression in macrophages. FIG. 2A shows the gene expression analysis of SIRPα in A31A7 macrophages, BMDMs, and THP1-derived macrophages that were left unpolarized (M0) or polarized to M1-like or M2-like, evaluated by qRT-PCR. FIG. 2B shows the luminescence intensity measured as relative luminescence units (RLU) of A31A7 cells transfected with SIRPα-luc reporter plasmid in the absence or presence of NextA. FIG. 2C shows the cell surface SIRPα expression quantification in A31A7 macrophages and BMDMs. FIG. 2D shows Western Blotting evaluating changes in SIRPα expression in A31A7 macrophages upon NextA treatment. FIG. 2E shows, immunofluorescence microscopy representing changes in SIRPα expression in BMDMs upon NextA treatment. Scale bars represent 50 μm. *, P<0.05; **, P<0.01; ***, P<0.001; ****, P<0.0001; ns, non-significant.

FIG. 3A-FIG. 3L show that HDAC6 controlled CD47 expression in melanoma cells. FIG. 3A shows the comparison of CD47 basal expression levels in SM1 and B16 cells by flow cytometry. FIG. 3B shows Cd47 gene expression analysis of SM1 and B16 cells upon stimulation with IFNγ in the absence or presence of NextA. FIG. 3C shows cell surface CD47 expression of SM1 cells upon stimulation with IFNγ in the absence or presence of NextA. FIG. 3D shows luminescence intensity measured as relative luminescence units (RLU) of SM1 cells transfected with CD47-luc reporter plasmid upon IFNγ stimulation in the absence or presence of NextA. Western Blotting (FIG. 3E) and gene expression (FIG. 3F) analysis of HDAC6 after performing a partial knock-down (KD) in B16 cells. Gene expression (FIG. 3G) and flow cytometry (FIG. 3H) analyses of CD47 in non-target control (NT) and HDAC6 KD B16 cells and WM164 cells (FIG. 3I). Gene expression analyses of WM164 NT and HDAC6 KD cells upon IFNγ stimulation (FIG. 3J), WM1361A cells in the presence of IFNγ and NextA at 2.5 μM, 5 μM, and 10 μM (FIG. 3K), and WM793 cells in the presence of IFNγ and 5 μM NextA (FIG. 3L). *, P<0.05; **, P<0.01; ***, P<0.001; ****, P<0.0001; ns, non-significant.

FIG. 4A-FIG. 4J show that Nexturastat A enhanced the phagocytic capacity of macrophages. FIG. 4A shows CD47 antibody titration in SM1 cells by flow cytometry. FIG. 4B is a schematic representation of flow cytometry-based phagocytosis assays. FIG. 4C shows phagocytosis rates of unpolarized, M1-like or M2-like BMDMs that were co-cultured with CFSE stained SM1 cells. FIG. 4D-FIG. 4G show phagocytosis assays of untreated or NextA treated BMDMs co-cultured with CFSE stained SM1 cells at a 2:1 ratio in the presence of isotype IgG control or CD47 blocking antibody (25 μg/mL). FIG. 4H-FIG. 4I show corroboration of results comparing BMDMs harvested from wild type C57/Bl6 mice or HDAC6 knockout (KO) mice. FIG. 4J shows flow cytometry-based phagocytosis assays of THP1-derived macrophages co-cultured with WM164 human melanoma cells. All flow cytometry-based phagocytosis assays are quantified as the percentage of CFSE+ F4/80+ or CFSE+ CD11b+ cells out of total F4/80+ or CD11b+ cells. *, P<0.05; **, P<0.01; ***, P<0.001; ****, P<0.0001; ns, non-significant.

FIG. 5A-FIG. 5C show that Nexturastat A enhanced the antitumor efficacy of anti-CD47 in SM1 melanoma-bearing mice. FIG. 5A is a schematic representation of combination therapy. FIG. 5B shows tumor growth of SM1 melanoma tumors (n=9-12 mice per group) treated with vehicle control, Nexturastat A (NextA, 20 mg/kg, IP), αCD47 (100 μg IT), or combination. FIG. 5C shows individual tumor growth kinetics in the different groups. *, P<0.05; **, P<0.01.

FIG. 6A-FIG. 6J show that the combination of Nexturastat A and anti-CD47 modulated innate immune populations in the tumor microenvironment. Immunophenotyping of tumors performed by flow cytometry, including total CD45+ immune cells (FIG. 6A), different macrophage phenotypes (FIG. 6B), T cells (FIG. 6C-FIG. 6H), and NK cells (FIG. 6I, FIG. 6J) with their respective markers. Cell markers used to determine immune cell populations are described on the y axis of every graph. *, P<0.05; **P<0.01; ***P<0.001; ****P<0.0001; ns, non-significant.

FIG. 7A-FIG. 7H show the validation of macrophage phenotype results using other HDAC6 inhibitors. FIG. 7A show confocal images of A31A7 macrophages stained for CD68, a pan-macrophage marker, to corroborate their identity. FIG. 7B-FIG. 7C show gene expression analysis to assess concentration-dependent effects of Nexturastat A (NextA) in modulating macrophage phenotype. FIG. 7D-FIG. 7E show gene expression analysis to assess the effects of Tubacin and Tubastatin A in A31A7 macrophage polarization. FIG. 7F show Western Blotting of A31A7 macrophages polarized to M2 to compare NextA, Tubacin, and Tubastatin A. FIG. 7G show cell surface expression of CD11b, CD14, and CD16 in THP1 monocytes and THP1-derived macrophages after PMA treatment. FIG. 7H shows gene expression analysis to evaluate the effects of Tubacin and Tubastatin A in THP1-derived macrophage M2-like polarization. *, P<0.05; **, P<0.01; ***, P<0.001; ****, P<0.0001; ns, non-significant.

FIG. 8A-FIG. 8E show HDAC6 inhibitors regulate expression of anti-phagocytic and pro-phagocytic signals on macrophages. FIG. 8A shows confocal images showing SIRPα expression in A31A7 macrophages that were left unpolarized or were polarized in the absence or presence of NextA. Scale bars represent 50 μm. FIG. 8B shows gene expression analysis of Sirpα expression in A31A7 macrophages treated with Tubacin or Tubastatin A. Gene expression analysis of pro-phagocytic signals upon treatment with NextA in BMDMs (FIG. 8C) and THP1 (FIG. 8D), or upon treatment with Tubacin or Tubastatin A in THP1-derived macrophages (FIG. 8E). *, P<0.05; **, P<0.01; ***, P<0.001; ****, P<0.0001; ns, non-significant.

FIG. 9A-FIG. 9B show CD47 gene expression analysis at different time points. Gene expression analysis of SM1 cells (FIG. 9A) and B16 NT and HDAC6 KD cells (FIG. 9B) stimulated with IFNγ at different time points.

FIG. 10A-FIG. 10F show the effect of the combination of Nexturastat A and anti-CD47 in B16-bearing mice. FIG. 10A shows tumor growth of B16F10 melanoma tumors (n=15/group) treated with vehicle control, Nexturastat A (NextA, 25 mg/kg, IP), αCD47 (50 μg IT), or combination. Immunophenotyping of tumors performed by flow cytometry to identify macrophage (FIG. 10B), T cell (FIG. 10C, FIG. 10D) and NK cell (FIG. 10E) populations with their respective markers. FIG. 10F shows the comparison of total CD45+ immune cell infiltration in B16 and SM1 tumors. *, P<0.05; **, P<0.01; ***, P<0.001; ****, P<0.0001; ns, non-significant.

FIG. 11A-FIG. 11C show the gating strategies used to perform flow cytometry-based immunophenotyping of tumors. Gating strategies used to identify multiple macrophage (FIG. 11A), T cell (FIG. 11B), and NK cell (FIG. 11C) populations in tumors harvested from mice after combination therapy.

### BRIEF SUMMARY

Disclosed herein is a method of treating a human subject having a cancer, the method comprising administering to a subject in need thereof a therapeutically effective amount of a HDAC6 inhibitor; and optionally administering to the subject a therapeutically effective amount of a CD47 inhibitor. Disclosed herein is a method of treating a human subject having a cancer, the method comprising administering to a subject in need thereof a therapeutically effective amount of a CD47 inhibitor; and optionally administering to the subject a therapeutically effective amount of a HDAC6 inhibitor. Disclosed herein is a method of treating a human subject having a cancer, the method comprising administering to a subject in need thereof a therapeutically effective amount of a HDAC6 inhibitor; and administering to the subject a therapeutically effective amount of a CD47 inhibitor. Disclosed herein is a method of treating a human subject having a cancer, the method comprising administering to the subject in need thereof a therapeutically effective amount of composition comprising a HDAC6 inhibitor and a CD47 inhibitor. Disclosed herein is a method of treating a human subject having a cancer, the method comprising administering to a subject in need thereof a therapeutically effective amount of a HDAC6 inhibitor; and optionally administering to the subject a therapeutically effective amount of a CD47 inhibitor; wherein the cancer is treated. Disclosed herein is a method of treating a human subject having a cancer, the method comprising administering to a subject in need thereof a therapeutically effective amount of a CD47 inhibitor; and optionally administering to the subject a therapeutically effective amount of a HDAC6 inhibitor; wherein the cancer is treated. Disclosed herein is a method of treating a human subject having a cancer, the method comprising administering to a subject in need thereof a therapeutically effective amount of a HDAC6 inhibitor; and administering to the subject a therapeutically effective amount of a CD47 inhibitor; wherein the cancer is treated. Disclosed herein is a method of treating a human subject having a cancer, the method comprising administering to the subject in need thereof a therapeutically effective amount of composition comprising a HDAC6 inhibitor and a CD47 inhibitor; wherein the cancer is treated.

Disclosed herein is a method of slowing and/or prevent disease progression, the method comprising administering to a subject in need thereof a therapeutically effective amount of a HDAC6 inhibitor; and optionally administering to the subject a therapeutically effective amount of a CD47 inhibitor. Disclosed herein is a method of slowing and/or prevent disease progression, the method comprising administering to a subject in need thereof a therapeutically effective amount of a CD47 inhibitor; and optionally administering to the subject a therapeutically effective amount of a HDAC6 inhibitor. Disclosed herein is a method of slowing and/or prevent disease progression, the method comprising administering to a subject having cancer a therapeutically effective amount of a HDAC6 inhibitor; and administering to the subject a therapeutically effective amount of a CD47 inhibitor. Disclosed herein is a method of slowing and/or prevent disease progression, the method comprising administering to a subject having cancer a therapeutically effective amount of a composition comprising a HDAC6 inhibitor and a CD47 inhibitor. Disclosed herein is a method of slowing and/or prevent disease progression, the method comprising administering to a subject in need thereof a therapeutically effective amount of a HDAC6 inhibitor; and optionally administering to the subject a therapeutically effective amount of a CD47 inhibitor; wherein the cancer is treated. Disclosed herein is a method of slowing and/or prevent disease progression, the method comprising administering to a subject in need thereof a therapeutically effective amount of a CD47 inhibitor; and optionally administering to the subject a therapeutically effective amount of a HDAC6 inhibitor; wherein the cancer is treated. Disclosed herein is a method of slowing and/or prevent disease progression, the method comprising administering to a subject having cancer a therapeutically effective amount of a HDAC6 inhibitor; and administering to the subject a therapeutically effective amount of a CD47 inhibitor; wherein the cancer is treated. Disclosed herein is a method of slowing and/or prevent disease progression, the method comprising administering to a subject having cancer a therapeutically effective amount of a composition comprising a HDAC6 inhibitor and a CD47 inhibitor; wherein the cancer is treated.

Disclosed herein is a method of improving and/or enhancing a subject's innate antitumor immunity; the method comprising administering to a subject having cancer a therapeutically effective amount of a HDAC6 inhibitor; and optionally administering to the subject a therapeutically effective amount of a CD47 inhibitor. Disclosed herein is a method of improving and/or enhancing a subject's innate antitumor immunity; the method comprising administering to a subject having cancer a therapeutically effective amount of a CD47 inhibitor; and optionally administering to the subject a therapeutically effective amount of a HDAC6 inhibitor. Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor. Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor. Disclosed herein is a method of improving and/or enhancing a subject's innate antitumor immunity; the method comprising administering to a subject having cancer a therapeutically effective amount of a HDAC6 inhibitor; and optionally administering to the subject a therapeutically effective amount of a CD47 inhibitor; wherein the cancer is treated. Disclosed herein is a method of improving and/or enhancing a subject's innate antitumor immunity; the method comprising administering to a subject having cancer a therapeutically effective amount of a CD47 inhibitor; and optionally administering to the subject a therapeutically effective amount of a HDAC6 inhibitor; wherein the cancer is treated. Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor; wherein the cancer is treated. Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor; wherein the cancer is treated.

Disclosed herein are HDAC6 inhibitors for use in one or more disclosed methods. Disclosed herein are HDAC6 inhibitors for use in one or more disclosed kits. Disclosed herein are CD47 inhibitors for use in one or more disclosed methods. Disclosed herein are CD47 inhibitors for use in one or more disclosed kits.

Disclosed herein is a pharmaceutical formulation comprising a disclosed HDAC6 inhibitor, and one or more pharmaceutically acceptable carriers and/or excipients. Disclosed herein is a pharmaceutical formulation comprising one or more disclosed CD47 inhibitors, and one or more pharmaceutically acceptable carriers and/or excipients. Disclosed herein is a pharmaceutical formulation comprising one or more disclosed HDAC6 inhibitors, and one or more pharmaceutically acceptable carriers and/or excipients. Disclosed herein is a pharmaceutical formulation comprising one or more disclosed CD47 inhibitors, and one or more pharmaceutically acceptable carriers and/or excipients. Disclosed herein is a pharmaceutical formulation comprising a disclosed HDAC6 inhibitor and a disclosed CD47 inhibitor, and one or more pharmaceutically acceptable carriers and/or excipients.

Disclosed herein is a pharmaceutical formulation comprising one or more disclosed HDAC6 inhibitors and one or more disclosed CD47 inhibitors, and one or more pharmaceutically acceptable carriers and/or excipients. Disclosed herein is a pharmaceutical formulation comprising a composition comprising a disclosed HDAC6 inhibitor, and one or more pharmaceutically acceptable carriers and/or excipients. Disclosed herein is a pharmaceutical formulation comprising a composition comprising one or more disclosed CD47 inhibitors, and one or more pharmaceutically acceptable carriers and/or excipients. Disclosed herein is a pharmaceutical formulation comprising a composition comprising one or more disclosed HDAC6 inhibitors, and one or more pharmaceutically acceptable carriers and/or excipients. Disclosed herein is a pharmaceutical formulation comprising a composition comprising one or more disclosed CD47 inhibitors, and one or more pharmaceutically acceptable carriers and/or excipients. Disclosed herein is a pharmaceutical formulation comprising a composition comprising a disclosed HDAC6 inhibitor and a disclosed CD47 inhibitor, and one or more pharmaceutically acceptable carriers and/or excipients. Disclosed herein is a pharmaceutical formulation comprising a composition comprising one or more disclosed HDAC6 inhibitors and one or more disclosed CD47 inhibitors, and one or more pharmaceutically acceptable carriers and/or excipients.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5 and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration of a disclosed HDAC6 inhibitor, a disclosed CD47 inhibitor, a disclosed pharmaceutical formulation, and/or a disclosed composition. In an aspect, a subject can be a human subject. In an aspect, a subject can have cancer or cancerous cells or tumor cells. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, geriatric, adult, adolescent, and child subjects, as well as fetuses, whether male or female, are intended to be covered.

As used herein, the term “diagnosed” means having been subjected to an examination by a person of skill, for example, a physician, and found to have a condition (such as cancer or one or more tumors) that can be diagnosed or treated by one or more of a disclosed HDAC6 inhibitor, disclosed CD47 inhibitor, a disclosed pharmaceutical formulation, and/or a disclosed composition or by one or more of the disclosed methods. For example, “diagnosed with cancer” means having been subjected to an examination by a person of skill, for example, a physician, and found to have a condition (e.g., one or more cancers and one or more tumors) that can be treated by one or more of disclosed HDAC6 inhibitors, disclosed CD47 inhibitors, disclosed pharmaceutical formulations, and/or disclosed compositions or by one or more of the disclosed methods. For example, “suspected of having a disease or disorder characterized by cancer” can mean having been subjected to an examination by a person of skill, for example, a physician, and found to have a condition (e.g., cancer) that can likely be treated by one or more of the disclosed compositions or by one or more of the disclosed methods. In an aspect, an examination can be physical, can involve various tests (e.g., blood tests, genotyping, biopsies, etc.), diagnostic evaluations (e.g., X-ray, CT scan, etc.), and assays (e.g., enzymatic assay), or a combination thereof. In an aspect, an examination can be objective and/or subjective.

A “patient” refers can refer to a subject afflicted with a disease or disorder such as one or more cancers or one or more tumors. In an aspect, a patient can refer to a subject that has been diagnosed with or is suspected of having a cancer. In an aspect, a patient can refer to a subject that has been diagnosed with or is suspected of having cancer and is seeking treatment or receiving treatment for the cancer. In an aspect, a “patient” can refer to a subject afflicted with cancer. In an aspect, a patient can refer to a subject that has been diagnosed with or is suspected of having cancer or one or more tumors. In an aspect, a patient can refer to a subject that has been diagnosed with or is suspected of having cancer and is seeking treatment or receiving treatment for an inflammatory disease or disorder.

As used herein, the phrase “identified to be in need of treatment,” or the like, refers to selection of a subject based upon need for treatment of cancer or one or more tumors. For example, a subject can be identified as having a need for treatment based upon an earlier diagnosis by a person of skill and thereafter subjected to treatment for cancer or one or more tumors. In an aspect, the identification can be performed by a person different from the person making the diagnosis. In an aspect, the administration can be performed by one who performed the diagnosis.

The term “ex vivo” refers generally to activities that take place outside an organism such as experimentation, modification, differentiation, manipulation, and/or measurement done in or on living tissue in an artificial environment outside the organism. In an aspect, ex vivo experimentation, ex vivo modification, ex vivo differentiation, ex vivo manipulation, and/or ex vivo measurement can occur with a minimum alteration of the natural conditions. In an aspect, “ex vivo” can comprise living cells or tissues taken from an organism and cultured in a laboratory apparatus, usually under sterile conditions, and typically for a limited duration of time (e.g., a few hours or up to about 24 hours, up to about 48 hours, up to about 72 hours, up to about 96 hours, up to about 120 hours, up to about 144 hours, up to about 168 hours, or more depending on the circumstances and/or the desired characteristics. In an aspect, such tissues or cells can be collected, frozen, and later thawed for ex vivo treatment.

The term “in vivo” refers generally to activities that take place inside an organism.

As used herein, “inhibit,” “inhibiting”, and “inhibition” mean to diminish or decrease an activity, level, response, condition, severity, disease, or other biological parameter. In an aspect, “inhibiting” can refer to diminishing the intensity, the duration, the amount, or a combination thereof of symptoms, complications, issues due to a subject's one or more cancers and/or one or more tumors. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having one or more cancers and/or one or more tumors) or to the level prior to the onset of one or more cancers and/or one or more tumors. Thus, in an aspect, the inhibition or reduction can be a 10%, 20%, 30%, 40%, 50%, 60%, 70%, 80%, 90%, 100%, or any amount of reduction in between as compared to native or control levels or to the subject's level prior to the development of one or more cancers and/or one or more tumors. In an aspect, the inhibition or reduction can be 10-20%, 20-30%, 30-40%, 40-50%, 50-60%, 60-70%, 70-80%, 80-90%, or 90-100% as compared to native or control levels or to the subject's level prior to the development of one or more cancers and/or one or more tumors. In an aspect, the inhibition or reduction can be 0-25%, 25-50%, 50-75%, or 75-100% as compared to native or control levels or to the subject's level prior to development of one or more cancers and/or one or more tumors.

The words “treat” or “treating” or “treatment” include palliative treatment, that is, treatment designed for the relief of symptoms rather than the curing of one or more cancers and/or one or more tumors; preventative treatment, that is, treatment directed to minimizing or partially or completely inhibiting the development of one or more cancers and/or one or more tumors; and supportive treatment, that is, treatment employed to supplement another specific therapy directed toward the improvement of one or more cancers and/or one or more tumors. In an aspect, the terms cover any treatment of a subject, including a mammal (e.g., a human), and includes: (i) preventing the undesired physiological change and/or pathological condition from occurring in a subject that can be predisposed to one or more cancers and/or one or more tumors but has not yet been diagnosed as having it; (ii) inhibiting the physiological change and/or pathological condition (one or more cancers and/or one or more tumors); or (iii) relieving the physiological change and/or pathological condition, i.e., causing regression of a disease or disorder characterized by one or more cancers and/or one or more tumors. For example, in an aspect, treating one or more cancers and/or one or more tumors can reduce the severity of an established a disease or disorder in a subject by 1%-100% as compared to a control (such as, for example, an individual not having one or more cancers and/or one or more tumors). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or a disorder or a condition (such as one or more cancers and/or one or more tumors). For example, treating a disease or a disorder can reduce one or more symptoms of a disease or disorder in a subject by 1%-100% as compared to a control (such as, for example, an individual not having one or more cancers and/or one or more tumors). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or a disorder or a condition (e.g., one or more cancers and/or one or more tumors). It is understood that treatment does not necessarily refer to a cure or complete ablation or eradication of one or more cancers and/or one or more tumors. However, in an aspect, treatment can refer to a cure or complete ablation or eradication of one or more cancers and/or one or more tumors.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing and/or treating and/or controlling one or more cancers and/or one or more tumors is intended. The words “prevent” and “preventing” and “prevention” also refer to prophylactic or preventative measures for protecting or precluding a subject (e.g., an individual) not having a cancer-related and/or a tumor-related complication from progressing to that complication.

As used herein, the terms “administering” and “administration” refer to any method of providing one or more of the disclosed compositions (such as, for example, a disclosed HDAC6 inhibitor, a disclosed CD47 inhibitor, a disclosed pharmaceutical formulation, and/or a disclosed composition). Such methods are well-known to those skilled in the art and include, but are not limited to, the following: oral administration, transdermal administration, administration by inhalation, nasal administration, topical administration, in utero administration, intrahepatic administration, intravaginal administration, epidural administration (such as epidural injection), intracerebroventricular (ICV) administration, ophthalmic administration, intraaural administration, depot administration, topical (skin) administration, otic administration, intra-articular (such as joint or vertebrate injection), intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration. Administration can be continuous or intermittent. Administration can comprise a combination of one or more route. In an aspect, a disclosed HDAC6 inhibitor, a disclosed CD47 inhibitor, a disclosed pharmaceutical formulation, and/or a disclosed composition can be concurrently and/or serially administered to a subject via multiple routes of administration. Various combinations of administration are known to the art.

By “determining the amount” is meant both an absolute quantification of a particular analyte (e.g., a cancer biomarker or the activity/expression level of SIRPα or CD47, etc.) or a determination of the relative abundance of a particular analyte (e.g., a cancer biomarker or the activity/expression level of SIRPα or CD47, etc.). The phrase includes both direct or indirect measurements of abundance or both. In an aspect, determining the amount can refer to measuring the expression of a cancer biomarker.

As used herein, “modifying the method” can comprise modifying or changing one or more features or aspects of one or more steps of a disclosed method. In an aspect, a method can be altered by changing the amount of one or more of the disclosed compositions (e.g., a disclosed HDAC6 inhibitor, a disclosed CD47 inhibitor, a disclosed pharmaceutical formulation, and/or a disclosed composition) used in a disclosed method, or by changing the frequency of administration of one or more disclosed compositions (e.g., a disclosed HDAC6 inhibitor, a disclosed CD47 inhibitor, a disclosed pharmaceutical formulation, and/or a disclosed composition) in a disclosed method, by changing the duration of time that one or more disclosed HDAC6 inhibitors, one or more disclosed CD47 inhibitors, one or more disclosed pharmaceutical formulations, and/or one or more disclosed compositions is administered in a disclosed method, or by substituting for one or more of the disclosed components and/or reagents with a similar or equivalent component and/or reagent.

“Acetyl” means a group of formula CH3C(O)—.

“Alkoxy” means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

“Alkyl” means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH2-, —CH2CH2-, —CH2CH2CHC(CH3)-, and —CH₂CH(CH₂CH₃)CH₂—.

“Aryl,” means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, and the like. The aryl is attached to the parent molecular moiety through any carbon atom contained within the aryl ring system. In certain embodiments, the aryl group is phenyl or naphthyl. In certain other embodiments, the aryl group is phenyl.

“Cycloalkyl” as used herein, means a monocyclic cycloalkyl ring system.

Monocyclic ring systems are cyclic hydrocarbon groups containing from 3 to 6 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl and cyclohexenyl.

“Halo” or “halogen” means —Cl, —Br, —I or —F.

“Haloalkyl” means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

“Heteroaryl” means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a phenyl ring, then the bicyclic heteroaryl group is connected to the parent molecular moiety through any carbon atom or nitrogen atom within the bicyclic ring system.

Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia. In certain embodiments of the disclosure, the heteroaryl group is furyl, imidazolyl, pyridinyl, pyrimidinyl, pyrazinyl, pyrazolyl, pyrrolyl, thiazolyl, thienyl, triazolyl, benzimidazolyl, benzofuranyl, indazolyl, indolyl, quinolinyl, and the like.

“Heterocyclyl” means a monocyclic 5 or 6 membered heterocyclic ring containing at least one N atom and optionally one or more additional heteroatoms independently selected from the group consisting of O, N, and S where the ring is saturated or unsaturated, but not aromatic. Representative examples of monocyclic heterocycle include, but are not limited to, imidazolinyl, imidazolidinyl, isothiazolinyl, isothiazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, thiopyranyl. In certain embodiments, the heterocyclyl is imidazolinyl, pyrrolidinyl, piperidinyl, or piperazinyl.

As used herein, “concurrently” means (1) simultaneously in time, or (2) at different times during a common treatment schedule.

The term “contacting” as used herein refers to bringing one or more of the disclosed compositions (e.g., a disclosed HDAC6 inhibitor, a disclosed CD47 inhibitor, a disclosed pharmaceutical formulation, and/or a disclosed composition) together with a target area or intended target area (e.g., a population of macrophages or a population in cancer cells) in such a manner that the disclosed compositions can exert an effect on the intended target or targeted area either directly or indirectly. A target area or intended target area can be one or more cells (e.g., a population of macrophages or a population in cancer cells) and/or one or more cancerous cells or cancerous tissues, or any combination thereof. In an aspect, a target area or intended target area can be any cell or any organ affected by cancerous cells or cancer. In an aspect, a target area or intended target area can be any organ, tissue, or cells that are affected by cancer or tumors.

As used herein, “determining” can refer to measuring or ascertaining the presence and severity of one or more cancers and/or one or more tumors. “Determining” can refer to measuring or ascertaining an expression level of a protein or gene of interest, for example, expressed in or by one or more cancers or tumors. “Determining” can refer to measuring or ascertaining the expression level and/or activity level of one or more cancer biomarkers. “Determining” can refer to ascertaining or measuring some type of neurologic, physiologic, and/or metabolic function and/or response.

As used herein, RNA interference (RNAi) is a sequence-specific RNA degradation process that provides a relatively easy and direct way to knock down, or silence, theoretically any gene. In naturally occurring RNAi, a double-stranded RNA (dsRNA) is cleaved by an RNase III/helicase protein, Dicer, into small interfering RNA (siRNA) molecules, a dsRNA of 19-27 nucleotides (nt) with 2-nt overhangs at the 3′ ends. These siRNAs are incorporated into a multicomponent-ribonuclease called RNA-induced silencing complex (RISC). One strand of siRNA remains associated with RISC and guides the complex toward a cognate RNA that has sequence complementary to the guider ss-siRNA in RISC. This siRNA-directed endonuclease digests the RNA, thereby inactivating it. Recent studies have revealed that chemically synthesized 21-27-nt siRNAs exhibit RNAi effects in mammalian cells, and the thermodynamic stability of siRNA hybridization (at terminals or in the middle) plays a central role in determining the molecule's function. These and other characteristics of RISC, siRNA molecules, and RNAi have been described. Applications of RNAi in mammalian cells in the laboratory or, potentially, in therapeutic settings, use either chemically synthesized siRNAs or endogenously expressed molecules. The endogenous siRNA is first expressed as small hairpin RNAs (shRNAs) by an expression vector (plasmid or virus vector) and is then processed by Dicer into siRNAs.

Methods and techniques used to determine the presence and/or severity of one or more cancers and/or one or more tumors are typically known to the medical arts. For example, the art is familiar with the ways to identify and/or diagnose the presence, severity, or both of one or more cancers and/or one or more tumors. Methods can be based on objective and/or subjective means.

The term “antibody” is used to mean an immunoglobulin molecule that recognizes and specifically binds to a target, such as a protein, polypeptide, peptide, carbohydrate, polynucleotide, lipid, or combinations of the foregoing etc., through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, fusion proteins comprising an antibody portion, and any other modified immunoglobulin molecule comprising an antigen recognition site so long as the antibodies exhibit the desired biological activity. An antibody can be of any the five major classes of immunoglobulins: IgA, IgD. IgE, IgG, and IgM, or subclasses (isotypes) thereof (e.g., IgG1. IgG2, IgG3. IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to-other molecules such as toxins, radioisotopes, etc.

As used herein, the term “antibody fragment” refers to a portion of an intact antibody and refers to the antigenic determining variable regions of an intact antibody. Examples of antibody fragments include, but are not limited to Fab, Fab′, F(ab′)2, and Fv fragments, linear antibodies, single chain antibodies, and multi-specific antibodies formed from antibody fragments.

A “monoclonal antibody” as used herein refers to homogenous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including, but not limited to, by hybridoma, phage selection, recombinant expression, and transgenic animals.

As used herein, the term “humanized antibody” refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues from the complementary determining region (CDR) are replaced by residues from the CDR of a non-human species (e.g., mouse, rat, rabbit, hamster, etc.) that have the desired specificity, affinity, and capability. In some instances, the Fv framework region (FR) residues of a human immunoglobulin are replaced with the corresponding residues in an antibody from a non-human species that has the desired specificity, affinity, and capability. The humanized antibody can be further modified by the substitution of additional residue either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin.

That an antibody “selectively binds” or “specifically binds” to an epitope or receptor means that the antibody reacts or associates more frequently, more rapidly, with greater duration, with greater affinity, or with some combination of the above to the epitope or receptor than with alternative substances, including unrelated proteins. “Selectively binds” or “specifically binds” means, for instance, that an antibody binds to a protein with a KD of about 0.1 mM or less, more usually about 1 μM or less. “Selectively binds” or “specifically binds” means at times that an antibody binds to a protein with a KD of about 0.1 mM or less, at times about 1 μM or less, at times about 0.1 μM or less, at times about 0.01 μM or less, and at times about 1 nM or less. It is understood that, in certain embodiments, an antibody or binding moiety that specifically binds to a first target may or may not specifically bind to a second target. As such, “specific binding” does not necessarily require (although it can include) exclusive binding, e.g., binding to a single target. Thus, an antibody may, in an aspect, specifically bind to more than one target (e.g., human CD47). In an aspect, the multiple targets may be bound by the same antigen-binding site on the antibody.

Polyclonal antibodies can be prepared by any known method. Polyclonal antibodies are raised by immunizing an animal (e.g., a rabbit, rat, mouse, donkey, goat, etc.) by multiple subcutaneous or intraperitoneal injections of the relevant antigen (a purified peptide fragment, full-length recombinant protein, fusion protein, etc.) optionally conjugated to keyhole limpet hemocyanin (KLH), serum albumin, etc. diluted in sterile saline and combined with an adjuvant (e.g., Complete or Incomplete Freund's Adjuvant) to form a stable emulsion. The polyclonal antibody is then recovered from blood, ascites and the like, of an animal so immunized. Collected blood is clotted, and the serum decanted, clarified by centrifugation, and assayed for antibody titer. The polyclonal antibodies can be purified from serum or ascites according to standard methods in the art including affinity chromatography, ion-exchange chromatography, gel electrophoresis, dialysis, etc.

As used herein, “lipid nanoparticles” or “LNPs” can deliver nucleic acid (e.g., DNA or RNA), protein (e.g., RNA-guided DNA binding agent), or nucleic acid together with protein. LNPs can comprise biodegradable, ionizable lipids. For example, LNPs can comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. In an aspect, the term cationic and ionizable in the context of LNP lipids can be used interchangeably, e.g., wherein ionizable lipids are cationic depending on the pH.

As used herein, “effective amount” and “amount effective” can refer to an amount that is sufficient to achieve the desired result such as, for example, the treatment and/or prevention of a disease or disorder characterized by excessive and/or uncontrollable inflammation. As used herein, the terms “effective amount” and “amount effective” can refer to an amount that is sufficient to achieve the desired an effect on an undesired condition (e.g., one or more cancers and/or one or more tumors). For example, a “therapeutically effective amount” refers to an amount that is sufficient to achieve the desired therapeutic result or to have an effect on undesired symptoms, but is generally insufficient to cause adverse side effects.

In an aspect, “therapeutically effective amount” means an amount of the disclosed composition that (i) treats a disease or disorder characterized by excessive and/or uncontrollable inflammation, (ii) attenuates, ameliorates, or eliminates one or more symptoms associated with a disease or disorder characterized by excessive and/or uncontrollable inflammation, or (iii) delays the onset of one or more symptoms of a disease or disorder characterized by excessive and/or uncontrollable inflammation. The specific therapeutically effective dose level for any particular patient will depend upon a variety of factors including the disease or disorder characterized by excessive and/or uncontrollable inflammation being treated; the disclosed compositions employed; the disclosed methods employed; the age, body weight, general health, sex and diet of the patient; the time of administration; the route of administration; the rate of excretion of the disclosed compositions employed; the duration of the treatment; drugs used in combination or coincidental with the disclosed compositions employed, and other like factors well known in the medical arts. For example, it is well within the skill of the art to start doses of the disclosed compositions at levels lower than those required to achieve the desired therapeutic effect and to gradually increase the dosage until the desired effect is achieved. If desired, then the effective daily dose can be divided into multiple doses for purposes of administration. Consequently, a single dose of a disclosed HDAC6 inhibitor, a disclosed CD47 inhibitor, a disclosed pharmaceutical formulation, and/or a disclosed composition can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a sign or symptom associated with a disease or disorder characterized by excessive and/or uncontrollable inflammation.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, the terms “cancer” and “cancerous” refer to or describe the physiological condition in mammals in which a population of cells are characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancer.

The terms “proliferative disorder” and “proliferative disease” refer to disorders associated with abnormal cell proliferation such as cancer.

“Tumor” and “neoplasm” as used herein refer to any mass of tissue that result from excessive cell growth or proliferation, either benign (noncancerous) or malignant (cancerous) including pre-cancerous lesions. “Metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell” or “tumor stem cell” or “solid tumor stem cell” are used interchangeably herein and refer to a population of cells from a solid tumor that: (1) have extensive proliferative capacity; (2) are capable of asymmetric cell division to generate one or more kinds of differentiated progeny with reduced proliferative or developmental potential; and (3) are capable of symmetric cell divisions for self-renewal or self-maintenance. These properties of “cancer stem cells” or “tumor stem cells” or “solid tumor stem cells” confer on those cancer stem cells the ability to form palpable tumors upon serial transplantation into an immunocompromised mouse compared to the majority of tumor cells that fail to form tumors. Cancer stem cells undergo self-renewal versus differentiation in a chaotic manner to form tumors with abnormal cell types that can change over time as mutations occur.

The terms “cancer cell” or “tumor cell” and grammatical equivalents refer to the total population of cells derived from a tumor including both non-tumorigenic cells, which comprise the bulk of the tumor cell population, and tumorigenic stem cells (cancer stem cells).

As used herein “tumorigenic” refers to the functional features of a solid tumor stem cell including the properties of self-renewal (giving rise to additional tumorigenic cancer stem cells) and proliferation to generate all other tumor cells (giving rise to differentiated and thus non-tumorigenic tumor cells) that allow solid tumor stem cells to form a tumor.

As used herein, the “tumorigenicity” of a tumor refers to the ability of a random sample of cells from the tumor to form palpable tumors upon serial transplantation into immunocompromised mice.

Disclosed are the components to be used to prepare the disclosed compositions, disclosed pharmaceutical formulations, disclosed therapeutic agents, or a combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. HDAC6

HDAC6 (Histone Deacetylase 6) is a protein coding gene. There are 2 isoforms produced by alternative splicing. The canonical isoform has 1215 amino acids. Diseases associated with HDAC6 include chondrodysplasia with platyspondyly, distinctive brachydactyly, hydrocephaly, and microphthalmia and Alexander Disease. Among its related pathways are constitutive signaling by NOTCH1 HD+PEST domain mutants and selective autophagy. Gene Ontology (GO) annotations related to this gene include enzyme binding and ubiquitin protein ligase binding. An important paralog of this gene is HDAC10. HDAC6 is responsible for the deacetylation of lysine residues on the N-terminal part of the core histones (H2A, H2B, H3 and H4). HDAC6 is also identify by HGNC 14064, NCBI Entrez Gene 10013, Ensembl ENSG00000094631, OMIM® 300272, UniProtKB/Swiss-Prot Q9UBN7, and Open Targets Platform ENSG00000094631.

In an aspect, a disclosed HDAC6 can comprise the amino acid sequence set forth in any one of SEQ ID NO:15, SEQ ID NO: 16, SEQ ID NO:17, or any fragment thereof. In an aspect, a disclosed HDAC6 can be encoded by the mRNA sequence set forth in any one of SEQ ID NO:18, SEQ ID NO:19, or a fragment thereof. In an aspect, a disclosed HDAC6 can be encoded by the sequence set forth in SEQ ID NO:20, SEQ ID NO:21, or a fragment thereof.

### C. CD47

CD47 is a protein code gene. The CD47 gene encodes a membrane protein, which is involved in the increase in intracellular calcium concentration that occurs upon cell adhesion to extracellular matrix. The encoded protein is also a receptor for the C-terminal cell binding domain of thrombospondin, and it may play a role in membrane transport and signal transduction. This gene has broad tissue distribution and is reduced in expression on Rh erythrocytes. Alternatively spliced transcript variants have been found for this gene. Diseases associated with CD47 include Hereditary Spherocytosis and Glanzmann Thrombasthenia 1. Among its related pathways are innate immune system and integrin pathway. CD47 is identified by HGNC 1682, NCBI Entrez Gene 961, Ensembl ENSG 00000196776, OMIM® 601028, and UniProtKB/Swiss-Prot Q08722.

In an aspect, a disclosed CD47 can comprise the amino acid sequence set forth in any one of SEQ ID NO:07, SEQ ID NO:08, SEQ ID NO:09, SEQ ID NO:10, or any fragment thereof. In an aspect, a disclosed CD47 can be encoded by the mRNA sequence set forth in any one of SEQ ID NO:11, SEQ ID NO:12, SEQ ID NO:13, or a fragment thereof. In an aspect, a disclosed HDAC6 can be encoded by the sequence set forth in SEQ ID NO:14 or a fragment thereof.

### D. SIRPα

SIRPα is a protein coding gene. Diseases associated with SIRPα include glioblastoma and tenosynovial giant cell tumor. Among its related pathways are innate immune system and cell junction organization. Gene Ontology (GO) annotations related to this gene include SH3 domain binding. An important paralog of this gene is SIRPB1. SIRPα is an immunoglobulin-like cell surface receptor for CD47 and acts as docking protein and induces translocation of PTPN6, PTPN11 and other binding partners from the cytosol to the plasma membrane. SIRPα is identified by HGNC 9662, NCBI Entrez Gene 140885, Ensembl ENSG00000198053, OMIM® 602461, and UniProtKB/Swiss-Prot P78324.

In an aspect, a disclosed SIRPα can comprise the amino acid sequence set forth in any one of SEQ ID NO:22, SEQ ID NO:23, SEQ ID NO:24, or any fragment thereof. In an aspect, a disclosed SIRPα can be encoded by the mRNA sequence set forth in any one of SEQ ID NO:25, SEQ ID NO:26, SEQ ID NO:27, SEQ ID NO:28, or a fragment thereof. In an aspect, a disclosed HDAC6 can be encoded by the sequence set forth in SEQ ID NO:29 or a fragment thereof.

### E. Macrophages

Macrophages constitute a heterogeneous cell population representing innate immunity. Macrophages have been identified in all tissues and their chief competences are phagocytic activity and antigen presentation. Macrophages continuously monitor their microenvironments for the presence of pathogens, unfit cells, debris, and toxic metabolites, and release a variety of active substances including growth factors and cytokines. Human macrophages express a number of markers including CD14, CD16, CD68, CD163, CD11b, CD86, and CD206.

By contrast with many other cell types, macrophages cannot be traced to a single origin. The modern concept of macrophage origin includes three developmental sources of these cells, which correspond to the three generations of hematopoietic stem cells. The first generation develops from the extraembryonic yolk sac posterior plate mesoderm in the blood islands. These cells apparently give rise to the microglia of the central nervous system. The second wave of hematopoietic progenitors, which develops from the yolk sac hematogenic endothelium, is called erythro-myeloid precursors. After the onset of blood circulation, these cells colonize the embryonic liver. Erythro-myeloid precursors give rise to granulocyte, monocyte, and macrophage lineages. The third generation of hematopoietic cells is derived from endothelium in the aorto-gonado-mesonephral zone; these cells colonize the fetal liver where they establish hematopoiesis and the red bone marrow where they produce the bone marrow hematopoietic stem cell lineages. During embryogenesis, macrophages in organs are predominantly represented by cells of the second and third generations, while postnatal development is marked by increasing percentage of macrophages derived from the third generation.

The most-discussed current classification of macrophages is based on the M1/M2 paradigm, which is related to their pro- and anti-inflammatory properties. The M1/M2 paradigm states that macrophages can switch their phenotypes from the pro-inflammatory M1 to the anti-inflammatory M2 and vice versa, depending on the needs of the microenvironment, or maintain the naïve state M0 in the absence of external signals. The classical activation of macrophages is promoted by lipopolysaccharide (LPS), interferon gamma (IFN-γ), and granulocyte-macrophage colony-stimulating factor (GM-CSF). The resulting classical (M1) phenotype is characterized by expression of TLR-2, TLR-4, CD80, and CD86. M1 macrophages secrete pro-inflammatory cytokines (interleukins IL-1β, IL-12, IL-18 and IL-23, and tumor necrosis factor alpha TNF-α) that modulate the Th1-mediated antigen-specific inflammatory reactions. M1 macrophages have also been demonstrated to enhance the expression of inducible nitric oxide synthase (NOS2 or iNOS) to facilitate the production of NO from L-arginine.

TABLE 1
Human Macrophage Phenotype Markers.

| | M1 | M2 |
|---|---|---|
| CD163 | ↓ | ↑ |
| Mannose receptor (CD206) | ↓ | ↑ |
| AMAC1 (CCL-18) | ↓ | ↑ |
| IL-10 | ↓ | ↑ |
| TNFα | ↑ | ↓ |
| iNOS | ↑ | ↓ |
| IL-12 | ↑ | ↓ |
| IL-6 | ↑ | ↓ |
| MCP-1 (CCL2) | ↑ | ↓ |

Activation of macrophages towards M2 phenotypes can be induced by antigen-antibody complexes, invading helminths, complement system components, apoptotic cells, interleukins (IL-4, IL-13, and IL-10), and transforming growth factor beta (TGF-β). Activation with these inducers drives macrophages towards the increased secretion of IL-10 and reduced secretion of IL-12 typical of the M2 phenotypes. M2 macrophages show diverse gene expression signatures, and distinct M2a, M2b, M2c, and M2d macrophage subpopulations have been identified by transcriptome analysis. The corresponding M2 phenotypes are generally characterized by high levels of mannose receptor CD206 and scavenger receptor CD163. Arginase 1, which converts arginine into ornithine—an important building block for collagen synthesis, is a relevant marker of M2 macrophage polarization in rats and mice.

To date, the most common macrophage sources are bone marrow, spleen, and peritoneal cavity. Compared to bone marrow-derived macrophages (BMDMs) and splenic macrophages (SPMs), peritoneal macrophages (PMs) appear to be more mature with higher expression of inducible cytokines and are more stable in their functionality and phenotype. Therefore, PMs isolated from the peritoneal cavity are the common source of macrophages for various in vitro assays, including stimulation with Toll-like receptor (TLR) ligands, cell signaling assay, phagocytosis, cytokine production, chemokine production, and toxicology study.

PMs are the major cell type of peritoneal cells (more than 30%). PMs can be classified into classically activated macrophages (M1) and alternatively activated macrophages (M2) following stimulation. This classification method is mainly based on cell phenotype and function. Notably, M1-polarized PMs have long been identified to play an important role in host defense, which express Th1 cytokines and inflammatory cytokines, including tumor necrosis factor-α (TNF-α), interleukin-2 (IL-2), and interferon-γ (IFN-γ). M2-polarized PMs predominantly express a large amount of Th2 cytokines and anti-inflammatory cytokines, including IL-4, IL-13, IL-10, and transforming growth factor-β (TGF-β), thereby downregulating inflammatory processes.

Accumulating studies have demonstrated that PMs in the peritoneal cavity strongly express CD206 mRNA, which is the characteristic phenotype of M2-polarized macrophages. Therefore, M2-polarized PMs are the major composition of PMs. Additionally, PMs can be classified into another two subsets based on morphology: large PMs and small PMs. These two macrophage subsets exhibit distinct origin and morphology. On the one hand, large PMs have been characterized as fetal-originated tissue resident macrophages with a high level of F4/80 and a low level of major histocompatibility complex II (MHC-II). Under steady condition, large PMs compose the major population of PMs and are characterized with high expression of transcription factor GATA6. It has been proven that the GATA6 expressed in large PMs selectively regulates the level of aspartoacylase and therefore control the survival, differentiation, and metabolism of resident PMs. On the other hand, small PMs appear to be generated from embryogenic precursors with a low level of F4/80 and a high level of MHC-II. It has been reported that the PMs have a cross-talk between T lymphocytes, which are enriched in IL-17 receptor A and express a proangiogenic gene profile, and therefore directly promote ovarian cancer cell proliferation.

### F. Immunity

The human immune system is a complex and powerful defense mechanism. The primary function of the immune system is to defend the body from pathogens, which are disease-causing organisms such as viruses and bacteria. Tissues, cells, and proteins in the immune system work together to achieve this function.

To fight infections, the immune system must be able to identify pathogens. Pathogens have molecules called antigens on their surface. Antigens provide a unique signature for the pathogen that enables immune system cells to recognize different pathogens and distinguish pathogens from the body's own cells and tissues. When a pathogen gets into the body, the immune system reacts in two ways.

The innate immune response is a rapid reaction. Innate immune cells recognize certain molecules found on many pathogens. These cells also react to signaling molecules released by the body in response to infection. Through these actions, innate immune cells quickly begin fighting an infection. This response results in inflammation. The cells involved in this reaction can kill pathogens and can also help activate cells involved in adaptive immunity.

The adaptive immune response is slower than the innate response but is better able to target specific pathogens. There are two main cell types involved in this response: T cells and B cells. Some T cells kill pathogens and infected cells. Other T cells help control the adaptive immune response. The main function of B cells is to make antibodies against specific antigens. Antibodies, also known as immunoglobulins, are proteins that attach themselves to pathogens. This signals immune cells to destroy the pathogen.

It takes time for T and B cells to respond to the new antigens when a pathogen causes an infection. Once exposed to the pathogen, these cells develop a memory for the pathogen so that they are ready for the next infection. As part of the adaptive immune response, some T and B cells change into memory cells. Memory cells mostly stay in the lymph nodes and the spleen and “remember” particular antigens. If a person becomes infected with the same pathogen again, these cells are able to quickly and vigorously begin fighting the infection.

### G. Compositions for Use in the Disclosed Methods

### 1. HDAC6 Inhibitors

Disclosed herein are HDAC6 inhibitors for use in one or more disclosed methods.

Disclosed herein are HDAC6 inhibitors for use in one or more disclosed kits.

In an aspect, a disclosed HDAC6 inhibitor can comprise any disclosed HDAC6 inhibitor. In an aspect, a disclosed HDAC6 inhibitor can comprise a small molecule, a peptide, a polynucleotide, an antibody or fragment thereof, an antisense oligonucleotide, siRNA, RNAi, or any combination thereof.

In an aspect, a disclosed HDAC6 inhibitor can comprise Nexturastat A, Tubastatin A, KA2507, Ricolinostat (ACY-1215), Citarinostat (ACY-241), Tubacin, CAY10603, WT161, ACY-738, ACY-775, HPOB, SKLB-23bb, SS-208, Suprastat HDAC6 degrader-1 (PROTAC), HDAC6 degrader-3 (PROTAC), J22352 (PROTAC), HPB, HDAC6-IN-12 (compound GZ), HDAC6/8/BRPF1-IN-1, QTX125, CG347B, BRD73954, AES-135, AES-350, KH-259, SW-100, HPOB, Droxinostat (NS 41080), Bufexamac, KA2507, MC2625, MPT0G211, MPT0G211 mesylate, WT-161, ACY-738, ACY-775, XP5, HDAC-IN-35 (Compound 14), HDAC6-IN-15, HDAC6-IN-14, HDAC6-IN-13 (Compound 35m), HDAC6-IN-11 (Compound 9), HDAC6-IN-10, HDAC6-IN-9 (compound 12c), HDAC6-IN-8, HDAC6-IN-7 (TCS HDAC6 20b), HDAC6-IN-6 (compound 6a), HDAC6-IN-5 (compound 11b), HDAC6-IN-4 (C10), HDAC-IN-4, HDAC-IN-40, HDAC6-IN-3 (Compound 14), HDAC3/6-IN-2 (compound 15), or any combination thereof. In an aspect, a disclosed HDAC6 inhibitor can comprise Nexturastat A.

In an aspect, a disclosed HDAC6 inhibitor can be any inhibitor described in U.S. Pat. App. Pub. No. 2020/0115350, the disclosure of which is incorporated herein in its entirety. In an aspect, a disclosed HDAC6 inhibitor can be a compound having Formula I:

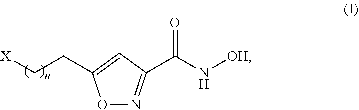

or a pharmaceutically acceptable salt, solvate, or prodrug thereof,
wherein X comprises

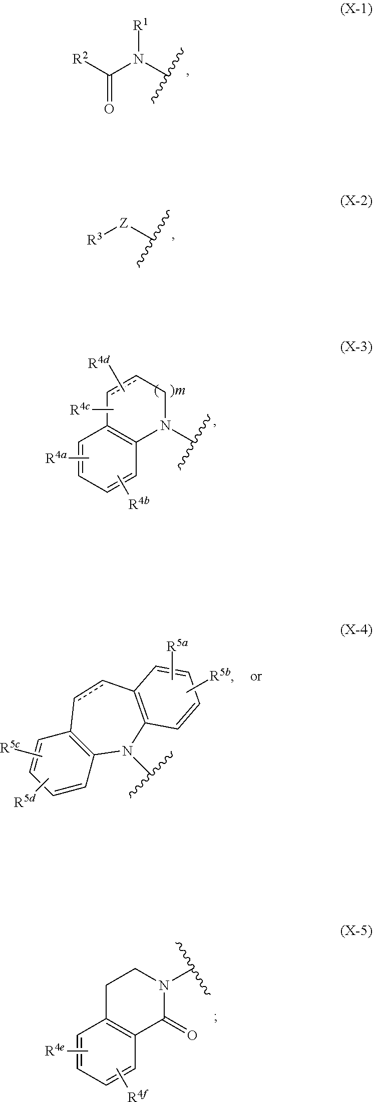

wherein R¹ comprises hydrogen or C₁₋₄ alkyl; wherein R² comprises optionally substituted C₆-C₁₄ aryl or aralkyl;
wherein R³ comprises optionally substituted C₆-C₁₄ aryl, optionally substituted 5- to 14-membered heteroaryl, or —C(═O)NR^{d}R^{e};
wherein R^{4a}, R^{4b}, R^{4c}, and R^{4f} are independently hydrogen, halogen, hydroxy, nitro, cyano, —NR^{a}R^{b}, —C(═O)NR^{a}R^{b}, —C(═O)R^{c}, C₁₋₆ alkyl, C₂₋₄ alkenyl, C₂₋₆ alkynyl, C₁₋₆ alkoxy, C₁₋₆ haloalkyl, or haloalkoxy;
wherein R^{4c} and R^{4d} are independently hydrogen or C₁₋₄ alkyl or wherein R^{4c} and R^{4d} taken together form a —C(═O)— with the carbon atom to which they are attached;
wherein R^{5a}, R^{5b}, R^{5c}, and R^{5d} are independently hydrogen, halogen, hydroxy, nitro, cyano, —NR^{a}R^{b}, —C(═O)NR^{a}R^{b}, —C(═O)R, C₁₋₆ alkyl, C₂₋₆ alkenyl, C₂₋₆ alkynyl, C₁₋₆ alkoxy, C₁₋₆ haloalkyl, or haloalkoxy;
wherein Z is —O—, —N(R⁵)—, or —C(═O)—, or wherein Z is absent;
wherein R⁸ is hydrogen, C₁₋₄ alkyl, optionally substituted C₃₋₆ cycloalkyl, optionally substituted C₆-C₁₄ aryl, aralkyl, optionally substituted 5- to 14-membered heteroaryl, or heteroaralkyl;
wherein m is 0, 1, or 2;
wherein n is 1, 2, 3, 4, 5, or 6;
wherein [symbol] represents a single or double bond;
wherein R^{a}, R^{b}, R^{d}, and R^{e} are independently hydrogen, C₁₋₆ alkyl, optionally substituted C₃-6 cycloalkyl, optionally substituted C₆-C₁₄ aryl, or optionally substituted 5- to 14-membered heteroaryl, or
wherein R^{a} and R^{b} taken together with the nitrogen atom to which they are attached form an optionally substituted 3- to 12-membered heterocyclo, or
wherein R^{d} and R^{e} taken together with the nitrogen atom to which they are attached form an optionally substituted 3- to 12-membered heterocyclo; and
wherein R^{c} is C₁₋₄ alkyl, with the proviso that when Z is absent, R³ is a bicyclic or tricyclic C₁₀₋₁₄ aryl, a bicyclic or tricyclic 9- to 14-membered heteroaryl, or —C(═O)NR^{d}R^{e}.

In an aspect, a disclosed HDAC6 inhibitor can be 5-(2-benzamidoethyl)-N-hydroxyisoxazole-3-carboxamide; 5-(2-(3,4-dichlorobenzamido)ethyl)-N-hydroxyisoxazole-3-carboxamide; 5-(2-(2-naphthamido)ethyl)-N-hydroxyisoxazole-3-carboxamide; 5-(2-([1,1′-biphenyl]-3-carboxamido)ethyl)-N-hydroxyisoxazole-3-carboxamide; 5-(4-(5,6-dichloro-1H-indol-1-yl)butyl)-N-hydroxyisoxazole-3-carboxamide; 5-(4-(6-chloro-3,4-dihydroquinolin-1(2H)-yl)butyl)-N-hydroxyisoxazole-3-carboxamide; 5-(4-(6-chloro-4,4-dimethyl-3,4-dihydroquinolin-1(2H)-yl)butyl)-N-hydroxyisoxazole-3-carboxamide; 5-(3-(3,4-dichlorophenoxy)propyl)-N-hydroxyisoxazole-3-carboxamide; 5-(4-(2,8-dichloro-10,11-dihydro-5H-dibenzo[b,f]azepin-5-yl)butyl)-N-hydroxyisoxazole-3-carboxamide; 5-(2-(4-bromobenzamido)ethyl)-N-hydroxyisoxazole-3-carboxamide; 5-(2-(4-fluorobenzamido)ethyl)-N-hydroxyisoxazole-3-carboxamide; 5-(2-(4-chlorobenzamido)ethyl)-N-hydroxyisoxazole-3-carboxamide; N-hydroxy-5-(2-(4-methoxybenzamido)ethyl)isoxazole-3-carboxamide; 5-(2-(4-(dimethylamino)benzamido)ethyl)-N-hydroxyisoxazole-3-carboxamide; 5-(2-(4-cyclopropylbenzamido)ethyl)-N-hydroxyisoxazole-3-carboxamide; 5-(2-(3,4-difluorobenzamido)ethyl)-N-hydroxyisoxazole-3-carboxamide; 5-(2-(3-chloro-4-fluorobenzamido)ethyl)-N-hydroxyisoxazole-3-carboxamide; 5-(2-(4-chloro-3-fluorobenzamido)ethyl)-N-hydroxyisoxazole-3-carboxamide; 5-(2-(3-(dimethylamino)benzamido)ethyl)-N-hydroxyisoxazole-3-carboxamide; N-hydroxy-5-(2-(3-(pyridin-3-yl)benzamido)ethyl)isoxazole-3-carboxamide; 5-(3-benzamidopropyl)-N-hydroxyisoxazole-3-carboxamide; N-hydroxy-5-(2-(4-(trifluoromethoxy)benzamido)ethyl)isoxazole-3-carboxamide; 5-(2-(4,5-dichloroindoline-1-carboxamido)ethyl)-N-hydroxyisoxazole-3-carboxamide; 5-(2-((6,7-dichloroisoquinolin-3-yl)amino)ethyl)-N-hydroxyisoxazole-3-carboxamide; 5-(3-(5,6-dichloro-1H-benzo[d]imidazol-2-yl)propyl)-N-hydroxyisoxazole-3-carboxamide; 5-(2-((5,6-dichloro-1-methyl-1H-benzo[d]imidazol-2-yl)oxy)ethyl)-N-hydroxyisoxazole-3-carboxamide; N-hydroxy-5-(2-(4-((trifluoromethyl)thio)benzamido)ethyl)isoxazole-3-carboxamide; 5-(4-(4,5-dichloroindolin-1-yl)-4-oxobutyl)-N-hydroxyisoxazole-3-carboxamide; 5-(2-((6,7-dichloroquinolin-2-yl)amino)ethyl)-N-hydroxyisoxazole-3-carboxamide; 5-(3-(5,6-dichlorobenzo[d]thiazol-2-yl)propyl)-N-hydroxyisoxazole-3-carboxamide; 5-(3-(5,6-dichlorobenzo[d]oxazol-2-yl)propyl)-N-hydroxyisoxazole-3-carboxamide; N-hydroxy-5-(2-(4-(trifluoromethyl)benzamido)ethyl)isoxazole-3-carboxamide; 2-(3-(hydroxycarbamoyl)isoxazol-5-yl)ethyl 4,5-dichloroindoline-1-carboxylate; 5-(2-((6,7-dichloronaphthalen-2-yl)amino)ethyl)-N-hydroxyisoxazole-3-carboxamide; 5-(2-((5,6-dichlorobenzo[d]thiazol-2-yl)amino)ethyl)-N-hydroxyisoxazole-3-carboxamide; N-hydroxy-5-(2-(phenanthridin-6-ylamino)ethyl)isoxazole-3-carboxamide; 5-(2-(2-(3,4-dichlorophenyl)acetamido)ethyl)-N-hydroxyisoxazole-3-carboxamide; 5-(2-(6,7-dichloro-1-oxo-3,4-dihydroisoquinolin-2(1H)-yl)ethyl)-N-hydroxyisoxazole-3-carboxamide; 5-(2-((5,6-dichloroisoquinolin-1-yl)amino)ethyl)-N-hydroxyisoxazole-3-carboxamide; N-hydroxy-5-(2-(2-phenylacetamido)ethyl)isoxazole-3-carboxamide; 2-(3-(hydroxycarbamoyl)isoxazol-5-yl)ethyl (3,4-dichlorophenyl)(methyl) carbamate; 5-(2-((5,6-dichloroisoquinolin-1-yl)oxy)ethyl)-N-hydroxyisoxazole-3-carboxamide; 5-(2-(N-butylbenzamido)ethyl)-N-hydroxyisoxazole-3-carboxamide; 5-(4-((3,4-dichlorophenyl)amino)butyl)-N-hydroxyisoxazole-3-carboxamide; 5-(3-((3,4-dichlorophenyl)amino)propyl)-N-hydroxyisoxazole-3-carboxamide; N-hydroxy-5-(3-(naphthalen-1-ylamino)propyl)isoxazole-3-carboxamide; N-hydroxy-5-(3-(quinolin-8-ylamino)propyl)i sox azole-3-carboxamide; 5-(4-(8-chloro-2-methyl-1,2,3,4-tetrahydro-5H-pyrido[4,3-b]indol-5-yl)butyl)-N-hydroxyisoxazole-3-carboxamide; 5-(4-((4-chlorophenylxcyclohexyl)amino)butyl)-N-hydroxyisoxazole-3-carboxamide; 5-(4-(bis(4-chlorophenyl)amino)butyl)-N-hydroxyisoxazole-3-carboxamide; 5-(4-((4-chlorobenzyl)(4-chlorophenyl)amino)butyl)-N-hydroxyisoxazole-3-carboxamide; N-hydroxy-5-(3-(naphthalen-1-yloxy)propyl)isoxazole-3-carboxamide; N-hydroxy-5-(3-(quinolin-8-yloxy)propyl)isoxazole-3-carboxamide; or any combination thereof.

In an aspect, a disclosed HDAC6 inhibitor can be an inhibitor described in Intl. Pat. App. Pub. No. WO 2021/263171, the disclosure of which is incorporated herein in its entirety.

In an aspect, a disclosed HDAC6 inhibitor can comprise a compound having Formula II:

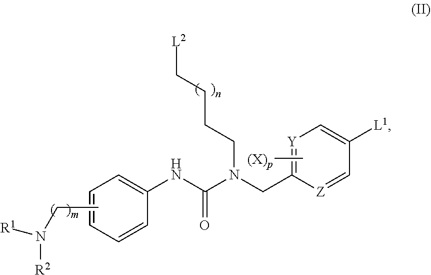

or a pharmaceutically acceptable salt, solvate, or prodrug thereof,
wherein R¹ and R² are independently hydrogen or C₁-C₆ alkyl, or wherein R¹ and R² are joined to form a 3-7 membered heterocyclyl;
wherein L¹ is CO₂H, C(O)NH₂, C(O)NHOH, or B(OH)₂;
wherein L² is H or OR³;
wherein R³ is hydrogen, acetyl, C₁-C₆ alkyl, C₁-C₆ haloalkyl, C₃-C₆ cycloalkyl, aryl, heteroaryl, or C₅-C₆ heterocyclyl;
wherein each X is independently hydrogen or halogen;
wherein p is 0, 1, 2, or 3;
wherein Y and Z are independently carbon or nitrogen;
wherein m is 1, 2, 3 or 4; and
wherein n is 0, 1 or 2.

In an aspect, a disclosed HDAC6 inhibitor can be

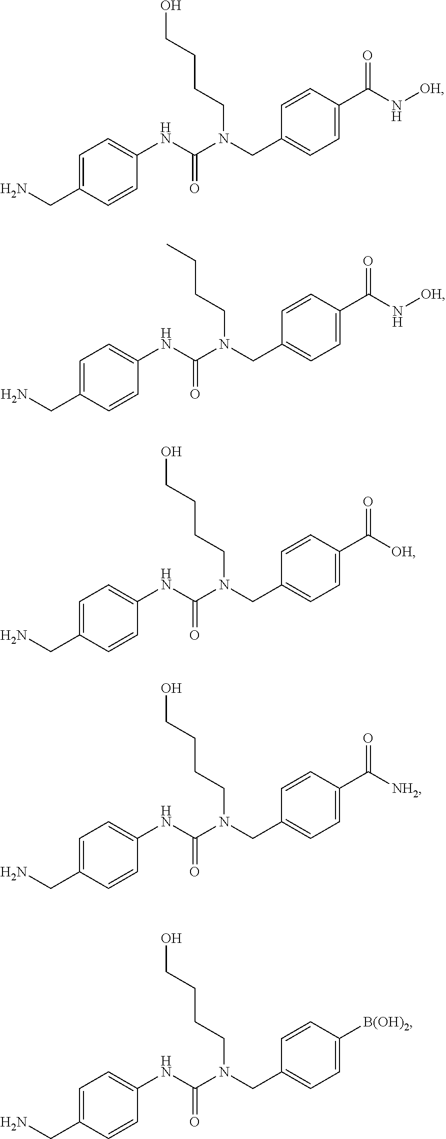 [SMILES: NCc1ccc(NC(=O)N(CCCCO)Cc2ccc(C(=O)NO)cc2)cc1 ; CCCCN(Cc1ccc(C(=O)NO)cc1)C(=O)Nc1ccc(CN)cc1 ; NCc1ccc(NC(=O)N(CCCCO)Cc2ccc(C(=O)O)cc2)cc1 ; NCc1ccc(NC(=O)N(CCCCO)Cc2ccc(C(N)=O)cc2)cc1 ; NCc1ccc(NC(=O)N(CCCCO)Cc2ccc(B(O)O)cc2)cc1]

or any combination thereof.

In an aspect, a disclosed HDAC6 inhibitor can be formulated as a composition. In an aspect, a disclosed composition comprising a disclosed HDAC6 inhibitor can further comprise a disclosed CD47 inhibitor. In an aspect, a disclosed HDAC6 inhibitor can be formulated as a pharmaceutical formulation. In an aspect, a disclosed pharmaceutical formulation comprising a disclosed HDAC6 inhibitor can further comprise a disclosed CD47 inhibitor.

In an aspect, a disclosed HDAC6 antisense oligonucleotide can target one or more aspects of HDAC6. Human HDAC6 is known to the art. The nucleotide sequences for HDAC6 are known. For example, HDAC6 is identified by HGNC 14064, NCBI Entrez Gene 10013, Ensembl ENSG00000094631, OMIM® 300272, UniProtKB/Swiss-Prot Q9UBN7, and Open Targets Platform ENSG00000094631.

In an aspect, a disclosed HDAC6 antisense oligonucleotide can target one or more parts or any part of an HDAC6 mRNA or pre-mRNA. In an aspect, a disclosed HDAC6 mRNA or pre-mRNA can comprise the sequence set forth in SEQ ID NO:18, SEQ ID NO:19, or a fragment thereof. In an aspect, a disclosed HDAC6 inhibitor can comprise siRNA. In an aspect, a disclosed siRNA can target one or more parts or any part of the sequence set forth in SEQ ID NO:18, SEQ ID NO:19, or a fragment thereof. In an aspect, a disclosed HDAC6 can comprise the amino acid sequence set forth in any one of SEQ ID NO: 15, SEQ ID NO:16, SEQ ID NO: 17, or any fragment thereof. In an aspect, an mRNA sequence for a disclosed HDAC6 can comprise the sequence set forth in any one of SEQ ID NO:18, SEQ ID NO:19, or a fragment thereof. In an aspect, a disclosed HDAC6 can be encoded by the sequence set forth in SEQ ID NO:20, SEQ ID NO:21, or a fragment thereof.

In an aspect, a therapeutically effective amount of a disclosed HDAC6 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed HDAC6 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed HDAC6 inhibitor can comprise reduce, inhibit, and/or prevent the activity level and/or expression level of HDAC6 in the subject. In an aspect, a disclosed HDAC6 inhibitor can comprise reduce, inhibit, and/or prevent the activity level and/or expression level of HDAC6 in one or more macrophage populations in a subject. In an aspect, a disclosed HDAC6 inhibitor can modulate the phenotype and/or phagocytic function of one or more macrophage populations in a subject. In an aspect, a disclosed HDAC6 inhibitor can reduce, inhibit, and/or prevent the activity level and/or expression level of CD47 in one or more cancer cells or one or more populations of cancer cells.

In an aspect, a disclosed HDAC6 inhibitor can (i) reduce and/or prevent tumor growth; (ii) reduce or slow tumor metastasis; (iii) prevent and/or delay recurrence of the cancer; (iv) extend and/or prolong disease-free or tumor-free survival time; (v) increasing and/or lengthening overall survival time; (vi) reduce and/or minimize the frequency of treatment; (vii) relieve and/or ameliorate one or more symptoms of the cancer; (viii) reduce and/or decrease tumor burden, (ix) prevent and/or facilitate surgical intervention; (x) increase and/or enhance phagocytosis of cancer cells or tumor cells; (xi) increase and/or enhance immune cell infiltration in and/or around tumor microenvironment; (xii) enhance the subject's innate antitumor immunity; (xiii) drive and/or facilitate the M1 or pro-inflammatory phenotype of macrophages, (xiv) disrupt the CD47-SIRPα axis in one or more cancer cells or tumor cells; or (xv) any combination thereof.

In an aspect, a disclosed HDAC6 inhibitor can decrease the expression level and/or activity level of SIRPα. In an aspect, a disclosed decrease can be a partial decrease or a complete decrease.

In an aspect, a disclosed HDAC6 inhibitor can comprise any commercially available HDAC6 inhibitor or a combination of commercially available HDAC6 inhibitors.

In an aspect, a disclosed HDAC6 inhibitor can comprise a selective HDAC6 inhibitor or HDAC6 selective inhibitor and can refer to a compound that preferentially inhibits histone deacetylase 6 over one or more other histone deacetylase isoforms, e.g., HDAC1, HDAC2, HDAC3, HDAC4, HDAC5, HDAC7, HDAC8, HDAC9, HDAC10, and/or HDCA11 in a cell-based in vitro assay. For example, a compound having a HDAC6 IC₅₀=5 nM and a HDAC1 IC₅₀ of 500 nM is a selective HDAC6 inhibitor that is 100-fold more selective over HDAC1; a compound having a HDAC6 ICso=5 nM, a HDAC1 ICso=500 nM, and a HDAC3 ICso=50 nM is a selective HDAC6 inhibitor that is 100-fold more selective over HDAC1 and 10-fold more selective over HDAC3; and so on. In an aspect, a disclosed selective HDAC6 inhibitor can preferentially inhibit HDAC6 over HDAC1, HDAC2, HDAC3, HDAC4, HDAC5, HDAC7, HDAC8, HDAC9, HDAC10, and/or HDAC11 In an aspect, a disclosed selective HDAC6 inhibitor can preferentially inhibit HDAC6 over one or more other HDAC isoforms. In an aspect, a disclosed selective HDAC6 inhibitor can be at least about 5-fold more selective over one or more other HDAC isoforms. In an aspect, a disclosed selective HDAC6 inhibitor can be at least about 10-fold more selective over one or more other HDAC isoforms. In an aspect, a disclosed selective HDAC6 inhibitor can be at least about 15-fold more selective over one or more other HDAC isoforms. In an aspect, a disclosed selective HDAC6 inhibitor can be at least about 20-fold more selective over one or more other HDAC isoforms. In an aspect, a disclosed selective HDAC6 inhibitor can be at least about 30-fold more selective over one or more other HDAC isoforms. In an aspect, a disclosed selective HDAC6 inhibitor can be at least about 40-fold more selective over one or more other HDAC isoforms. In an aspect, a disclosed selective HDAC6 inhibitor can be at least about 50-fold more selective over one or more other HDAC isoforms. In an aspect, a disclosed selective HDAC6 inhibitor can be at least about 100-fold more selective over one or more other HDAC isoforms. In an aspect, a disclosed selective HDAC6 inhibitor can be at least about 150-fold more selective over one or more other HDAC isoforms. In an aspect, the selective HDAC6 inhibitor can be at least about 200-fold more selective over one or more other HDAC isoforms. In an aspect, a disclosed selective HDAC6 inhibitor can be at least about 250-fold more selective over one or more other HDAC isoforms. In an aspect, a disclosed selective HDAC6 inhibitor can be at least about 500-fold more selective over one or more other HDAC isoforms. In an aspect, a disclosed selective HDAC6 inhibitor can be at least about 750-fold more selective over one or more other HDAC isoforms. In an aspect, a disclosed selective HDAC6 inhibitor can be at least about 1000-fold more selective over one or more other HDAC isoforms. In an aspect, a disclosed selective HDAC6 inhibitor can be at least about 2000-fold more selective over one or more other HDAC isoforms. In an aspect, a disclosed selective HDAC6 inhibitor can be at least about 3000-fold more selective over one or more other HDAC isoforms. HDAC6 selectivity over the other HDAC isoforms in cell-based assays can be determined using methods known in the art. In another aspect, the selective HDAC6 inhibitor can be at about 10-fold to about 3000-fold more selective over one or more other HDAC isoforms. In an aspect, a disclosed selective HDAC6 inhibitor can be at about 20-fold to about 3000-fold more selective over one or more other HDAC isoforms. In an aspect, a disclosed selective HDAC6 inhibitor can be at about 50-fold to about 3000-fold more selective over one or more other HDAC isoforms. In an aspect, a disclosed selective HDAC6 inhibitor can be at about 100-fold to about 3000-fold more selective over one or more other HDAC isoforms. In an aspect, a disclosed selective HDAC6 inhibitor can be at about 500-fold to about 3000-fold more selective over one or more other HDAC isoforms.

In an aspect, a disclosed HDAC6 inhibitor can be formulated as lipid nanoparticles.

### 2. CD47 Inhibitors

Disclosed herein are CD47 inhibitors for use in one or more disclosed methods. Disclosed herein are CD47 inhibitors for use in one or more disclosed kits.

In an aspect, a disclosed CD47 inhibitor can comprise any disclosed CD47 inhibitor. In an aspect, a disclosed CD47 inhibitor can comprise a small molecule, a peptide, a polynucleotide, an antibody or fragment thereof, an antisense oligonucleotide, siRNA, RNAi, or any combination thereof.

In an aspect, a disclosed CD47 inhibitor can be a CD47 specific chimeric antigen receptor T (CAR-T) cell.

In an aspect, a disclosed CD47 inhibitor can comprise RRx-001, a dinitroazetidine derivative, Hu5F9-G4, CC-90002, TTI-621, ALX148, SRF231, SHR-1603, IB1188, ST-1901, SGNCD-47M, or any combination thereof. In an aspect, a disclosed CD47 inhibitor can comprise Gentulizumab, CC-90002 (INBRX 103), Hu5F9-G4 (5F9), Magrolimab, STI-6643, TI-061, AO-176, SRF231, AK117, IB1188, IMC-002, SHR-1603, TJ011133, ZL-1201, evorpacept (ALX148), TTI-621, TTI-G22, or any combination thereof.

In an aspect, a disclosed CD47 inhibitor can be an anti-CD47 antibody or fragment thereof capable of binding to CD47. In an aspect, a disclosed CD47 inhibitor can be a humanized antibody. In an aspect, a disclosed CD47 inhibitor can be a monoclonal antibody. In an aspect, a disclosed CD47 inhibitor can be a bispecific antibody. In an aspect, a disclosed bispecific antibody can comprise two binding sites directed at two different antigens or two different epitopes on the same antigen. For example, in an aspect, a disclosed bispecific antibody can target two epitopes on CD47. In an aspect, a disclosed bispecific antibody can target at least one epitope on CD47 and at least one epitope on another protein target.

In an aspect, a disclosed bispecific antibody can target CD47 and SIRPα. In an aspect, a disclosed bispecific antibody can target CD47 and PD-1. In an aspect, a disclosed bispecific antibody can target CD47 and PD-1L. In an aspect, a disclosed bispecific antibody can target CD47 and 41BB. In an aspect, a disclosed bispecific antibody can target CD47 and CD20.

In an aspect, a disclosed bispecific antibody can target CD47 and CD19. In an aspect, a disclosed CD47 bispecific antibody can comprise CP0107, DSP107, HX009, IB1322, IMM0306, PF-07257876, TG-1801, or any combination thereof.

In an aspect, a disclosed CD47 inhibitor can be formulated as a composition. In an aspect, a disclosed composition comprising a disclosed CD47 inhibitor can further comprise a disclosed HDAC6 inhibitor. In an aspect, a disclosed CD47 inhibitor can be formulated as a pharmaceutical formulation. In an aspect, a disclosed pharmaceutical formulation comprising a disclosed CD47 inhibitor can further comprise a disclosed HDAC6 inhibitor.

In an aspect, a disclosed CD47 antisense oligonucleotide can target one or more aspects of HDAC6. Human CD47 is known to the art. The nucleotide sequences for CD47 are known. For example, CD47 is identified by HGNC 1682, NCBI Entrez Gene 961, Ensembl ENSG 00000196776, OMIM® 601028, and UniProtKB/Swiss-Prot Q08722.

In an aspect, a disclosed CD47 antisense oligonucleotide can target one or more parts or any part of an CD47 mRNA or pre-mRNA. In an aspect, a disclosed CD47 mRNA or pre-mRNA can comprise the sequence set forth in SEQ ID NO:11, SEQ ID NO:12, SEQ ID NO:13, or a fragment thereof. In an aspect, a disclosed CD47 inhibitor can comprise siRNA. In an aspect, a disclosed siRNA can target one or more parts or any part of the sequence set forth in SEQ ID NO:11, SEQ ID NO: 12, SEQ ID NO:13, or a fragment thereof. In an aspect, a disclosed CD47 can comprise the amino acid sequence set forth in any one of SEQ ID NO:07, SEQ ID NO:08, SEQ ID NO:09, or any fragment thereof. In an aspect, an mRNA sequence for a disclosed CD47 can comprise the sequence set forth in any one of SEQ ID NO:11, SEQ ID NO:12, SEQ ID NO:13, or a fragment thereof. In an aspect, a disclosed CD47 can be encoded by the sequence set forth in SEQ ID NO:14 or a fragment thereof.

In an aspect, a therapeutically effective amount of a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed CD47 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed CD47 inhibitor can comprise reduce, inhibit, and/or prevent the activity level and/or expression level of CD47 in the subject. In an aspect, a disclosed CD47 inhibitor can comprise reduce, inhibit, and/or prevent the activity level and/or expression level of CD47 in one or more macrophage populations in a subject. In an aspect, a disclosed CD47 inhibitor can modulate the phenotype and/or phagocytic function of one or more macrophage populations in a subject. In an aspect, a disclosed CD47 inhibitor can reduce, inhibit, and/or prevent the activity level and/or expression level of HDAC in one or more cancer cells or one or more populations of cancer cells.

In an aspect, a disclosed CD47 inhibitor can (i) reduce and/or prevent tumor growth; (ii) reduce or slow tumor metastasis; (iii) prevent and/or delay recurrence of the cancer; (iv) extend and/or prolong disease-free or tumor-free survival time; (v) increasing and/or lengthening overall survival time; (vi) reduce and/or minimize the frequency of treatment; (vii) relieve and/or ameliorate one or more symptoms of the cancer; (viii) reduce and/or decrease tumor burden, (ix) prevent and/or facilitate surgical intervention; (x) increase and/or enhance phagocytosis of cancer cells or tumor cells; (xi) increase and/or enhance immune cell infiltration in and/or around tumor microenvironment; (xii) enhance the subject's innate antitumor immunity; (xiii) drive and/or facilitate the M1 or pro-inflammatory phenotype of macrophages, (xiv) disrupt the CD47-SIRPα axis in one or more cancer cells or tumor cells; or (xv) any combination thereof.

In an aspect, a disclosed CD47 inhibitor can comprise any commercially available CD47 inhibitor or a combination of commercially available CD47 inhibitors.

In an aspect, a disclosed CD47 inhibitor can be formulated as lipid nanoparticles.

### 3. Formulations

Disclosed herein is a pharmaceutical formulation comprising a disclosed HDAC6 inhibitor, and one or more pharmaceutically acceptable carriers and/or excipients. Disclosed herein is a pharmaceutical formulation comprising one or more disclosed CD47 inhibitors, and one or more pharmaceutically acceptable carriers and/or excipients. Disclosed herein is a pharmaceutical formulation comprising one or more disclosed HDAC6 inhibitors, and one or more pharmaceutically acceptable carriers and/or excipients. Disclosed herein is a pharmaceutical formulation comprising one or more disclosed CD47 inhibitors, and one or more pharmaceutically acceptable carriers and/or excipients. Disclosed herein is a pharmaceutical formulation comprising a disclosed HDAC6 inhibitor and a disclosed CD47 inhibitor, and one or more pharmaceutically acceptable carriers and/or excipients. Disclosed herein is a pharmaceutical formulation comprising one or more disclosed HDAC6 inhibitors and one or more disclosed CD47 inhibitors, and one or more pharmaceutically acceptable carriers and/or excipients. Disclosed herein is a pharmaceutical formulation comprising a composition comprising a disclosed HDAC6 inhibitor, and one or more pharmaceutically acceptable carriers and/or excipients. Disclosed herein is a pharmaceutical formulation comprising a composition comprising one or more disclosed CD47 inhibitors, and one or more pharmaceutically acceptable carriers and/or excipients. Disclosed herein is a pharmaceutical formulation comprising a composition comprising one or more disclosed HDAC6 inhibitors, and one or more pharmaceutically acceptable carriers and/or excipients. Disclosed herein is a pharmaceutical formulation comprising a composition comprising one or more disclosed CD47 inhibitors, and one or more pharmaceutically acceptable carriers and/or excipients. Disclosed herein is a pharmaceutical formulation comprising a composition comprising a disclosed HDAC6 inhibitor and a disclosed CD47 inhibitor, and one or more pharmaceutically acceptable carriers and/or excipients. Disclosed herein is a pharmaceutical formulation comprising a composition comprising one or more disclosed HDAC6 inhibitors and one or more disclosed CD47 inhibitors, and one or more pharmaceutically acceptable carriers and/or excipients.

In an aspect, a disclosed pharmaceutical formulation can further comprise one or more excipients. In an aspect, a disclosed excipient can refer to an inert substance that is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

In an aspect, a disclosed pharmaceutically acceptable carrier can refer to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

In an aspect, a disclosed pharmaceutical formulation can (i) reduce and/or prevent tumor growth; (ii) reduce or slow tumor metastasis; (iii) prevent and/or delay recurrence of the cancer; (iv) extend and/or prolong disease-free or tumor-free survival time; (v) increasing and/or lengthening overall survival time; (vi) reduce and/or minimize the frequency of treatment; (vii) relieve and/or ameliorate one or more symptoms of the cancer; (viii) reduce and/or decrease tumor burden, (ix) prevent and/or facilitate surgical intervention; (x) increase and/or enhance phagocytosis of cancer cells or tumor cells; (xi) increase and/or enhance immune cell infiltration in and/or around tumor microenvironment; (xii) enhance the subject's innate antitumor immunity; (xiii) drive and/or facilitate the M1 or pro-inflammatory phenotype of macrophages, (xiv) disrupt the CD47-SIRPα axis in one or more cancer cells or tumor cells; or (xv) any combination thereof.

In an aspect, a disclosed pharmaceutical formulation can comprise reduce, inhibit, and/or prevent the activity level and/or expression level of HDAC6 in the subject. In an aspect, a disclosed pharmaceutical formulation can comprise reduce, inhibit, and/or prevent the activity level and/or expression level of HDAC6 in one or more macrophage populations in a subject. In an aspect, a disclosed pharmaceutical formulation can modulate the phenotype and/or phagocytic function of one or more macrophage populations in a subject. In an aspect, a disclosed pharmaceutical formulation can reduce, inhibit, and/or prevent the activity level and/or expression level of CD47 in one or more cancer cells or one or more populations of cancer cells. In an aspect, a disclosed pharmaceutical formulation can comprise reduce, inhibit, and/or prevent the activity level and/or expression level of SIRPα in the subject.

In an aspect, a disclosed pharmaceutical formulation can comprise lipid nanoparticles. In an aspect, a disclosed pharmaceutical formulation can be formulated as lipid nanoparticles.

In an aspect, a disclosed pharmaceutical formulation can be used in a disclosed method.

### 4. Kits

Disclosed herein is a kit comprising one or more disclosed HDAC6 inhibitors, one or more disclosed CD47 inhibitors, one or more disclosed pharmaceutical formulations, one or more disclosed compositions, one or more disclosed therapeutic agents and/or disclosed active agents, or any combination thereof.

In an aspect, a disclosed kit can comprise at least two components constituting the kit. Together, the components constitute a functional unit for a given purpose (such as, for example, performing any aspect of a disclosed method including preparing the components used in a disclosed method). Individual member components can be physically packaged together or separately. For example, a kit comprising an instruction for using the kit can or cannot physically include the instruction with other individual member components. Instead, the instruction can be supplied as a separate member component, either in a paper form or an electronic form which can be supplied on computer readable memory device or downloaded from an internet website, or as recorded presentation. In an aspect, a kit for use in a disclosed method can comprise one or more containers holding a disclosed HDAC6 inhibitor, a disclosed CD47 inhibitor, a disclosed composition, a disclosed pharmaceutical formulation, a disclosed therapeutic agent, and a label or package insert with instructions for use. In an aspect, suitable containers include, for example, bottles, vials, syringes, blister pack, etc. The containers can be formed from a variety of materials such as glass or plastic. The container can hold a disclosed composition, a disclosed pharmaceutical formulation, a disclosed therapeutic agent, or a combination thereof, and can have a sterile access port (for example the container can be an intravenous solution bag or a vial having a stopper pierceable by a hypodermic injection needle). In an aspect, a disclosed kit can comprise a “package insert”. In an aspect, a package insert can refer to instructions customarily included in commercial packages of therapeutic products, that contain information about the indications, usage, dosage, administration, contraindications and/or warnings concerning the use of such therapeutic products. The label or package insert can how a disclosed component can be used. A kit can comprise additional components necessary for administration such as, for example, other buffers, diluents, filters, needles, and syringes.

In an aspect, a disclosed kit can be used to (i) reduce and/or prevent tumor growth; (ii) reduce or slow tumor metastasis; (iii) prevent and/or delay recurrence of the cancer; (iv) extend and/or prolong disease-free or tumor-free survival time; (v) increasing and/or lengthening overall survival time; (vi) reduce and/or minimize the frequency of treatment; (vii) relieve and/or ameliorate one or more symptoms of the cancer; (viii) reduce and/or decrease tumor burden, (ix) prevent and/or facilitate surgical intervention; (x) increase and/or enhance phagocytosis of cancer cells or tumor cells; (xi) increase and/or enhance immune cell infiltration in and/or around tumor microenvironment; (xii) enhance the subject's innate antitumor immunity; (xiii) drive and/or facilitate the M1 or pro-inflammatory phenotype of macrophages, (xiv) disrupt the CD47-SIRPα axis in one or more cancer cells or tumor cells; or (xv) any combination thereof. In an aspect, a disclosed kit can be used to decrease the expression level and/or activity level of SIRPα.

In an aspect, a disclosed kit can be used in a disclosed method of treating a subject. In an aspect, a disclosed kit can be used in a disclosed method of slowing and/or preventing diseases progression. In an aspect, a disclosed kit can be used in a disclosed method of improving and/or enhancing a subject's innate antitumor immunity.

### H. Methods Using Disclosed Compositions

### 1. Methods of Treating a Subject

Disclosed herein is a method of treating a human subject having a cancer, the method comprising administering to a subject in need thereof a therapeutically effective amount of a HDAC6 inhibitor; and optionally administering to the subject a therapeutically effective amount of a CD47 inhibitor.

Disclosed herein is a method of treating a human subject having a cancer, the method comprising administering to a subject in need thereof a therapeutically effective amount of a CD47 inhibitor; and optionally administering to the subject a therapeutically effective amount of a HDAC6 inhibitor.

Disclosed herein is a method of treating a human subject having a cancer, the method comprising administering to a subject in need thereof a therapeutically effective amount of a HDAC6 inhibitor; and administering to the subject a therapeutically effective amount of a CD47 inhibitor.

Disclosed herein is a method of treating a human subject having a cancer, the method comprising administering to the subject in need thereof a therapeutically effective amount of composition comprising a HDAC6 inhibitor and a CD47 inhibitor.

Disclosed herein is a method of treating a human subject having a cancer, the method comprising administering to a subject in need thereof a therapeutically effective amount of a HDAC6 inhibitor; and optionally administering to the subject a therapeutically effective amount of a CD47 inhibitor; wherein the cancer is treated.

Disclosed herein is a method of treating a human subject having a cancer, the method comprising administering to a subject in need thereof a therapeutically effective amount of a CD47 inhibitor; and optionally administering to the subject a therapeutically effective amount of a HDAC6 inhibitor; wherein the cancer is treated.

Disclosed herein is a method of treating a human subject having a cancer, the method comprising administering to a subject in need thereof a therapeutically effective amount of a HDAC6 inhibitor; and administering to the subject a therapeutically effective amount of a CD47 inhibitor; wherein the cancer is treated.

Disclosed herein is a method of treating a human subject having a cancer, the method comprising administering to the subject in need thereof a therapeutically effective amount of composition comprising a HDAC6 inhibitor and a CD47 inhibitor; wherein the cancer is treated.

In an aspect of a disclosed method, treating the human subject can comprise (i) reducing and/or preventing tumor growth; (ii) reducing or slowing tumor metastasis; (iii) preventing and/or delaying recurrence of the cancer; (iv) extending and/or prolonging disease-free or tumor-free survival time; (v) increasing and/or lengthening overall survival time; (vi) reducing and/or minimizing the frequency of treatment; (vii) relieving and/or ameliorating one or more symptoms of the cancer; (viii) reducing and/or decreasing tumor burden, (ix) preventing and/or facilitating surgical intervention; (x) increasing and/or enhancing phagocytosis of cancer cells or tumor cells; (xi) increasing and/or enhancing immune cell infiltration in and/or around tumor microenvironment; (xii) enhancing the subject's innate antitumor immunity; (xiii) driving and/or facilitating the M1 or pro-inflammatory phenotype of macrophages, (xiv) disrupting the CD47-SIRPα axis in one or more cancer cells or tumor cells; or (xv) any combination thereof.

In an aspect, a disclosed method can decrease the expression level and/or activity level of SIRPα. In an aspect, a disclosed decrease can be a partial decrease or a complete decrease.

In an aspect of a disclosed method, a disclosed HDAC6 inhibitor can comprise any disclosed HDAC6 inhibitor. In an aspect, a disclosed HDAC6 inhibitor can comprise a small molecule, a peptide, a polynucleotide, an antibody or fragment thereof, an antisense oligonucleotide, siRNA, RNAi, or any combination thereof. In an aspect, a disclosed HDAC6 inhibitor can comprise an RNA therapeutic.

In an aspect of a disclosed method, a disclosed HDAC6 inhibitor can comprise Nexturastat A, Tubastatin A, KA2507, Ricolinostat (ACY-1215), Citarinostat (ACY-241), Tubacin, CAY10603, WT161, ACY-738, ACY-775, HPOB, SKLB-23bb, SS-208, Suprastat HDAC6 degrader-1 (PROTAC), HDAC6 degrader-3 (PROTAC), J22352 (PROTAC), HPB, HDAC6-IN-12 (compound GZ), HDAC6/8/BRPF1-IN-1, QTX125, CG347B, BRD73954, AES-135, AES-350, KH-259, SW-100, HPOB, Droxinostat (NS 41080), Bufexamac, KA2507, MC2625, MPT0G211, MPT0G211 mesylate, WT-161, ACY-738, ACY-775, XP5, HDAC-IN-35 (Compound 14), HDAC6-IN-15, HDAC6-IN-14, HDAC6-IN-13 (Compound 35m), HDAC6-IN-11 (Compound 9), HDAC6-IN-10, HDAC6-IN-9 (compound 12c), HDAC6-IN-8, HDAC6-IN-7 (TCS HDAC6 20b), HDAC6-IN-6 (compound 6a), HDAC6-IN-5 (compound 11b), HDAC6-IN-4 (C10), HDAC-IN-4, HDAC-IN-40, HDAC6-IN-3 (Compound 14), HDAC3/6-IN-2 (compound 15), or any combination thereof.

In an aspect of a disclosed method, a disclosed HDAC6 antisense oligonucleotide can target one or more parts or any part of an HDAC6 mRNA or pre-mRNA. In an aspect, a disclosed HDAC6 mRNA or pre-mRNA can comprise the sequence set forth in SEQ ID NO: 18, SEQ ID NO:19, or a fragment thereof. In an aspect, a disclosed HDAC6 inhibitor can comprise siRNA. In an aspect, a disclosed siRNA can target one or more parts or any part of the sequence set forth in SEQ ID NO:18, SEQ ID NO:19, or a fragment thereof. In an aspect, a disclosed HDAC6 can comprise the amino acid sequence set forth in any one of SEQ ID NO:15, SEQ ID NO:16, SEQ ID NO:17, or any fragment thereof. In an aspect, an mRNA sequence for a disclosed HDAC6 can comprise the sequence set forth in any one of SEQ ID NO:18, SEQ ID NO:19, or a fragment thereof. In an aspect, a disclosed HDAC6 can be encoded by the sequence set forth in SEQ ID NO:20, SEQ ID NO:21, or a fragment thereof.

In an aspect of a disclosed method, a disclosed CD47 inhibitor can comprise any disclosed CD47 inhibitor. In an aspect, a disclosed CD47 inhibitor can comprise a small molecule, a peptide, a polynucleotide, an antibody or fragment thereof, an antisense oligonucleotide, siRNA, RNAi, or any combination thereof. In an aspect, a disclosed CD47 inhibitor can comprise a RNA therapeutic.

In an aspect of a disclosed method, a disclosed CD47 inhibitor can comprise RRx-001, a dinitroazetidine derivative, Hu5F9-G4, CC-90002, TTI-621, ALX148, SRF231, SHR-1603, IB1188, ST-1901, SGNCD-47M, or any combination thereof. In an aspect, a disclosed CD47 inhibitor can comprise Gentulizumab, CC-90002 (INBRX 103), Hu5F9-G4 (5F9), Magrolimab, STI-6643, TI-061, AO-176, SRF231, AK117, 1B1188, IMC-002, SHR-1603, TJ011133, ZL-1201, evorpacept (ALX148), TTI-621, TTI-G22, or any combination thereof.

In an aspect of a disclosed method, a disclosed CD47 antisense oligonucleotide can target one or more parts or any part of an CD47 mRNA or pre-mRNA. In an aspect, a disclosed CD47 mRNA or pre-mRNA can comprise the sequence set forth in SEQ ID NO:11, SEQ ID NO:12, SEQ ID NO:13, or a fragment thereof. In an aspect, a disclosed CD47 inhibitor can comprise siRNA. In an aspect, a disclosed siRNA can target one or more parts or any part of the sequence set forth in SEQ ID NO: 11, SEQ ID NO:12, SEQ ID NO:13, or a fragment thereof. In an aspect, a disclosed CD47 can comprise the amino acid sequence set forth in any one of SEQ ID NO:07, SEQ ID NO:08, SEQ ID NO:09, or any fragment thereof. In an aspect, an mRNA sequence for a disclosed CD47 can comprise the sequence set forth in any one of SEQ ID NO:11, SEQ ID NO:12, SEQ ID NO: 13, or a fragment thereof. In an aspect, a disclosed CD47 can be encoded by the sequence set forth in SEQ ID NO:14 or a fragment thereof.

In an aspect, a disclosed CD47 inhibitor can be administered prior to, concurrent with, or after the administration of a disclosed HDAC6 inhibitor. In an aspect, a disclosed CD47 inhibitor can be administered about 3 months, about 2 months, or about 1 month prior to or after the administering of a disclosed HDAC6 inhibitor. In an aspect, a disclosed CD47 inhibitor can be administered about 8 weeks, about 7 weeks, about 6 weeks, about 5 weeks, about 4 weeks, about 3 weeks, about 2 weeks, or about 1 week prior to or after the administering of a disclosed HDAC6 inhibitor. In an aspect, a disclosed CD47 inhibitor can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to or after the administering of a disclosed HDAC6 inhibitor. In an aspect, a disclosed CD47 inhibitor can be administered about 24, 23, 22, 21, 20, 19, 18, 17, 16, 15, 14, 13, 12, 11, 10, 9, 8, 7, 6, 5, 4, 3, 2, or 1 hours prior to or after the administering of a disclosed HDAC6 inhibitor.

In an aspect, a disclosed method can further comprise repeating the administering of a disclosed HDAC6 inhibitor. In an aspect, a disclosed method can further comprise repeating the administering of a disclosed CD47 inhibitor.

In an aspect, a disclosed method can further comprise repeating the administering of a disclosed composition comprising a disclosed HDAC6 inhibitor and a disclosed CD47 inhibitor.

In an aspect of a disclosed method, administering a disclosed HDAC6 inhibitor can comprise systemic or direct administration. In an aspect of a disclosed method, administering a disclosed CD47 inhibitor can comprise systemic or direct administration. In an aspect of a disclosed method, administering a disclosed a composition comprising a disclosed HDAC6 inhibitor and a disclosed CD47 inhibitor can comprise systemic or direct administration.

In an aspect, administering a disclosed HDAC6 inhibitor can comprise oral administration, intravenous administration, intratumoral administration, intraperitoneal administration, or any combination thereof.

In an aspect, administering a disclosed HDAC6 inhibitor can comprise administration via any route of administration known to the art. In an aspect, administering a disclosed HDAC6 inhibitor can comprise administration via multiple routes of administration known to the art. In an aspect, administering a disclosed CD47 inhibitor comprise oral administration, intravenous administration, intratumoral administration, intraperitoneal administration, or any combination thereof. In an aspect, administering a disclosed CD47 inhibitor can comprise administration via any route of administration known to the art. In an aspect, administering a disclosed CD47 inhibitor can comprise administration via multiple routes of administration known to the art.

In an aspect, administering a disclosed composition comprising a disclosed HDAC6 inhibitor and a disclosed CD47 inhibitor can comprise oral administration, intravenous administration, intratumoral administration, intraperitoneal administration, or any combination thereof. In an aspect, administering a disclosed composition comprising a disclosed HDAC6 inhibitor and a disclosed CD47 inhibitor can comprise administration via any route of administration known to the art. In an aspect, administering a disclosed composition comprising a disclosed HDAC6 inhibitor and a disclosed CD47 inhibitor can comprise administration via multiple routes of administration known to the art.

In an aspect, local administration can comprise delivery to one or more of the subject's body systems having cancerous cells or tumorous growth. In an aspect, the subject's one or more body systems having cancerous cells or tumorous growth can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof.

In an aspect of a disclosed method, administering a disclosed HDAC6 inhibitor can comprise a single dose or multiple doses (such as 2, 3, 4, 5, 6, 7, 8, 9, 10, or more than 10 doses). In an aspect of a disclosed method, administering a disclosed CD47 inhibitor can comprise a single dose or multiple doses (such as 2, 3, 4, 5, 6, 7, 8, 9, 10, or more than 10 doses). In an aspect of a disclosed method, administering a disclosed composition comprising a disclosed HDAC6 inhibitor and a disclosed CD47 inhibitor can comprise a single dose or multiple doses (such as 2, 3, 4, 5, 6, 7, 8, 9, 10, or more than 10 doses).

In an aspect, a therapeutically effective amount of a disclosed HDAC6 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed HDAC6 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a therapeutically effective amount of a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed CD47 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a therapeutically effective amount of a disclosed composition comprising a disclosed HDAC6 inhibitor and a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed composition comprising a disclosed HDAC6 inhibitor and a disclosed CD47 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed method can further comprise monitoring the subject for adverse effects. In an aspect, in the absence of adverse effects, a disclosed method can further comprise continuing to treat the subject. In an aspect, continuing to treat the subject can comprise continuing to administer a disclosed HDAC6 inhibitor and/or continuing to administer a disclosed CD47 inhibitor and/or continuing to administer a disclosed composition comprising a disclosed HDAC6 inhibitor and a disclosed CD47 inhibitor.

In an aspect, in the presence of adverse effects, a disclosed method can further comprise modifying one or more steps of the method. In an aspect, modifying one or more steps of a disclosed method can comprise modifying one or more administering steps. In an aspect, modifying one or more disclosed administering steps can comprise changing the amount of a disclosed HDAC6 inhibitor and/or a disclosed CD47 inhibitor administered to the subject, changing the frequency of a disclosed HDAC6 inhibitor and/or a disclosed CD47 inhibitor administration, changing the duration of a disclosed HDAC6 inhibitor and/or a disclosed CD47 inhibitor administration, changing the route of a disclosed HDAC6 inhibitor and/or a disclosed CD47 inhibitor administration, or any combination thereof. In an aspect, modifying one or more disclosed administering steps can comprise changing the amount a disclosed composition comprising a disclosed HDAC6 inhibitor and a disclosed CD47 inhibitor administered to the subject, changing the frequency of a disclosed composition comprising a disclosed HDAC6 inhibitor and a disclosed CD47 inhibitor administration, changing the duration of a disclosed composition comprising a disclosed HDAC6 inhibitor and a disclosed CD47 inhibitor administration, changing the route of a disclosed composition comprising a disclosed HDAC6 inhibitor and a disclosed CD47 inhibitor administration, or any combination thereof.

In an aspect, a disclosed method can further comprise administering to the subject one or more additional anti-cancer therapies. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, innotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method can further comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL12, CCR2, CCR7, CCR8, CCR9, CXCR4, CX3CL1, CX3CR1, or any combination thereof.

In an aspect, cancerous cells and/or tumorous cells can comprise ovarian cancer cells, ovarian adenocarcinoma cells, ovarian teratocarcinoma cells, lung cancer cells, small cell lung cancer (SCLC) cells, non-small cell lung cancer (NSCLC) cells, squamous cell lung carcinoma cells, adenocarcinoma cells, gastric cancer cells, breast cancer cells, hepatic cancer cells, pancreatic cancer cells, skin cancer cells, in particular basal cell carcinoma and squamous cell carcinoma cells, malignant melanoma cells, head and neck cancer cells, malignant pleomorphic adenoma cells, sarcoma cells, synovial sarcoma cells, carcinosarcoma cells, bile duct cancer cells, bladder cancer cells, transitional cell carcinoma cells, papillary carcinoma cells, kidney cancer cells, renal cell carcinoma cells, clear cell renal cell carcinoma cells, papillary renal cell carcinoma cells, colon cancer cells, small bowel cancer cells, small bowel adenocarcinoma cells, adenocarcinoma of the ileum cells, testicular embryonal carcinoma cells, placental choriocarcinoma cells, cervical cancer cells, testicular cancer cells, testicular seminoma cells, testicular teratoma cells, embryonic testicular cancer cells, uterine cancer cells, teratocarcinoma cells, embryonal carcinoma cells, or any combination thereof.

In an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a disclosed method can further comprise surgically resecting the tumor from the subject.

In an aspect, a disclosed method can further comprise continuing to administer to the subject a therapeutically effective amount of one or more disclosed anti-cancer therapies.

In an aspect, a disclosed method can further comprise subjecting the subject to one or more invasive or non-invasive diagnostic assessments. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination. In an aspect, a disclosed invasive diagnostic assessment can comprise a tissue biopsy.

In an aspect, a disclosed HDAC6 inhibitor can comprise reduce, inhibit, and/or prevent the activity level and/or expression level of HDAC6 in the subject. In an aspect, a disclosed HDAC6 inhibitor can comprise reduce, inhibit, and/or prevent the activity level and/or expression level of HDAC6 in one or more macrophage populations in a subject. In an aspect, a disclosed HDAC6 inhibitor can modulate the phenotype and/or phagocytic function of one or more macrophage populations in a subject. In an aspect, a disclosed HDAC6 inhibitor can reduce, inhibit, and/or prevent the activity level and/or expression level of CD47 in one or more cancer cells or one or more populations of cancer cells.

In an aspect, a disclosed method can further comprise administering to the subject a therapeutically effective amount of M1 macrophages. In an aspect, a disclosed method can further comprise contacting a population of M0 or naïve macrophages with a polarizing agent. In an aspect, a disclosed polarizing agent can drive the M0 or naïve macrophages to a M1 or pro-inflammatory phenotype. In an aspect, a disclosed polarizing agent can comprise lipopolysaccharide (LPS), interferon gamma (IFN-γ), granulocyte-macrophage colony-stimulating factor (GM-CSF), or any combination thereof. In an aspect of a disclosed method, contacting a population of polarized M1 or pro-inflammatory macrophages with one or more disclosed polarizing agents can enhance and/or improve the M1 phenotype of the polarized macrophages. In an aspect, a disclosed population of M1 macrophages can be macrophages that were treated ex vivo with one or more disclosed polarizing agents. In an aspect of a disclosed method, contacting a population of M0 or naïve macrophages with a disclosed polarizing agent can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of M0 or naïve macrophages with a disclosed polarizing agent can comprise more than 24 hours.

In an aspect of a disclosed method, contacting a population of M0 or naïve macrophages with a disclosed polarizing agent can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect of a disclosed method, contacting a population of M0 or naïve macrophages with a disclosed polarizing agent can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of M0 or naïve macrophages with a disclosed polarizing agent can comprise more than 24 hours. In an aspect of a disclosed method, contacting a population of M0 or naïve macrophages with a disclosed polarizing agent can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect, M0 or naïve macrophages can be contacted with a disclosed polarizing agent one or more times, and then polarized to M1 or pro-inflammatory macrophages.

In an aspect of a disclosed method, M1 or pro-inflammatory macrophages can be administered to a subject prior to, concurrent with, or after the administering a disclosed HDAC6 inhibitor and/or a disclosed CD47 inhibitor. In an aspect of a disclosed method, M1 or pro-inflammatory macrophages can be administered to a subject prior to, concurrent with, or after a disclosed composition comprising a disclosed HDAC6 inhibitor and a disclosed CD47 inhibitor.

In an aspect, a subject can be male or female. In an aspect, a subject can be an adult, a teenager, an adolescent, a child, a toddler, a baby, or an infant. In an aspect, a subject has received treatment for one or more disclosed cancers. In an aspect, a subject can be in treatment for one or more disclosed cancers. In an aspect, a subject can be treatment-naïve for one or more disclosed cancers.

In an aspect, a disclosed method can further comprise collecting one or more blood samples from a subject at the same time or at different times. For example, in an aspect, a blood sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample can be collected from a subject prior to treatment, during treatment, after treatment, or any combination thereof. In an aspect, a blood sample can be collected from a subject at any time deemed medically and/or clinically appropriate by the skilled clinician. In an aspect, a disclosed blood sample can be assayed and/or evaluated for one or more biomarkers of cancer, one or more biomarkers of disease progression, or any combination thereof.

In an aspect, a disclosed HDAC6 inhibitor can comprise any commercially available HDAC6 inhibitor or a combination of commercially available HDAC6 inhibitors. In an aspect, a disclosed HDAC6 inhibitor can comprise a selective HDAC6 inhibitor or HDAC6 selective inhibitor and can refer to a compound that preferentially inhibits histone deacetylase 6 over one or more other histone deacetylase isoforms, e.g., HDAC1, HDAC2, HDAC3, HDAC4, HDAC5, HDAC7, HDAC8, HDAC9, HDAC10, and/or HDCA11 in a cell-based in vitro assay. For example, a compound having a HDAC6 IC₅₀=5 nM and a HDAC1 IC₅₀ of 500 nM is a selective HDAC6 inhibitor that is 100-fold more selective over HDAC1; a compound having a HDAC6 ICso=5 nM, a HDAC1 ICso=500 nM, and a HDAC3 ICso=50 nM is a selective HDAC6 inhibitor that is 100-fold more selective over HDAC1 and 10-fold more selective over HDAC3; and so on. In an aspect, a disclosed selective HDAC6 inhibitor can preferentially inhibit HDAC6 over HDAC1, HDAC2, HDAC3, HDAC4, HDAC5, HDAC7, HDAC8, HDAC9, HDAC10, and/or HDAC11 In an aspect, a disclosed selective HDAC6 inhibitor can preferentially inhibit HDAC6 over one or more other HDAC isoforms. In an aspect, a disclosed selective HDAC6 inhibitor can be at least about 5-fold more selective over one or more other HDAC isoforms. In an aspect, a disclosed selective HDAC6 inhibitor can be at least about 10-fold more selective over one or more other HDAC isoforms. In an aspect, a disclosed selective HDAC6 inhibitor can be at least about 15-fold more selective over one or more other HDAC isoforms. In an aspect, a disclosed selective HDAC6 inhibitor can be at least about 20-fold more selective over one or more other HDAC isoforms. In an aspect, a disclosed selective HDAC6 inhibitor can be at least about 30-fold more selective over one or more other HDAC isoforms. In an aspect, a disclosed selective HDAC6 inhibitor can be at least about 40-fold more selective over one or more other HDAC isoforms. In an aspect, a disclosed selective HDAC6 inhibitor can be at least about 50-fold more selective over one or more other HDAC isoforms. In an aspect, a disclosed selective HDAC6 inhibitor can be at least about 100-fold more selective over one or more other HDAC isoforms. In an aspect, a disclosed selective HDAC6 inhibitor can be at least about 150-fold more selective over one or more other HDAC isoforms. In an aspect, the selective HDAC6 inhibitor can be at least about 200-fold more selective over one or more other HDAC isoforms. In an aspect, a disclosed selective HDAC6 inhibitor can be at least about 250-fold more selective over one or more other HDAC isoforms. In an aspect, a disclosed selective HDAC6 inhibitor can be at least about 500-fold more selective over one or more other HDAC isoforms. In an aspect, a disclosed selective HDAC6 inhibitor can be at least about 750-fold more selective over one or more other HDAC isoforms. In an aspect, a disclosed selective HDAC6 inhibitor can be at least about 1000-fold more selective over one or more other HDAC isoforms. In an aspect, a disclosed selective HDAC6 inhibitor can be at least about 2000-fold more selective over one or more other HDAC isoforms. In an aspect, a disclosed selective HDAC6 inhibitor can be at least about 3000-fold more selective over one or more other HDAC isoforms. HDAC6 selectivity over the other HDAC isoforms in cell-based assays can be determined using methods known in the art. In another aspect, the selective HDAC6 inhibitor can be at about 10-fold to about 3000-fold more selective over one or more other HDAC isoforms. In an aspect, a disclosed selective HDAC6 inhibitor can be at about 20-fold to about 3000-fold more selective over one or more other HDAC isoforms. In an aspect, a disclosed selective HDAC6 inhibitor can be at about 50-fold to about 3000-fold more selective over one or more other HDAC isoforms. In an aspect, a disclosed selective HDAC6 inhibitor can be at about 100-fold to about 3000-fold more selective over one or more other HDAC isoforms. In an aspect, a disclosed selective HDAC6 inhibitor can be at about 500-fold to about 3000-fold more selective over one or more other HDAC isoforms.

In an aspect, a disclosed composition can comprise any disclosed HDAC6 inhibitor and any disclosed CD47 inhibitor. In an aspect, a disclosed composition can comprise any disclosed any commercially available HDAC6 inhibitor and any disclosed commercially available CD47 inhibitor.

In an aspect, a disclosed method can reduce the risk of developing one or more metastases. A reduction in the risk of developing one or more metastases comprises a reduction of 10%, 20%, 30%, 40%, 50%, 60%, 70%, 80%, 90%, 100%, or any amount in the risk of metastases when compared to a control subject (such as, for example, a subject that has not received a disclosed HDAC6 inhibitor and/or a disclosed CD47 inhibitor or a composition thereof).

In an aspect, a disclosed method can comprise improving and/or enhancing the subject's quality of life and/or movement. In an aspect of a disclosed method, a disclosed improvement and/or enhancement (such as, for example, in the subject's quality of life and/or movement) can comprise a 10%, 20%, 30%, 40%, 50%, 60%, 70%, 80%, 90%, 100%, or any amount of improvement and/or enhancement when compared to a control subject (such as, for example, a subject that has not received a disclosed HDAC6 inhibitor and/or a disclosed CD47 inhibitor or a composition thereof). In an aspect, a disclosed improvement and/or enhancement (such as, for example, in the subject's quality of life and/or movement) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not received a disclosed HDAC6 inhibitor and/or a disclosed CD47 inhibitor or a composition thereof).

In an aspect, a disclosed diminishment (such as, for example, the size of one or more tumors) can comprise a 10%, 20%, 30%, 40%, 50%, 60%, 70%, 80%, 90%, 100%, or any amount of a decrease when compared to a control subject (such as, for example, a subject that has not received a disclosed HDAC6 inhibitor and/or a disclosed CD47 inhibitor or a composition thereof). In an aspect, a disclosed diminishment (such as, for example, the size of one or more tumors) can comprise a 10-20%, 20-30%, 30-40%, 40-50%, 50-60%, 60-70%, 70-80%, 80-90%, or 90-100% or any amount of a decrease when compared to a control subject (such as a subject that has not received a disclosed HDAC6 inhibitor and/or a disclosed CD47 inhibitor or a composition thereof).

In an aspect, a disclosed method can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinical can decide to change an aspect of the subject's treatment and/or change the subject's diagnosis or prognosis.

In an aspect, a disclosed method can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed HDAC6 inhibitor and/or a disclosed CD47 inhibitor and/or a composition comprising a disclosed HDAC6 inhibitor and a disclosed CD47 inhibitor. In an aspect, metabolic and/or physiologic data can inform the clinician when make subsequent treatment decisions.

In an aspect, a disclosed method can comprise administering to the subject am anti-PD1 molecule. In an aspect, a disclosed anti-PD1 molecule can comprise a PD1 antibody, a PDL1 antibody, or any combination thereof. In an aspect, a disclosed PD1 antibody can comprise a monoclonal antibody, a humanized monoclonal antibody, or a fragment thereof. In an aspect, a disclosed PD1 antibody can comprise a polyclonal antibody, a humanized polyclonal antibody, or a fragment thereof. In an aspect, a disclosed PD1 antibody can comprise any antibody or antibody fragment that specifically recognizes PD1. In an aspect, a disclosed PDL1 antibody can comprise a monoclonal antibody, a humanized monoclonal antibody, or a fragment thereof. In an aspect, a disclosed PDL1 antibody can comprise a polyclonal antibody, a humanized polyclonal antibody, or a fragment thereof. In an aspect, a disclosed PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed PD1 antibody can comprise nivolumab, pembrolizumab, STI-A1014, pidilizumab, or any combination thereof.

In an aspect of a disclosed method, a disclosed PD1 antibody, a disclosed PDL1 antibody, or a combination thereof can be a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of a disclosed anti-PD1 molecule. In an aspect, a disclosed anti-PD1 molecule can be administered prior to, concurrent with, or after the administration of a disclosed HDAC6 inhibitor and/or a disclosed CD47 inhibitor.

In an aspect, a disclosed method can comprise diagnosing a subject in need of a disclosed composition.

In an aspect, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

In an aspect of a disclosed method, a disclosed method can further comprise administering to the subject one or more SIRPα inhibitors. In an aspect, a disclosed SIRPα inhibitor can comprise a small molecule, a peptide, a polynucleotide, an antibody or fragment thereof, an antisense oligonucleotide, siRNA, RNAi, or any combination thereof. In an aspect, a disclosed SIRPα antisense oligonucleotide can target one or more parts or any part of an SIRPα mRNA or pre-mRNA. In an aspect, a disclosed SIRPα mRNA or pre-mRNA can comprise the sequence set forth in SEQ ID NO:25, SEQ ID NO:26, SEQ ID NO:27, SEQ ID NO:28, or a fragment thereof. In an aspect, a disclosed SIRPα inhibitor can comprise siRNA. In an aspect, a disclosed siRNA can target one or more parts or any part of the sequence set forth in SEQ ID NO:25, SEQ ID NO:26, SEQ ID NO:27, SEQ ID NO:28, or a fragment thereof. In an aspect, a disclosed SIRPα can comprise the amino acid sequence set forth in any one of SEQ ID NO:22, SEQ ID NO:23, SEQ ID NO:24, or any fragment thereof. In an aspect, an mRNA sequence for a disclosed SIRPα can comprise the sequence set forth in any one of SEQ ID NO:25, SEQ ID NO:26, SEQ ID NO:27, SEQ ID NO:28, or a fragment thereof. In an aspect, a disclosed SIRPα can be encoded by the sequence set forth in SEQ ID NO:29 or a fragment thereof.

2. Methods of Slowing and/or Preventing Disease Progression

Disclosed herein is a method of slowing and/or prevent disease progression, the method comprising administering to a subject in need thereof a therapeutically effective amount of a HDAC6 inhibitor; and optionally administering to the subject a therapeutically effective amount of a CD47 inhibitor.

Disclosed herein is a method of slowing and/or prevent disease progression, the method comprising administering to a subject in need thereof a therapeutically effective amount of a CD47 inhibitor; and optionally administering to the subject a therapeutically effective amount of a HDAC6 inhibitor.

Disclosed herein is a method of slowing and/or prevent disease progression, the method comprising administering to a subject having cancer a therapeutically effective amount of a HDAC6 inhibitor; and administering to the subject a therapeutically effective amount of a CD47 inhibitor.

Disclosed herein is a method of slowing and/or prevent disease progression, the method comprising administering to a subject having cancer a therapeutically effective amount of a composition comprising a HDAC6 inhibitor and a CD47 inhibitor.

Disclosed herein is a method of slowing and/or prevent disease progression, the method comprising administering to a subject in need thereof a therapeutically effective amount of a HDAC6 inhibitor; and optionally administering to the subject a therapeutically effective amount of a CD47 inhibitor; wherein the cancer is treated.

Disclosed herein is a method of slowing and/or prevent disease progression, the method comprising administering to a subject in need thereof a therapeutically effective amount of a CD47 inhibitor; and optionally administering to the subject a therapeutically effective amount of a HDAC6 inhibitor; wherein the cancer is treated.

Disclosed herein is a method of slowing and/or prevent disease progression, the method comprising administering to a subject having cancer a therapeutically effective amount of a HDAC6 inhibitor; and administering to the subject a therapeutically effective amount of a CD47 inhibitor; wherein the cancer is treated.

Disclosed herein is a method of slowing and/or prevent disease progression, the method comprising administering to a subject having cancer a therapeutically effective amount of a composition comprising a HDAC6 inhibitor and a CD47 inhibitor; wherein the cancer is treated.

In an aspect of a disclosed method, treating the human subject can comprise (i) reducing and/or preventing tumor growth; (ii) reducing or slowing tumor metastasis; (iii) preventing and/or delaying recurrence of the cancer; (iv) extending and/or prolonging disease-free or tumor-free survival time; (v) increasing and/or lengthening overall survival time; (vi) reducing and/or minimizing the frequency of treatment; (vii) relieving and/or ameliorating one or more symptoms of the cancer; (viii) reducing and/or decreasing tumor burden, (ix) preventing and/or facilitating surgical intervention; (x) increasing and/or enhancing phagocytosis of cancer cells or tumor cells; (xi) increasing and/or enhancing immune cell infiltration in and/or around tumor microenvironment; (xii) enhancing the subject's innate antitumor immunity; (xiii) driving and/or facilitating the M1 or pro-inflammatory phenotype of macrophages, (xiv) disrupting the CD47-SIRPα axis in one or more cancer cells or tumor cells; or (xv) any combination thereof. In an aspect, a disclosed method can decrease the expression level and/or activity level of SIRPα. In an aspect, a disclosed decrease can be a partial decrease or a complete decrease.

In an aspect of a disclosed method, a disclosed HDAC6 inhibitor can comprise any disclosed HDAC6 inhibitor. In an aspect, a disclosed HDAC6 inhibitor can comprise a small molecule, a peptide, a polynucleotide, an antibody or fragment thereof, an antisense oligonucleotide, siRNA, RNAi, or any combination thereof. In an aspect, a disclosed HDAC6 inhibitor can comprise a RNA therapeutic.

In an aspect of a disclosed method, a disclosed HDAC6 inhibitor can comprise Nexturastat A, Tubastatin A, KA2507, Ricolinostat (ACY-1215), Citarinostat (ACY-241), Tubacin, CAY10603, WT161, ACY-738, ACY-775, HPOB, SKLB-23bb, SS-208, Suprastat HDAC6 degrader-1 (PROTAC), HDAC6 degrader-3 (PROTAC), J22352 (PROTAC), HPB, HDAC6-IN-12 (compound GZ), HDAC6/8/BRPF1-IN-1, QTX125, CG347B, BRD73954, AES-135, AES-350, KH-259, SW-100, HPOB, Droxinostat (NS 41080), Bufexamac, KA2507, MC2625, MPT0G211, MPT0G211 mesylate, WT-161, ACY-738, ACY-775, XP5, HDAC-IN-35 (Compound 14), HDAC6-IN-15, HDAC6-IN-14, HDAC6-IN-13 (Compound 35m), HDAC6-IN-11 (Compound 9), HDAC6-IN-10, HDAC6-IN-9 (compound 12c), HDAC6-IN-8, HDAC6-IN-7 (TCS HDAC6 20b), HDAC6-IN-6 (compound 6a), HDAC6-IN-5 (compound 11b), HDAC6-IN-4 (C10), HDAC-IN-4, HDAC-IN-40, HDAC6-IN-3 (Compound 14), HDAC3/6-IN-2 (compound 15), or any combination thereof.

In an aspect of a disclosed method, a disclosed HDAC6 antisense oligonucleotide can target one or more parts or any part of an HDAC6 mRNA or pre-mRNA. In an aspect, a disclosed HDAC6 mRNA or pre-mRNA can comprise the sequence set forth in SEQ ID NO: 18, SEQ ID NO:19, or a fragment thereof. In an aspect, a disclosed HDAC6 inhibitor can comprise siRNA. In an aspect, a disclosed siRNA can target one or more parts or any part of the sequence set forth in SEQ ID NO:18, SEQ ID NO:19, or a fragment thereof. In an aspect, a disclosed HDAC6 can comprise the amino acid sequence set forth in any one of SEQ ID NO:15, SEQ ID NO:16, SEQ ID NO:17, or any fragment thereof. In an aspect, an mRNA sequence for a disclosed HDAC6 can comprise the sequence set forth in any one of SEQ ID NO:18, SEQ ID NO:19, or a fragment thereof. In an aspect, a disclosed HDAC6 can be encoded by the sequence set forth in SEQ ID NO:20, SEQ ID NO:21, or a fragment thereof.

In an aspect of a disclosed method, a disclosed CD47 inhibitor can comprise RRx-001, a dinitroazetidine derivative, Hu5F9-G4, CC-90002, TTI-621, ALX148, SRF231, SHR-1603, IBI188, ST-1901, SGNCD-47M, or any combination thereof. In an aspect, a disclosed CD47 inhibitor can comprise Gentulizumab, CC-90002 (INBRX 103), Hu5F9-G4 (5F9), Magrolimab, STI-6643, TI-061, AO-176, SRF231, AK117, IB1188, IMC-002, SHR-1603, TJ011133, ZL-1201, evorpacept (ALX148), TTI-621, TTI-G22, or any combination thereof.

In an aspect of a disclosed method, a disclosed CD47 inhibitor can comprise any disclosed CD47 inhibitor. In an aspect, a disclosed CD47 inhibitor can comprise a small molecule, a peptide, a polynucleotide, an antibody or fragment thereof, an antisense oligonucleotide, siRNA, RNAi, or any combination thereof. In an aspect, a disclosed CD47 inhibitor can comprise a RNA therapeutic.

In an aspect of a disclosed method, a disclosed CD47 antisense oligonucleotide can target one or more parts or any part of an CD47 mRNA or pre-mRNA. In an aspect, a disclosed CD47 mRNA or pre-mRNA can comprise the sequence set forth in SEQ ID NO:11, SEQ ID NO:12, SEQ ID NO:13, or a fragment thereof. In an aspect, a disclosed CD47 inhibitor can comprise siRNA. In an aspect, a disclosed siRNA can target one or more parts or any part of the sequence set forth in SEQ ID NO: 11, SEQ ID NO:12, SEQ ID NO:13, or a fragment thereof. In an aspect, a disclosed CD47 can comprise the amino acid sequence set forth in any one of SEQ ID NO:07, SEQ ID NO:08, SEQ ID NO:09, or any fragment thereof. In an aspect, an mRNA sequence for a disclosed CD47 can comprise the sequence set forth in any one of SEQ ID NO:11, SEQ ID NO:12, SEQ ID NO: 13, or a fragment thereof. In an aspect, a disclosed CD47 can be encoded by the sequence set forth in SEQ ID NO:14 or a fragment thereof.

In an aspect, a disclosed CD47 inhibitor can be administered prior to, concurrent with, or after the administration of a disclosed HDAC6 inhibitor. In an aspect, a disclosed CD47 inhibitor can be administered about 3 months, about 2 months, or about 1 month prior to or after the administering of a disclosed HDAC6 inhibitor. In an aspect, a disclosed CD47 inhibitor can be administered about 8 weeks, about 7 weeks, about 6 weeks, about 5 weeks, about 4 weeks, about 3 weeks, about 2 weeks, or about 1 week prior to or after the administering of a disclosed HDAC6 inhibitor. In an aspect, a disclosed CD47 inhibitor can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to or after the administering of a disclosed HDAC6 inhibitor. In an aspect, a disclosed CD47 inhibitor can be administered about 24, 23, 22, 21, 20, 19, 18, 17, 16, 15, 14, 13, 12, 11, 10, 9, 8, 7, 6, 5, 4, 3, 2, or 1 hours prior to or after the administering of a disclosed HDAC6 inhibitor.

In an aspect, a disclosed method can further comprise repeating the administering of a disclosed HDAC6 inhibitor. In an aspect, a disclosed method can further comprise repeating the administering of a disclosed CD47 inhibitor.

In an aspect, a disclosed method can further comprise repeating the administering of a disclosed composition comprising a disclosed HDAC6 inhibitor and a disclosed CD47 inhibitor.

In an aspect of a disclosed method, administering a disclosed HDAC6 inhibitor can comprise systemic or direct administration. In an aspect of a disclosed method, administering a disclosed CD47 inhibitor can comprise systemic or direct administration. In an aspect of a disclosed method, administering a disclosed a composition comprising a disclosed HDAC6 inhibitor and a disclosed CD47 inhibitor can comprise systemic or direct administration.

In an aspect, administering a disclosed HDAC6 inhibitor can comprise oral administration, intravenous administration, intratumoral administration, intraperitoneal administration, or any combination thereof.

In an aspect, administering a disclosed HDAC6 inhibitor can comprise administration via any route of administration known to the art. In an aspect, administering a disclosed HDAC6 inhibitor can comprise administration via multiple routes of administration known to the art. In an aspect, administering a disclosed CD47 inhibitor comprise oral administration, intravenous administration, intratumoral administration, intraperitoneal administration, or any combination thereof. In an aspect, administering a disclosed CD47 inhibitor can comprise administration via any route of administration known to the art. In an aspect, administering a disclosed CD47 inhibitor can comprise administration via multiple routes of administration known to the art.

In an aspect, administering a disclosed composition comprising a disclosed HDAC6 inhibitor and a disclosed CD47 inhibitor can comprise oral administration, intravenous administration, intratumoral administration, intraperitoneal administration, or any combination thereof. In an aspect, administering a disclosed composition comprising a disclosed HDAC6 inhibitor and a disclosed CD47 inhibitor can comprise administration via any route of administration known to the art. In an aspect, administering a disclosed composition comprising a disclosed HDAC6 inhibitor and a disclosed CD47 inhibitor can comprise administration via multiple routes of administration known to the art.

In an aspect, local administration can comprise delivery to one or more of the subject's body systems having cancerous cells or tumorous growth. In an aspect, the subject's one or more body systems having cancerous cells or tumorous growth can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof.

In an aspect of a disclosed method, administering a disclosed HDAC6 inhibitor can comprise a single dose or multiple doses (such as 2, 3, 4, 5, 6, 7, 8, 9, 10, or more than 10 doses). In an aspect of a disclosed method, administering a disclosed CD47 inhibitor can comprise a single dose or multiple doses (such as 2, 3, 4, 5, 6, 7, 8, 9, 10, or more than 10 doses). In an aspect of a disclosed method, administering a disclosed composition comprising a disclosed HDAC6 inhibitor and a disclosed CD47 inhibitor can comprise a single dose or multiple doses (such as 2, 3, 4, 5, 6, 7, 8, 9, 10, or more than 10 doses).

In an aspect, a therapeutically effective amount of a disclosed HDAC6 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed HDAC6 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed HDAC6 inhibitor can comprise reduce, inhibit, and/or prevent the activity level and/or expression level of HDAC6 in the subject. In an aspect, a disclosed HDAC6 inhibitor can comprise reduce, inhibit, and/or prevent the activity level and/or expression level of HDAC6 in one or more macrophage populations in a subject. In an aspect, a disclosed HDAC6 inhibitor can modulate the phenotype and/or phagocytic function of one or more macrophage populations in a subject. In an aspect, a disclosed HDAC6 inhibitor can reduce, inhibit, and/or prevent the activity level and/or expression level of CD47 in one or more cancer cells or one or more populations of cancer cells.

In an aspect, a therapeutically effective amount of a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed CD47 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a therapeutically effective amount of a disclosed composition comprising a disclosed HDAC6 inhibitor and a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed composition comprising a disclosed HDAC6 inhibitor and a disclosed CD47 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed method can further comprise monitoring the subject for adverse effects. In an aspect, in the absence of adverse effects, a disclosed method can further comprise continuing to treat the subject. In an aspect, continuing to treat the subject can comprise continuing to administer a disclosed HDAC6 inhibitor and/or continuing to administer a disclosed CD47 inhibitor and/or continuing to administer a disclosed composition comprising a disclosed HDAC6 inhibitor and a disclosed CD47 inhibitor.

In an aspect, in the presence of adverse effects, a disclosed method can further comprise modifying one or more steps of the method. In an aspect, modifying one or more steps of a disclosed method can comprise modifying one or more administering steps. In an aspect, modifying one or more disclosed administering steps can comprise changing the amount of a disclosed HDAC6 inhibitor and/or a disclosed CD47 inhibitor administered to the subject, changing the frequency of a disclosed HDAC6 inhibitor and/or a disclosed CD47 inhibitor administration, changing the duration of a disclosed HDAC6 inhibitor and/or a disclosed CD47 inhibitor administration, changing the route of a disclosed HDAC6 inhibitor and/or a disclosed CD47 inhibitor administration, or any combination thereof. In an aspect, modifying one or more disclosed administering steps can comprise changing the amount a disclosed composition comprising a disclosed HDAC6 inhibitor and a disclosed CD47 inhibitor administered to the subject, changing the frequency of a disclosed composition comprising a disclosed HDAC6 inhibitor and a disclosed CD47 inhibitor administration, changing the duration of a disclosed composition comprising a disclosed HDAC6 inhibitor and a disclosed CD47 inhibitor administration, changing the route of a disclosed composition comprising a disclosed HDAC6 inhibitor and a disclosed CD47 inhibitor administration, or any combination thereof.

In an aspect, a disclosed method can further comprise administering to the subject one or more additional anti-cancer therapies. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, innotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method can further comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL12, CCR2, CCR7, CCR8, CCR9, CXCR4, CX3CL1, CX3CR1, or any combination thereof.

In an aspect, cancerous cells and/or tumorous cells can comprise ovarian cancer cells, ovarian adenocarcinoma cells, ovarian teratocarcinoma cells, lung cancer cells, small cell lung cancer (SCLC) cells, non-small cell lung cancer (NSCLC) cells, squamous cell lung carcinoma cells, adenocarcinoma cells, gastric cancer cells, breast cancer cells, hepatic cancer cells, pancreatic cancer cells, skin cancer cells, in particular basal cell carcinoma and squamous cell carcinoma cells, malignant melanoma cells, head and neck cancer cells, malignant pleomorphic adenoma cells, sarcoma cells, synovial sarcoma cells, carcinosarcoma cells, bile duct cancer cells, bladder cancer cells, transitional cell carcinoma cells, papillary carcinoma cells, kidney cancer cells, renal cell carcinoma cells, clear cell renal cell carcinoma cells, papillary renal cell carcinoma cells, colon cancer cells, small bowel cancer cells, small bowel adenocarcinoma cells, adenocarcinoma of the ileum cells, testicular embryonal carcinoma cells, placental choriocarcinoma cells, cervical cancer cells, testicular cancer cells, testicular seminoma cells, testicular teratoma cells, embryonic testicular cancer cells, uterine cancer cells, teratocarcinoma cells, embryonal carcinoma cells, or any combination thereof.

In an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a disclosed method can further comprise surgically resecting the tumor from the subject.

In an aspect, a disclosed method can further comprise continuing to administer to the subject a therapeutically effective amount of one or more disclosed anti-cancer therapies.

In an aspect, a disclosed method can further comprise subjecting the subject to one or more invasive or non-invasive diagnostic assessments. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination. In an aspect, a disclosed invasive diagnostic assessment can comprise a tissue biopsy.

In an aspect, a disclosed method can further comprise administering to the subject a therapeutically effective amount of M1 macrophages. In an aspect, a disclosed method can further comprise contacting a population of M0 or naïve macrophages with a polarizing agent. In an aspect, a disclosed polarizing agent can drive the M0 or naïve macrophages to a M1 or pro-inflammatory phenotype. In an aspect, a disclosed polarizing agent can comprise lipopolysaccharide (LPS), interferon gamma (IFN-γ), granulocyte-macrophage colony-stimulating factor (GM-CSF), or any combination thereof. In an aspect of a disclosed method, contacting a population of polarized M1 or pro-inflammatory macrophages with one or more disclosed polarizing agents can enhance and/or improve the M1 phenotype of the polarized macrophages. In an aspect, a disclosed population of M1 macrophages can be macrophages that were treated ex vivo with one or more disclosed polarizing agents. In an aspect of a disclosed method, contacting a population of M0 or naïve macrophages with a disclosed polarizing agent can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of M0 or naïve macrophages with a disclosed polarizing agent can comprise more than 24 hours.

In an aspect of a disclosed method, contacting a population of M0 or naïve macrophages with a disclosed polarizing agent can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect of a disclosed method, contacting a population of M0 or naïve macrophages with a disclosed polarizing agent can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of M0 or naïve macrophages with a disclosed polarizing agent can comprise more than 24 hours. In an aspect of a disclosed method, contacting a population of M0 or naïve macrophages with a disclosed polarizing agent can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect, M0 or naïve macrophages can be contacted with a disclosed polarizing agent one or more times, and then polarized to M1 or pro-inflammatory macrophages.

In an aspect of a disclosed method, M1 or pro-inflammatory macrophages can be administered to a subject prior to, concurrent with, or after the administering a disclosed HDAC6 inhibitor and/or a disclosed CD47 inhibitor. In an aspect of a disclosed method, M1 or pro-inflammatory macrophages can be administered to a subject prior to, concurrent with, or after a disclosed composition comprising a disclosed HDAC6 inhibitor and a disclosed CD47 inhibitor.

In an aspect, a subject can be male or female. In an aspect, a subject can be an adult, a teenager, an adolescent, a child, a toddler, a baby, or an infant. In an aspect, a subject has received treatment for one or more disclosed cancers. In an aspect, a subject can be in treatment for one or more disclosed cancers. In an aspect, a subject can be treatment-naïve for one or more disclosed cancers.

In an aspect, a disclosed method can further comprise collecting one or more blood samples from a subject at the same time or at different times. For example, in an aspect, a blood sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample can be collected from a subject prior to treatment, during treatment, after treatment, or any combination thereof. In an aspect, a blood sample can be collected from a subject at any time deemed medically and/or clinically appropriate by the skilled clinician. In an aspect, a disclosed blood sample can be assayed and/or evaluated for one or more biomarkers of cancer, one or more biomarkers of disease progression, or any combination thereof.

In an aspect, a disclosed HDAC6 inhibitor can comprise any commercially available HDAC6 inhibitor or a combination of commercially available HDAC6 inhibitors. In an aspect, a disclosed HDAC6 inhibitor can comprise a selective HDAC6 inhibitor or HDAC6 selective inhibitor and can refer to a compound that preferentially inhibits histone deacetylase 6 over one or more other histone deacetylase isoforms, e.g., HDAC1, HDAC2, HDAC3, HDAC4, HDAC5, HDAC7, HDAC8, HDAC9, HDAC10, and/or HDCA11 in a cell-based in vitro assay. For example, a compound having a HDAC6 IC₅₀=5 nM and a HDAC1 IC₅₀ of 500 nM is a selective HDAC6 inhibitor that is 100-fold more selective over HDAC1; a compound having a HDAC6 ICso=5 nM, a HDAC1 ICso=500 nM, and a HDAC3 ICso=50 nM is a selective HDAC6 inhibitor that is 100-fold more selective over HDAC1 and 10-fold more selective over HDAC3; and so on. In an aspect, a disclosed selective HDAC6 inhibitor can preferentially inhibit HDAC6 over HDAC1, HDAC2, HDAC3, HDAC4, HDAC5, HDAC7, HDAC8, HDAC9, HDAC10, and/or HDAC11 In an aspect, a disclosed selective HDAC6 inhibitor can preferentially inhibit HDAC6 over one or more other HDAC isoforms. In an aspect, a disclosed selective HDAC6 inhibitor can be at least about 5-fold more selective over one or more other HDAC isoforms. In an aspect, a disclosed selective HDAC6 inhibitor can be at least about 10-fold more selective over one or more other HDAC isoforms. In an aspect, a disclosed selective HDAC6 inhibitor can be at least about 15-fold more selective over one or more other HDAC isoforms. In an aspect, a disclosed selective HDAC6 inhibitor can be at least about 20-fold more selective over one or more other HDAC isoforms. In an aspect, a disclosed selective HDAC6 inhibitor can be at least about 30-fold more selective over one or more other HDAC isoforms. In an aspect, a disclosed selective HDAC6 inhibitor can be at least about 40-fold more selective over one or more other HDAC isoforms. In an aspect, a disclosed selective HDAC6 inhibitor can be at least about 50-fold more selective over one or more other HDAC isoforms. In an aspect, a disclosed selective HDAC6 inhibitor can be at least about 100-fold more selective over one or more other HDAC isoforms. In an aspect, a disclosed selective HDAC6 inhibitor can be at least about 150-fold more selective over one or more other HDAC isoforms. In an aspect, the selective HDAC6 inhibitor can be at least about 200-fold more selective over one or more other HDAC isoforms. In an aspect, a disclosed selective HDAC6 inhibitor can be at least about 250-fold more selective over one or more other HDAC isoforms. In an aspect, a disclosed selective HDAC6 inhibitor can be at least about 500-fold more selective over one or more other HDAC isoforms. In an aspect, a disclosed selective HDAC6 inhibitor can be at least about 750-fold more selective over one or more other HDAC isoforms. In an aspect, a disclosed selective HDAC6 inhibitor can be at least about 1000-fold more selective over one or more other HDAC isoforms. In an aspect, a disclosed selective HDAC6 inhibitor can be at least about 2000-fold more selective over one or more other HDAC isoforms. In an aspect, a disclosed selective HDAC6 inhibitor can be at least about 3000-fold more selective over one or more other HDAC isoforms. HDAC6 selectivity over the other HDAC isoforms in cell-based assays can be determined using methods known in the art. In another aspect, the selective HDAC6 inhibitor can be at about 10-fold to about 3000-fold more selective over one or more other HDAC isoforms. In an aspect, a disclosed selective HDAC6 inhibitor can be at about 20-fold to about 3000-fold more selective over one or more other HDAC isoforms. In an aspect, a disclosed selective HDAC6 inhibitor can be at about 50-fold to about 3000-fold more selective over one or more other HDAC isoforms. In an aspect, a disclosed selective HDAC6 inhibitor can be at about 100-fold to about 3000-fold more selective over one or more other HDAC isoforms. In an aspect, a disclosed selective HDAC6 inhibitor can be at about 500-fold to about 3000-fold more selective over one or more other HDAC isoforms.

In an aspect, a disclosed composition can comprise any disclosed HDAC6 inhibitor and any disclosed CD47 inhibitor. In an aspect, a disclosed composition can comprise any disclosed any commercially available HDAC6 inhibitor and any disclosed commercially available CD47 inhibitor.

In an aspect, a disclosed method can reduce the risk of developing one or more metastases. A reduction in the risk of developing one or more metastases comprises a reduction of 10%, 20%, 30%, 40%, 50%, 60%, 70%, 80%, 90%, 100%, or any amount in the risk of metastases when compared to a control subject (such as, for example, a subject that has not received a disclosed HDAC6 inhibitor and/or a disclosed CD47 inhibitor or a composition thereof).

In an aspect, a disclosed method can comprise improving and/or enhancing the subject's quality of life and/or movement. In an aspect of a disclosed method, a disclosed improvement and/or enhancement (such as, for example, in the subject's quality of life and/or movement) can comprise a 10%, 20%, 30%, 40%, 50%, 60%, 70%, 80%, 90%, 100%, or any amount of improvement and/or enhancement when compared to a control subject (such as, for example, a subject that has not received a disclosed HDAC6 inhibitor and/or a disclosed CD47 inhibitor or a composition thereof). In an aspect, a disclosed improvement and/or enhancement (such as, for example, in the subject's quality of life and/or movement) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not received a disclosed HDAC6 inhibitor and/or a disclosed CD47 inhibitor or a composition thereof).

In an aspect, a disclosed diminishment (such as, for example, the size of one or more tumors) can comprise a 10%, 20%, 30%, 40%, 50%, 60%, 70%, 80%, 90%, 100%, or any amount of a decrease when compared to a control subject (such as, for example, a subject that has not received a disclosed HDAC6 inhibitor and/or a disclosed CD47 inhibitor or a composition thereof). In an aspect, a disclosed diminishment (such as, for example, the size of one or more tumors) can comprise a 10-20%, 20-30%, 30-40%, 40-50%, 50-60%, 60-70%, 70-80%, 80-90%, or 9 0-100% or any amount of a decrease when compared to a control subject (such as a subject that has not received a disclosed HDAC6 inhibitor and/or a disclosed CD47 inhibitor or a composition thereof).

In an aspect, a disclosed method can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinical can decide to change an aspect of the subject's treatment and/or change the subject's diagnosis or prognosis.

In an aspect, a disclosed method can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed HDAC6 inhibitor and/or a disclosed CD47 inhibitor and/or a composition comprising a disclosed HDAC6 inhibitor and a disclosed CD47 inhibitor. In an aspect, metabolic and/or physiologic data can inform the clinician when make subsequent treatment decisions.

In an aspect, a disclosed method can comprise diagnosing a subject in need of a disclosed composition.

In an aspect, a disclosed method can comprise administering to the subject an anti-PD1 molecule. In an aspect, a disclosed anti-PD1 molecule can comprise a PD1 antibody, a PDL1 antibody, or any combination thereof. In an aspect, a disclosed PD1 antibody can comprise a monoclonal antibody, a humanized monoclonal antibody, or a fragment thereof. In an aspect, a disclosed PD1 antibody can comprise a polyclonal antibody, a humanized polyclonal antibody, or a fragment thereof. In an aspect, a disclosed PD1 antibody can comprise any antibody or antibody fragment that specifically recognizes PD1. In an aspect, a disclosed PDL1 antibody can comprise a monoclonal antibody, a humanized monoclonal antibody, or a fragment thereof. In an aspect, a disclosed PDL1 antibody can comprise a polyclonal antibody, a humanized polyclonal antibody, or a fragment thereof. In an aspect, a disclosed PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed PD1 antibody can comprise nivolumab, pembrolizumab, STI-A1014, pidilizumab, or any combination thereof.

In an aspect of a disclosed method, a disclosed PD1 antibody, a disclosed PDL1 antibody, or a combination thereof can be a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of a disclosed anti-PD1 molecule. In an aspect, a disclosed anti-PD1 molecule can be administered prior to, concurrent with, or after the administration of a disclosed HDAC6 inhibitor and/or a disclosed CD47 inhibitor.

In an aspect, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

In an aspect of a disclosed method, a disclosed method can further comprise administering to the subject one or more SIRPα inhibitors. In an aspect, a disclosed SIRPα inhibitor can comprise a small molecule, a peptide, a polynucleotide, an antibody or fragment thereof, an antisense oligonucleotide, siRNA, RNAi, or any combination thereof. In an aspect, a disclosed SIRPα antisense oligonucleotide can target one or more parts or any part of an SIRPα mRNA or pre-mRNA. In an aspect, a disclosed SIRPα mRNA or pre-mRNA can comprise the sequence set forth in SEQ ID NO:25, SEQ ID NO:26, SEQ ID NO:27, SEQ ID NO:28, or a fragment thereof. In an aspect, a disclosed SIRPα inhibitor can comprise siRNA. In an aspect, a disclosed siRNA can target one or more parts or any part of the sequence set forth in SEQ ID NO:25, SEQ ID NO:26, SEQ ID NO:27, SEQ ID NO:28, or a fragment thereof. In an aspect, a disclosed SIRPα can comprise the amino acid sequence set forth in any one of SEQ ID NO:22, SEQ ID NO:23, SEQ ID NO:24, or any fragment thereof. In an aspect, an mRNA sequence for a disclosed SIRPα can comprise the sequence set forth in any one of SEQ ID NO:25, SEQ ID NO:26, SEQ ID NO:27, SEQ ID NO:28, or a fragment thereof. In an aspect, a disclosed SIRPα can be encoded by the sequence set forth in SEQ ID NO:29 or a fragment thereof.

3. Methods of Improving and/or Enhancing a Subject's Innate Antitumor Immunity

Disclosed herein is a method of improving and/or enhancing a subject's innate antitumor immunity; the method comprising administering to a subject having cancer a therapeutically effective amount of a HDAC6 inhibitor; and optionally administering to the subject a therapeutically effective amount of a CD47 inhibitor.

Disclosed herein is a method of improving and/or enhancing a subject's innate antitumor immunity; the method comprising administering to a subject having cancer a therapeutically effective amount of a CD47 inhibitor; and optionally administering to the subject a therapeutically effective amount of a HDAC6 inhibitor.

Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor.

Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor.

Disclosed herein is a method of improving and/or enhancing a subject's innate antitumor immunity; the method comprising administering to a subject having cancer a therapeutically effective amount of a HDAC6 inhibitor; and optionally administering to the subject a therapeutically effective amount of a CD47 inhibitor; wherein the cancer is treated.

Disclosed herein is a method of improving and/or enhancing a subject's innate antitumor immunity; the method comprising administering to a subject having cancer a therapeutically effective amount of a CD47 inhibitor; and optionally administering to the subject a therapeutically effective amount of a HDAC6 inhibitor; wherein the cancer is treated.

Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor; wherein the cancer is treated.

Disclosed herein is a method of improving and/or enhancing a subject's innate antitumor immunity; the method comprising administering to a subject having cancer a therapeutically effective amount of a HDAC6 inhibitor; and administering to the subject a therapeutically effective amount of a CD47 inhibitor; wherein the cancer is treated.

In an aspect of a disclosed method, treating the human subject can comprise (i) reducing and/or preventing tumor growth; (ii) reducing or slowing tumor metastasis; (iii) preventing and/or delaying recurrence of the cancer; (iv) extending and/or prolonging disease-free or tumor-free survival time; (v) increasing and/or lengthening overall survival time; (vi) reducing and/or minimizing the frequency of treatment; (vii) relieving and/or ameliorating one or more symptoms of the cancer; (viii) reducing and/or decreasing tumor burden, (ix) preventing and/or facilitating surgical intervention; (x) increasing and/or enhancing phagocytosis of cancer cells or tumor cells; (xi) increasing and/or enhancing immune cell infiltration in and/or around tumor microenvironment; (xii) enhancing the subject's innate antitumor immunity; (xiii) driving and/or facilitating the M1 or pro-inflammatory phenotype of macrophages, (xiv) disrupting the CD47-SIRPα axis in one or more cancer cells or tumor cells; or (xv) any combination thereof. In an aspect, a disclosed method can decrease the expression level and/or activity level of SIRPα. In an aspect, a disclosed decrease can be a partial decrease or a complete decrease.

In an aspect of a disclosed method, a disclosed HDAC6 inhibitor can comprise any disclosed HDAC6 inhibitor. In an aspect, a disclosed HDAC6 inhibitor can comprise a small molecule, a peptide, a polynucleotide, an antibody or fragment thereof, an antisense oligonucleotide, siRNA, RNAi, or any combination thereof.

In an aspect of a disclosed method, a disclosed HDAC6 inhibitor can comprise a RNA therapeutic. In an aspect, a disclosed HDAC6 inhibitor can comprise Nexturastat A, Tubastatin A, KA2507, Ricolinostat (ACY-1215), Citarinostat (ACY-241), Tubacin, CAY10603, WT161, ACY-738, ACY-775, HPOB, SKLB-23bb, SS-208, Suprastat HDAC6 degrader-1 (PROTAC), HDAC6 degrader-3 (PROTAC), J22352 (PROTAC), HPB, HDAC6-IN-12 (compound GZ), HDAC6/8/BRPF1-IN-1, QTX125, CG347B, BRD73954, AES-135, AES-350, KH-259, SW-100, HPOB, Droxinostat (NS 41080), Bufexamac, KA2507, MC2625, MPT0G211, MPT0G211 mesylate, WT-161, ACY-738, ACY-775, XP5, HDAC-IN-35 (Compound 14), HDAC6-IN-15, HDAC6-IN-14, HDAC6-IN-13 (Compound 35m), HDAC6-IN-11 (Compound 9), HDAC6-IN-10, HDAC6-IN-9 (compound 12c), HDAC6-IN-8, HDAC6-IN-7 (TCS HDAC6 20b), HDAC6-IN-6 (compound 6a), HDAC6-IN-5 (compound 11b), HDAC6-IN-4 (C10), HDAC-IN-4, HDAC-IN-40, HDAC6-IN-3 (Compound 14), HDAC3/6-IN-2 (compound 15), or any combination thereof.

In an aspect of a disclosed method, a disclosed HDAC6 antisense oligonucleotide can target one or more parts or any part of an HDAC6 mRNA or pre-mRNA. In an aspect, a disclosed HDAC6 mRNA or pre-mRNA can comprise the sequence set forth in SEQ ID NO: 18, SEQ ID NO:19, or a fragment thereof. In an aspect, a disclosed HDAC6 inhibitor can comprise siRNA. In an aspect, a disclosed siRNA can target one or more parts or any part of the sequence set forth in SEQ ID NO:18, SEQ ID NO:19, or a fragment thereof. In an aspect, a disclosed HDAC6 can comprise the amino acid sequence set forth in any one of SEQ ID NO:15, SEQ ID NO:16, SEQ ID NO:17, or any fragment thereof. In an aspect, an mRNA sequence for a disclosed HDAC6 can comprise the sequence set forth in any one of SEQ ID NO:18, SEQ ID NO:19, or a fragment thereof. In an aspect, a disclosed HDAC6 can be encoded by the sequence set forth in SEQ ID NO:20, SEQ ID NO:21, or a fragment thereof.

In an aspect of a disclosed method, a disclosed CD47 inhibitor can comprise RRx-001, a dinitroazetidine derivative, Hu5F9-G4, CC-90002, TTI-621, ALX148, SRF231, SHR-1603, IB1188, ST-1901, SGNCD-47M, or any combination thereof. In an aspect, a disclosed CD47 inhibitor can comprise Gentulizumab, CC-90002 (INBRX 103), Hu5F9-G4 (5F9), Magrolimab, STI-6643, TI-061, AO-176, SRF231, AK117, IB1188, IMC-002, SHR-1603, TJ011133, ZL-1201, evorpacept (ALX148), TTI-621, TTI-G22, or any combination thereof.

In an aspect of a disclosed method, a disclosed CD47 inhibitor can comprise any disclosed CD47 inhibitor. In an aspect, a disclosed CD47 inhibitor can comprise a small molecule, a peptide, a polynucleotide, an antibody or fragment thereof, an antisense oligonucleotide, siRNA, RNAi, or any combination thereof. In an aspect, a disclosed CD47 inhibitor can comprise a RNA therapeutic.

In an aspect of a disclosed method, a disclosed CD47 antisense oligonucleotide can target one or more parts or any part of an CD47 mRNA or pre-mRNA. In an aspect, a disclosed CD47 mRNA or pre-mRNA can comprise the sequence set forth in SEQ ID NO:11, SEQ ID NO:12, SEQ ID NO:13, or a fragment thereof. In an aspect, a disclosed CD47 inhibitor can comprise siRNA. In an aspect, a disclosed siRNA can target one or more parts or any part of the sequence set forth in SEQ ID NO: 11, SEQ ID NO:12, SEQ ID NO:13, or a fragment thereof. In an aspect, a disclosed CD47 can comprise the amino acid sequence set forth in any one of SEQ ID NO:07, SEQ ID NO:08, SEQ ID NO:09, or any fragment thereof. In an aspect, an mRNA sequence for a disclosed CD47 can comprise the sequence set forth in any one of SEQ ID NO:11, SEQ ID NO:12, SEQ ID NO: 13, or a fragment thereof. In an aspect, a disclosed CD47 can be encoded by the sequence set forth in SEQ ID NO:14 or a fragment thereof.

In an aspect, a disclosed CD47 inhibitor can be administered prior to, concurrent with, or after the administration of a disclosed HDAC6 inhibitor. In an aspect, a disclosed CD47 inhibitor can be administered about 3 months, about 2 months, or about 1 month prior to or after the administering of a disclosed HDAC6 inhibitor. In an aspect, a disclosed CD47 inhibitor can be administered about 8 weeks, about 7 weeks, about 6 weeks, about 5 weeks, about 4 weeks, about 3 weeks, about 2 weeks, or about 1 week prior to or after the administering of a disclosed HDAC6 inhibitor. In an aspect, a disclosed CD47 inhibitor can be administered about 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 day prior to or after the administering of a disclosed HDAC6 inhibitor. In an aspect, a disclosed CD47 inhibitor can be administered about 24, 23, 22, 21, 20, 19, 18, 17, 16, 15, 14, 13, 12, 11, 10, 9, 8, 7, 6, 5, 4, 3, 2, or 1 hours prior to or after the administering of a disclosed HDAC6 inhibitor.

In an aspect, a disclosed method can further comprise repeating the administering of a disclosed HDAC6 inhibitor. In an aspect, a disclosed method can further comprise repeating the administering of a disclosed CD47 inhibitor.

In an aspect, a disclosed method can further comprise repeating the administering of a disclosed composition comprising a disclosed HDAC6 inhibitor and a disclosed CD47 inhibitor.

In an aspect of a disclosed method, administering a disclosed HDAC6 inhibitor can comprise systemic or direct administration. In an aspect of a disclosed method, administering a disclosed CD47 inhibitor can comprise systemic or direct administration. In an aspect of a disclosed method, administering a disclosed a composition comprising a disclosed HDAC6 inhibitor and a disclosed CD47 inhibitor can comprise systemic or direct administration.

In an aspect, administering a disclosed HDAC6 inhibitor can comprise oral administration, intravenous administration, intratumoral administration, intraperitoneal administration, or any combination thereof.

In an aspect, administering a disclosed HDAC6 inhibitor can comprise administration via any route of administration known to the art. In an aspect, administering a disclosed HDAC6 inhibitor can comprise administration via multiple routes of administration known to the art. In an aspect, administering a disclosed CD47 inhibitor comprise oral administration, intravenous administration, intratumoral administration, intraperitoneal administration, or any combination thereof. In an aspect, administering a disclosed CD47 inhibitor can comprise administration via any route of administration known to the art. In an aspect, administering a disclosed CD47 inhibitor can comprise administration via multiple routes of administration known to the art.

In an aspect, administering a disclosed composition comprising a disclosed HDAC6 inhibitor and a disclosed CD47 inhibitor can comprise oral administration, intravenous administration, intratumoral administration, intraperitoneal administration, or any combination thereof. In an aspect, administering a disclosed composition comprising a disclosed HDAC6 inhibitor and a disclosed CD47 inhibitor can comprise administration via any route of administration known to the art. In an aspect, administering a disclosed composition comprising a disclosed HDAC6 inhibitor and a disclosed CD47 inhibitor can comprise administration via multiple routes of administration known to the art.

In an aspect, local administration can comprise delivery to one or more of the subject's body systems having cancerous cells or tumorous growth. In an aspect, the subject's one or more body systems having cancerous cells or tumorous growth can comprise the subject's cardiovascular system, the subject's digestive system, the subject's endocrine system, the subject lymphatic system, the subject's muscular system, the subject's nervous system, the subject's reproductive system, the subject's respiratory system, the subject's skeletal system, the subject's urinary system, the subject's integumentary system, or any combination thereof.

In an aspect of a disclosed method, administering a disclosed HDAC6 inhibitor can comprise a single dose or multiple doses (such as 2, 3, 4, 5, 6, 7, 8, 9, 10, or more than 10 doses). In an aspect of a disclosed method, administering a disclosed CD47 inhibitor can comprise a single dose or multiple doses (such as 2, 3, 4, 5, 6, 7, 8, 9, 10, or more than 10 doses). In an aspect of a disclosed method, administering a disclosed composition comprising a disclosed HDAC6 inhibitor and a disclosed CD47 inhibitor can comprise a single dose or multiple doses (such as 2, 3, 4, 5, 6, 7, 8, 9, 10, or more than 10 doses).

In an aspect, a therapeutically effective amount of a disclosed HDAC6 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed HDAC6 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed HDAC6 inhibitor can comprise reduce, inhibit, and/or prevent the activity level and/or expression level of HDAC6 in the subject. In an aspect, a disclosed HDAC6 inhibitor can comprise reduce, inhibit, and/or prevent the activity level and/or expression level of HDAC6 in one or more macrophage populations in a subject. In an aspect, a disclosed HDAC6 inhibitor can modulate the phenotype and/or phagocytic function of one or more macrophage populations in a subject. In an aspect, a disclosed HDAC6 inhibitor can reduce, inhibit, and/or prevent the activity level and/or expression level of CD47 in one or more cancer cells or one or more populations of cancer cells.

In an aspect, a therapeutically effective amount of a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect, a therapeutically effective amount of a disclosed CD47 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a therapeutically effective amount of a disclosed composition comprising a disclosed HDAC6 inhibitor and a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a therapeutically effective amount of a disclosed composition comprising a disclosed HDAC6 inhibitor and a disclosed CD47 inhibitor can comprise about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, about 100 μg/kg body weight/day to about 10 mg/kg body, or about 1 mg/kg body weight/day to about 100 mg/kg body weight/day. In an aspect a therapeutically effective amount of a disclosed composition comprising a disclosed HDAC6 inhibitor and a disclosed CD47 inhibitor can comprise about 10 mg/kg body weight/day, about 20 mg/kg body weight/day, about 30 mg/kg body weight/day, about 40 mg/kg body weight/day, about 50 mg/kg body weight/day, about 60 mg/kg body weight/day, about 70 mg/kg body weight/day, about 80 mg/kg body weight/day, about 90 mg/kg body weight/day, or about 100 mg/kg body weight/day.

In an aspect, a disclosed method can further comprise monitoring the subject for adverse effects. In an aspect, in the absence of adverse effects, a disclosed method can further comprise continuing to treat the subject. In an aspect, continuing to treat the subject can comprise continuing to administer a disclosed HDAC6 inhibitor and/or continuing to administer a disclosed CD47 inhibitor and/or continuing to administer a disclosed composition comprising a disclosed HDAC6 inhibitor and a disclosed CD47 inhibitor.

In an aspect, in the presence of adverse effects, a disclosed method can further comprise modifying one or more steps of the method. In an aspect, modifying one or more steps of a disclosed method can comprise modifying one or more administering steps. In an aspect, modifying one or more disclosed administering steps can comprise changing the amount of a disclosed HDAC6 inhibitor and/or a disclosed CD47 inhibitor administered to the subject, changing the frequency of a disclosed HDAC6 inhibitor and/or a disclosed CD47 inhibitor administration, changing the duration of a disclosed HDAC6 inhibitor and/or a disclosed CD47 inhibitor administration, changing the route of a disclosed HDAC6 inhibitor and/or a disclosed CD47 inhibitor administration, or any combination thereof. In an aspect, modifying one or more disclosed administering steps can comprise changing the amount a disclosed composition comprising a disclosed HDAC6 inhibitor and a disclosed CD47 inhibitor administered to the subject, changing the frequency of a disclosed composition comprising a disclosed HDAC6 inhibitor and a disclosed CD47 inhibitor administration, changing the duration of a disclosed composition comprising a disclosed HDAC6 inhibitor and a disclosed CD47 inhibitor administration, changing the route of a disclosed composition comprising a disclosed HDAC6 inhibitor and a disclosed CD47 inhibitor administration, or any combination thereof.

In an aspect, a disclosed method can further comprise administering to the subject one or more additional anti-cancer therapies. In an aspect, a disclosed anti-cancer therapy can comprise endocrine therapy, radiotherapy, hormone therapy, gene therapy, thermal therapy, ultrasound therapy, or any combination thereof. In an aspect, a disclosed anti-cancer therapy can comprise one or more chemotherapeutic agents. In an aspect, a disclosed chemotherapeutic agent can comprise an anthracycline, a vinca alkaloid, an alkylating agent, an immune cell antibody, an antimetabolite, a TNFR glucocorticoid induced TNFR related protein (GITR) agonist, a proteasome inhibitor, an immunomodulator, or any combination thereof. In an aspect, a disclosed chemotherapeutic agent can comprise 5-fluorouracil (Adrucil, Efudex), 6-mercaptopurine (Purinethol), 6-thioguanine, aclarubicin or aclacinomycin A, alemtuzamab (Lemtrada), anastrozole (Arimidex), bicalutamide (Casodex), bleomycin sulfate (Blenoxane), bortezomib (Velcade), busulfan (Myleran), busulfan injection (Busulfex), capecitabine (Xeloda), carboplatin (Paraplatin), carmustine (BiCNU), chlorambucil (Leukeran), cisplatin (Platinol), cladribine (Leustatin), Cosmegan, cyclophosphamide (Cytoxan or Neosar), cyclophosphamide, cytarabine liposome injection (DepoCyt), cytarabine, cytosine arabinoside (Cytosar-U), dacarbazine (DTIC-Dome), dactinomycin (Cosmegen), daunorubicin citrate liposome injection (DaunoXome), daunorubicin hydrochloride (Cerubidine), dexamethasone, docetaxel (Taxotere), doxorubicin hydrochloride (Adriamycin, Rubex), etoposide (Vepesid), fludarabine phosphate (Fludara), flutamide (Eulexin), folic acid antagonists, gemcitabine (difluorodeoxycitidine), gemtuzumab, gliotoxin, hydroxyurea (Hydrea), Idarubicin (Idamycin), ifosfamide (IFEX), ifosfamide, irinotecan (Camptosar), L-asparaginase (ELSPAR), lenalidomide), leucovorin calcium, melphalan (Alkeran), melphalan, methotrexate (Folex), mitoxantrone (Novantrone), mylotarg, N4-pentoxycarbonyl-5 deoxy-5-fluorocytidine, nab-paclitaxel (Abraxane), paclitaxel (Taxol), pentostatin, phoenix (Yttrium90/MX-DTPA), polifeprosan 20 with carmustine implant (Gliadel), purine analogs and adenosine deaminase inhibitors (fludarabine), pyrimidine analogs, rituximab, tamoxifen citrate (Nolvadex), temozolomide), teniposide (Vumon), tezacitibine, thalidomide or a thalidomide derivative, thiotepa, tirapazamine (Tirazone), topotecan hydrochloride for injection (Hycamptin), tositumomab), vinblastine (Velban), vinblastine, vincristine (Oncovin), vindesine, vinorelbine (Navelbine), or any combination thereof.

In an aspect, a disclosed method can further comprise administering to the subject an anti-chemokine therapy. In an aspect, a disclosed anti-chemokine therapy can comprise one or more antibodies against CCL1, CCL2, CCL4, CCL17, CCL19, CCL21, CCL22, CCL25, CXCL12, CCR2, CCR7, CCR8, CCR9, CXCR4, CX3CL1, CX3CR1, or any combination thereof.

In an aspect, cancerous cells and/or tumorous cells can comprise ovarian cancer cells, ovarian adenocarcinoma cells, ovarian teratocarcinoma cells, lung cancer cells, small cell lung cancer (SCLC) cells, non-small cell lung cancer (NSCLC) cells, squamous cell lung carcinoma cells, adenocarcinoma cells, gastric cancer cells, breast cancer cells, hepatic cancer cells, pancreatic cancer cells, skin cancer cells, in particular basal cell carcinoma and squamous cell carcinoma cells, malignant melanoma cells, head and neck cancer cells, malignant pleomorphic adenoma cells, sarcoma cells, synovial sarcoma cells, carcinosarcoma cells, bile duct cancer cells, bladder cancer cells, transitional cell carcinoma cells, papillary carcinoma cells, kidney cancer cells, renal cell carcinoma cells, clear cell renal cell carcinoma cells, papillary renal cell carcinoma cells, colon cancer cells, small bowel cancer cells, small bowel adenocarcinoma cells, adenocarcinoma of the ileum cells, testicular embryonal carcinoma cells, placental choriocarcinoma cells, cervical cancer cells, testicular cancer cells, testicular seminoma cells, testicular teratoma cells, embryonic testicular cancer cells, uterine cancer cells, teratocarcinoma cells, embryonal carcinoma cells, or any combination thereof.

In an aspect, a subject can have, be diagnosed with, or be suspected of having ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

In an aspect, a disclosed method can further comprise surgically resecting the tumor from the subject. In an aspect, a disclosed method can further comprise continuing to administer to the subject a therapeutically effective amount of one or more disclosed anti-cancer therapies.

In an aspect, a disclosed method can further comprise subjecting the subject to one or more invasive or non-invasive diagnostic assessments. In an aspect, a disclosed non-invasive diagnostic assessment can comprise x-rays, computerized tomography (CT) scans, magnetic resonance imaging (MRI) scans, ultrasounds, positron emission tomography (PET) scans, or any combination. In an aspect, a disclosed invasive diagnostic assessment can comprise a tissue biopsy.

In an aspect, a disclosed method can further comprise administering to the subject a therapeutically effective amount of M1 macrophages. In an aspect, a disclosed method can further comprise contacting a population of M0 or naïve macrophages with a polarizing agent. In an aspect, a disclosed polarizing agent can drive the M0 or naïve macrophages to a M1 or pro-inflammatory phenotype. In an aspect, a disclosed polarizing agent can comprise lipopolysaccharide (LPS), interferon gamma (IFN-γ), granulocyte-macrophage colony-stimulating factor (GM-CSF), or any combination thereof. In an aspect of a disclosed method, contacting a population of polarized M1 or pro-inflammatory macrophages with one or more disclosed polarizing agents can enhance and/or improve the M1 phenotype of the polarized macrophages. In an aspect, a disclosed population of M1 macrophages can be macrophages that were treated ex vivo with one or more disclosed polarizing agents. In an aspect of a disclosed method, contacting a population of M0 or naïve macrophages with a disclosed polarizing agent can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of M0 or naïve macrophages with a disclosed polarizing agent can comprise more than 24 hours.

In an aspect of a disclosed method, contacting a population of M0 or naïve macrophages with a disclosed polarizing agent can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect of a disclosed method, contacting a population of M0 or naïve macrophages with a disclosed polarizing agent can comprise about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hour, about 16 hours, about 17 hours, about 18 hours, about 19 hours, about 20 hours, about 21 hours, about 22 hours, about 23 hours, about 24 hours, or any amount in between. In an aspect of a disclosed method, contacting a population of M0 or naïve macrophages with a disclosed polarizing agent can comprise more than 24 hours. In an aspect of a disclosed method, contacting a population of M0 or naïve macrophages with a disclosed polarizing agent can comprise about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, or any amount in between. In an aspect, M0 or naïve macrophages can be contacted with a disclosed polarizing agent one or more times, and then polarized to M1 or pro-inflammatory macrophages.

In an aspect of a disclosed method, M1 or pro-inflammatory macrophages can be administered to a subject prior to, concurrent with, or after the administering a disclosed HDAC6 inhibitor and/or a disclosed CD47 inhibitor. In an aspect of a disclosed method, M1 or pro-inflammatory macrophages can be administered to a subject prior to, concurrent with, or after a disclosed composition comprising a disclosed HDAC6 inhibitor and a disclosed CD47 inhibitor.

In an aspect, a subject can be male or female. In an aspect, a subject can be an adult, a teenager, an adolescent, a child, a toddler, a baby, or an infant. In an aspect, a subject has received treatment for one or more disclosed cancers. In an aspect, a subject can be in treatment for one or more disclosed cancers. In an aspect, a subject can be treatment-naïve for one or more disclosed cancers.

In an aspect, a disclosed method can further comprise collecting one or more blood samples from a subject at the same time or at different times. For example, in an aspect, a blood sample can be collected from a subject at a pre-determined interval. In an aspect, a pre-determined interval can be once a week, once every 2 weeks, once every 3 weeks, once every 4 weeks, once every 5 weeks, once every 6 weeks, once every 7 weeks, once every 8 weeks, or at a longer interval. In an aspect, a pre-determined interval can be once a month, once every 2 months, once every 3 months, once every 5 months, once every 5 months, once every 6 months, or at a longer interval. In an aspect, a blood sample can be collected from a subject prior to treatment, during treatment, after treatment, or any combination thereof. In an aspect, a blood sample can be collected from a subject at any time deemed medically and/or clinically appropriate by the skilled clinician. In an aspect, a disclosed blood sample can be assayed and/or evaluated for one or more biomarkers of cancer, one or more biomarkers of disease progression, or any combination thereof.

In an aspect, a disclosed HDAC6 inhibitor can comprise any commercially available HDAC6 inhibitor or a combination of commercially available HDAC6 inhibitors. In an aspect, a disclosed HDAC6 inhibitor can comprise a selective HDAC6 inhibitor or HDAC6 selective inhibitor and can refer to a compound that preferentially inhibits histone deacetylase 6 over one or more other histone deacetylase isoforms, e.g., HDAC1, HDAC2, HDAC3, HDAC4, HDAC5, HDAC7, HDAC8, HDAC9, HDAC10, and/or HDCA11 in a cell-based in vitro assay. For example, a compound having a HDAC6 IC₅₀=5 nM and a HDAC1 IC₅₀ of 500 nM is a selective HDAC6 inhibitor that is 100-fold more selective over HDAC1; a compound having a HDAC6 ICso=5 nM, a HDAC1 ICso=500 nM, and a HDAC3 ICso=50 nM is a selective HDAC6 inhibitor that is 100-fold more selective over HDAC1 and 10-fold more selective over HDAC3; and so on. In an aspect, a disclosed selective HDAC6 inhibitor can preferentially inhibit HDAC6 over HDAC1, HDAC2, HDAC3, HDAC4, HDAC5, HDAC7, HDAC8, HDAC9, HDAC10, and/or HDAC11 In an aspect, a disclosed selective HDAC6 inhibitor can preferentially inhibit HDAC6 over one or more other HDAC isoforms. In an aspect, a disclosed selective HDAC6 inhibitor can be at least about 5-fold more selective over one or more other HDAC isoforms. In an aspect, a disclosed selective HDAC6 inhibitor can be at least about 10-fold more selective over one or more other HDAC isoforms. In an aspect, a disclosed selective HDAC6 inhibitor can be at least about 15-fold more selective over one or more other HDAC isoforms. In an aspect, a disclosed selective HDAC6 inhibitor can be at least about 20-fold more selective over one or more other HDAC isoforms. In an aspect, a disclosed selective HDAC6 inhibitor can be at least about 30-fold more selective over one or more other HDAC isoforms. In an aspect, a disclosed selective HDAC6 inhibitor can be at least about 40-fold more selective over one or more other HDAC isoforms. In an aspect, a disclosed selective HDAC6 inhibitor can be at least about 50-fold more selective over one or more other HDAC isoforms. In an aspect, a disclosed selective HDAC6 inhibitor can be at least about 100-fold more selective over one or more other HDAC isoforms. In an aspect, a disclosed selective HDAC6 inhibitor can be at least about 150-fold more selective over one or more other HDAC isoforms. In an aspect, the selective HDAC6 inhibitor can be at least about 200-fold more selective over one or more other HDAC isoforms. In an aspect, a disclosed selective HDAC6 inhibitor can be at least about 250-fold more selective over one or more other HDAC isoforms. In an aspect, a disclosed selective HDAC6 inhibitor can be at least about 500-fold more selective over one or more other HDAC isoforms. In an aspect, a disclosed selective HDAC6 inhibitor can be at least about 750-fold more selective over one or more other HDAC isoforms. In an aspect, a disclosed selective HDAC6 inhibitor can be at least about 1000-fold more selective over one or more other HDAC isoforms. In an aspect, a disclosed selective HDAC6 inhibitor can be at least about 2000-fold more selective over one or more other HDAC isoforms. In an aspect, a disclosed selective HDAC6 inhibitor can be at least about 3000-fold more selective over one or more other HDAC isoforms. HDAC6 selectivity over the other HDAC isoforms in cell-based assays can be determined using methods known in the art. In another aspect, the selective HDAC6 inhibitor can be at about 10-fold to about 3000-fold more selective over one or more other HDAC isoforms. In an aspect, a disclosed selective HDAC6 inhibitor can be at about 20-fold to about 3000-fold more selective over one or more other HDAC isoforms. In an aspect, a disclosed selective HDAC6 inhibitor can be at about 50-fold to about 3000-fold more selective over one or more other HDAC isoforms. In an aspect, a disclosed selective HDAC6 inhibitor can be at about 100-fold to about 3000-fold more selective over one or more other HDAC isoforms. In an aspect, a disclosed selective HDAC6 inhibitor can be at about 500-fold to about 3000-fold more selective over one or more other HDAC isoforms.

In an aspect, a disclosed composition can comprise any disclosed HDAC6 inhibitor and any disclosed CD47 inhibitor. In an aspect, a disclosed composition can comprise any disclosed any commercially available HDAC6 inhibitor and any disclosed commercially available CD47 inhibitor.

In an aspect, a disclosed method can reduce the risk of developing one or more metastases. A reduction in the risk of developing one or more metastases comprises a reduction of 10%, 20%, 30%, 40%, 50%, 60%, 70%, 80%, 90%, 100%, or any amount in the risk of metastases when compared to a control subject (such as, for example, a subject that has not received a disclosed HDAC6 inhibitor and/or a disclosed CD47 inhibitor or a composition thereof).

In an aspect, a disclosed method can comprise improving and/or enhancing the subject's quality of life and/or movement. In an aspect of a disclosed method, a disclosed improvement and/or enhancement (such as, for example, in the subject's quality of life and/or movement) can comprise a 10%, 20%, 30%, 40%, 50%, 60%, 70%, 80%, 90%, 100%, or any amount of improvement and/or enhancement when compared to a control subject (such as, for example, a subject that has not received a disclosed HDAC6 inhibitor and/or a disclosed CD47 inhibitor or a composition thereof). In an aspect, a disclosed improvement and/or enhancement (such as, for example, in the subject's quality of life and/or movement) can comprise a 10-20%, 20-30%, 30-40%, 40-50%, 50-60%, 60-70%, 70-80%, 80-90%, or 90-100% or any amount of an improvement and/or enhancement when compared to a control subject (such as a subject that has not received a disclosed HDAC6 inhibitor and/or a disclosed CD47 inhibitor or a composition thereof).

In an aspect, a disclosed diminishment (such as, for example, the size of one or more tumors) can comprise a 10%, 20%, 30%, 40%, 50%, 60%, 70%, 80%, 90%, 100%, or any amount of a decrease when compared to a control subject (such as, for example, a subject that has not received a disclosed HDAC6 inhibitor and/or a disclosed CD47 inhibitor or a composition thereof). In an aspect, a disclosed diminishment (such as, for example, the size of one or more tumors) can comprise a 10-20%, 20-30%, 30-40%, 40-50%, 50-60%, 60-70%, 70-80%, 80-90%, or 90-100% or any amount of a decrease when compared to a control subject (such as a subject that has not received a disclosed HDAC6 inhibitor and/or a disclosed CD47 inhibitor or a composition thereof).

In an aspect, a disclosed method can further comprise monitoring the subject following the administering step and/or the treating step to generate a compilation of biochemical and/or physiological and/or behavioral data. In an aspect, a disclosed compilation of data can be used to identify a trend or a pattern. In an aspect, a disclosed compilation of data can be used to guide and/or inform a skilled clinician in a decision-making process regarding treatment and/or testing. In an aspect, for example, a clinical can decide to change an aspect of the subject's treatment and/or change the subject's diagnosis or prognosis.

In an aspect, a disclosed method can further comprise monitoring the subject's metabolic and/or physiologic improvement following the administering and/or treating step and/or following the administering and/or treating steps. In an aspect, a clinician can measure and/or determine the subject's metabolic and/or physiologic status over time to identify one or more improvements and/or one or more diminishments. In an aspect of a disclosed method, a clinician can use the subject's metabolic and/or physiologic status and/or the trend of the subject's metabolic and/or physiological status and/or trend to make a treatment decision and/or to modify an aspect of a disclosed method and/or to continue treating the subject and/or continue to administer a disclosed HDAC6 inhibitor and/or a disclosed CD47 inhibitor and/or a composition comprising a disclosed HDAC6 inhibitor and a disclosed CD47 inhibitor. In an aspect, metabolic and/or physiologic data can inform the clinician when make subsequent treatment decisions.

In an aspect, a disclosed method can comprise diagnosing a subject in need of a disclosed composition.

In an aspect, a disclosed method can comprise administering to the subject an anti-PD1 molecule. In an aspect, a disclosed anti-PD1 molecule can comprise a PD1 antibody, a PDL1 antibody, or any combination thereof. In an aspect, a disclosed PD1 antibody can comprise a monoclonal antibody, a humanized monoclonal antibody, or a fragment thereof. In an aspect, a disclosed PD1 antibody can comprise a polyclonal antibody, a humanized polyclonal antibody, or a fragment thereof. In an aspect, a disclosed PD1 antibody can comprise any antibody or antibody fragment that specifically recognizes PD1. In an aspect, a disclosed PDL1 antibody can comprise a monoclonal antibody, a humanized monoclonal antibody, or a fragment thereof. In an aspect, a disclosed PDL1 antibody can comprise a polyclonal antibody, a humanized polyclonal antibody, or a fragment thereof. In an aspect, a disclosed PDL1 antibody can comprise any antibody or antibody fragment that specifically recognizes PDL1. Antibodies and methods of preparing antibodies are known in the art. Similarly, recombinant antibodies and methods of preparing recombinant antibodies are known in the art.

In an aspect, a disclosed PD1 antibody can comprise nivolumab, pembrolizumab, STI-A1014, pidilizumab, or any combination thereof.

In an aspect of a disclosed method, a disclosed PD1 antibody, a PDL1 antibody, or a combination thereof can be a dose of about 1 ng/kg body weight/day to about 100 ng/kg body weight/day, about 10 ng/kg body weight/day to about 1 μg/kg body, about 100 ng/kg body weight/day to about 10 μg/kg body, about 1 μg/kg body weight/day to about 100 μg/kg body, about 10 μg/kg body weight/day to about 1 mg/kg body, or about 100 μg/kg body weight/day to about 10 mg/kg body.

In an aspect, a disclosed method can comprise repeating the administering of a disclosed anti-PD1 molecule. In an aspect, a disclosed anti-PD1 molecule can be administered prior to, concurrent with, or after the administration of a disclosed HDAC6 inhibitor and/or a disclosed CD47 inhibitor.

In an aspect, administering a disclosed anti-PD1 molecule can comprise systemic or direct administration. In an aspect, administering a disclosed anti-PD1 molecule can comprise intravenous administration, intratumoral administration, intraperitoneal administration, or any combination thereof. In an aspect, administering a disclosed can be administered by any method of administration disclosed herein. In an aspect, a disclosed anti-PD1 molecule can be administered via multiple routes either concurrently or sequentially. For example, in an aspect, a disclosed anti-PD1 molecule can be first administered intratumorally and then be administered intravenously. In an aspect, administering a disclosed anti-PD1 molecule can be first administered intratumorally and then be administered orally. A skilled clinician can determine the best route of administration for a subject at a given time.

In an aspect of a disclosed method, a disclosed method can further comprise administering to the subject one or more SIRPα inhibitors. In an aspect, a disclosed SIRPα inhibitor can comprise a small molecule, a peptide, a polynucleotide, an antibody or fragment thereof, an antisense oligonucleotide, siRNA, RNAi, or any combination thereof. In an aspect, a disclosed SIRPα antisense oligonucleotide can target one or more parts or any part of an SIRPα mRNA or pre-mRNA. In an aspect, a disclosed SIRPα mRNA or pre-mRNA can comprise the sequence set forth in SEQ ID NO:25, SEQ ID NO:26, SEQ ID NO:27, SEQ ID NO:28, or a fragment thereof. In an aspect, a disclosed SIRPα inhibitor can comprise siRNA. In an aspect, a disclosed siRNA can target one or more parts or any part of the sequence set forth in SEQ ID NO:25, SEQ ID NO:26, SEQ ID NO:27, SEQ ID NO:28, or a fragment thereof. In an aspect, a disclosed SIRPα can comprise the amino acid sequence set forth in any one of SEQ ID NO:22, SEQ ID NO:23, SEQ ID NO:24, or any fragment thereof. In an aspect, an mRNA sequence for a disclosed SIRPα can comprise the sequence set forth in any one of SEQ ID NO:25, SEQ ID NO:26, SEQ ID NO:27, SEQ ID NO:28, or a fragment thereof. In an aspect, a disclosed SIRPα can be encoded by the sequence set forth in SEQ ID NO:29 or a fragment thereof.

### EXAMPLES

The Examples that follow are illustrative of specific aspects of the invention, and various uses thereof. They set forth for explanatory purposes only and are not to be taken as limiting the invention.

The CD47/SIRPα axis is a critical innate immune checkpoint that regulates phagocytosis of cancer cells by macrophages and other myeloid cells and is thus exploited by tumors to escape innate immunity. CD47 is a ubiquitously expressed protein known as a “do not eat me” signal. CD47 prevents the phagocytosis of cancer cells upon interaction with signal regulatory protein alpha (SIRPα), expressed on macrophages (Oldenborg P A, et al. (2000) Science. 288:2051-2054; Matlung H L, et al. (2017) Immunol. Rev. 276:145-164). CD47 blockade induces antibody dependent cellular phagocytosis (ADCP) and T cell mediated destruction of immunogenic tumors, while targeting SIRPα potentiates innate and adaptive antitumor immunity.

In this study, the role of highly selective HDAC6 inhibitors in modulating the phenotype and phagocytic function of macrophages was examined. As described herein, HDAC6 inhibition promoted the M1-like phenotype and downregulated the M2-like macrophage phenotype. Furthermore, HDAC6 inhibitors such as Nexturastat A (NextA) downregulated SIRPα on various macrophage phenotypes and modulated CD47 expression in melanoma cells. Modulating the CD47/SIRPα by HDAC6 inhibitors led to an increase in phagocytosis in NextA treated macrophages, which was further enhanced in the presence of CD47 blocking antibody. Lastly, the antitumor properties of the combination of NextA and αCD47 in vivo using the SM1 melanoma mouse model were assessed. The combination of NextA and αCD47 significantly decreased tumor growth and increased immune cell infiltration in the tumor microenvironment (TME). This combination mainly modulated innate immunity, including macrophages and natural killer cells.

The data described herein examined several questions: whether the combination of HDAC6 inhibitors and anti-CD47 antibodies could (i) modulate the CD47/SIRPα axis, (ii) enhance phagocytosis of tumor cells by macrophages, and (iii) modulate immunosuppression and macrophage phenotype to potentially decrease tumor growth by enhancing the antitumor immune response.

### A. Materials and Methods.

### 1. Cell Culture.

The bone marrow-derived macrophage cell line A3.1A7 was kindly provided by Dr. Kenneth L. Rock (University of Massachusetts Medical School, Worcester, MA). A3.1A7 cells were cultured in RPMI 1640 media supplemented with 10% fetal bovine serum (FBS), 1% Penicillin-Streptomycin (30-002-CI, Corning), 1% HEPES buffer (H3537, Sigma Aldrich), 1% L-glutamine (25030081, Corning), and 1% non-essential amino acids (NEAA, 25-025-Cl, Corning) at 37° C. and 5% CO₂. THP1 monocytes were obtained from ATCC (TIB-202) and cultured in complete RPMI 1640 medium containing 10% FBS, 1% NEAA, and 1% penicillin-streptomycin. SM1 cells were obtained from Dr. Antoni Ribas at University of California, Los Angeles. B16 and WM164 melanoma cells were obtained from ATCC. SM1 and B16 mouse melanoma cell lines as well as WM164 human melanoma cells were cultured in complete RPMI 1640 medium containing 10% FBS, 1% NEAA, and 1% penicillin-streptomycin.

### 2. Macrophage Isolation and Polarization.

Bone marrow-derived macrophages were isolated from the femurs and tibias of wild-type C57/Bl6, HDAC6 knockout (C57BL/6J-Hdac6em2Lutzy/J, 029318, Jackson Laboratory), or UBC-GFP (C57BL/6-Tg(UBC-GFP)30Scha/J, 004353, Jackson Laboratory). Isolated bone marrow cells were cultured in RPMI 1640 complete medium with 10% FBS, 1% NEAA, and 1% penicillin-streptomycin in 5% CO₂ at 37° C. Medium was supplemented with murine recombinant M-CSF (20 ng/mL, 576404, Biolegend) to differentiate monocytes into macrophages. Three days after bone marrow isolation, undifferentiated and floating cells were washed with PBS and replaced with fresh RPMI media. A3.1A7 cells and BMDMs were pretreated with 5 μM of HDAC6 inhibitor NextA or vehicle prior to adding polarizing cytokines.

Macrophages were polarized to M1-like phenotype using 50 ng/mL of interferon gamma (IFNγ, 575306, Biolegend) and 100 ng/mL of bacterial lipopolysaccharide (LPS, L2880, Sigma Aldrich, St. Louis, MO) for 24 hours. To polarize macrophages towards the M2-like phenotype, macrophages were stimulated with 20 ng/mL of IL-4 (214-14, PeproTech) and 20 ng/mL of IL-13 (575904, Biolegend). THP1 monocytes were treated with 5 ng/mL of phorbol 12-myristate 13-acetate (PMA) to induce differentiation into macrophages. Once converted to macrophages, media was replaced to remove PMA prior to treatment and/or polarization. Polarization towards M1 and M2 phenotypes was performed with respective recombinant human cytokines from PeproTech: 50 ng/mL of IFNγ (300-02), 20 ng/mL IL-4 (200-04), and 20 ng/mL IL-13 (200-13). 100 ng/mL LPS was also used to polarize toward the M1 phenotype. 3. In vivo Studies.

Animal experiments were performed in accordance with protocol A354 approved by the Institutional Care and Use Committee (IACUC) at The George Washington University. Female C57BL/6 mice (4-6 weeks old) were purchased from Charles River Laboratories (Wilmington, Massachusetts, USA). Mice were injected subcutaneously in their right flank with 10⁶ in vivo passaged SM1 melanoma cells suspended in 100 μL of PBS (Corning, 21-040-CV) at day 0. Cages were randomly assigned to different treatment groups, and mice were treated intraperitoneally with 100 μL of vehicle control or NextA at 20 mg/kg every other day when tumors became palpable, which was a week after tumor implantation.

Six days after the first NextA treatment, mice were also treated intratumorally with 100 μg of αCD47 every two days (clone miap301, BE0270, BioXCell, Lebanon, NH, USA) until the end of the study. Tumor measurements were taken every other day using caliper measurements and calculated using the formula L×W²/2. All animals were routinely monitored for early signs of toxicity. Emphasis was given to mortality, body weight, and food consumption. At the endpoint, a post-mortem evaluation was performed, including a gross visual examination of organs.

### 4. Phagocytosis Assays.

To evaluate phagocytosis by flow cytometry, SM1 melanoma cells were co-cultured with A3.1A7 cells or BMDMs derived from wild-type C57/Bl6 mice at a 2:1 ratio for 2 hr. or 4 hr. in serum-free media. Twenty-four (24) hours prior to co-culture, macrophages were polarized to their respective phenotypes as previously described in the presence or absence of NextA, and melanoma cells were untreated or treated with NextA. On the day of the co-culture, melanoma cells were stained with 1 μM of CellTrace™ CFSE Cell Proliferation Kit (ThermoFisher, C34554) and were washed extensively to remove excess dye. Phagocytosis assays were carried out in the presence of anti-mouse CD47 (clone miap301, 127502, Biolegend, San Diego, CA) and its respective isotype control (IgG, 400502, Biolegend), or anti human CD47 (clone CC2C6, 323102, Biolegend) or isotype control (MOPC-21, BE0083, Biolegend) at 25 μg/mL. Co-culture was set up in non-cell culture treated 96 U-well plates. After co-culture, cells were collected and stained with Brilliant Violet 785 anti-mouse F4/80 (clone BM8, Biolegend) or Brilliant Violet 785 anti-human CD11b (clone ICRF44, 301346, Biolegend).

Flow cytometry data was acquired a BD FACS Celesta Cell Analyzer, and data analysis was performed with FlowJo software version 10.3. Phagocytosis was determined as the percentage of CFSE+F4/80+ or CFSE+ CD11b+ cells out of total F4/80+ or total CD11b+ cells.

Phagocytosis was also evaluated by confocal microscopy and live imaging.

BMDMs derived from UBC-GFP mice were plated in Nunc Lab-Tek™ 8-well chamber slides (177402PK, ThermoFisher). The same treatment and polarization protocols as previously described were followed for these experiments. The day of the co-culture, SM1 melanoma cells were stained using CellTrace™ Far Red Cell Proliferation Kit (C34564, ThermoFisher Scientific), following manufacturer's protocol. Cells were extensively washed to remove any excess dye. SM1 cells were co-cultured with BMDMs at a 2:1 ratio, respectively, for 1 hr. in the presence of IgG control or αCD47 (Biolegend).

After co-culture, slides were fixed in 4% paraformaldehyde. Slides were incubated overnight with primary anti-GFP antibody (Cell Signaling, 2956) at 4° C. Slides were incubated with goat anti-rabbit IgG conjugated to Alexa Fluor 488 (A11008, ThermoFisher). Slides were mounted using VECTASHIELD Antifade Mounting Medium with DAPI (H-1200-10, Vector Laboratories, Burlingame, CA). Slides were imaged using a Zeiss 710 confocal microscope. Phagocytosis was quantified using Fiji by Image J, and the percentage of phagocytosis was calculated by dividing the number of GFP+ macrophages containing red dye in their vesicles out of total GFP+ macrophages. Live imaging was performed following a similar protocol and images were acquired through ImageXpress Pico Automated Cell Imaging System (Molecular Devices, San Jose, CA, USA).

### 5. Immunofluorescence.

A3.1A7 cells or BMDMs derived from wild-type C57/Bl6 mice were plated in Nunc Lab-Tek™ 8-well chamber slides (177402PK, ThermoFisher). The same treatment and polarization protocols as previously described were followed for these experiments. Twenty-four (24) hours after polarization, slides were fixed using 4% paraformaldehyde. The antibody against ARG-1 required an antigen retrieval step, where slides were boiled in citrate-based antigen unmasking buffer (H-3300-250, Vector Laboratories) for 10 min in a microwave. Slides were then incubated overnight at 4° C. with their respective antibodies: anti-iNOS (PA3-030A, ThermoFisher), anti-ARG1 (93668, Cell Signaling Technologies), anti-SIRPα (13379, Cell Signaling Technologies), and anti-CD68 (137001, Biolegend). The next day, slides were incubated with goat anti-rabbit IgG conjugated to Alexa Fluor 488 (A11008, ThermoFisher) or goat anti-rat IgG conjugated to Alexa Fluor 555 (A-21434, ThermoFisher) for 1 hr. at room temperature. Slides were mounted using VECTASHIELD Antifade Mounting Medium with DAPI (H-1200-10, Vector Laboratories, Burlingame, CA). Slides were imaged using a Zeiss 710 confocal microscope.

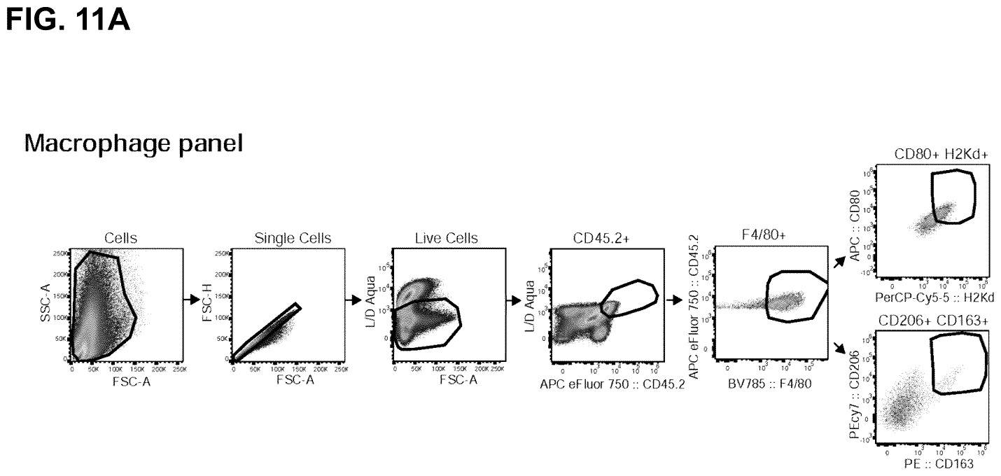

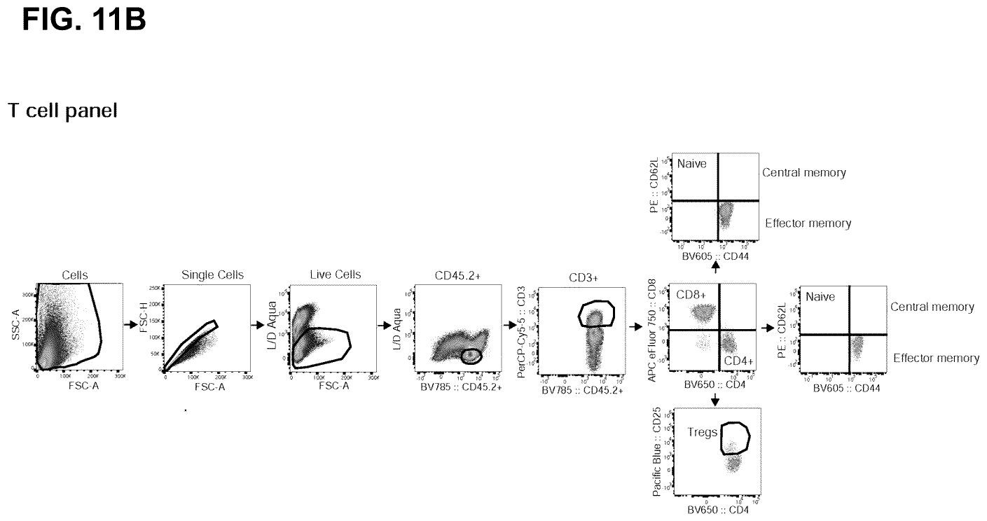

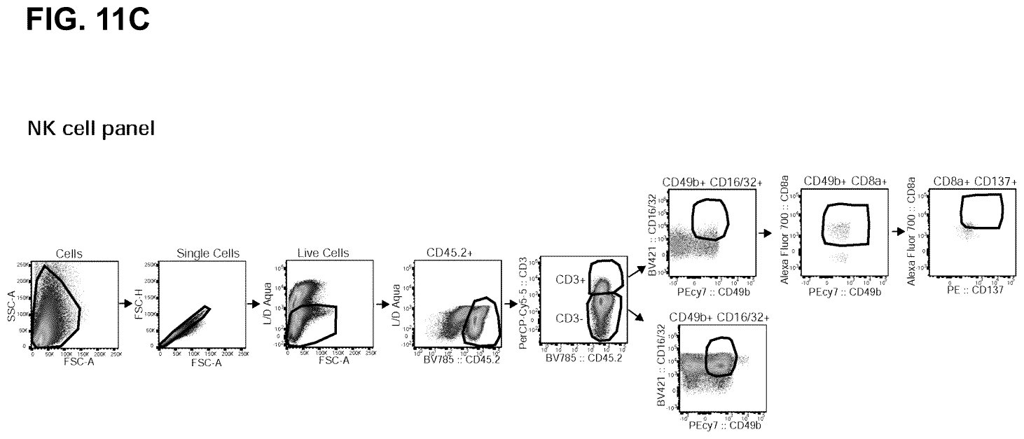

Flow Cytometry: Immunophenotyping of tumors by flow cytometry was performed following the protocol previously described (Knox T, et al. (2019) Sci. Rep. 9:6136; Banik D, et al. (2020) Cancer Res. 80:3649-3662). When control tumors reached the endpoint (2000 mm³), mice were euthanized following the IACUC protocol at The George Washington University, and tumors were processed into a single-cell suspension for analysis by flow cytometry with tumor digestion buffer containing collagenases I and IV, hyaluronidase V, and DNAse I. Dead cells were discriminated using LIVE/DEAD™ Fixable Aqua Dead Cell Stain Kit (ThermoFisher Scientific, L34965). The antibodies used to stain cell surface markers were all purchased from BioLegend (San Diego, CA) unless otherwise mentioned. Myeloid cell surface markers include: APC/Fire 750 anti-mouse CD45.2 (clone 104), Brilliant Violet 421 anti-mouse/human CD11b (clone M1/70), Brilliant Violet 605 anti-mouse Ly-6G/Ly-6C (Gr-1) (clone RB6-8C5), Brilliant Violet 785 anti-mouse F4/80 (clone BM8), APC anti-mouse CD80 (clone 16-10A1), PerCP/Cyanine5.5 anti-mouse H-2Kd (clone SF1-1.1), PE/Cy7 anti-mouse CD206 (MMR) (clone C068C2), PE anti-mouse CD163 (clone 5B11), and Alexa Fluor 700 anti-mouse CD3 (clone 17A2). T cell surface markers are as follows: Brilliant Violet 785 anti-mouse CD45.2 (clone 104), PerCP/Cy5.5 anti-mouse CD3 (clone 17A2), APC/Fire 750 anti-mouse CD8a (clone 53-6.7), Brilliant Violet 650 anti-mouse CD4 (clone GK1.5), Brilliant Violet 605 anti-mouse CD44 (clone IM7), PE anti-mouse CD62L (clone MEL-14), and Pacific Blue anti-mouse CD25 (clone PC61). NK cell surface markers are as follows: Brilliant Violet 785 anti-mouse CD45.2 (clone 104), PerCP/Cy5.5 anti-mouse CD3 (clone 17A2), PE/Cy7 anti-mouse CD49b (pan-NK cells) (clone DX5), Brilliant Violet 421 anti-mouse CD16/32 (clone 93), Alexa Fluor 700 anti-mouse CD8a (clone 53-6.7), and PE anti-mouse 4-1BB (clone 17B5). Multicolor flow data was acquired using a BD FACS Celesta Cell Analyzer, and data analysis was performed with FlowJo software version 10.3. The gating strategies for each immune cell panel can be found in FIG. 11A-FIG. 11C.

Cell surface expression of CD47 in melanoma cells was evaluated using PE anti-mouse CD47 (clone miap301, 127508200, Biolegend), PE anti-human CD47 (clone CC2C6, 323108, Biolegend), and their respective isotype controls (400508, 400113, respectively, Biolegend). SIRPα was evaluated using APC anti-mouse CD172a (SIRPα) antibody (clone P84, 144014, Biolegend) or APC anti-human CD172a/b (SIRPα/β) antibody (clone SE5A5, 323810, Biolegend) and their respective isotype controls (400411, Biolegend).

Titration of αCD47 in vitro: The blockade and saturation of the mouse anti-CD47 blocking antibody were evaluated following the protocol described by Yang Li et al (Li Y, et al. (2020) Nat. Commun. 11:581). Briefly, anti-mouse CD47 (clone miap301, 127502, Biolegend) was serially diluted to block cell surface CD47 in SM1 melanoma cells. After incubation for 1 hr., cells were washed and stained with PE anti-mouse CD47 (clone miap301, 127508200, Biolegend) to evaluate free cell surface CD47 or with PE anti-rat IgG2a antibody (clone Poly4054, 405406, Biolegend) to evaluate CD47 antigen saturation after blockade.

qRT-PCR: Total RNA was extracted from cells using the Trizol reagent according to manufacturer's instructions (ThermoFisher Scientific, 15596018). Samples were then processed immediately or stored at −80° C. RNA was quantified using a NanoDrop One Spectrophotometer (ThermoFisher Scientific). The 260/280 ratios were routinely over 1.8. Sample cDNA was produced using the iScript cDNA synthesis kit (Bio-Rad, 1708891). Target mRNA was quantified using MyIQ single-color real-time PCR detection system from Bio-Rad (Bio-Rad, Hercules, CA) and iQ SYBR green Supermix (Bio-Rad, 1708882). The following primers were synthesized by Invitrogen (Waltham, MA) with sequences designed by Origene (Rockville, MD): mouse R-actin (MP200232), mouse Nos2 (MP208933), mouse MRC1 (MP207800), human R-actin (HP204660), human IL-1β (HP200544), human MRC1 (HP206121), human NOS2 (HP200591), human CD209 (HP214086), mouse SIRPα (MP215450), mouse LRP1 (MP207604), human LRP1 (HP206040), mouse MFGE8 (MP208190), human MFGE8 (HP225757), mouse CD36 (MP201923), human CD36 (HP200058), and mouse CD47 (MP201932). Primers for mouse ARG-1 (PPM31770C) and human SIRPα (QT01031051) were purchased from Qiagen (Hilden, Germany). Human CD47 primer sequences were obtained from Sudo T, et al and are 5′-GGCAATGACGAAGGAGGTTA-3′ (sense) (SEQ ID NO:01) and 5′-ATCCGGTGGTATGGATGAGA-3′ (antisense) (SEQ ID NO:02) (Sudo T, et al. (2017) Oncol. Lett. 14:801-809). Human CD86 primer sequences are 5′-CAACACAATGGAGAGGGAAGA-3′ (sense) (SEQ ID NO:03) and 5′-TTAAAAACACGCTGGGCTTC-3′ (antisense) (SEQ ID NO:04). Mouse CD80 sequences are 5′-GATGCTCACGTGTCAGAGGA-3′ (sense) (SEQ ID NO:05) and 5′-CAACGATGACGACGACTGTT-3′ (antisense) (SEQ ID NO:06). Cycling conditions were set as per manufacturer's instructions. Single product amplification was confirmed by melting curve analysis. Quantification is expressed in arbitrary units and target mRNA levels were normalized to R-actin expression.

### 6. Immunoblotting.

Cells were treated with the respective compounds for 24 h. Then, cells were lysed using RIPA buffer (ThermoFisher Scientific Pierce, 89901, Waltham, MA) supplemented with 1× protease and phosphatase inhibitor (ThermoFisher Scientific, A32961). Lysates were sonicated at 4° C. for 8 minutes (8 cycles of 30 sec on, 30 sec off). Protein concentration was quantified using a BCA protein assay (ThermoFisher Scientific, 23225) following manufacturer's instructions. Samples were mixed with 6× Laemmli SDS sample reducing buffer (Alfa Aesar, J61337AC, Tewksbury, MA), and boiled at 80° C. for 10 min. Next, samples were loaded on to 4%-20% gels (Bio-Rad, 4561096, Hercules, CA), transferred to PVDF membranes and blocked with Odyssey Blocking Buffer (LI-COR Biosciences, 927-40000, Lincoln, NE). The following primary antibodies used for immunoblotting were purchased from Cell Signaling Technologies (Danvers, MA): tubulin (3873), acetylated tubulin (3971), arginase-1 (93668), and SIRPα (13379). Primary antibody recognizing HDAC6 was purchased from Assay Biotechnology (C0226, Fremont, CA), and iNOS antibody was purchased from ThermoFisher Scientific (PA3-030A). Bands were detected using Azure Biosystems Imaging System c600 (Azure Biosystems, Dublin, CA).

### 7. Luciferase Assays.

The mouse CD47 or SIRPα promoters were cloned into a promoterless pGL4.20[luc2/puro] vector using Genentech's cloning services (San Francisco, CA, USA). Vectors were transfected into SM1 and B16 melanoma cells using Lipofectamine 3000 (L3000008, ThermoFisher) and transfected cells were selected using puromycin. A31A7 macrophages were transiently transfected using jetOPTIMUS DNA transfection reagent (Polyplus, NY, USA). Luminescence was measured using the Luciferase Assay System (E4550, Promega) in a SpectraMax i3× Multi-Mode Microplate Reader (Molecular Devices).

### 8. Statistical Analysis.

Data were analyzed using GraphPad Prism (version 7; San Diego, CA) and presented as mean values±SD or mean values±SEM from a minimum of three experimental replicates. Representative data is presented from experiments that were performed at least two or three times. Tumor growing curves were analyzed by two-way ANOVA among groups. The level of significant differences in group means was assessed by student's t-test or by two-way ANOVA, and a p value of ≤0.05 was considered significant in all analyses herein.

### Example 1

### HDAC6 Inhibitors Modulated the Phenotype of Macrophages

Macrophages can be phenotypically classified as M1 (pro-inflammatory, anti-tumoral) or M2 (anti-inflammatory, pro-tumoral). This is a simplistic classification as macrophages are highly plastic cells and can display heterogeneity in their phenotype. While the M1 phenotype is derived from T-helper 1 (Th1) derived cytokines, the M2 phenotype is induced by Th2 derived cytokines. Although the M1 phenotype is typically induced in vitro upon stimulation with IFNγ and LPS,

Here, two M1-like macrophage phenotypes were included: M1 (polarized with IFNγ and LPS) and M1(IFNγ) (polarized with IFNγ only). The two different M1 phenotypes were included because LPS is a potent inducer of inflammation as it is an important component of bacterial cell walls (Tucureanu M M, et al. (2017) Int. J. Nanomedicine 13:63-76; Ngkelo A, et al. (2012) J. Inflamm. Lond. Engl. 9:1) and thus not present in the TME. Therefore, an IFNγ-primed only M1 phenotype should be included as IFNγ alone has been shown to induce macrophage activation (Wu C, et al. (2014) J. Immunol. Baltim. Md. 1950 193:3036-3044). The M2-like phenotype was induced with IL-4 and IL-13, which are cytokines widely used in previous publications to induce this phenotype (Zhang M Z, et al. (2017) Kidney Int. 91:375-386).

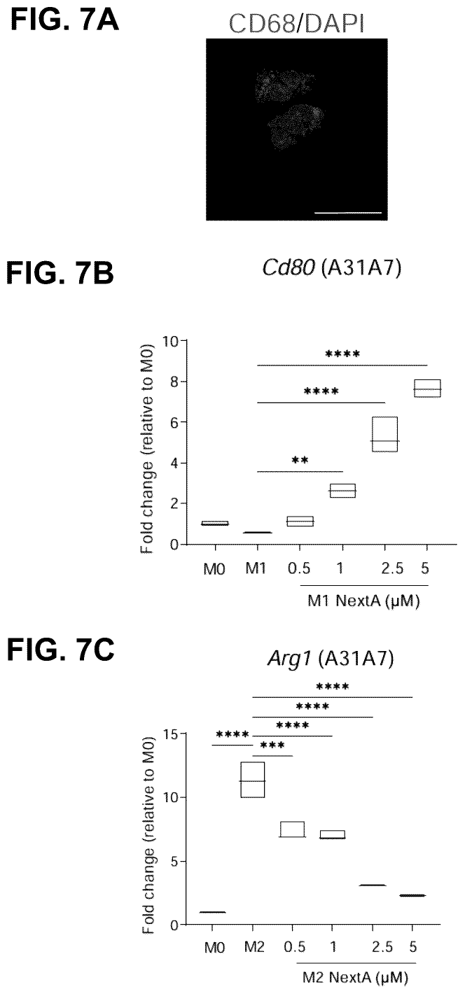

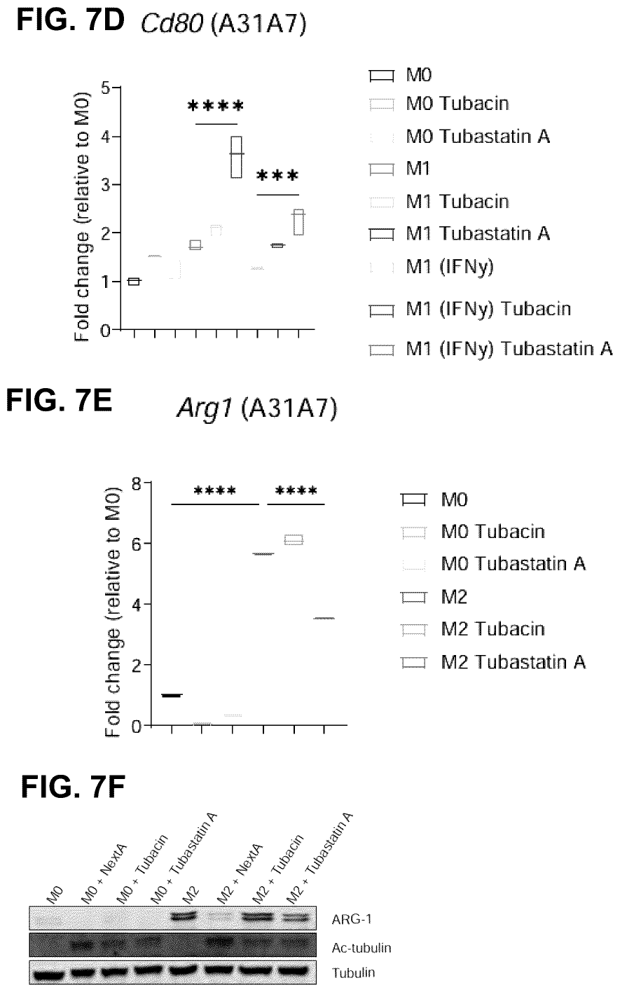

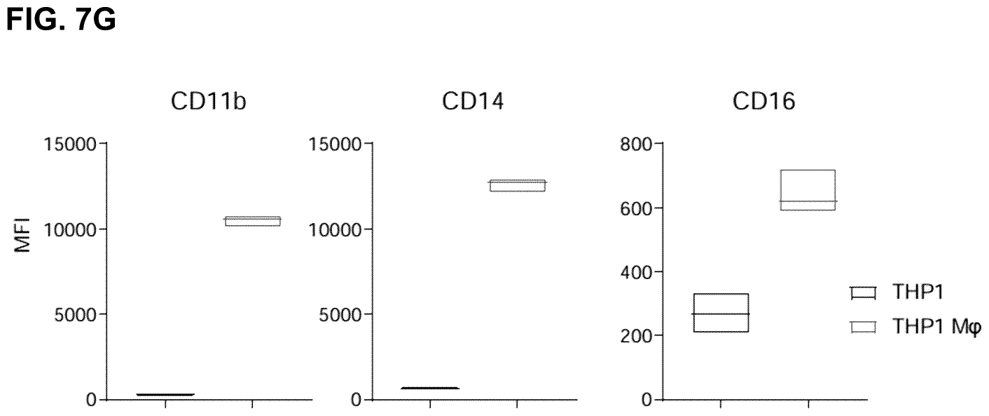

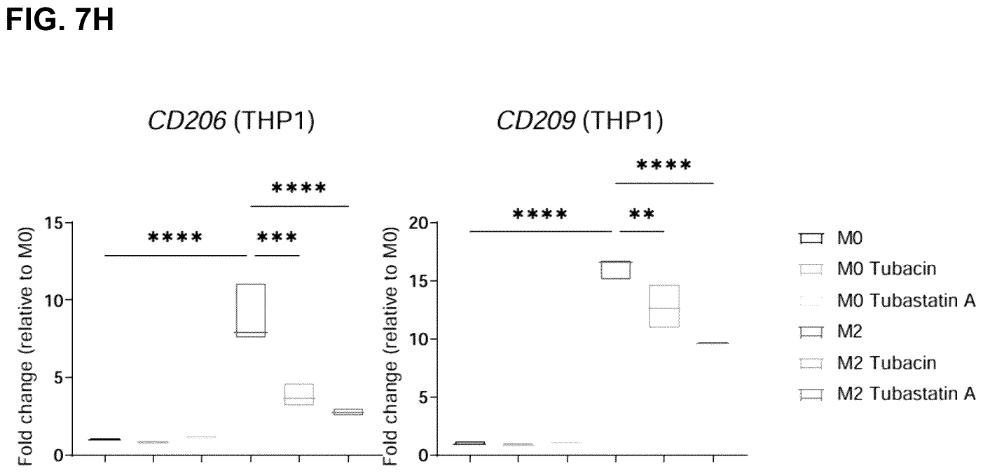

As described herein, the role of HDAC6 inhibitors (HDAC6i) in modulating the phenotype of macrophages in vitro using bone marrow derived macrophages (BMDMs) isolated from C57/Bl6 mice and the bone marrow-derived macrophage cell line (A31A7) was examined. Macrophages were left unpolarized (M0) or were polarized to M1, M1(IFNγ), or M2 as described above, and in the presence or absence of the highly selective HDAC6 inhibitor NextA. The identity of the A31A7 cell line was verified by evaluating the expression of the pan-macrophage marker CD68 by confocal microscopy (FIG. 7A). Mouse A31A7 macrophages were polarized towards the M1 or M2 phenotypes in the presence of different concentrations of NextA, and a concentration-dependent effect on the expression of Cd80 in M1 polarized macrophages (FIG. 7B) and Arg1 in M2 polarized macrophages was observed by qRT-PCR (FIG. 7C). These results demonstrated that 5 μM of NextA elicited observable phenotypic changes in both macrophage polarizations.

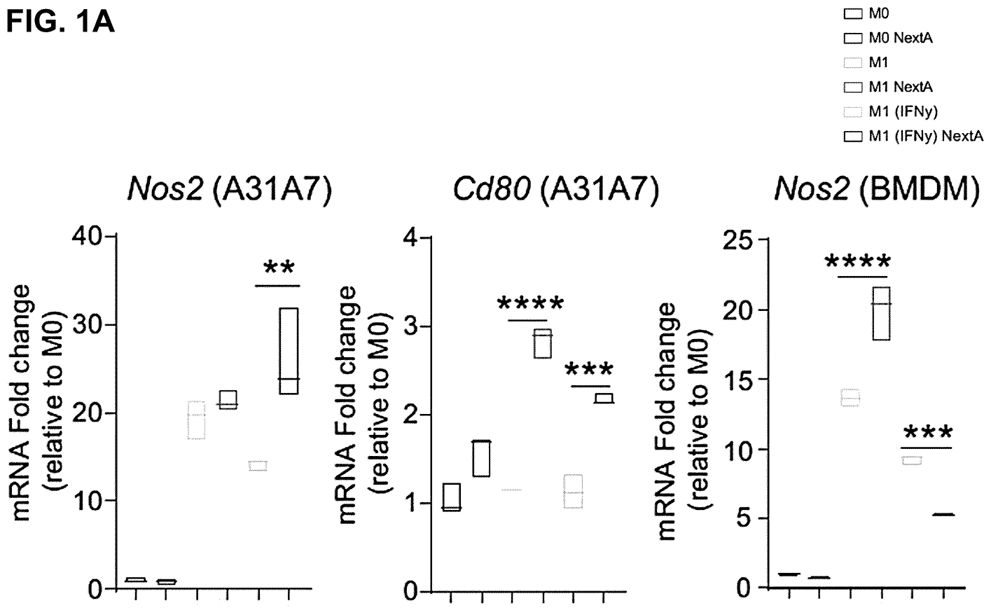

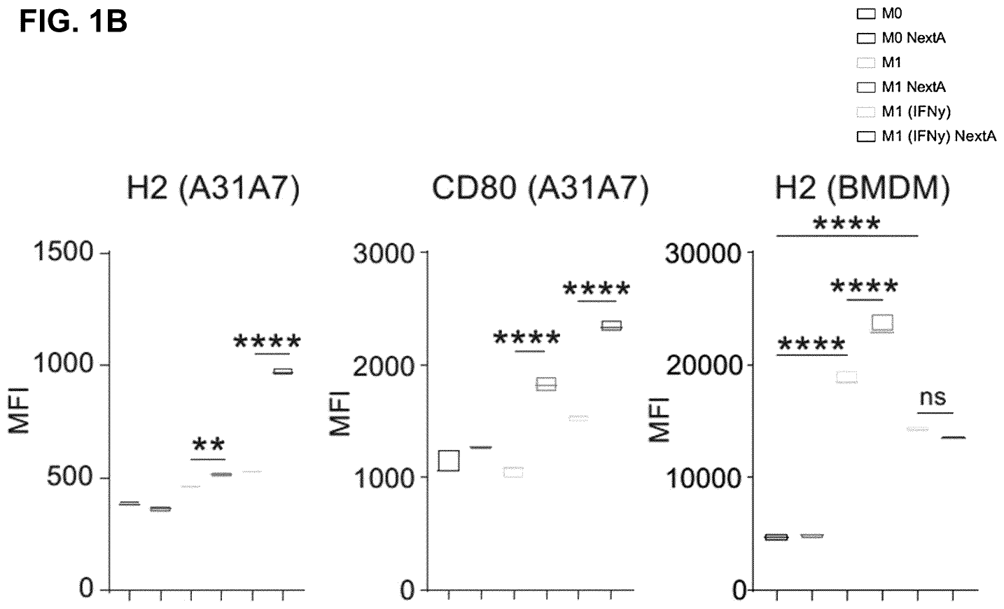

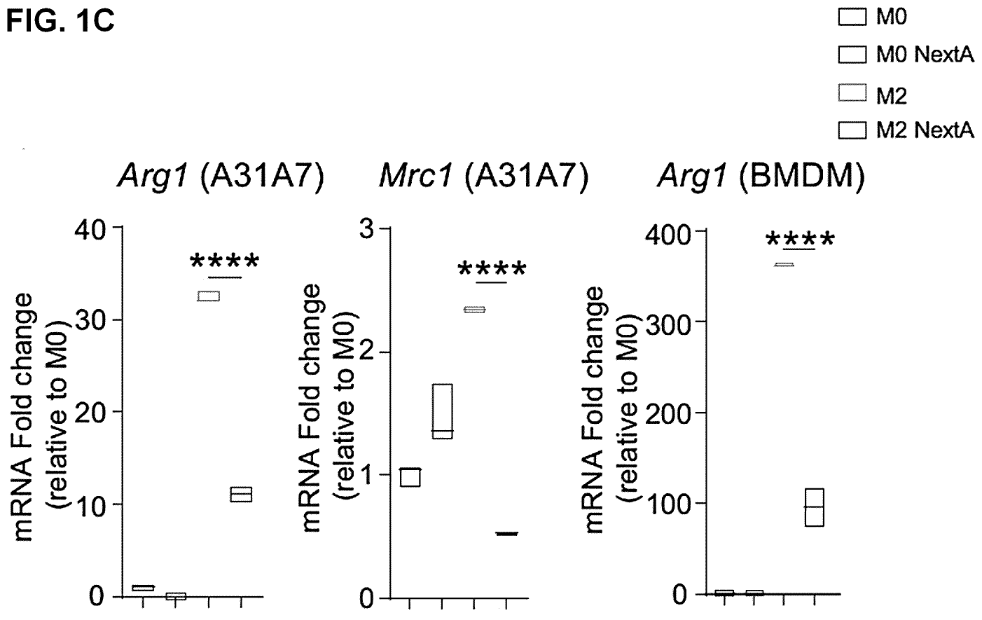

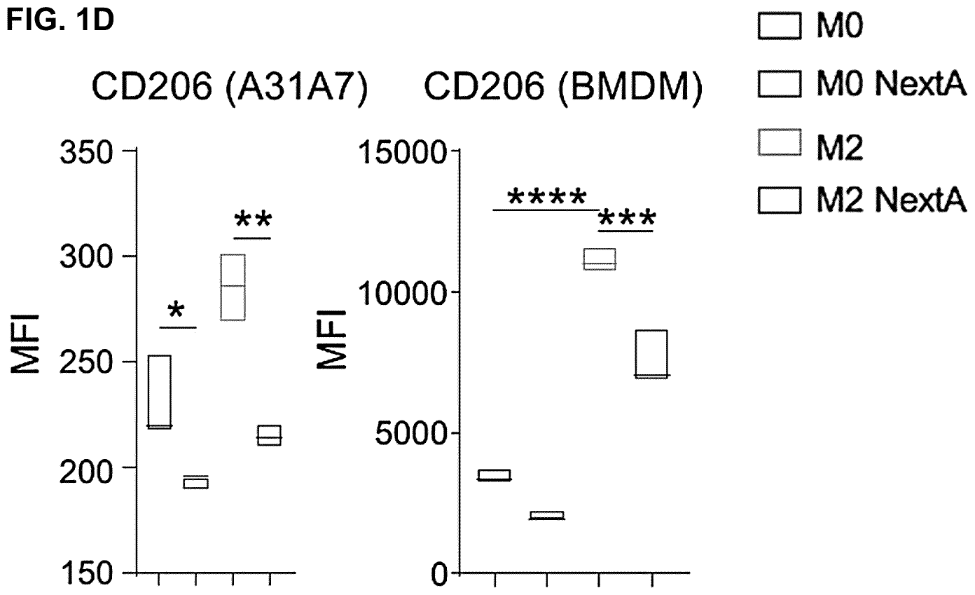

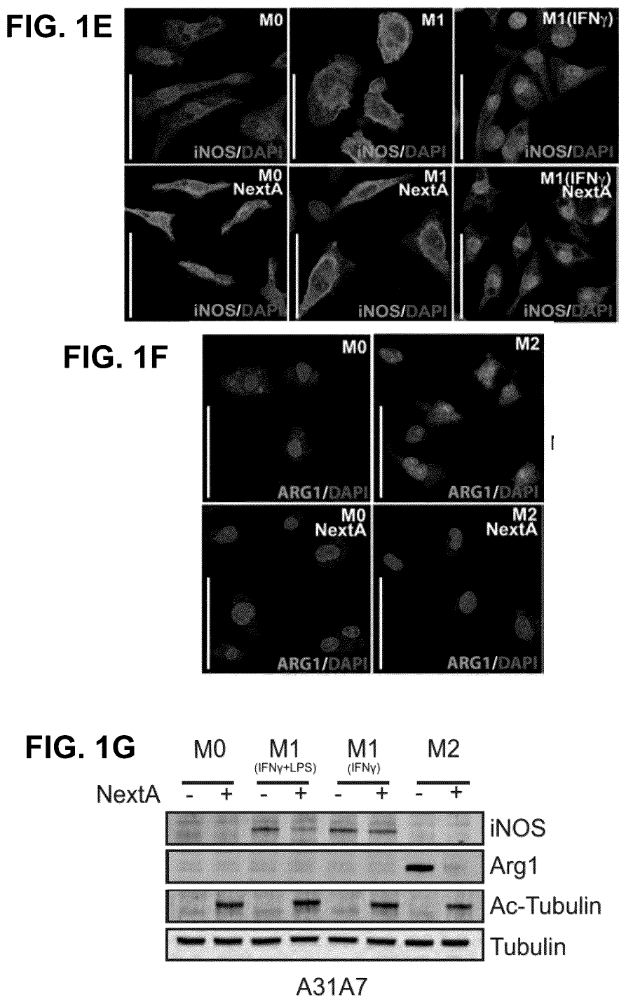

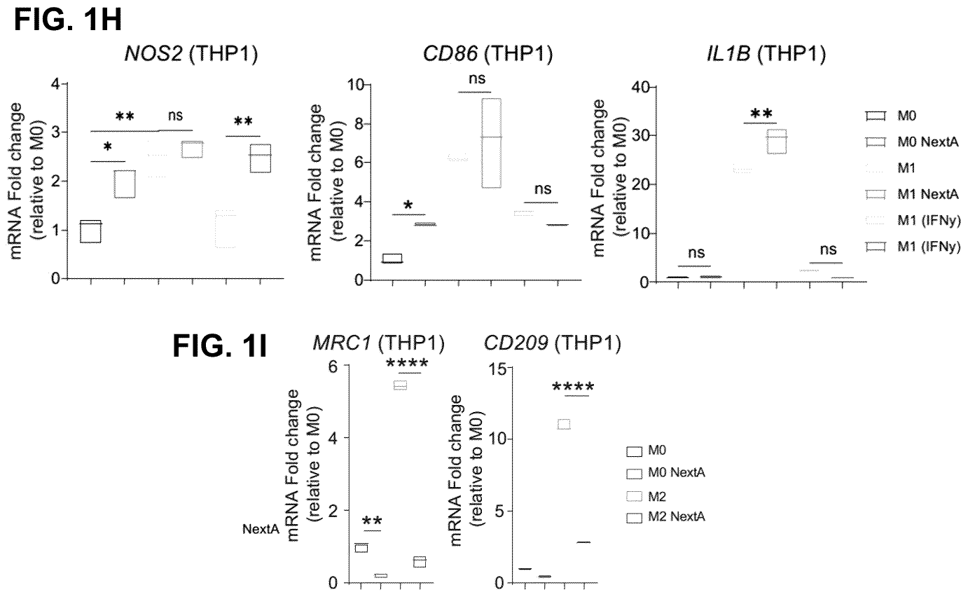

Other M1-associated markers in mouse A31A7 macrophages and primary BMDMs that were polarized to M1 or M1(IFNγ) were then evaluated. NextA treatment tended to significantly upregulate Nos2 and Cd80 at the transcriptional level (FIG. 1A). The cell surface expression of CD80 and H2 was also evaluated by flow cytometry in mouse A31A7 and BMDMs polarized to M1-like phenotypes, and there was an upregulation of CD80 and H2 upon NextA treatment (FIG. 1B). In contrast to the M1 phenotype, NextA significantly downregulated the expression of M2-associated markers such as Arg1 and Mrc1 by qRT-PCR (FIG. 1C) as well as CD206 surface expression (FIG. 1D) in A31A7 cells and BMDMs.

The protein expression of iNOS and ARG1 in BMDMs polarized to M1-like or in BMDMs polarized to M2-like phenotypes was examined (FIG. 1E). There was no change in iNOS and there was a substantial decrease in ARG1. These markers were also evaluated by Western Blotting in A31A7 cells (FIG. 1G) and similar results were obtained.

The effect of the enzymatic inhibition of HDAC6 over the macrophage phenotype was also validated by testing other highly selective HDAC6 inhibitors, such as Tubacin and Tubastatin A in A31A7 macrophages. Treatment with Tubacin and Tubastatin A upregulated Cd80 expression in M1-like macrophages and downregulated Arg1 in M2 polarized macrophages as evaluated by qRT-PCR (FIG. 7D-FIG. 7E). The selectivity and ability of NextA, Tubacin, and Tubastatin A to downregulate ARG1 expression in M2 macrophages was evaluated by Western Blot (FIG. 7F). From the Western Blot results, NextA was determined to be the most potent HDAC6 inhibitor as shown by (i) a robust increase in acetylated tubulin, a marker for HDAC6 inhibition (Zhang Y, et al. (2003) EMBO J. 22:1168-1179), and (ii) a decrease in ARG1 expression. NextA was chosen for the ensuing experiments.

Lastly, whether modulatory effects of HDAC6i translated from mouse to human macrophages was also examined. Using the human THP1 monocytic cell line, THP1 monocytes were differentiated into macrophages using 5 ng/mL of PMA, which were then polarized to their respective phenotypes in the presence or absence of 5 μM NextA. The differentiation of THP1 monocytes to macrophages upon PMA treatment was subsequently verified by evaluating morphological changes and cell surface expression of CD11b, CD14, and CD16 by flow cytometry (FIG. 7G), as previously described (Chen R F, et al. (2012) BMC Infect. Dis. 12:340). The M1-associated markers NOS2, CD86, and IL1B were either unaffected or significantly upregulated upon NextA treatment (FIG. 1H). Furthermore, NextA treatment significantly downregulated the expression of M2-associated markers such as CD206 and CD209 at the transcriptional level (FIG. 1I). These results were also verified using the other HDAC6i Tubacin and Tubastatin A (FIG. 7H). Together, these results indicate that HDAC6 plays an important role in controlling macrophage polarization, and that its inhibition can be used to modulate the macrophage phenotype in vitro.

### Example 2

### Inhibition of HDAC6 Decreased SIRPα Expression on Macrophages

Immune checkpoint (IC) expression is controlled by master regulators or transcription factors that regulate the expression of various genes involved in immune checkpoint phenomena and immunosuppression (Venkatraman S, et al. (2020) Vaccines. 8:735 (2020)). The role of HDAC6 in modulating other IC, such as the PD-1/PD-L1 pathway has been examined (Lienlaf M, et al. (2016) Mol. Oncol. 10:735-750), and that combination therapies that target both arms of the axis have led to a decrease in tumor growth in mouse melanoma and breast cancer models (Knox T, et al. (2019) Sci. Rep. 9:6136; Noonepalle S, et al. (2020) J. Med. Chem. 63:10246-10262; Banik D, et al. (2020) Cancer Res. 80:3649-3662). Therefore, after testing the modulatory role of HDAC6 on macrophage phenotype, the effects of HDAC6 inhibition in modulating the innate immune checkpoint CD47/SIRPα in both melanoma cells and macrophages was examined (and described herein).

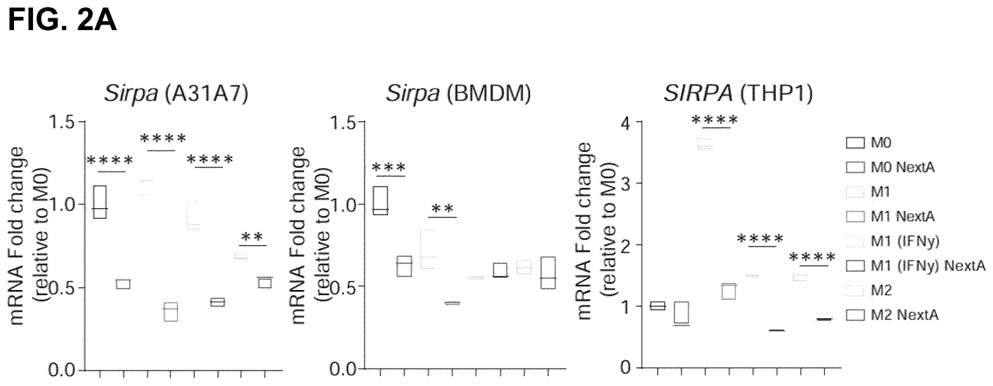

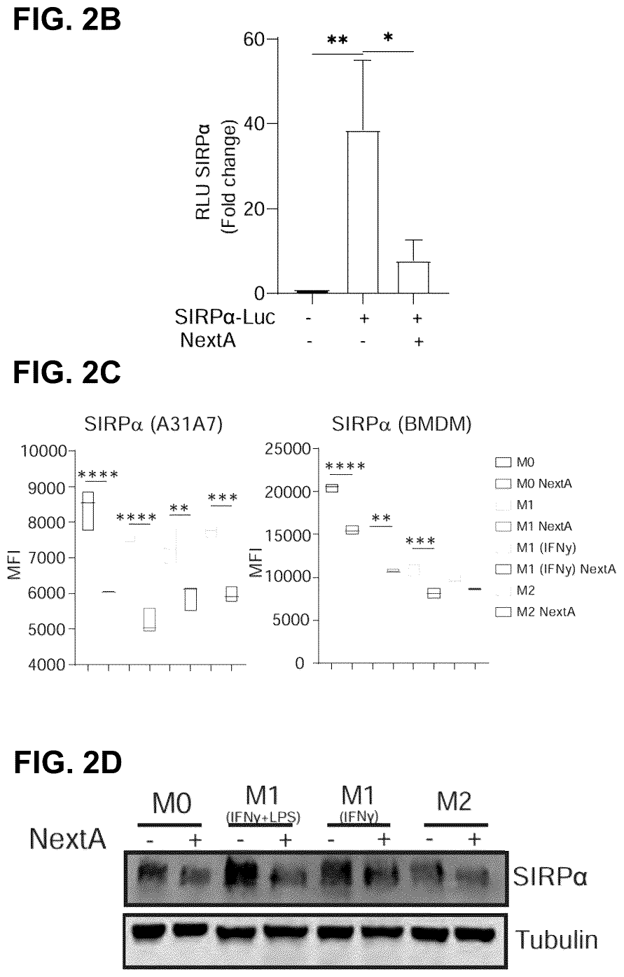

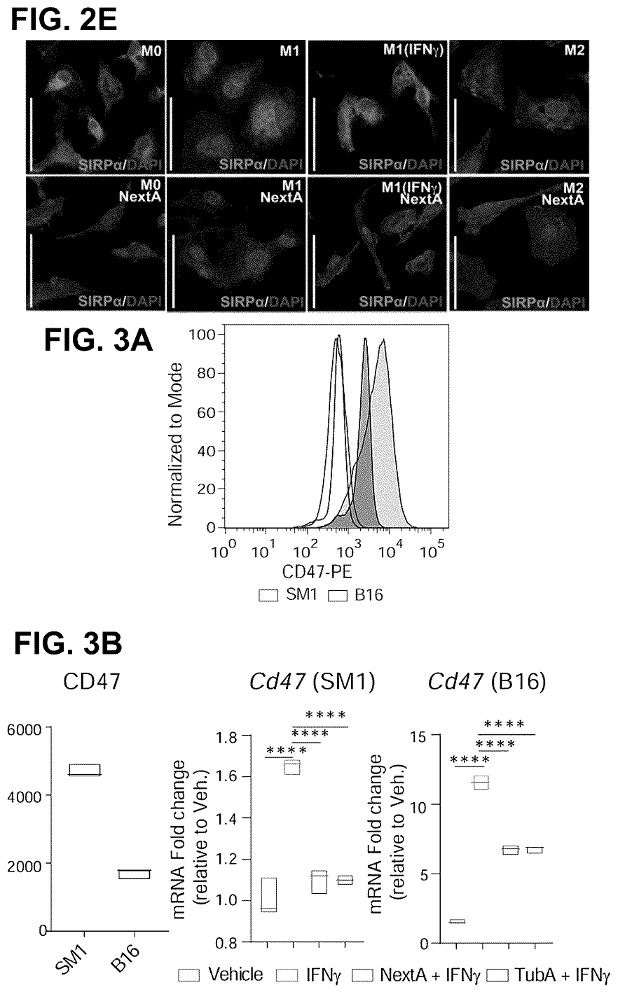

Despite the functional differences among macrophage phenotypes, SIRPα expression has been found to be similarly expressed by M1 and M2 macrophages (Lin Y, et al. (2018) Front. Immunol. 9:1744). Here, HDAC6 inhibition downregulated the expression of SIRPα on primary BMDMs and mouse and human macrophage cell lines (FIG. 2A). Treatment with NextA significantly downregulated Sirpa expression at the transcriptional level as evaluated by qRT-PCR (FIG. 2A). Interestingly, Sirpa downregulation appeared to be due to HDAC6 inhibition and not dependent on the macrophage phenotype as both unpolarized and polarized macrophages have decreased SIRPα expression upon NextA treatment.

To investigate whether HDAC6 controlled SIRPα expression at the transcriptional level, the mouse SIRPα promoter was cloned into a promoterless pGL4.20 [luc2/puro] vector to create a SIRPα-Luc reporter plasmid. e Nex, A31A7 macrophages were transiently transfected in the absence or presence of 10 μM of NextA and luminescence was evaluated. A31A7 cells were successfully transfected with the SIRPα-luciferase reporter vector as evidenced by a 32.2-fold increase in relative luminescence units (RLU) compared to untransfected cells (FIG. 2B). Treatment with NextA significantly decreased SIRPα expression as shown by a decrease in RLU compared to untreated cells (32.2-fold versus 7.7-fold, respectively). Overall, these results indicated that HDAC6 regulated SIRPα expression at the transcriptional level.

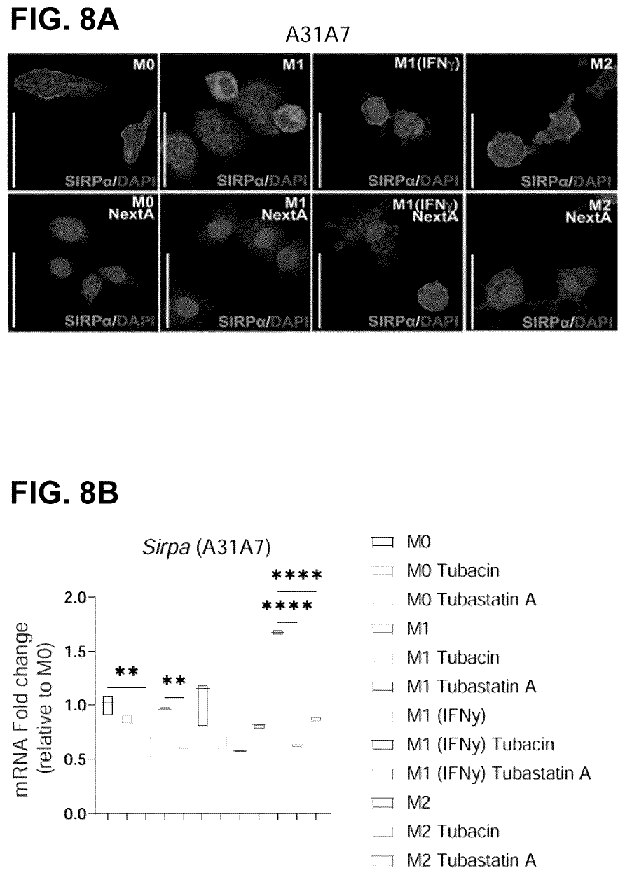

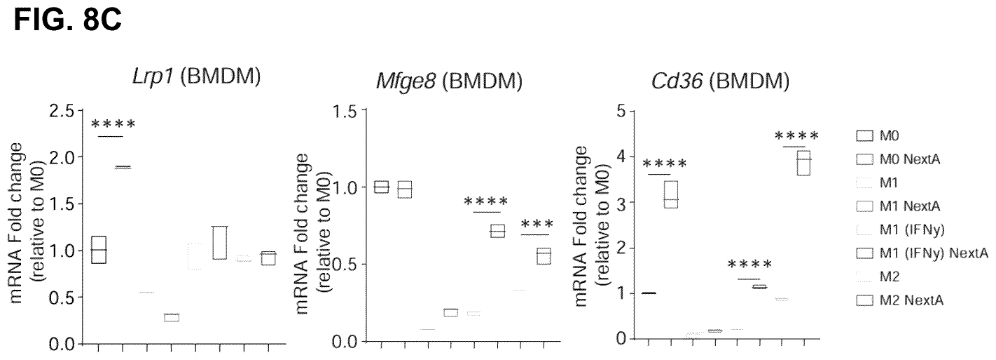

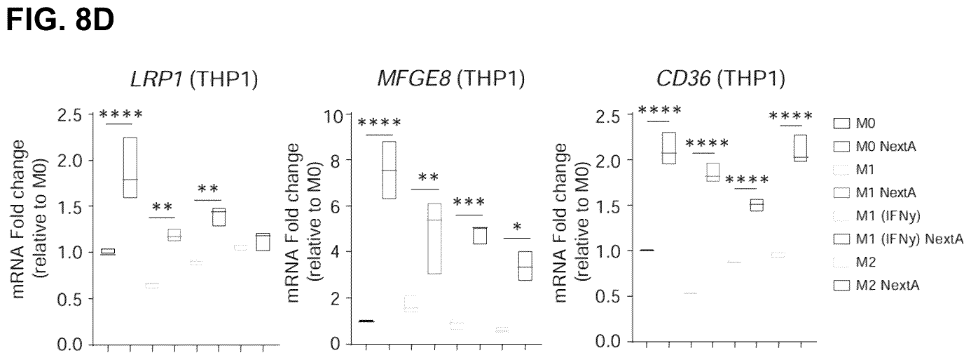

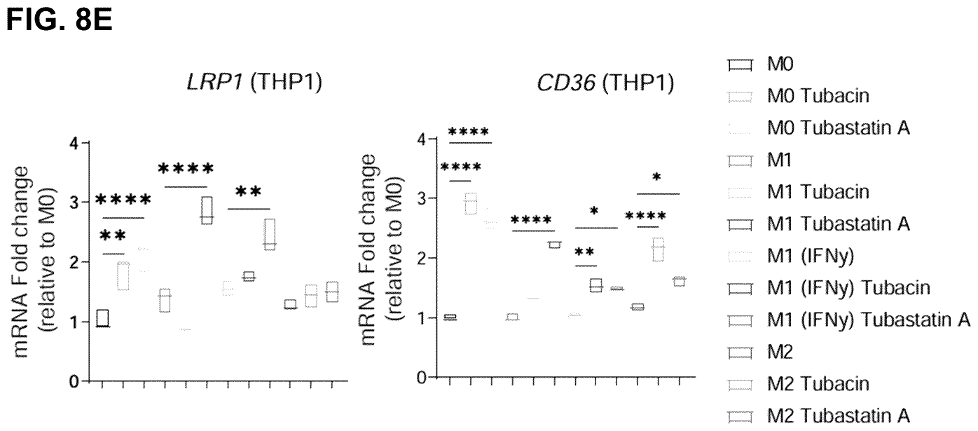

Whether the results observed at the transcriptional level correlated to the protein levels was next examined. Treatment with NextA significantly decreased SIRPα expression on the cell surface as evaluated by flow cytometry (FIG. 2C). The results were corroborated by assessing SIRPα expression at the protein level by Western Blot in A31A7 macrophages (FIG. 2D), and by confocal microscopy in primary BMDMs (FIG. 2E) and A31A7 cells (FIG. 8A). Lastly, to determine whether the downregulation of Sirpa was exclusive to NextA, Sirpa expression was evaluated upon treatment with other HDAC6 inhibitors, Tubacin and Tubastatin A (TubA) using qRT-PCR in A31A7 cells. Treatment with Tubacin and Tubastatin A generated similar results indicating that the observed effects were not an off-target effect of the HDAC6i NextA (FIG. 8B).

In addition to evaluating the modulation of the anti-phagocytic signal SIRPα, the role of HDAC6 inhibitors in regulating the expression of additional pro-phagocytic signals such as Lrp1, Cd36, and Mfge8 was examined. Lrp1 is the receptor for calreticulin (Orr A W, et al. (2003) J. Cell Biol. 161:1179-1189), a pro-phagocytic signal expressed on target cells, while Cd36 and Mfge8 are adaptor proteins that facilitate the binding of phosphatidylserine on target cells to the phosphatidylserine receptor on macrophages (Fadok V A, et al. (1998) J. Immunol. Baltim. Md. 1950 161:6250-6257; Borisenko G G, et al. (2004) Cell Death Differ. 11:943-945). Using primary mouse BMDMs and human THP1-derived macrophages (FIG. 2C-FIG. 2D, respectively), a significant increase in the expression of Lrp1, Cd36, and Mfge8 was observed upon HDAC6 inhibition with NextA. There results were corroborated using human THP1-derived macrophages upon treatment with Tubacin and Tubastatin A (FIG. 8E). Interestingly, these effects were more pronounced in human macrophages than same cells of mouse origin. Collectively, these results indicate that HDAC6 inhibitors can be used to modulate the phagocytosis of macrophages as these inhibitors downregulate the expression of anti-phagocytic signals (i.e., SIRPα) and upregulate pro-phagocytic signals on macrophages.

### Example 3

### Pharmacological and Genetic Abrogation of HDAC6 Controlled CD47 Expression in Melanoma

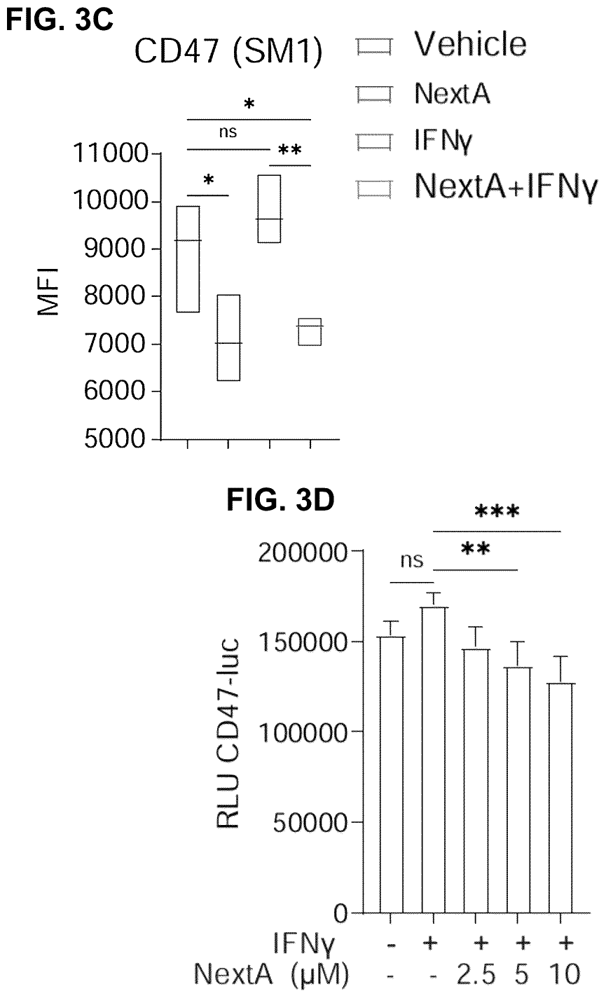

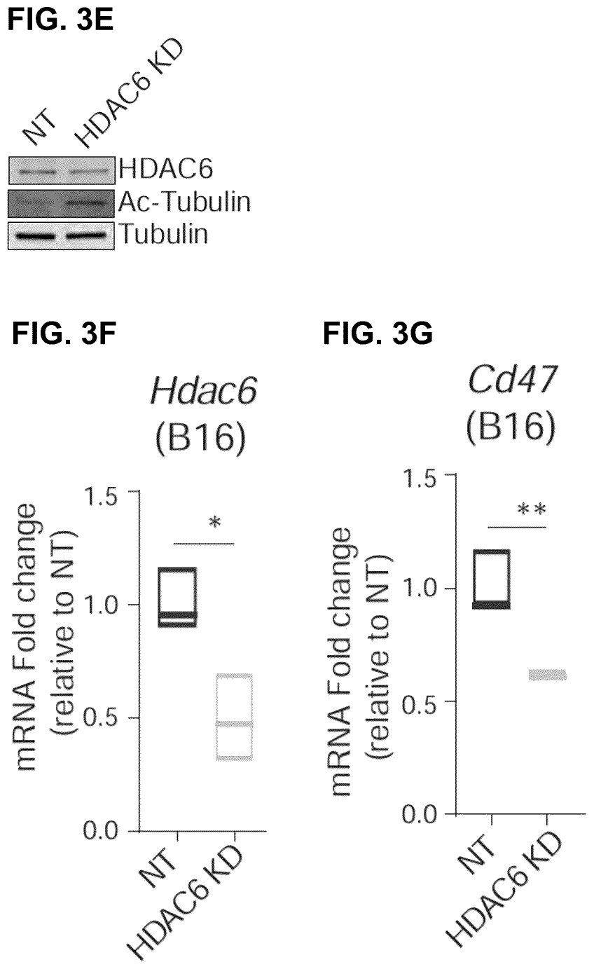

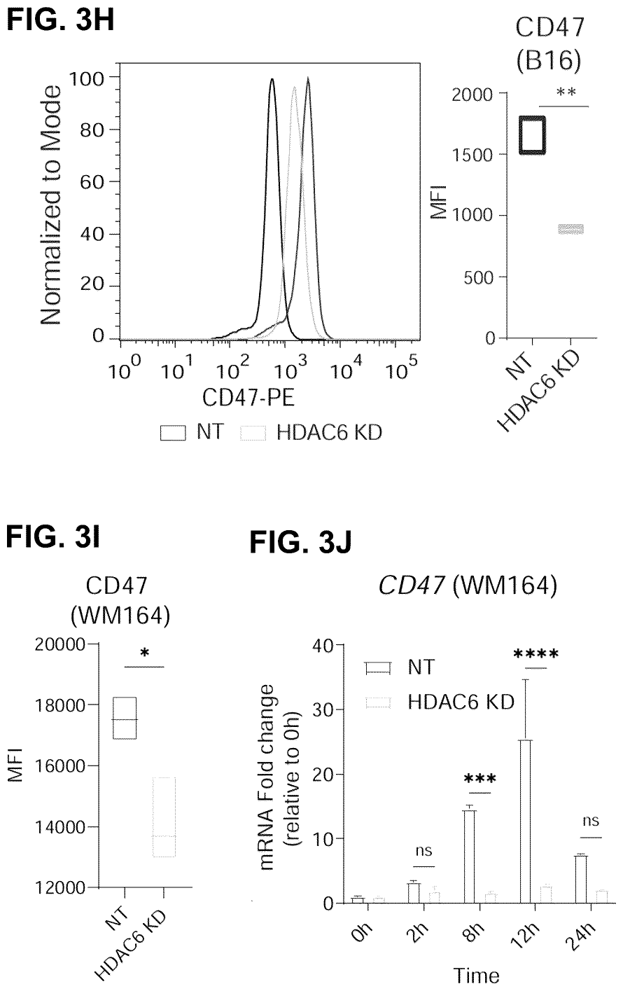

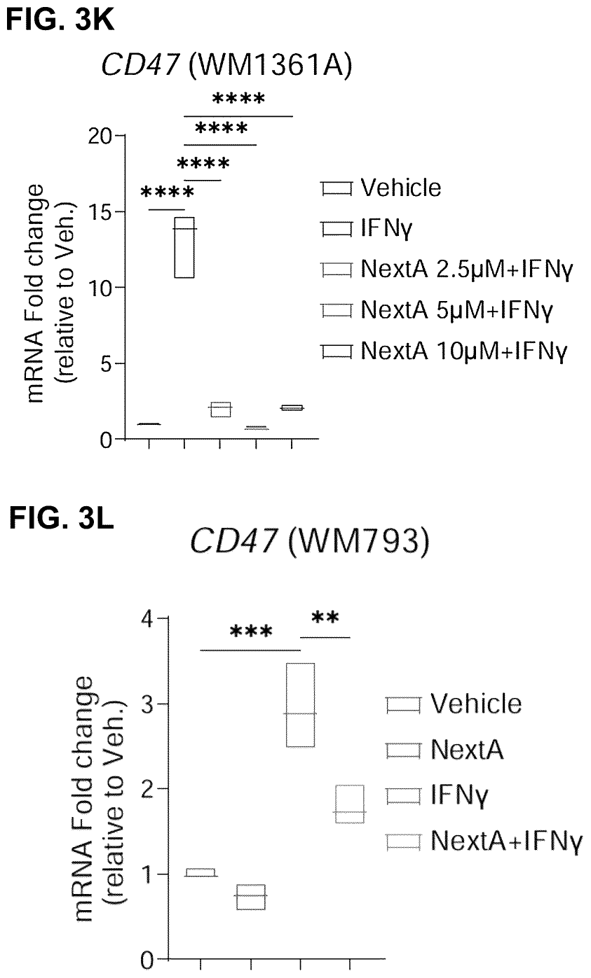

CD47 is upregulated in tumor cells to evade phagocytosis by macrophages. CD47 is overexpressed in hematological malignancies and solid tumors (Oldenborg P A, et al. (2000) Science. 288:2051-2054; Ye Z H, et al. (2021) Transl. Oncol. 14:101162). Compared to other cancer types, skin cutaneous melanoma ranks within the middle range of CD47 mRNA expression (Gupta A, et al. (2020) Cancer Drug Resist. 3:550-562). There was limited therapeutic efficacy of CD47 blockade in mice bearing the B16F10 melanoma tumor model (Ingram J R, et al. (2017) Proc. Natl. Acad. Sci. 114:10184-10189; Sockolosky J T, et al. (2016) Proc. Natl. Acad. Sci. U.S.A. 113:E2646-E2654). B16F10 melanoma tumors are not representative of human melanoma as they lack mutations present in melanoma patients and represent a poorly immunogenic tumor model, which provides a rationale as to why B16 models are not responsive to ICB (Mosely SIS, et al. (2017) Cancer Immunol. Res. 5:29-41). There is a better antitumor response to immunomodulatory agents such as anti-PD-1 in the SM1 tumor model than in the B16 models (Knox T, et al. (2019) Sci. Rep. 9:6136). Therefore, this study evaluated CD47 cell surface expression in both mouse melanoma models, SM1 and B16F10 (abbreviated B16). Interestingly, SM1 cells expressed CD47 at much higher levels than B16F10 cells (FIG. 3A). These data indicate that the SM1 model can be the more effective model for evaluating the antitumor properties of CD47 blocking antibodies than the B16 model.

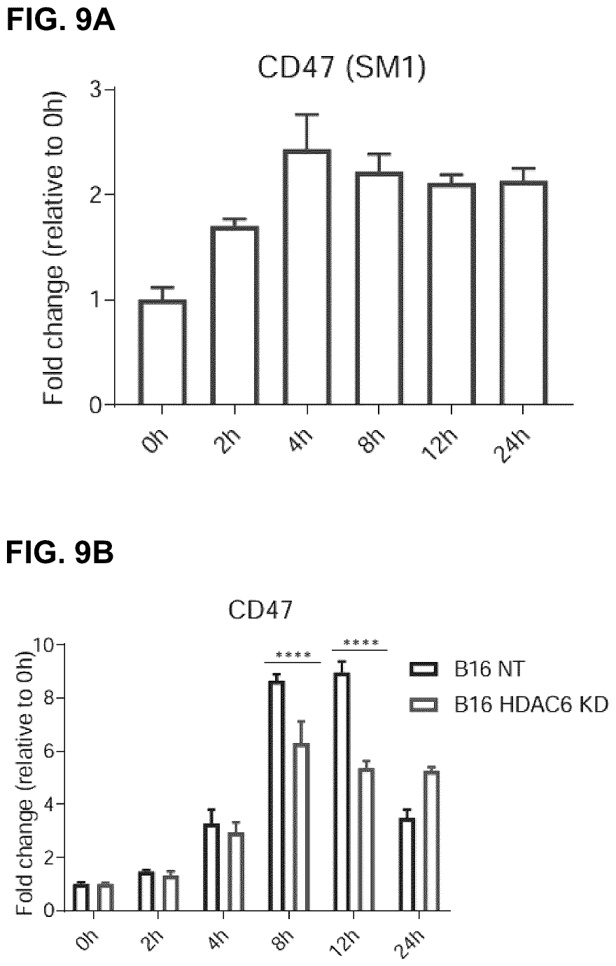

CD47 can be upregulated in response to numerous cytokines (Huang C Y, et al. (2020) Transl. Oncol. 13:100862). CD47 is upregulated in an inflammatory setting mediated by IFNγ, and thus promote immune evasion (Sockolosky J T, et al. (2016) Proc. Natl. Acad. Sci. U.S.A. 113:E2646-E2654; Ye Z H, et al. (2021) Transl. Oncol. 14:101162; Huang C Y, et al. (2020) Transl. Oncol. 13:100862; Basile M S, et al. (2019) PloS One. 14:e0210276). Although the mechanism by which IFNγ drives CD47 upregulation is unclear, IFNγ expression correlates to CD47 expression in skin cutaneous melanoma (Ye Z H, et al. (2021) Transl. Oncol. 14:101162). The kinetics of Cd47 mRNA in SM1 and B16 cells upon IFNγ stimulation (100 ng/mL) were evaluated and these mouse melanoma models were determined to have different kinetics (FIG. 9A-FIG. 9B). It was determined that 12 hours was a time point for both models to sustain the IFNγ-mediated upregulation. In FIG. 9B, the role of HDAC6 inhibitors NextA and Tubastatin A in abrogating Cd47 upregulation upon stimulation with IFNγ for 12 hours was evaluated. IFNγ significantly upregulated Cd47 at different levels in SM1 and B16 melanoma cells (1.6 vs. 10-fold, respectively). HDAC6 inhibition using NextA and Tubastatin A repressed these effects at the transcriptional level in both models. The lower endogenous Cd47 expression in B16 cells when compared to SM1, which allows for a greater upregulation upon stimuli, can explain these data.

These data were validated using SM1 melanoma cells to evaluate cell surface expression of CD47 by flow cytometry 24 hours post-IFNγ stimulation (FIG. 3C). Furthermore, to investigate whether HDAC6 controls CD47 expression at the transcriptional level, the mouse CD47 promoter was cloned into a promoterless pGL4.20[luc2/puro] vector. SM1 melanoma cells were transfected with the CD47-luciferase reporter vector and stimulated with IFNγ in the absence or presence of NextA at different concentrations and measured luminescence. Treatment with NextA repressed IFNγ-mediated upregulation of Cd47 (FIG. 3D).

To evaluate the possibility of off-target effects of the pharmacological inhibition of HDAC6, a genetic knock-down (KD) of Hdac6 in B16 melanoma cells (HDAC6 KD) was performed. A partial genetic KD of HDAC6, as evidenced by decreased HDAC6 expression by Western Blotting and an increase in acetylated tubulin (FIG. 3E), was obtained. The partial KD was also confirmed by qRT-PCR (FIG. 3F). The modulatory effects of HDAC6 on CD47 after performing the partial HDAC6 KD were evaluated, and it was observed that HDAC6 KD cells significantly downregulated Cd47 expression at the mRNA and cell surface levels using qRT-PCR (FIG. 3G) and flow cytometry (FIG. 3H). Partial loss of HDAC6 repressed IFNγ-driven Cd47 upregulation at different time points (FIG. 9B).

This phenomenon was further examined in human melanoma cell lines to test the translational application of the results obtained using mouse models. HDAC6 knock-down (KD) in human WM164 melanoma cells downregulated cell surface CD47 expression compared to the non-target (NT) control (FIG. 3I). Furthermore, the effects of HDAC6 KD on IFNγ-driven CD47 upregulation was analyzed. For these purposes, WM164 NT and HDAC6 KD cells were stimulated with 100 ng/mL of IFNγ, and RNA was collected at different time points. Genetic abrogation of HDAC6 significantly prevented CD47 upregulation upon IFNγ stimulation, especially at 8 hr. and 12 hr. time points (FIG. 3J). From this kinetic evaluation, the best results were obtained at 8 hr. and 12 hr. after stimulation; thus, these time points were used for the following experiments. The effects of pharmacological inhibition of HDAC6 in other human melanoma cell lines (WM1361A and WM793) were also assessed. WM1361A melanoma cells were stimulated with IFNγ in the absence or presence of NextA at different concentrations (2.5 μM, 5 μM, and 10 μM). As shown in FIG. 3K, NextA prevented IFNγ-driven CD47 upregulation at different concentrations 12 hrj. post-stimulation. Finally, these results were corroborated in WM793 human melanoma cells (FIG. 3L). Altogether, these results indicate that HDAC6 may play a role in regulating CD47 expression in mouse and human melanoma cells.

### Example 4

### HDAC6i-Treated Macrophages have Enhanced Phagocytic Capacity

Blockade of CD47 in the cell surface of tumor cells enhances phagocytosis by preventing ligation between CD47 and SIRPα on macrophages, thus allowing the “eat me” signals to dominate (Morrissey M A, et al. (2020) Immunity. 53:290-302.e6; Willingham S B, et al. (2012) Proc. Natl. Acad. Sci. U.S.A. 109:6662-6667). HDAC6 inhibitors decreased SIRPα expression on macrophages, indicating that the combination of CD47 blocking antibodies and HDAC6 inhibition could further enhance the dominance of the pro-phagocytic signals in the phagocytic synapse. Thus, whether macrophages treated with NextA would be more phagocytic than untreated macrophages was examined.

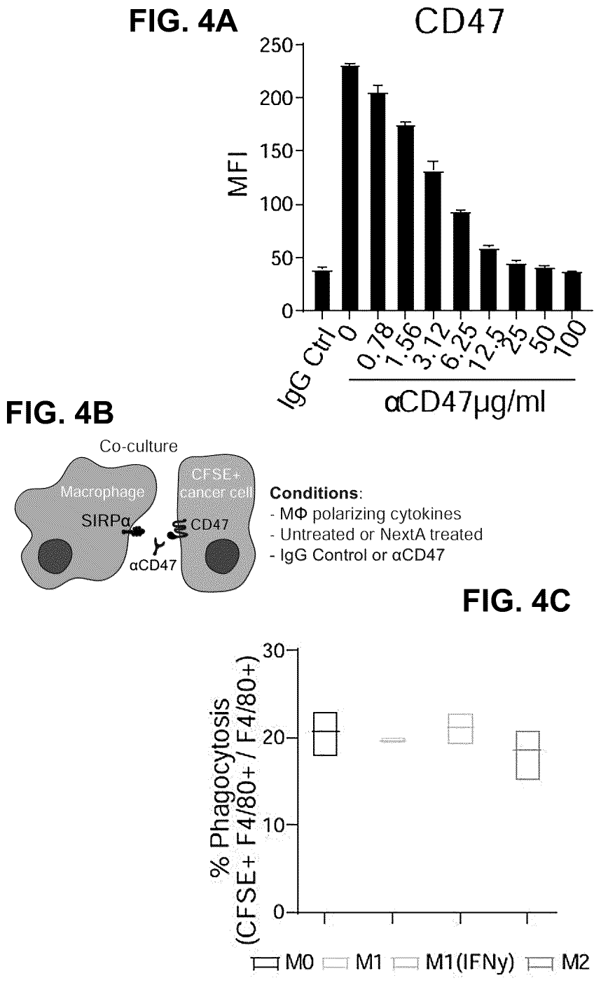

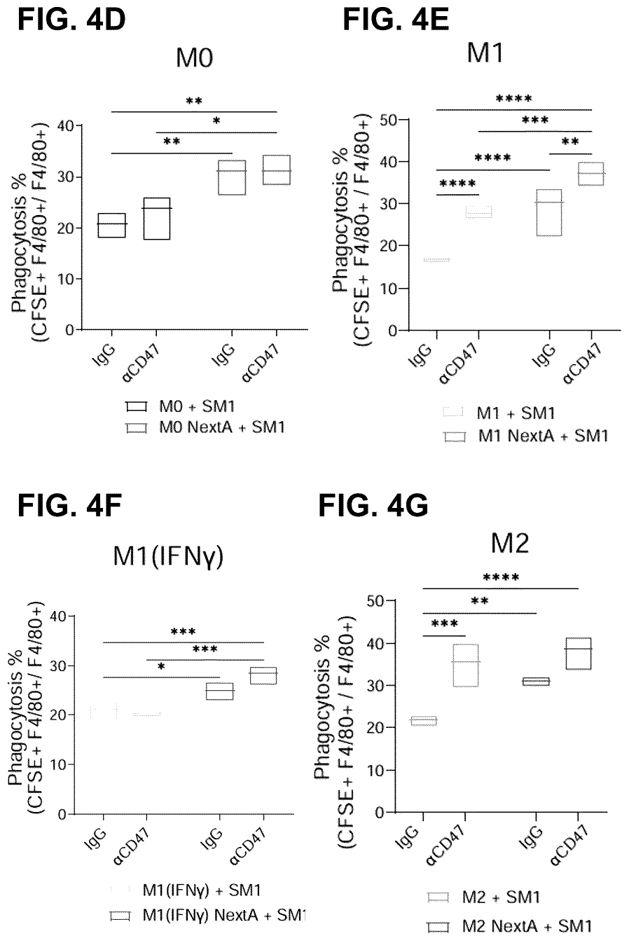

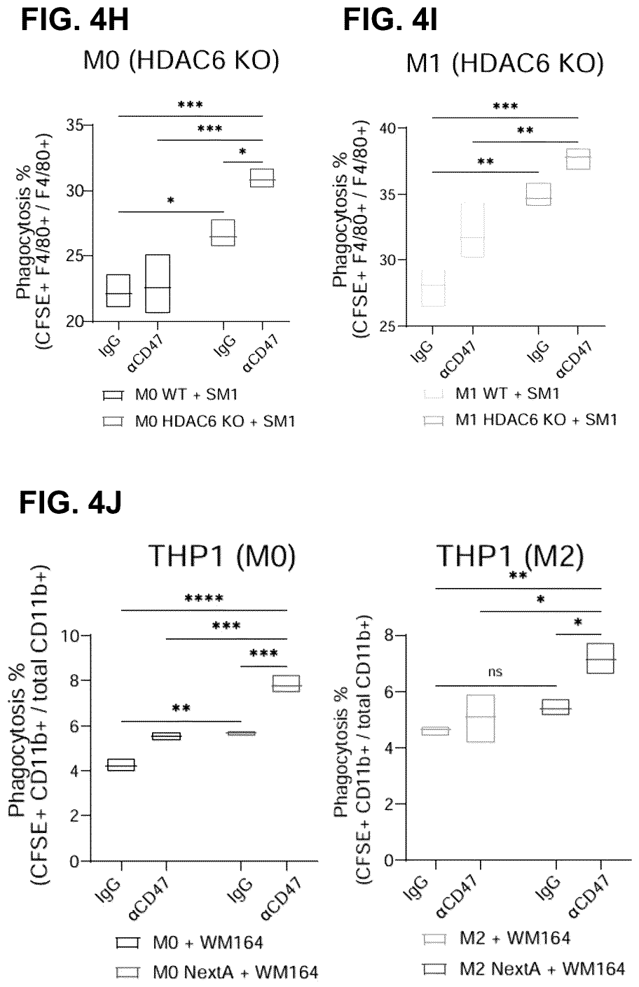

The αCD47 blocking antibody was first titrated to determine the concentration that allowed for the greatest CD47 blockade and epitope saturation in the SM1 mouse melanoma model using a previously described method (Li Y, et al. (2020) Nat. Commun. 11:581). From the CD47 blocking antibody titration, it was determined that 25 μg/mL was the optimal concentration for SM1 cells as it was the lowest antibody concentration that provided maximum surface CD47 blockade and epitope saturation at levels comparable to the respective negative controls (FIG. 4A). After determining the optimal concentration, flow cytometry-based phagocytosis assays were performed. SM1 cells were stained with carboxyfluorescein N-succinimidyl ester (CFSE) prior to co-culture with BMDMs that were left unpolarized or were polarized to different phenotypes in the presence or absence of NextA (FIG. 4B). Co-culture was performed for 2 hrs. or 4 hrs. in the presence of CD47 blocking antibody or isotype (IgG) control at 25 μg/mL. After incubation, co-cultured cells were stained for F4/80, a macrophage marker (Austyn J M, et al. (1981) Eur. J. Immunol. 11:805-815). The phagocytic capacity of primary BMDMs left unpolarized (M0) or polarized to M1, M1(IFNγ), or M2 were then compared. Different macrophage phenotypes were found to phagocytose cancer cells at similar rates independently of their polarization state (FIG. 4C).

How treatment with NextA modulated the phagocytic capacity of the different phenotypes in the presence of isotype (IgG) control or αCD47 was then evaluated. Interestingly, unpolarized (M0) and M1(IFNγ) macrophages did not significantly enhance phagocytosis upon CD47 blockade, whereas M1 and M2 macrophage phagocytosis significantly increased in the presence of αCD47 (FIG. 4D-FIG. 4G).

A genetic knockout approach was used to confirm these results and explore off-target effects from the pharmacological inhibition of HDAC6. BMDMs were harvested from HDAC6 knockout (KO) mice and polarized as previously described. HDAC6 KO naïve (M0) and M1-like macrophages had a higher phagocytic capacity than their wild-type counterparts (FIG. 4H-FIG. 4I). Phagocytosis was further increased in M1 HDAC6 KO BMDMs in the presence of anti-CD47. These results rule out the presence of off-target effects of the pharmacological inhibition of HDAC6 using NextA.

The role of HDAC6 inhibition on phagocytosis was also assessed using human THP1-derived macrophages and WM164 melanoma cells. Similar results, where unpolarized THP-1 derived macrophages that were treated with NextA were significantly more phagocytic than untreated macrophages, were observed (FIG. 4J).

To further characterize this phenomenon, phagocytosis was evaluated using confocal microscopy (FIG. 4K). For the microscopy experiments, BMDMs were harvested from UBC-GFP mice and polarized to the M1 phenotype in the presence or absence of NextA and IgG control or anti-CD47. SM1 cells were stained with CellTrace Far Red, and co-cultured with M1-like macrophages for 2 hr. Phagocytosis was determined by the internalization of the CellTrace Far Red dye in the GFP macrophages. M1 NextA+anti-CD47 had the highest number of phagocytic events compared to either treatment alone (FIG. 4K).

To assess the phagocytosis kinetics in those groups, live cell imaging was performed and phagocytosis was tracked for 4 hr. after the macrophages and SM1 cells were co-cultured for an hour. Images were acquired every four minutes thereafter.

As shown in FIG. 4L, M1 macrophages had a similar phagocytosis rate in the IgG control and the anti-CD47 group, although the latter had slightly enhanced phagocytosis. Interestingly, M1 NextA treated macrophages in the IgG control group had a steady increase in phagocytosis over time, which was maintained after the maximum of approximately 20% was reached. Lastly, M1 NextA treated macrophages in the presence of anti-CD47 had the highest phagocytosis rate. Although it decreased over time, the M1 NextA+anti-CD47 group maintained the highest phagocytosis rate in comparison to the other groups. These results indicate that NextA treatment of macrophages and CD47 blockade on the melanoma cells induces a more robust phagocytosis rate before a plateau is reached. The microscopy studies corroborated the phagocytosis results obtained by flow cytometry. In summary, the data described in this exampled indicate that HDAC6 inhibition enhanced the phagocytosis of cancer cells by macrophages, an effect that is further increased upon CD47 blockade. This effect of pharmacological inhibition of HDAC6 can be mediated by the downregulation of SIRPα on macrophages.

### Example 5

### Nexturastat A Synergized with αCD47 to Decrease Melanoma Tumor Growth

Treatment with CD47 blocking antibodies aims to enhance phagocytosis by tumor associated macrophages (TAMs), which display an M2-like phenotype (Lecoultre M, et al. (2020) J. Immunother. Cancer 8:e001408). It is likely that tumor-mediated release of immunosuppressive factors prevents CD47 blockade-mediated phagocytosis by TAMs to elicit an adaptive immune response (Lecoultre M, et al. (2020) J. Immunother. Cancer 8:e001408). This supports that CD47 blockade alone is not sufficient to decrease tumor burden in melanoma-bearing mice and generate adaptive antitumor immunity (Ingram J R, et al. (2017) Proc. Natl. Acad. Sci. 114:10184-10189; Jiang Z, et al. (2021) J. Hematol. Oncol. J Hematol Oncol 14:180; Sockolosky J T, et al. (2016) Proc. Natl. Acad. Sci. U.S.A. 113:E2646-E2654). However, this limitation of CD47 blockade as a stand-alone therapy can be overcome by combining it with other therapeutic approaches to stimulate antitumor immunity, such as antibodies targeting tumor-specific antigens or other immunosuppressive pathways like the PD-1/PD-L1 axis (Ingram J R, et al. (2017) Proc. Natl. Acad. Sci. 114:10184-10189; Sockolosky J T, et al. (2016) Proc. Natl. Acad. Sci. U.S.A. 113:E2646-E2654; Ngo M, et al. (2016) Cell Rep. 16:1701-1716).

HDAC inhibitors such as suberoylanilide hydroxamic acid (SAHA, inhibits classes I and II) enhance trastuzumab-mediated phagocytosis and thus are strong candidates to use in combination with therapies aiming to enhance phagocytosis (Laengle J, et al. (2020) J. Immunother. Cancer 8:e000195). However, there are no studies to date evaluating the effects of highly selective epigenetic modifiers such as HDAC6 (class IIb) inhibitors in potentiating these effects. Whether combining HDAC6 inhibitors such as NextA with anti-CD47 results in decreased tumor growth and enhanced phagocytic activity through SIRPα downregulation and modulation of the macrophage phenotype in vivo was explored.

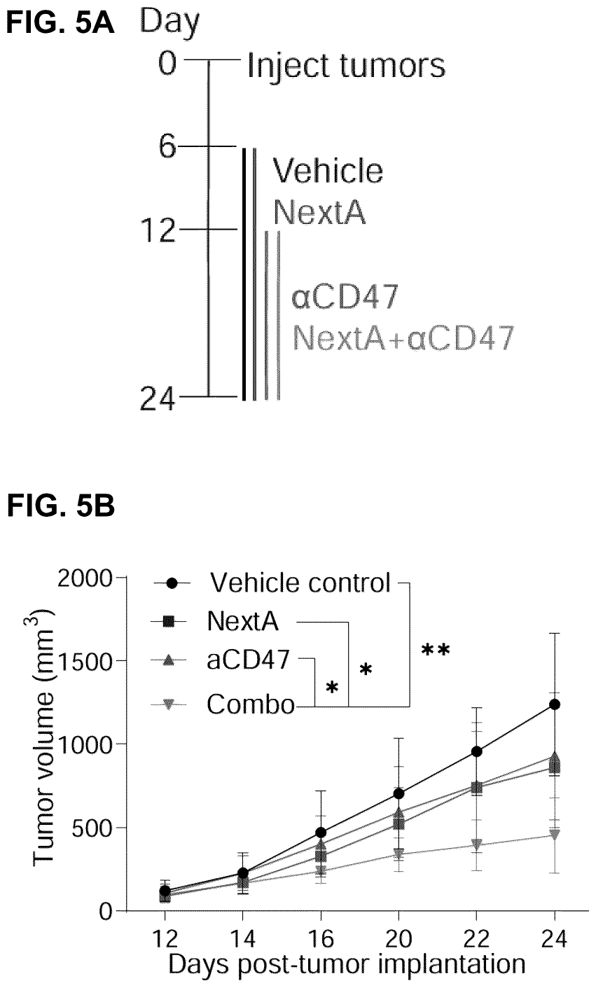

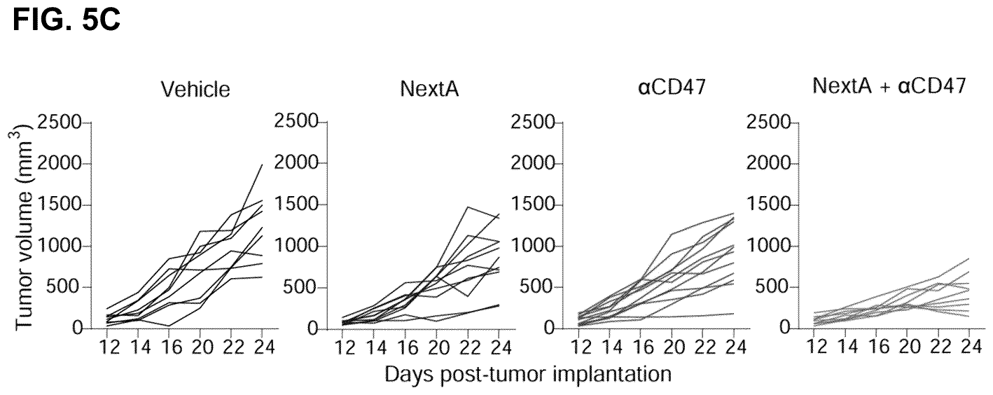

First, C57/Bl6 mice were inoculated with SM1 melanoma cells (n=15 mice per group). Six days after tumor inoculation, 20 mg/kg of NextA was administered intraperitoneally to the mice every other day until the experimental endpoint in the NextA alone and combination groups. Five days after starting treatment with NextA, 100 μg of anti-CD47 was injected intratumorally every other day for the duration of the study (FIG. 5A). The CD47 blocking antibody used in this study is MIAP301, which has been shown to partially exhibit tumor inhibition in syngeneic C57/Bl6 mouse models (Liu X, et al. (2015) Nat. Med. 21:1209-1215). CD47 blockade was administered intratumorally to prevent antigen sink and off-tumor effects, such as anemia. The concentrations of NextA and anti-CD47 were determined by performing drug and antibody titrations. Tumor growth was monitored every other day from when the tumors became palpable until the experimental endpoint. The data indicated that although NextA and αCD47 moderately decreased tumor growth as stand-alone therapies compared to the vehicle control, the combination of NextA and αCD47 therapy acted synergistically to reduce tumor growth in SM1-bearing mice (FIG. 5B-FIG. 5C).

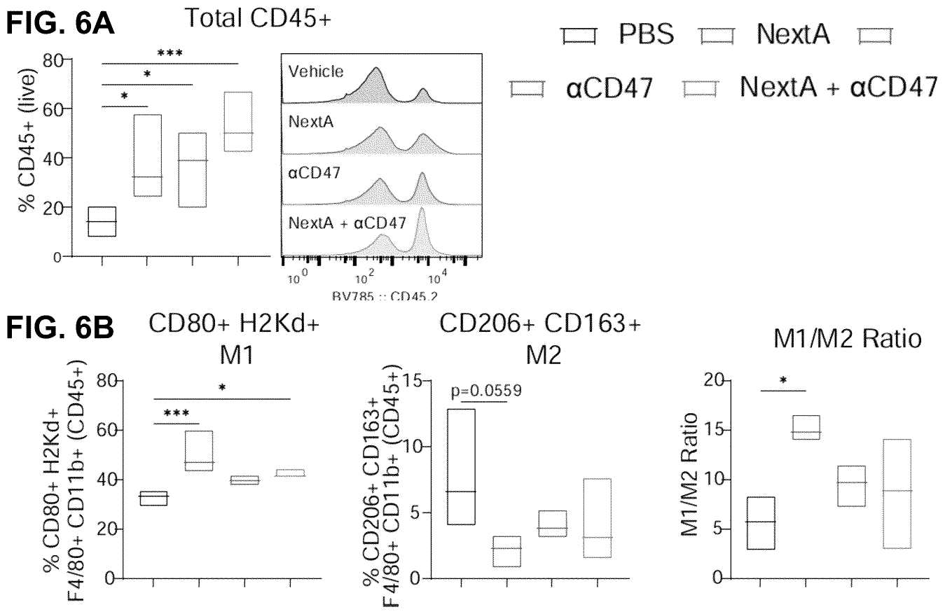

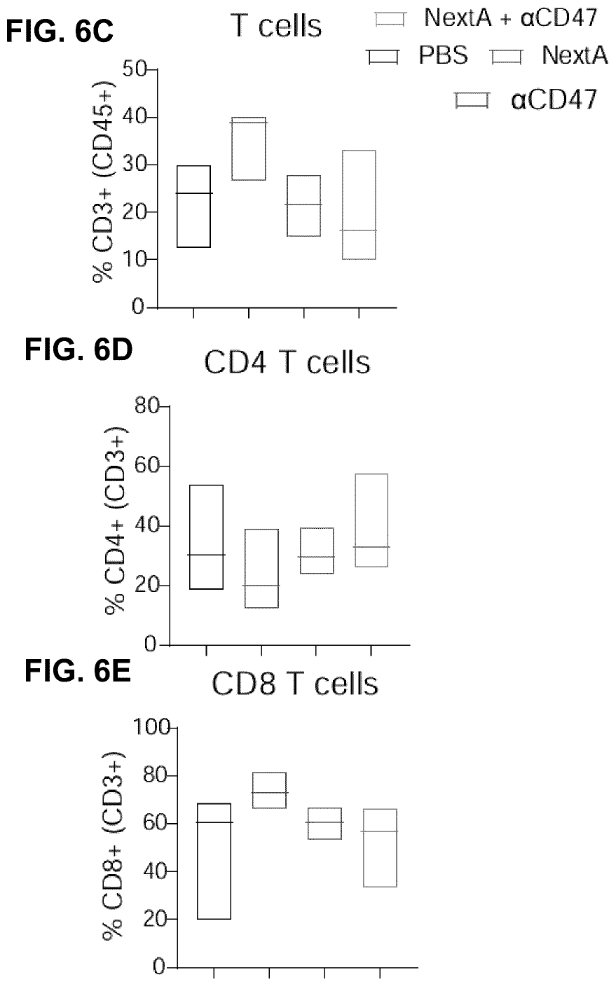

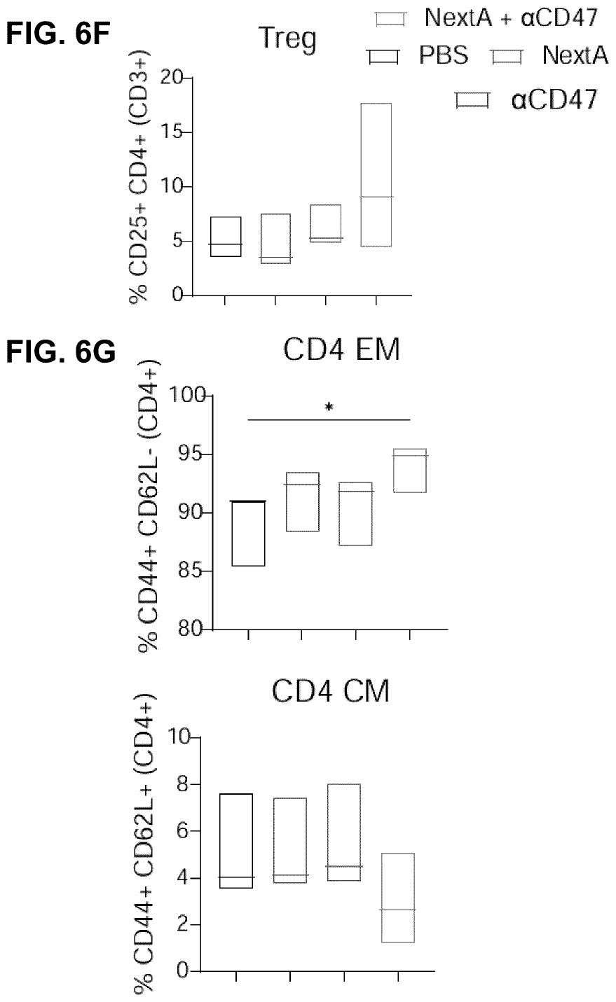

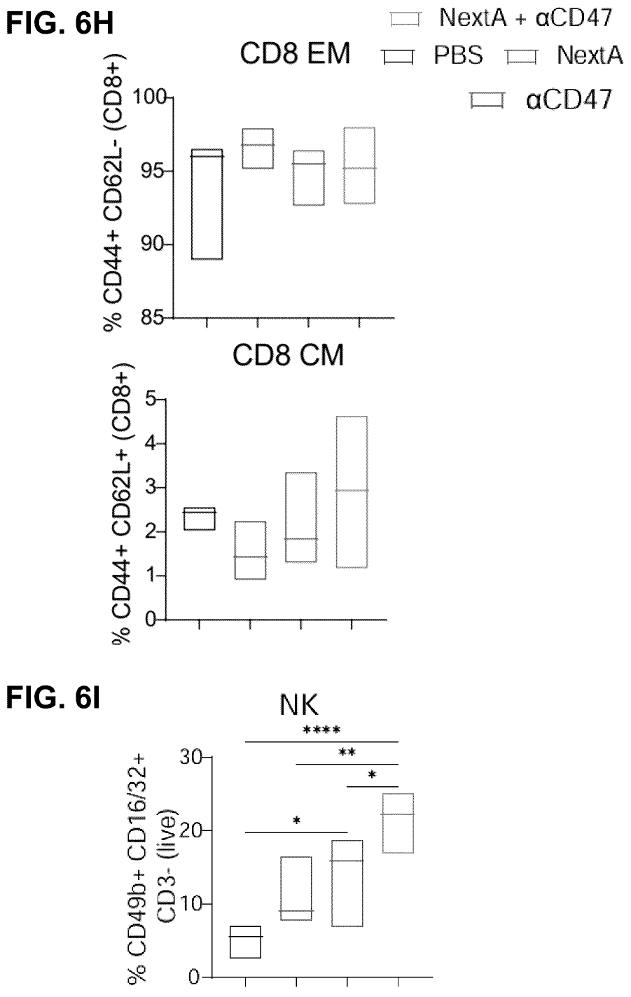

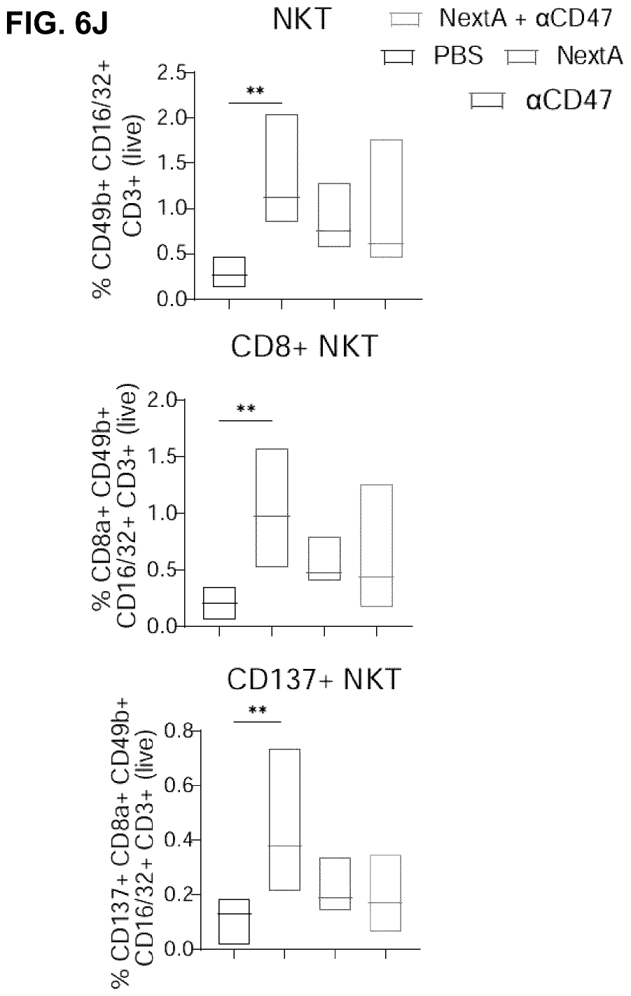

To evaluate the effects of the single arms and combination therapy, the tumors were harvested at the experimental endpoint and immunophenotyping was performed by flow cytometry. Overall, the combination of NextA and anti-CD47 had immunomodulatory effects and enhanced immune cell infiltration in the TME. Interestingly, single treatment arms significantly increased total immune cell (CD45+) infiltration, which was significantly increased in the combination therapy (FIG. 6A). The different macrophage populations in the tumors were evaluated (FIG. 6B). NextA treatment and the combination significantly increased M1-like macrophages (CD80+, H2Kd+, F4/80+), while NextA significantly decreased M2-like macrophages (CD206+, CD163+, F4/80+). NextA treatment increased the M1/M2 ratio. These results indicate that NextA and anti-CD47 can act synergistically in vivo to decrease tumor growth and to modulate the phenotype of TAMs.

Regarding total T cell populations, NextA increased CD3 T cell infiltration (FIG. 6C). Similarly, CD4 and CD8 T cell infiltration in the TME was not altered in any treatment groups compared to the control (FIG. 5D-FIG. 5E). Like CD4 T cells, regulatory T cells (Tregs, CD25+, CD4+, CD3+) were not significantly affected by the single arm therapy or by combination therapy (FIG. 6F). Interestingly, the combination therapy significantly increased CD4 effector memory T cells (CD44+, CD62L−, CD4+, CD3+, FIG. 5G). However, no significant changes were observed in CD4 central memory T cells (CD44+, CD62L+, CD4+, CD3+). There were no observed changes in CD8 central and effector memory T cells in either the single arms therapy or combination groups (FIG. 6H).

Notably, a significant increase in the infiltration of total NK cells (CD49b+, CD16/32+, CD3−) was observed with αCD47 treatment alone. Total NK cell infiltration was further significantly enhanced in the combination group compared to every other group (FIG. 6I). Evaluation of NKT cells (CD49b+, CD16/32+, CD3+) revealed that NextA alone significantly increased total NKT, CD8+ NKT, and CD137+ CD8+ NKT cells in the TME (FIG. 6J).

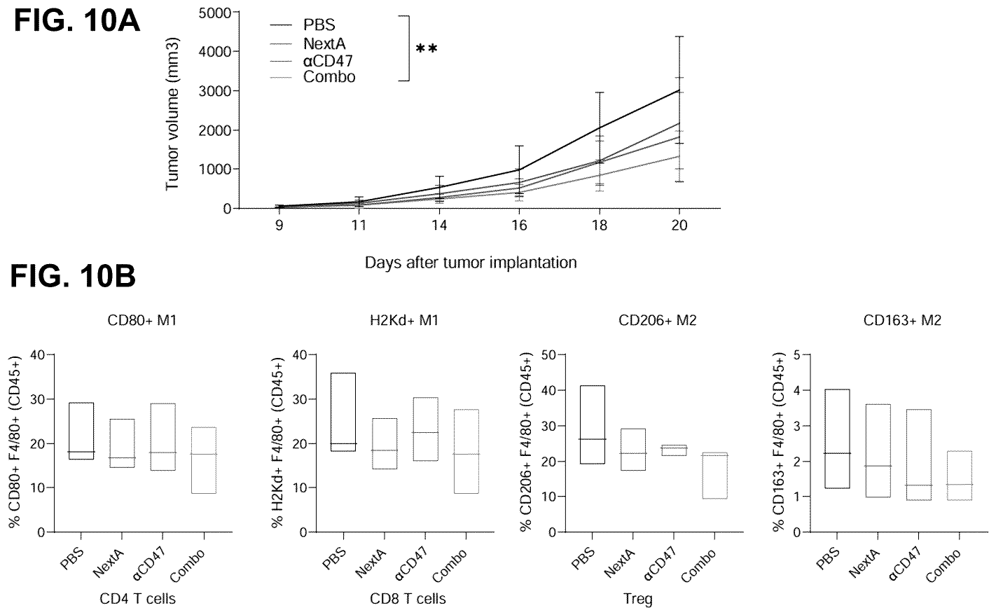

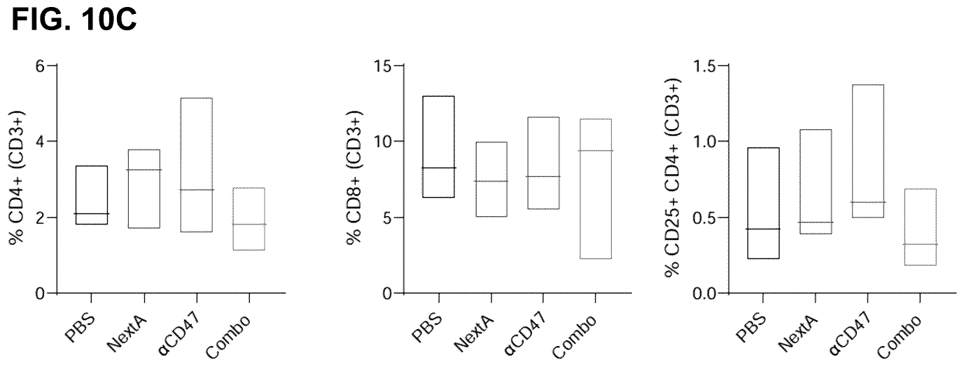

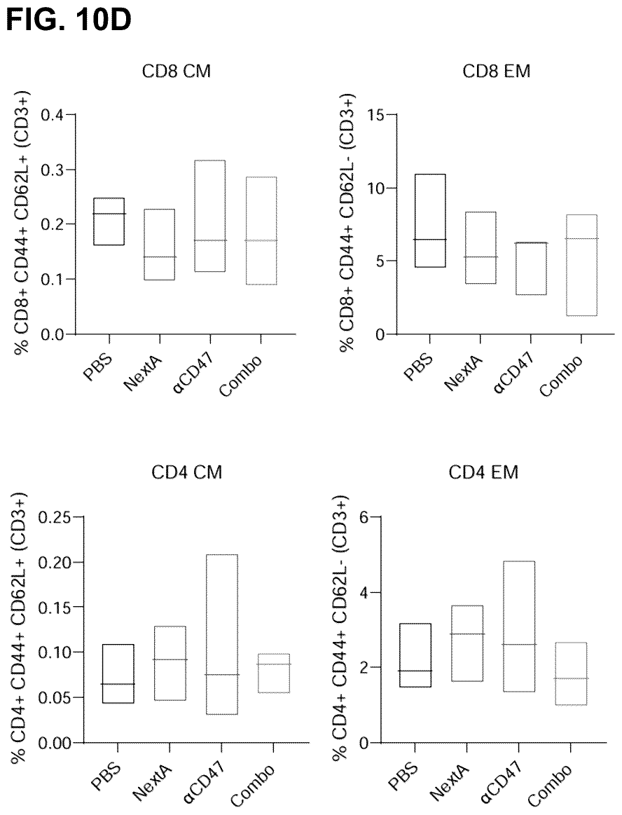

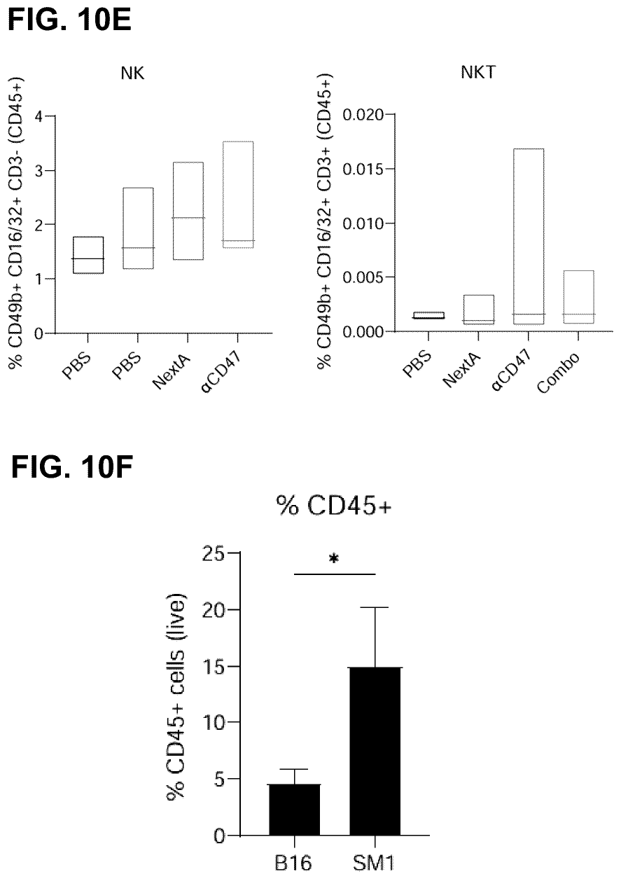

The antitumor effects of the combination therapy were also observed in the B16F10 melanoma model following the same experimental protocol as in the SM1 model. In contrast, NextA alone or αCD47 alone did not decrease B16 melanoma tumor growth (FIG. 10A). But the combination of NextA and αCD47 led to a significant decrease in tumor growth. Regarding the immunophenotyping of tumors, there were no significant differences in the immune cell populations in the B16 TME by flow cytometry (FIG. 10B-FIG. 10E). Thus, the discrepancies between the SM1 and B16F10 tumor models could be explained by the scarce infiltration of immune cells in the TME of B16F10 tumors compared to SM1 tumors (5% versus 15%, respectively, FIG. 10F) as well as the poor immunogenicity of B16 tumors.

In sum, these examples demonstrate that NextA and anti-CD47 act synergistically to reduce tumor growth in mouse melanoma models. Moreover, these therapies altered the SM1 melanoma TME, particularly innate immune cells such as macrophages and NK cells. The lack of differences in T cells among groups indicates that the therapies, whether individually or in combination, did not negatively affect T cell infiltration or populations in the TME. However, the antitumor effects observed in these studies indicate that a therapy including T cell-targeting strategies can be more potent and can generate a long-lasting antitumor response.

### Summary of Experiments

The work described herein considered whether HDAC6 inhibitors can modulate the macrophage phenotype and phagocytic function. The data provided herein indicates that the combination of NextA and anti-CD47 acted synergistically to reduce SM1 melanoma growth. Treatment with NextA increased the M1 phenotype and decreased the M2 phenotype of macrophages. NextA treatment tilted the balance to a pro-phagocytic milieu by decreasing the expression of SIRPα and other pro-phagocytic signals on macrophages (FIG. 2A-FIG. 2E). HDAC6 inhibition repressed the IFNγ-mediated upregulation of CD47 in mouse and human melanoma cell lines (FIG. 3). The downregulation of SIRPα by HDAC6 inhibitors led to an increase in phagocytosis in the presence of IgG control group. Interestingly, NextA treated macrophages that were co-cultured with SM1 cells in the presence of a CD47 blocking antibody had a more significant phagocytic activity (FIG. 4). Hdac6^{-/-} macrophages have defects in intracellular killing of phagocytosed bacteria and that loss of HDAC6 impairs the ability of macrophages to phagocytose bacteria. HDAC6i enhanced phagocytosis of SM melanoma cells, an event regulated by the CD47/SIRPα axis. Using a selective HDAC6 inhibitor, NextA downregulated SIRPα expression on NK cells. These data indicated that the CD47/SIRPα axis and HDAC6 play roles in the NK cell antitumor responses.

The data provided herein represent a novel approach to target the CD47/SIRPα axis by controlling the expression of these “don't eat me” signals on macrophages and on cancer cells such as melanoma cells. Furthermore, these results provided a rationale for the combination of CD47 blocking antibodies with HDAC6i to modulate macrophage phenotype, enhance phagocytosis, and potentiate antitumor NK cell responses in vivo.

TABLE 2
Sequences

| SEQ | |
|---|---|
| ID NO | Description |
| 1 | human CD47 primer - sense |
| 2 | human CD47 primer - antisense |
| 3 | human CD86 primer - sense |
| 4 | human CD86 primer - antisense |
| 5 | mouse CD80 primer - sense |
| 6 | mouse CD80 primer - sense |
| 7 | human CD47 (amino acid) |
| 8 | human CD47 (amino acid) |
| 9 | human CD47 (amino acid) |
| 10 | human CD47 (amino acid) |
| 11 | human CD47 (mRNA) |
| 12 | human CD47 (mRNA) |
| 13 | human CD47 (mRNA) |
| 14 | human CD47 (genomic) |
| 15 | human HDAC6 (amino acid) |
| 16 | human HDAC6 (amino acid) |
| 17 | human HDAC6 (amino acid) |
| 18 | human HDAC6 (mRNA) |
| 19 | human HDAC6 (mRNA) |
| 20 | human HDAC6 (genomic) |
| 21 | human HDAC6 (genomic) |
| 22 | human SIRPα (amino acid) |
| 23 | human SIRPα (amino acid) |
| 24 | human SIRPα (amino acid) |
| 25 | human SIRPα (mRNA) |
| 26 | human SIRPα (mRNA) |
| 27 | human SIRPα (mRNA) |
| 28 | human SIRPα (mRNA) |
| 29 | human SIRPα (genomic) |

The combination of HDAC6 inhibitors and CD47 inhibitors represents a novel approach to target the CD47/SIRPα axis by controlling the expression of “don't eat me” signals on cancer cells and macrophages. The use of epigenetic modifiers and blocking antibodies to target both arms of the “don't eat me” pathway underscores this innovative method to modulate and potentiate innate antitumor activity.

What is claimed is:

1. A method of treating a subject having a cancer, the method comprising:
administering to a subject in need thereof a therapeutically effective amount of a HDAC6 inhibitor; and
administering to the subject a therapeutically effective amount of a CD47 inhibitor.

2. The method of claim 1, wherein the HDAC6 inhibitor comprises a small molecule, a peptide, a polynucleotide, an antibody or fragment thereof, an antisense oligonucleotide, siRNA, RNAi, or any combination thereof.

3. The method of claim 2, wherein the HDAC6 inhibitor comprises Nexturastat A, Tubastatin A, KA2507, Ricolinostat (ACY-1215), Citarinostat (ACY-241), Tubacin, CAY10603, WT161, ACY-738, ACY-775, HPOB, SKLB-23bb, SS-208, Suprastat HDAC6 degrader-1 (PROTAC), HDAC6 degrader-3 (PROTAC), J22352 (PROTAC), HPB, HDAC6-IN-12 (compound GZ), HDAC6/8/BRPF1-IN-1, QTX125, CG347B, BRD73954, AES-135, AES-350, KH-259, SW-100, HPOB, Droxinostat (NS 41080), Bufexamac, KA2507, MC2625, MPT0G211, MPT0G211 mesylate, WT-161, ACY-738, ACY-775, XP5, HDAC-IN-35 (Compound 14), HDAC6-IN-15, HDAC6-IN-14, HDAC6-IN-13 (Compound 35m), HDAC6-IN-11 (Compound 9), HDAC6-IN-10, HDAC6-IN-9 (compound 12c), HDAC6-IN-8, HDAC6-IN-7 (TCS HDAC6 20b), HDAC6-IN-6 (compound 6a), HDAC6-IN-5 (compound 11b), HDAC6-IN-4 (C10), HDAC-IN-4, HDAC-IN-40, HDAC6-IN-3 (Compound 14), HDAC3/6-IN-2 (compound 15), or any combination thereof.

4. The method of claim 1, wherein the CD47 inhibitor comprises a small molecule, a peptide, a polynucleotide, an antibody or fragment thereof, an antisense oligonucleotide, siRNA, RNAi, or any combination thereof.

5. The method of claim 4, wherein the CD47 inhibitor comprises RRx-001, a dinitroazetidine derivative, Hu5F9-G4, CC-90002, TTI-621, ALX148, SRF231, SHR-1603, IBI188, ST-1901, SGNCD-47M, Gentulizumab, CC-90002 (INBRX 103), Hu5F9-G4 (5F9), Magrolimab, STI-6643, TI-061, AO-176, SRF231, AK117, IBI188, IMC-002, SHR-1603, TJ011133, ZL-1201, evorpacept (ALX148), TTI-621, TTI-G22, or any combination thereof.

6. The method of claim 1, wherein treating the subject comprises (i) reducing and/or preventing tumor growth; (ii) reducing or slowing tumor metastasis; (iii) preventing and/or delaying recurrence of the cancer; (iv) extending and/or prolonging disease-free or tumor-free survival time; (v) increasing and/or lengthening overall survival time; (vi) reducing and/or minimizing the frequency of treatment; (vii) relieving and/or ameliorating one or more symptoms of the cancer; (viii) reducing and/or decreasing tumor burden, (ix) preventing and/or facilitating surgical intervention; (x) increasing and/or enhancing phagocytosis of cancer cells or tumor cells; (xi) increasing and/or enhancing immune cell infiltration in and/or around tumor microenvironment; (xii) enhancing the subject's innate antitumor immunity; (xiii) driving and/or facilitating the M1 or pro-inflammatory phenotype of macrophages, (xiv) disrupting the CD47-SIRPα axis in one or more cancer cells or tumor cells; or (xv) any combination thereof.

7. The method of claim 1, wherein the CD47 inhibitor is administered prior to, concurrent with, or after the administration of the HDAC6 inhibitor.

8. The method of claim 1, further comprising repeating the administering to the subject of the HDAC6 inhibitor.

9. The method of claim 1, further comprising repeating the administering to the subject of the CD47 inhibitor.

10. The method of claim 1, wherein administering the HDAC6 inhibitor comprises systemic or direct administration.

11. The method of claim 1, wherein administering the CD47 inhibitor comprises systemic or direct administration.

12. The method of claim 1, wherein the therapeutically effective dose of the HDAC6 inhibitor comprises about 1 mg/kg body weight/day to about 100 mg/kg body weight/day.

13. The method of claim 1, wherein the therapeutically effective dose of the CD47 inhibitor comprises about 1 mg/kg body weight/day to about 100 mg/kg body weight/day.

14. The method of claim 1, further comprising monitoring the subject for adverse effects.

15. The method of claim 14, wherein (i) in the absence of adverse effects, the method further comprises continuing to treat the subject, or (ii) in the presence of adverse effects, the method further comprises modifying one or more steps of the method.

16. The method of claim 1, further comprising administering to the subject one or more additional anti-cancer therapies.

17. The method of claim 16, wherein the one or more anti-cancer therapies comprises endocrine therapy, radiotherapy, hormone therapy, gene therapy, thermal therapy, ultrasound therapy, or any combination thereof.

18. The method of claim 1, wherein the cancer comprises ovarian cancer, ovarian adenocarcinoma, ovarian teratocarcinoma, lung cancer, small cell lung cancer (SCLC), non-small cell lung cancer (NSCLC), squamous cell lung carcinoma, adenocarcinoma, gastric cancer, breast cancer, hepatic cancer, pancreatic cancer, skin cancer, in particular basal cell carcinoma and squamous cell carcinoma, malignant melanoma, head and neck cancer, malignant pleomorphic adenoma, sarcoma, synovial sarcoma, carcinosarcoma, bile duct cancer, bladder cancer, transitional cell carcinoma, papillary carcinoma, kidney cancer, renal cell carcinoma, clear cell renal cell carcinoma, papillary renal cell carcinoma, colon cancer, small bowel cancer, small bowel adenocarcinoma, adenocarcinoma of the ileum, testicular embryonal carcinoma, placental choriocarcinoma, cervical cancer, testicular cancer, testicular seminoma, testicular teratoma, embryonic testicular cancer, uterine cancer, teratocarcinoma, embryonal carcinoma, or any combination thereof.

19. A pharmaceutical formulation, comprising: a therapeutically effective amount of a HDAC6 inhibitor and a therapeutically effective amount of a CD47 inhibitor.

20. The pharmaceutical formulation of claim 19, wherein the HDAC6 inhibitor comprises Nexturastat A, Tubastatin A, KA2507, Ricolinostat (ACY-1215), Citarinostat (ACY-241), Tubacin, CAY10603, WT161, ACY-738, ACY-775, HPOB, SKLB-23bb, SS-208, Suprastat HDAC6 degrader-1 (PROTAC), HDAC6 degrader-3 (PROTAC), J22352 (PROTAC), HPB, HDAC6-IN-12 (compound GZ), HDAC6/8/BRPF1-IN-1, QTX125, CG347B, BRD73954, AES-135, AES-350, KH-259, SW-100, HPOB, Droxinostat (NS 41080), Bufexamac, KA2507, MC2625, MPT0G211, MPT0G211 mesylate, WT-161, ACY-738, ACY-775, XP5, HDAC-IN-35 (Compound 14), HDAC6-IN-15, HDAC6-IN-14, HDAC6-IN-13 (Compound 35m), HDAC6-IN-11 (Compound 9), HDAC6-IN-10, HDAC6-IN-9 (compound 12c), HDAC6-IN-8, HDAC6-IN-7 (TCS HDAC6 20b), HDAC6-IN-6 (compound 6a), HDAC6-IN-5 (compound 11b), HDAC6-IN-4 (C10), HDAC-IN-4, HDAC-IN-40, HDAC6-IN-3 (Compound 14), HDAC3/6-IN-2 (compound 15), or any combination thereof, and wherein the CD47 inhibitor comprises RRx-001, a dinitroazetidine derivative, Hu5F9-G4, CC-90002, TTI-621, ALX148, SRF231, SHR-1603, IBI188, ST-1901, SGNCD-47M, Gentulizumab, CC-90002 (INBRX 103), Hu5F9-G4 (5F9), Magrolimab, STI-6643, TI-061, AO-176, SRF231, AK117, IBI188, IMC-002, SHR-1603, TJ011133, ZL-1201, evorpacept (ALX148), TTI-621, TTI-G22, or any combination thereof.
